(19) United States
(12) Patent Application Publication
(10) Pub. No.: US 2024/0115742 A1
(43) Pub. Date: Apr. 11, 2024

(54) FLUORINATED FIBERS FOR MULTIMODAL 1H AND 19F MRI, ULTRASOUND, FLUORESCENCE DETECTION AND CONFORMATIONAL AND TEMPERATURE DETECTION BY 19F MRS

(71) Applicant: New York University, New York, NY (US)

(72) Inventors: Jin Kim Montclare, New York, NY (US); Dustin Britton, Brooklyn, NY (US); Youssef Zaim Wadghiri, New York, NY (US); Farbod Mahmoudinobar, Harrison, NJ (US)

(21) Appl. No.: 18/460,519

(22) Filed: Sep. 1, 2023

Related U.S. Application Data
(60) Provisional application No. 63/515,836, filed on Jul. 26, 2023.
(60) Provisional application No. 63/374,365, filed on Sep. 1, 2022.

(51) Int. Cl. A61K 49/18; A61K 49/14; A61K 49/22; C07K 14/245; G01N 24/08; G01N 29/34; G16B 40/00

(52) U.S. Cl. CPC A61K 49/1884; A61K 49/14; A61K 49/222; C07K 14/245; G01N 24/08; G01N 29/34; G16B 40/00; B82Y 40/00

(57) ABSTRACT

Provided are proteins/peptides. The proteins or peptides comprise a sequence designed by the methods described herein. The proteins and peptides may have one or more trifluoroleucine (L_{TF}, which may be referred to as TFL or LTF throughout) residues. The proteins may have or contain the following sequence:
VX₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇ (SEQ ID NO:1), where X₁ is A, E, D, R, H, K, Q, N, or S; X₂ is A, E, N, or Q; X₃ is A, V, L, I, Q, M, or L_{T}; X₄ is A, E, R, D, H, I, L, T, K, Q, N, or L_{T}; X₅ is A, F, Q, R, K, H, D, S, or E; X₆ is A, L, I, or L_{TF}; X₇ is A, K, or E; X₈ is A, K, E, D, R, H, Q, or N; X₉ is A, T, I, L, Q, or L_{TF}; X₁₀ is A, L, I, or LT; X₁₁ is A, E, D, H, P, I, L, K, Y, N, Q, R, or LT; X₁₂ is A, Q, H, E, D, K, R, or N; X₁₃ is A, M, I, L, Q, T, or L_{TF}; X₁₄ is A, L, D, E, K, I, or L_{TF}; X₁₅ is A, E, D, H, Y, I, L, R, K, Q, N, or Lu; X₁₆ is A, E, or Q; X₁₇ is A, L, M, I, V, or L_{TF}; X₁₈ is A, K, E, D, K, R, H, N, or Q; X₁₉ is A, N, D, K, R, H, Q, or E; X₂₀ is A, L, T, I, M, R, or L_{TF}; X₂₁ is A, N, or Q; X₂₂ is K, A, E, I, L, M, R, H, D, Q, N, S, or L_{TF}; X₂₃ is A, Q, N, I, L, or L_{TF}; X₂₄ is A, L, I, M, T, or LT; X₂₅ is A, H, Q, R, K, D, N, Y, I, E, L, T, or LT; X₂₆ is A, D, E, R, K, Q, H, N, or T; X₂₇ is A, V, I, Q, L, T, or L_{TF}; X₂₈ is A, R, E, D, K, H, N, Q, or T; X₂₉ is A, H, E, R, D, K, I, L, N, Q, T, Y, or L_{TF}; X₃₀ is L, A, D, K, I, N, Q, or L_{TF}; X₃₁ is A, L, Q, I, or L_{TF}; X₃₂ is E, D, K, H, N, Q, A, L, R, I, Y, or L_{TF}; X₃₃ is A, N, Q, D, E, H, K, R, or S; X₃₄ is Q, I, L, A, M, or LT; X₃₅ is S, A, P, or Q; X₃₆ is A, K, T, D, R, H, N, Q, or E; X₃₇ is A, L, I, K, D, N, Q, R, or L_{TF}; where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ or any L is replaced with L_{TF}. The proteins and peptides may have desirable self-assembling properties such that they form supramolecular structures (e.g., fibers or fibrils). The supramolecular structures may further gelate water such that a hydrogel is formed. The fibers and/or gels may be used to deliver drugs and/or as theranostic agents.

### CROSS REFERENCE TO RELATED APPLICATIONS

This application claims priority to U.S. Provisional Application No. 63/374,365, filed on Sep. 1, 2022 and U.S. Provisional Application No. 63/515,836, filed on Jul. 26, 2023, the entire disclosures of which are incorporated herein by reference.

### STATEMENT REGARDING FEDERALLY SPONSORED RESEARCH

This invention was made with government support under contract nos. DMR-1728858, DMR 1840984, and DMR-1420073 awarded by the National Science Foundation and 1S10OD018337-01, 5P30CA016087, and P41 EB017183 awarded by the National Institutes of Health. The government has certain rights in the invention.

### SEQUENCE LISTING

The instant application contains a Sequence Listing, which has been submitted in .xml format and is hereby incorporated by reference in its entirety. Said .xml copy was created on Sep. 1, 2023, is named “058636_00644_ST.xml”, and is 57,283 bytes in size.

### BACKGROUND OF THE DISCLOSURE

Protein-engineered biomaterials have become an increasingly popular choice for a variety of biomedical and materials-based applications including therapeutics and tissue-engineered scaffolds. An important subset of these materials includes those that have the ability to self-assemble. In particular, self-assembling protein biomaterials possess ideal drug delivery potential as they are both inherently biodegradable and can provide effective encapsulation of a variety of drugs and can easily be modified for targeted drug release. One such protein biomaterial that has garnered increasing interest are those that self-assemble into fibers. Formation of fibers is often the result of a variety of stimuli such as pH, temperature, concentration, ionic strength, and sequence; thus their formation can be readily tuned.

Previous work has demonstrated α-helical coiled-coil peptide domains can be designed to form nanofibers. Self-assembling cartilage oligomeric matrix protein coiled-coil (C) has been previously used to generate biomaterials with the potential to encapsulate and deliver a wide variety of small molecule therapeutics and form nanofibers approximately 10-15 nm in diameter. Included is the engineering of Q, based on removal of the C-terminal heptad and swapping of the N- and C-terminus of C, which results in a positively and negatively charged patch at the N- and C-termini of its homopentameric coiled-coil domain. Q displays the ability to form nanofibers up to 560 nm wide by the stacking of the oppositely charged ends of the domain, which is further capable of binding curcumin to form fibers of 16 μm in diameter.

Quantifying supramolecular assembling proteins has not yet been elucidated and therefore designing the proteins to undergo the specific function of fibril assembly is not clear.

The phenomena for supramolecular assembly of coiled-coil proteins into fibers has been limited to the realm of alternating charges and this method does not ensure proteins to undergo fibril or hydrogel assembly. Further, computational design of protein hydrogels has largely used modeling and simulation ex postfacto. Current computational workflows mostly reside in the simulation space where the ability to represent and model self-assembly of known sequences compared to a complete library of sequence combinations or a small library of rational mutants are the dominant approaches. Conversely, computationally driven designs have not been well-explored and those that have must still be screened using molecular dynamics (MD) simulations that come at a high computational cost. Developing quantitative metrics to correlate sequence directly to hydrogel function would help to avoid these pitfalls and bridge the gap between sequence and function. Thus, most protein-based hydrogels have existed in the realm of naturally occurring and recombinant protein fusions.

Self-assembling protein-based hydrogels have become increasingly appealing materials for biomedical applications. Higher-order assembly of these proteins commonly results in physical crosslinking in the form of junctions and arbitrary entanglements, resulting in their subsequent gelation. Protein-based hydrogels can be predominantly bifurcated into hydrogels that possess α-helical and β-sheet interactions. The nature of the protein-protein interactions dictates the impact of hydrophobicity, charge, and various external stimuli on the resulting gelation properties and overall hierarchical assembly as well as their biomedical utility.

Tuning gelation kinetics has been deemed useful for different biomedical applications, with applications often calling for gelation on a specific timescale. An important property inherent to these supramolecularly assembling proteins in clinical use is their responsiveness to various physiological conditions such as temperature, pH, and ionic strength.

Additionally, theranostic agents is a growing field in biomedicine that help to overcome limitations in biomaterials providing therapy and diagnosis of diseases. These materials help to monitor the development of disease after therapeutic treatment as well as provide a simultaneous diagnosis and treatment of a disease. Currently, theranostics largely focus on synthetic approaches while using inorganic materials such as quantum dots or radiolabeling to confer diagnostic properties. Quantum dots suffer from stability and aggregation, which greatly reduces their diagnostic sensitivity and limit their ability to effectively penetrate tissues with their signal. The practical application of radiolabeling can be challenging due to the short half-lives of radioactive isotopes, which impose logistic constraints. The resulting limited time window necessitates the use of efficient synthesis methods to ensure timely labeling. However, it also raises concerns about potential prolonged radiation exposure during the labeling process. Theranostics are also challenged by combining drug delivery techniques that possess targeting moieties with high specificity, thus reducing therapeutic efficacy and signal sensitivity. To create an ideal theranostic biomaterial, without compromising drug encapsulation, diagnostic imaging must be optimized for improved detection. One such method to improve this specificity is the incorporation of fluorine into biomaterials.

While chemotherapeutic agents such as doxorubicin (Dox) and paclitaxel (Ptx) are able to improve overall survival of metastatic breast cancer (MBC) patients, systemic off-target effects become a concern, including toxicity to healthy cells, cardiotoxicity, myelosuppression, peripheral neuropathy, alopecia, nausea, vomiting. Improved formulations such as pegylated liposomal doxorubicin (PLD) are characterized by favorable pharmacokinetics and specific accumulation in tumor tissues resulting in an improved therapeutic window. However, acute infusion reactions, mucositis and palmar plantar erythrodysesthesias still occur and require dose interruptions or reductions. Nab-paclitaxel (NP) is a soluble form of paclitaxel that is linked to albumin nanoparticles, which optimizes drug delivery and eliminates the need for the solvent but does not target specifically to tumor cells. Consequently, bone marrow and neurotoxicity remain a clinical problem and frequently require dose reductions or cessation of therapy. Although PLD and NP meet the design criteria of an optimal cargo, active targeting to tumor cells has been challenging for most chemotherapeutic drugs. Thus, there is an urgent need to develop a means to deliver Dox and Ptx more efficiently to specific cancer cells without toxicity, especially with longer duration of therapy, in order to increase the likelihood of success.

Despite recent development of various new treatment approaches, MBC remains a treatable but a non-curable subtype of breast cancer, accounting for 40,000 deaths annually in the US and for its worst prognosis subtype, TNBC, with an average survival of 18-24 months despite the use of current chemotherapeutics. Seventy-eight percent of early breast cancers have tumor-infiltrating immune cell PD-L1-positivity, and 21% have carcinoma cell positivity. Dox and Ptx are the most widely employed chemotherapeutics in the treatment of breast cancer. Although very active, these drugs are notorious for their systemic off-target effects because of the lack of specific tumor targeting, that include cardiotoxicity, myelosuppression, alopecia, nausea, vomiting. Additionally, Ptx bears issues of solubility, and solvent-based toxicity, rendering its therapeutic use for MBC problematic. Development of liposomal Dox (LD) and PLD has led to improved therapeutic effect while reducing cardiotoxicity based on clinical trials. Both systems increase the plasma half-life leading to Dox extravasation across leaky vessels and accumulation in MBC tissue as a result of enhanced permeation and retention effect while reducing toxicity due to decreased exposure to normal tissue when compared to free Dox. While both LD and PLD exploit passive targeting to deliver Dox, toxicity and selective release of the active agent to the tumor remain a challenge. Although Nab-Ptx has shown potential to leverage the therapeutic effects of Ptx, side effects such as neutropenia and peripheral neuropathy frequently require dose modification and discontinuation. Thus, there is an urgent need to develop a means to deliver therapeutic agents more efficiently to specific cancer cells without systemic toxicity, in order to increase the likelihood of successful therapy. Moreover, the ability to monitor and evaluate the drug delivery non-invasively is not possible with current systems.

Since fluorine is largely absent from organisms, yet exists in 100% natural abundance, it is useful as a contrast agent due to its specific signal in ¹⁹F MRS. In light of this, many ¹⁹F MRS materials have been developed for biomedical applications such as MRI cell tracking, tumor imaging, as well as monitoring tumor cell hypoxia and proliferation. These agents are often synthetically derived to create fluorine-based polymers or nanoemulsions.

### SUMMARY OF THE DISCLOSURE

In an aspect, the present disclosure provides proteins or peptides. The proteins or peptides comprise a sequence designed by the methods described herein. The proteins and peptides may have one or more trifluoroleucine (L_{TF}, which may be referred to as TFL or LTF throughout) residues. The proteins and peptides may have desirable self-assembling properties such that they form supramolecular structures (e.g., fibers or fibrils). The supramolecular structures may further gelate water such that a hydrogel is formed.

For example, the proteins or peptides may be fluorinated analogues of peptides derived from a variant of a coiled-coil domain of cartilage oligomeric matrix protein (COMPcc). The various proteins may be referred to as a Q protein/peptide following by a number, where each number corresponds to a different protein/peptide. For example, the present disclosure provides Q2, Q3, Q4, Q5, Q6, Q7, or Q8 or a protein comprising the sequences of Q2, Q3, Q4, Q5, Q6, Q7, or Q8, where the one or more leucine residues of the protein or peptide is replaced with trifluoroleucine. In various examples, the protein or peptide may comprise fluorinated sequences of Q2, Q3, Q4, Q5, Q6, Q7, or Q8. One, some, or all of the leucine residues of the protein or peptide may be replaced with trifluoroleucine.

A protein or peptide of the present disclosure may have or comprise the following sequence:

(SEQ ID NO: 1)
VX₁X₂X₃X₄X₅X₆X₇X₈X₉X₁₀X₁₁X₁₂X₁₃X₁₄X₁₅X₁₆X₁₇X₁₈X₁₉X₂₀X₂₁
X₂₂X₂₃X₂₄X₂₅X₂₆X₂₇X₂₈X₂₉X₃₀X₃₁X₃₂X₃₃X₃₄X₃₅X₃₆X₃₇,

where
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₆ is A, E, or Q;
X₁₇ is A, L, M, I, V, or L_{TF};
X₁₈ is A, K, E, D, K, R, H, N, or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ or any L is replaced with a trifluoroleucine residue. In various embodiments, one, some, or all L residues and/or X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ residues are replaced with L_{TF}. In various embodiments, a protein or peptide of the present disclosure further comprises MRGSHHHHHHGSIEGR (SEQ ID NO:2). Thus, for example, a peptide or protein may have or comprise the following sequence:

(SEQ ID NO: 26)
MRGSHHHHHHGSIEGRVX₁X₂X₃X₄X₅X₆X₇X₈X₉X₁₀X₁₁X₁₂X₁₃X₁₄
X₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃
X₃₄X₃₅X₃₆X₃₇

where
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R, or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₁ is A, E, or Q;
X₁₇ is A, L, M, I, V, or L_{TF};
X₁₈ is A, K, E, D, K, R, H, N. or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇, or any L is replaced with L_{TF}. In various embodiments, one, some, or all L residues and/or X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ residues are replaced with L_{TF}.

In an aspect, the present disclosure provides protein fibers or peptide fibers and gels comprising those fibers. The protein fibers and peptide fibers may be referred to fibers. These fibers may comprise one or more protofibers. The one or more protofibers may comprise a plurality of proteins and/or peptides of the present disclosure.

In an aspect, the present disclosure provides a composition. The composition comprises a protein or peptide designed using a method of the present disclosure. The composition may comprise a plurality of fibers comprising peptides or proteins of the present disclosure. The composition may be formulated into a gel and further comprise one or more compounds, which may be therapeutic agents.

In an aspect, the present disclosure provides a method for detecting the location of a compound in an individual. The compound may be bound to a protein/peptide of the present disclosure or a fiber of the present disclosure and the protein/peptide or fiber is administered to an individual. The compound may be a therapeutic agent.

In various examples, a method of the present disclosure comprises administering to an individual a protein/peptide or fiber of the present disclosure, where the protein/peptide or fiber has a compound bound thereto or associated therewith (such as, for example, when the compound is encapsulated by a gel comprising a plurality of the fibers). After administration, a signal of the peptide/protein or fiber can be detected. In various examples, the signal can be detected via spectroscopy (e.g., magnetic resonance imaging (MRI)). The spectroscopy may be ¹⁹F MR spectroscopy or ¹H MR spectroscopy). In various examples, the signal can be detected ultrasound.

In various embodiments, the method may further comprise a treating step. The treating may be used to treat wounds (e.g., diabetic wounds) or to dose chemotherapeutic agents directly to a tumor site. The treatment step may comprise administering a therapeutic compound bound to the fiber or encapsulated by a gel comprising the fibers.

In an aspect, the present disclosure provides machine learning models using the electrostatic potential energy of atoms responsible for the fibril assembly of a coiled-coil domain to quantify the likelihood of self-assembly and the subsequent use of the parameter for search and prediction of fiber size and mechanical properties of resulting hydrogels and the proteins that result from these methods.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a fuller understanding of the nature and objects of the disclosure, reference should be made to the following detailed description taken in conjunction with the accompanying figures.

FIG. 1. a. Heat map based on critical time to gelation of hydrogels formed by Q-derived protein sequences. Cooler colors represent faster gelation and hotter colors represent slower gelation. b. Accuracy of bivariate regression model for NEEbcf and CEEbcf to predict tc showing residual differences between measured tc and predicted tc using the bivariate linear regression model.

FIG. 2. a. Curcumin molecular structure b. Example of C as an coiled-coil example with curcumin docked in the favorable N-terminal position c. Coiled-coil library based on C and Q with sequence information and electrostatic potential maps with positive blue patches and negative red patches with scale −10 kbT to 10 kbT. Mutations to the solvent exposed residues in the b, c, and f helical wheel positions are colored green and mutations made to the hydrophobic pore in a and d helical wheel positions are colored in red. d. Schematic of coiled-coil stacking end-to-end to comprise a nanoscale fiber (example TEM image). FIG. 2c shows the following sequences:

(SEQ ID NO: 21)
MRGSHHHHHHGSIEGRAPQMLRELQETNAALQDVRELLRQQVKEITFLKN
TVMESDASGKL;
(SEQ ID NO: 22)
MRGSHHHHHHGSIEGRMQLAKHMLKELQKTNALQSVRTLLQQQVEEITQL
KETVENSDSS;
(SEQ ID NO: 23); SEQ ID NO: 11; and SEQ ID NO: 12
MRGSHHHHHHGSIEGRVKEITFLKNTAPQMLRELQETNAALQDVRELLRQ
QSKL.

FIG. 3. a. Representative TEM images of protein fiber variants in order of ascending size. b. Average fiber diameters of protein fiber variants (* indicates p-value <0.05 and ** indicates p-value <0.01 by unpaired t-test). and as plotted against cumulative electrostatic potential energy difference of each terminus of solvent exposed residues in the b, c, and f helical wheel positions (ΔEE_{bcf}). Average diameters are the result of twenty independent fiber measurements using ImageJ. Error bars represent standard deviation.

FIG. 4. Spectroscopic fluorescence of protein fiber variants at different protein:curcumin ratios. Fluorescence measured by excitation at 420 nm and emission at 520 nm. Error bars and confidence interval (CI@95%) represent the standard deviation of three independent results.

FIG. 5. a. Fluorescent confocal microscopy images of protein fiber variants after CCM binding using 460 nm excitation and a 470-550 nm detection window b. Average diameter of protein fiber variants after CCM binding measured by confocal microscopy and as correlated to average protein fiber diameter variants measured by TEM. Average diameters are the result of twenty independent fiber measurements. Error bars represent standard deviation.

FIG. 6. a. Circular dichroism spectra of protein fiber variants at 25° C. b. Melting temperatures of native (light blue) and CCM-bound (dark blue) protein fiber variants as measured by CD (* indicates p-value <0.05 by unpaired t-test). c. Representative ATR-FTIR spectra of native and CCM-bound protein fiber variants. Black line represents fit to raw data, red line represents deconvoluted α-helical peak, dark blue line represents antiparallel (3-sheet peak, light blue line represents 3-sheet peaks, green line represents aggregated strand peaks, and yellow line represents 3-10 helix peak. Error bars represent the standard deviation as a result of three independent trials.

FIG. 7. Correlation between percent difference in melting temperature (ΔTₘ%) after binding CCM as measured by circular dichroism to percent difference in protein Rosetta score (ΔRS %) after docking CCM.

FIG. 8. a. Structured content of protein fiber variants before CCM binding (light blue) and after CCM binding (dark blue) as measured by peak deconvolution of three independent ATR-FTIR measurements b. Correlated structured content loss to change in Tₘ after binding CCM. Error bars represent standard deviation of three independent trials.

FIG. 9. C protein (7.0 kDa) after purification. L: Ladder, FT: Flow-through, following are increasing mM concentrations of imidazole.

FIG. 10. C2 protein (7.0 kDa) after purification. L: Ladder, FT: Flow-through, following are increasing mM concentrations of imidazole.

FIG. 11. Q protein (6.4 kDa) after purification. L: Ladder, FT: Flow-through, following are increasing mM concentrations of imidazole.

FIG. 12. Q2 protein (6.5 kDa) after purification. L: Ladder, FT: Flow-through, following are increasing mM concentrations of imidazole.

FIG. 13. Q3 protein (6.5 kDa) after purification. L: Ladder, FT: Flow-through, following are increasing mM concentrations of imidazole.

FIG. 14. Transmission electron micrographs of several independent fibers of protein variants iat pH 4.0, 50 mM NaH₂PO₄. From left to right resolutions are 13.5 kX, 17.5 kX, and 36 kX with scale bars representing 1 μm, 500 nm, and 200 nm respectively.

FIG. 15. Sap scores calculated for protein fiber variants in Rosetta and compared to protein fiber diameter as measured by TEM.

FIG. 16. Confocal microscopy fluorescence images of several independent fibers of protein variants in the presence of equilibrium concentrations of curcumin at pH 4.0, 50 mM NaH₂PO₄ at various resolutions using 460 nm excitation and 470-550 nm detection window.

FIG. 17. Best scoring poses after docking of CCM with protein fiber variants using GALigandDock protocol in Rosetta. C pose indicates hydrogen bonding between C and CCM (red dashes).

FIG. 18. Path of supramolecular assembly for Q and Q2 hydrogels. Electrostatic potential maps for Q and Q2 show negative (red) and positive (blue) charged patches scaled from −10 kbT/e to 10 kbT/e. Sequences for Q and Q2 with position numbers. Mutations are highlighted for the hydrophobic pore (red) and charged solvent exposed surface (green). The sequences are depicted in this figure:

(SEQ ID NO: 24)
MRGSHHHHHHGSIEGRVKEITFLKNTAPQMLRELQETNAALQDVRELLRQ
QSKLN
and
(SEQ ID NO: 25)
MRGSHHHHHHGSIEGRVKLLFLKKTAEQMLEELKETNKALHDVRHLLENQ
SKLN

FIG. 19. Tube inversion images of 150 μL aliquots of Q2 at a. 0 h showing a liquid phase fallen to the bottom of the tube. b. 12 h showing the transition between a solution and gel state c. 36 h showing almost complete transition from solution to gel and d. complete transition to gel noted by zero visual movement of the gel at the top of the Eppendorf when inverted. e. Extent of gelation calculated by bivariate linear regression of a concentration-temperature phase diagram constructed from tube inversions showing gel behavior (black dots) and tube inversions showing solution behavior (red dots) after two weeks of incubation at 4 C.

FIG. 20. a. Representative log-log plot of MSD and lag time, tau, for Q2 determined by MPT. b. Time-cure superposition of MSD vs tau. c. Logarithmic shift factors for the vertical (log(a) in blue) and horizontal (log(b) in red) directions used in the time cure superposition to the determine the t_{gel}. d. Log-log plot of the shift factors and their distance from the t_{gel}, determined by the ratio of the logarithmic slopes of the horizontal to vertical shift factor.

FIG. 21. Rheology of Q2 measured using parallel plate rheometer setup for average storage modulus (G′, filled markers) and average loss modulus in (G″, empty markers) of Q2. Error bars represent standard deviation for three independent trials.

FIG. 22. a. Molar residue ellipticities in the far-UV region of Q2 in i) solution and as a ii) hydrogel. Spectra shown are averages of three independent trials. b. ATR-FTIR spectral analysis of Q2 secondary structure in i) solution state and as ii) hydrogel. Spectra are representative of three independent trials with overall spectra by deconvolution in black and individual peak deconvolutions in dotted gray lines.

FIG. 23. TEM images of Q2 at times a. 0 h, b. 12 h, c. 36 h, and d. 60 h.

FIG. 24. Q2 protein (6.48 kDa) after expression. L: Ladder, Pre: Pre induction with IPTG, Post: Post-induction with IPTG.

FIG. 25. Q2 protein (6.48 kDa) after purification. L: Ladder, FT: Flow-through, following are increasing mM concentrations of imidazole.

FIG. 26. 12% SDS-PAGE showing >99% purity of final Q2 protein (6.48 kDa) after concentration to 2 mM. L: Ladder.

FIG. 27. Extent of gelation for Q calculated by bivariate linear regression of a concentration-temperature phase diagram constructed from tube inversions showing gel behavior (black dots) and tube inversions showing solution behavior (red dots) after two weeks of incubation at 4 C. Created using data from Hill et al.¹

FIG. 28. Sigmoidal fit solved in MATLAB. Data is represented as the average and standard deviation of three independent trials. Logarithmic plateaus at the start and end of the curve used to determine solution and gelation equilibration.

FIG. 29. a. Log-log plot of MSD and lag time, τ, for independent trial (no. 2) of Q2 determined by MPT. b. Time-cure superposition of MSD vs τ. c. Logarithmic shift factors for the vertical (log(a) in blue) and horizontal (log(b) in red) directions used in the time cure superposition to the determine the t_{gel}. d. Log-log plot of the shift factors and their distance from the t_{gel}, determined by the ratio of the logarithmic slopes of the horizontal to vertical shift factor.

FIG. 30. a. Log-log plot of MSD and lag time, τ, for independent trial (no. 2) of Q2 determined by MPT. b. Time-cure superposition of MSD vs τ. c. Logarithmic shift factors for the vertical (log(a) in blue) and horizontal (log(b) in red) directions used in the time cure superposition to the determine the t_{gel}. d. Log-log plot of the shift factors and their distance from the t_{gel}, determined by the ratio of the logarithmic slopes of the horizontal to vertical shift factor.

FIG. 31. TEM images of Q at times a. 0 h, b. 24 h, c. 84 h, and d. 144 h.

FIG. 32. a. Scheme of L_{TF} incorporation and CCM encapsulation to generate Q2_{LTF} and Q2_{LTF}·CCM b. Q2_{LTF} protein (6.97 kDa) after expression. L: Ladder, Pre: Pre-induction with IPTG, Post: Post-induction with IPTG. c. Q2_{LTF} protein after purification. L: Ladder, FT: Flow-through, following are increasing concentrations of imidazole. d. Representative MALDI-TOF spectra showing incorporation of L_{TF} into Q2 by size increase to 6.97 kDa.

FIG. 33. a. CD wavelength scan of Q_{LTF} and Q2_{LTF} in water performed at 20° C. from 195 nm to 250 nm. Spectra are the average of three independent trials. b. Representative ATR-FTIR spectra of Q2_{LTF}. Overall spectra by deconvolution in black and individual peak deconvolutions in dotted red lines (α-helix), blue lines (β-sheet), and orange lines (random coil/turns). c. Transmission electron micrograph of Q2_{LTF} protein.

FIG. 34. a. Spectroscopic fluorescence of Q2_{LTF} at different protein:curcumin molar ratios. Fluorescence was measured by excitation at 450 nm and emission at 520 nm. Error bars represent standard deviation and are the result of three independent trials. b. Melting temperature of Q2_{LTF} at in the presence of phosphate buffer (PB) with and without CCM. Melting temperature is measured by CD and error bars are the result of three independent trials. c. Representative ATR-FTIR spectra of Q2_{LTF}·CCM. Overall spectra by deconvolution in black and individual peak deconvolutions in dotted red lines (α-helix), blue lines (β-sheet), and orange lines (random coil/turns). d. Fluorescent confocal micrograph of Q2_{LTF}·CCM showing fiber thickening to the mesoscale.

FIG. 35. a. NMR spectrum at 500 MHz (11.7-Tesla) of Q2_{LTF} at 1.5 mM showing two peaks (magenta and purple arrows) b. SNR of Q2_{LTF} and Q_{LTF} as a function of protein concentration c. Temperature dependence of SNR from independent peaks. d. Linear correlation of temperature with SNRT ratio showing ability to predict temperature from ¹⁹F MRS. e. Linear correlation of temperature with average (n=3) fraction folded of Q2_{LTF} as assessed by CD f. Linear correlation of average fraction folded (n=3) as assessed by CD with SNRT ratio showing ability to predict relative structure from ¹⁹F MRS.

FIG. 36. a. Representative in vitro TFA spectra (100%, 13 mM) acquired using our experimental setup and custom RF coil on a 7-T animal MRI scanner (300 MHz) employing a single pulse sequence. The spectra exhibit a chemical shift resonance at −74.3 ppm. b. Corresponding ¹⁹F SNRs at 7-T MRI are presented for serial dilutions of 100% TFA (green), progressively diluted until reaching the limit of detection (LOD, indicated by dashed lines). c. Representative ¹⁹F MR scan (scan time=4 min., TR=80 ms) showing a peak resonating at −81.6 ppm. d. SNR of Q2_{LTF} obtained from ¹⁹F MRS using a 7-T MRI scanner, with scans acquired under both 4-minute (red) and 1-minute (orange) scan times, while varying the TR.

FIG. 37. Ultrasound guided injection imaging: a. Sagittal view of the hindlimb right before the needle insertion where with the is adequately tilted at 450 to expose to joint and ease the infusion; b. Illustrates the needle insertion within the hindlimb knee joint; and c. Successful injection of Q2_{LTF} into the hindlimb knee joint appearing as an echogenic signal using high frequency ultrasound. Red arrow indicates the syringe tip and blue arrows indicate the presence of Q2_{LTF}. d. 3D rendering of ¹H MRI imaging of the mouse hindlegs where Q2_{LTF} fibers (highlighted in green) that appeared as a hypointense signal in the 3D MRI datasets in the injected hindleg. e. ¹⁹F MR spectroscopy performed in vivo after injection of Q2_{LTF} using 10 min scan (TR=100 ms, N_{AV}=6000).

FIG. 38. a. Transmit-receive volume linear birdcage radiofrequency (rf) coil (16 rungs, OD=72 mm, ID=42 mm, L=64 mm) tuned to resonate at 300.16 MHz, corresponding to the ¹H proton Larmor frequency at 7-Tesla. It ensures rf coverage for the entire mouse body. b. Rectangular flexible rf resonator (L=10 mm, W=30 mm) tuned the ¹⁹F nuclei (280 MHz) at 7-T using four distributed fixed capacitors. c. The flexible rf surface coil s is positioned within the inner part of the cylindrical birdcage to optimize inductive coupling enabling dual ¹H/¹⁹F resonance via a single port interfaced to a tune/match box. d. Illustration of the experimental setup enabling in vivo mouse MRI and MRS scanning of the mouse body.

FIG. 39. a. Q_{LTF} protein (6.80 kDa) after expression. L; Ladder, Pre: Pre-induction with IPTG, Post: Post-induction with IPTG. b. Q_{LTF} protein (6.80 kDa) after purification. L: Ladder, FT: Flow-through, following are increasing concentrations of imidazole.

FIG. 40. All MALDI-TOF spectra used in calculation of average L_{TF} incorporation for Q2_{LTF}.

FIG. 41. All MALDI-TOF spectra used in calculation of average L_{TF} incorporation for Q_{LTF}.

FIG. 42. Representative ATR-FTIR spectrum of Q_{LTF}. Overall spectrum by deconvolution in black and individual peak deconvolutions in dotted red lines (α-helix), blue lines (β-sheet), and orange lines (random coil/turns).

FIG. 43. Various resolution TEM images of Q2_{LTF}.

FIG. 44. Fluorescent confocal micrographs of Q2_{LTF} at various resolutions.

FIG. 45. CCM-bound fiber diameters measured by confocal microscopy compared to fiber diameters measured by TEM. Black dots represent previous fiber constructs and black line represents previous linear relationship. Green dot represents Q2_{LTF}. Error bars represent standard deviation of 20 fiber diameters.

FIG. 46. NMR spectra acquired 500 MHz for a. TFA (11.8 mM) in 10% D₂O b. 1.5 mM Q2_{LTF} and c. 1.5 mM Q_{LTF} without line broadening and with arrows indicating ¹⁹F peak signals 1-3 locations.

FIG. 47. The acoustic backscatter property of Q2_{LTF} was investigated in an ultrasound phantom using the Vevo 3100 high frequency ultrasound scanner (VisualSonics—Fujifilm). The ultrasound images were acquired using the MX550D 40 MHz transducer. Ultrasound gel (Aquasonic Clear, Parker laboratories, Fairfield, NJ) was applied on top of an ultrasound gel pad (Aquaflex, Parker laboratories, Fairfield, NJ) and the face of the transducer was lowered until touching the US gel. a. The B-mode image of the ultrasound gel shows an anaechoic region between the face of the transducer and the gel-gel pad interface. b. After pipetting the Q2_{LTF} into the US gel, B-mode image clearly demonstrates the Q2_{LTF} is highly echogenic (red arrows) resulting in ultrasound image contrast.

FIG. 48. In vivo ¹⁹F MR spectra with Q2_{LTF} injected within the joint after turning off the administration of isoflurane as an anesthetic. The resulting SNR of the isoflurane peak and the ratio of SNR between Q2_{LTF} peak and isoflurane peak overtime in comparison to respirations/min of the mouse. The red dashed line shows the point of separation used to distinguish Q2_{LTF} and isoflurane peak in SNR calculations.

FIG. 49. In vivo ¹⁹F MR spectra using 6 m 40 s scan time at 100 ms TR of Q2_{LTF}.

FIG. 50. a. Q hydrogel variant protein sequences with blue mutations highlighting differences made to the hydrophobic domain and green mutations highlighting differences to the coiled-coil surface compared to Q. b. Q hydrogel variant design order in which: i) Probabilistic Rosetta Score-based Monte Carlo Searches were aggregated to generate sequences for Q4 and Q5 and ii) a trimodal Monte Carlo search employing criteria for Rosetta score, NEE_{bef}, and CEE_{bef} were used to find a targeted ΔEE_{bcf} for Q6 and Q7 sequences. FIG. 50a shows the following sequences: SEQ ID NO:23, SEQ ID NO:11, SEQ ID NO:12; SEQ ID NO:13; SEQ ID NO:14; SEQ ID NO:15; and SEQ ID NO:16

FIG. 51. a. TEM images of Q and Q2-7 with scale bar at 500 nm. b. Average nanofiber diameters by TEM for Q hydrogel variants (n=100) and its correlation to ΔEE_{bcf} where error bars represent the standard deviation. c. Linear relationship between average fiber diameter and t_{c}. d. Linear relationship between t_{c} and ΔEE_{bcf} e. Bivariate linear regression model for all Q hydrogel variants for N EE_{bcf}, C EE_{bcf} and t_{c}.

FIG. 52. a. Q5 extent of gelation, η, calculated by bivariate linear regression of a concentration-temperature phase diagram constructed from tube inversions where black markers represent gel behavior and red markers represent solution behavior after two weeks of incubation at 4° C. b. Maximum upper critical solution temperature measured at the solubility limit for Q and Q2¹⁶, and Q3-7. c. Linear correlation of the temperature (dependence) coefficient calculated from respective phase diagrams and t_{c}.

FIG. 53. a. Average storage modulus (G′, filled markers) and loss modulus (G″, empty markers) of Q3-7 measured using a parallel plate rheometer at various frequencies and b. at 10 Hz. Error bars represent the standard deviation of three independent trials c. Representative FRAP imaging of the start (pre-bleach), bleach, and recovery (final post-bleach) and corresponding montage showing the pre-bleach and the first 29 frames post-bleach. d. FRAP recovery curves made by fitting to one-phase association equation in Graphpad. e. Linear correlation of Rosetta score to Y0 in one-phase association equation fit in Graphpad and f. linear correlation of temperature dependence coefficient solved in respective phase diagrams to halftime recovery in one-phase association equation fit in Graphad.

FIG. 54. a. Low q log-log SAXS spectra for Q5 at 3 mM and incubation at 4° C. from 0 h to 24 h. b. pair distance distribution function calculated in primus (ATSAS software) for Q5 at 0.15 mM and 0 h of incubation at 4° C. (black line) and Q5 at 3 mM and 24 h of incubation at 4° C. (purple line) with gaussian deconvolutions made in PeakFit software (magenta/pink dotted lines). c. Kratky plots of Q5 at 0.15 mM and 0 h of incubation at 4° C. and Q5 at 3 mM and incubation at 4° C. from 0 h to 24 h. d. Flory exponent, ν, calculated using MFF fit by the Sosnick group³¹. e. Representative cryo-EM image of Q5 at 2 mM and its f. Fast Fourier Transfer (FFT) output.

FIG. 55. Coarse-grained (CG) molecular dynamics (MD) of Q5 fibrils. a. Schematic of the Q5 coiled-coil (CC) CG model mapped to atomistic sites; the CG sites and virtual CG sites are represented as cyan and magenta spheres, respectively. b. Snapshot from a CGMD simulation (ϵ=40) of a 4 CC by 4 CC fibril consisting of protofibrils of length 14 CCs. Cyan, green, and blue colors are used to distinguish individual CCs. The schematic shows the staggered square lattice configuration where “circles” indicate co-planar protofibrils and “crosses” indicate co-planar protofibrils that are staggered with respect to “circles”. The solid blue and dashed red lines indicate “adjacent” and “diagonally-aligned” protofibrils, respectively. c. Phase diagram showing the average normalized fibril diameter predicted by CGMD simulations as a function of the dielectric constant and initial fibril size (N is the number of protofibrils used to construct the fibril based on an N×N grid). Here, circle (‘o’) and cross (‘x’) symbols represent fibrils that stay associated and become dissociated, respectively. d. The normalized radial density distribution (ρ̅(r)) between all CG sites (i.e., α carbon positions, yellow line) and between all centers-of-mass (COM, blue line) of each Q5 CC; the COM distribution is scaled by a factor of 20,000 to account for the relative difference in magnitudes of the two distributions due to differing total number densities. e. Schematic showing the inter-CC alignment observed from CGMD simulations (ϵ=40, 4×4×14 CCs); the surface representation is colored by charge and ranges between −0.75 e (red) to 0.0 e (white) to 0.75 e (blue).

FIG. 56. Q3 protein after purification. L: ladder, following are increasing mM concentrations of imidazole.

FIG. 57. Q4 protein after purification. L: ladder, following are increasing mM concentrations of imidazole.

FIG. 58. Q5 protein after purification. L: ladder, following are increasing mM concentrations of imidazole.

FIG. 59. Q6 protein after purification. L: ladder, following are increasing mM concentrations of imidazole.

FIG. 60. Q7 protein after purification. L: ladder, following are increasing mM concentrations of imidazole.

FIG. 61. Representative microrheological analysis using MPT for a. Q3, b. Q4, c. Q5, d. Q6 and e. Q7 showing i log-log plot of MSD and lag time, τ, ii. time-cure superposition of MSD vs τ, iii. Logarithmic shift factors for the vertical (log(a) in blue) and horizontal (log(b) in red) directions used in the time cure superposition to determine the t_{c} and iv. log-log plot of the shift factors and their distance from t_{c} determined by the ratio of the logarithmic slopes of the horizontal to vertical shift factor.

FIG. 62. Preliminary linear correlation for ΔEE_{bcf} to t_{c}.

FIG. 63. Accuracy of bivariate regression model for NEE_{bcf} and CEE_{bcf} to predict t_{c} showing residual differences between measured t_{c} and predicted t_{c} using the bivariate linear regression model.

FIG. 64. Extent of gelation, η, calculated by bivariate linear regression of a concentration-temperature phase diagram constructed from tube inversions where black dots represent gel behavior and red dots represent solution behavior after two weeks of incubation at 4° C. for a. Q3, b. Q4, c. Q6 d. and Q7.

FIG. 65. Start intensity of hydrogel variants prior to FRAP experiment. Error bars represent standard deviation of three independent trials.

FIG. 66. Average CD spectra for a. Q3, b. Q4, c. Q5, d. Q6, and e. Q7 in solution state (prior to incubation at 4° C., solid lines) and as a hydrogel (after incubation at 4° C., dotted lines). f. Comparison of negative MRE values at helical minima 222 nm and 208 nm. Error bars represent the standard deviation from three independent trails.

FIG. 67. a. Representative ATR-FTIR spectra for Q3-7 in a solution state (pre-incubation at 4° C.) and b. as a hydrogel (post-incubation at 4° C.). Linear correlation of Rosetta score to c. percent increase in structured content (α-helix and β-sheet) between solution state and hydrogel ATR-FTIR spectra and d. deconvoluted α-helicity of ATR-FTIR spectra as a hydrogel. e. Linear correlation of α-helicity of ATR-FTIR spectra as a hydrogel and percent increase in structured content (α-helix and β-sheet) between solution state and hydrogel ATR-FTIR spectra. f. Bivariate linear correlation between Rosetta score and hydrogel α-helicity and increase in structured content (α-helicity and β-sheet) as measured by deconvoluted ATR-FTIR spectra.

FIG. 68. Representative TEM images of Q5 after incubation at a. 3 h b. 6 h c. 12 h and d. 24 h incubated at 4° C.

FIG. 69. a. Representative cryo-EM images and b. corresponding histogram of measured fiber diameters of Q5 at 2 mM c. Representative cryo-EM images and d. corresponding histogram of measured fiber diameters of Q5 at 1 mM. e. Representative cryo-EM images and f. corresponding histogram of measured fiber diameters of Q5 at 0.5 mM.

FIG. 70. Energy surfaces for the CG model of Q5 calculated using LAMMPS. The red points indicate the initial structure used to run CG MD simulations.

FIG. 71. Radial density distributions for all CG fibril simulations.

FIG. 72. The normalized number density for each CG fibril simulation used to calculate the diameter of fibrils in FIG. 55c.

FIG. 73. a. TEM image of Q5 and b. Q5Eo indicating increasing physical crosslinking upon addition of exosomes to Q5. c. Circular dichroism spectroscopy of Q5 before incubation at 4° C. (sol) and after incubation at 4° C. with (Gel+Exo) ad without (Gel) exosomes. Increased signal dampening has been previously been shown to be associated with increased viscoelastic behavior in previous Q variants where addition of exosomes additionally dampens circular dichroism signal.

FIG. 74. a. Storage modulus (G′) and loss modulus (G″) of Q5 with and without exosomes as a function of frequency between 0.1 and 10 Hz after incubation at 4° C. b. G′ and G″ of Q5 and Q5Exo after incubation at 4° C. showing statistically insignificant difference between them. c. Multiple particle tracking (MPT) microrheological superposition analysis example of Q5Exo possessing average (n=3) t_{gel} of 5.4±1.2 h and n_{c} of 0.51±0.04 h. t_{gel} is a more than 2-fold increase compared to Q5 alone.

FIG. 75. Wound healing dynamics. a. Time-course images of Q5Exo on top of a wound indicating viscoelastic behavior over time. b. Photometric data capture of time to closure. c. % of open wound over time. d. Wound burden (area under the curve of c.).

FIG. 76. Protein engineered supramolecular assemblies: Using computational design, Q8 is identified with optimal electrostatic charge (lowest ΔEE_{bcf}) capable of physically crosslinking into hydrogels. To address the challenges of drug solubility or breakdown, rapid clearance, non-specific killing of healthy cells, and the inability to directly monitor chemotherapeutic agents such as doxorubicin (Dox) (representative example) and paclitaxel (Ptx)'s impact on diseased cells, our engineered biomaterials are able to encapsulate the drug, prevent side effect in healthy cells and remain at the cancer cell site by physical gelation while at the cancer site, and be imaged via ex/em 480/600 nm fluorescence and ultrasound.

FIG. 77. Characterization of Q8 structure and rheology. a. Frequency sweep with parallel plate rheometer at 5% strain of Q8 storage (G′) and loss (G″) moduli. b. Image of Q8 protein solution after gelation exhibiting shape retention on flat surface. c. Comparison of G′ of Q8 after addition of Dox and shearing through 26-gauge syringe at 4° C. and 37° C. d. Circular dichroism spectroscopy wavelength scan of Q8 before (sol) and after (gel) incubation at 4° C. ATR-FTIR spectra of Q8 e. before and f. after incubation at 4° C.

FIG. 78. Encapsulation of Dox and sustained release. a. Dox standard curve using absorbance at 490 nm used to measure relative encapsulation in Q8. b. TEM images of Q8 after encapsulation of Dox indicating increased physical crosslinking density of fibers. c. IVIS images of mice after subcutaneous injection with Q8 (gel only), Q8·Dox (S1-S3), or retroorbital injection of Dox alone over the course of 8 days. d. Average increase in mean red fluorescence over 8 days. Error bars represent the standard deviation of three independent mice for subcutaneous injections of Q8·Dox.

FIG. 79. Tumor suppression of Q8·Dox. a. Representative ultrasound slice used in 3D tumor volume measurements. b. Average percent tumor volume change after 1 week of treatment by subcutaneous injection of Q8 hydrogel, Q8·Dox hydrogel, and retroorbital injection of Dox. Error bars represent the standard deviation of 4 independent mice.

FIG. 80. Storage (G′) and loss (G″) moduli as a function of frequency using parallel plate rheology before and after binding 5 mM of Mn²⁺ highlighting G′ and G″ in the table below.

FIG. 81. Circular Dichroism spectroscopy of Q5 before (solid light blue line) and after incubation (dashed light blue line) at 4° C. and then following incubation and binding with 5 mM Mn²⁺ (dashed dark blue line) with double minima MRE values and ratios highlighted in the table below.

FIG. 82. Energy dispersive x-ray spectroscopy with scanning electron micrographs of Q5 after binding 5 mM Mn²⁺.

FIG. 83. Energy dispersive x-ray spectroscopy with scanning electron micrographs of Q5 after binding 5 mM Mn²⁺ with independent breakdown of different elements by color.

FIG. 84. R1 values using Bruker Minispec of Mn²⁺ in 50 mM Tris pH 8.0 buffer and Mn²⁺ bound Q5 at concentrations below 5 mM indicating complete binding of manganese and magnetic resonance imageability as a T1-brightening agent.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g., 90%, 95%, or more confidence interval from the mean), such as, for example, variations of +/−10% or less, +1-5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%, 0.5% to 2.4%, 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further disclosure. For example, if the value “about 10” is disclosed, then “10” is also disclosed.

As used herein, the terms “including,” “containing,” and “comprising” are used in their open, non-limiting sense.

As used in this disclosure, the singular forms include the plural forms and vice versa unless the context clearly indicates otherwise.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the individual.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

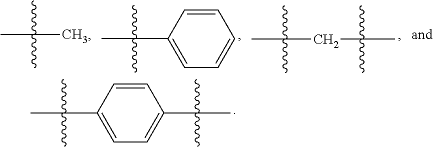

Amino acids and amino acid residues may be referred to herein by either their commonly known three letter symbols or by the one-letter symbols recommended by the IUPAC-IUB Biochemical Nomenclature Commission.

The present disclosure also provides sequences that have homology with the protein or peptides sequences (including antibody sequences) described herein. In various examples, the homologous sequences have at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with a protein or peptide sequence of the present disclosure.

In an aspect, the present disclosure provides proteins or peptides. The proteins or peptides comprise a sequence designed by the methods described herein. The proteins and peptides may have one or more trifluoroleucine (L_{TF}) residues. The proteins and peptides may have desirable self-assembling properties such that they form supramolecular structures (e.g., fibers or fibrils). The supramolecular structures may further gelate water such that a hydrogel is formed.

For example, the proteins or peptides may be fluorinated analogues of peptides derived from a variant of a coiled-coil domain of cartilage oligomeric matrix protein (COMPcc). The various proteins may be referred to as a Q protein/peptide following by a number, where each number corresponds to a different protein/peptide. For example, the present disclosure provides Q2, Q3, Q4, Q5, Q6, Q7, or Q8 or a protein comprising the sequences of Q2, Q3, Q4, Q5, Q6, Q7, or Q8, where the one or more leucine residues of the protein or peptide is replaced with trifluoroleucine. In various examples, the protein or peptide may comprise fluorinated sequences of Q2, Q3, Q4, Q5, Q6, Q7, or Q8. One, some, or all of the leucine residues of the protein or peptide may be replaced with trifluoroleucine.

A protein or peptide of the present disclosure may have or comprise the following sequence:

(SEQ ID NO: 1)
VX₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X
₂₄X₂₅X₂₆X₂₇X₂₈X₂₉X₃₀X₃₁X₃₂X₃₃X₃₄X₃₅X₃₆X₃₇,

where
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R, or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₆ is A, E, or Q;
X₁₇ is A, L, M, I, V, or L_{TF};
X₁₈ is A, K, E, D, K, R, H, N, or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ or any L is replaced with a trifluoroleucine residue. In various embodiments, one, some, or all L residues and/or X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ residues are replaced with L_{TF}. In various embodiments, a protein or peptide of the present disclosure further comprises MRGSHHHHHHGSIEGR (SEQ ID NO:2). Thus, for example, a peptide or protein may have or comprise the following sequence:

(SEQ ID NO: 26)
MRGSHHHHHHGSIEGRVX₁X₂X₃X₄X₅X₆X₇X₈X₉X₁₀X₁₁X₁₂X₁₃X₁₄X₁₅X₁₆
X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇

where
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R, or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₆ is A, E, or Q;
X₁₇ is A, L, M, I, V, or L_{TF};
X₁₈ is A, K, E, D, K, R, H, N, or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇, or any L is replaced with L_{TF}. In various embodiments, one, some, or all L residues and/or X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ residues are replaced with L_{TF}.

A protein or peptide of the present disclosure may have or comprise the following sequence:

VX₁EX₃X₄X₅X₆KX₈X₉X₁₀X₁₁X₁₂X₁₃X₁₄X₁₅EX₁₇X₁₈X₁₉X₂₀NX₂₂AX₂₄X₂₅
X₂₆X₂₇RX₂₉X₃₀LX₃₂X₃₃X₃₄X₃₅X₃₆X₃₇ (SEQ ID NO: 1, where X₂
is E, X₇ is K, X₁₆ is E, X₂₁ is N, X₂₃ is A, X₂₈ is
R, and X₃₁ is L)
or
(SEQ ID NO: 27)
MRGSHHHHHHGSIEGRVX₁EX₃X₄X₅X₆KX₈X₉X₁₀X₁₁X₁₂X₁₃X₁₄X₁₅EX₁₇
X₁₈X₁₉X₂₀NX₂₂AX₂₄X₂₅X₂₆X₂₇RX₂₉X₃₀LX₃₂X₃₃X₃₄X₃₅X₃₆X₃₇.

In various embodiments,
X₁ is K or S;
X₃ is L, I, Q, M, or L_{TF};
X₄ is L, I, T, K, or L_{TF};
X₅ is F or E;
X₆ is L, I, or L_{TF};
X₈ is K or N;
X₉ is T, Q, or L_{TF};
X₁₀ is A, L, I, or L_{TF};
X₁₁ is E, P, I, L, Y, or L_{TF};
X₁₂ is Q or H;
X₁₃ is M, I, L, Q, or L_{TF};
X₁₄ is A, L, I, or L_{TF};
X₁₅ is E, I, L, R, K, or L_{TF};
X₁₇ is L, M, I, or L_{TF};
X₁₈ is K or Q;
X₁₉ is N or E;
X₂₀ is L, T, I, or L_{TF};
X₂₂ is K, E, I, L, M, or L_{TF};
X₂₄ is L, I, or L_{TF};
X₂₅ is H, Q, Y, I, L, or L_{TF};
X₂₆ is D or T;
X₂₇ is V, I, L, or L_{TF};
X₂₉ is H, E, I, L, or L_{TF};
X₃₀ is L, A, I, Q, or L_{TF};
X₃₂ is E, Q, L, I, or L_{TF};
X₃₃ is N or Q;
X₃₄ is Q, I, L, M, or L_{TF};
X₃₅ is S or A;
X₃₆ is K or T;
X₃₇ is L, I, K, N, Q, or L_{TF}.

In various embodiments,
X₁ is K or S;
X₃ is L, I, Q, M, or L_{TF};
X₄ is L, T, K, or L_{TF};
X₅ is F or E;
X₆ is L, I, or L_{TF};
X₈ is K or N;
X₉ is T or Q;
X₁₀ is A or L;
X₁₁ is E, P, or Y;
X₁₂ is Q or H;
X₁₃ is M, I, or Q;
X₁₄ is A, L, or L_{TF};
X₁₅ is E, L, R, K, or L_{TF};
X₁₇ is L, M, I, or L_{TF};
X₁₈ is K or Q;
X₁₉ is N or E;
X₂₀ is T or I;
X₂₂ is K, E, or M;
X₂₄ is L, I, or L_{TF};
X₂₅ is H, Q, or Y;
X₂₆ is D or T;
X₂₇ is V or I;
X₂₉ is H or E;
X₃₀ is L, A, or Q;
X₃₂ is E or Q;
X₃₃ is N or Q;
X₃₄ is Q, I, or M;
X₃₅ is S or A;
X₃₆ is K or T;
X₃₇ is L, K, N, Q, or L_{TF}.

In various embodiments, a peptide or protein has or comprises the following sequence:

(SEQ ID NO: 3)
VKELLFLKKTAEQMLEELKETNKALHDVRHLLENQSKL;
(SEQ ID NO: 4)
VKEILFLKNTAYQMLLELKETNEALYDIRHLLQQQSKL;
(SEQ ID NO: 5)
VKEITFIKKTIEQIAEEMKEINKAIHDIRHALENISKK;
(SEQ ID NO: 6)
VKEQTFIKNTIEQMAEEIKEINKAIHDIRHQLENMSKQ;
(SEQ ID NO: 7)
VKEITFIKKQIEQIAEEMKEINKAIHDIRHALENISKK;
(SEQ ID NO: 8)
VKEMTFIKKTIEQQAEEMKEINKAIHDIRHALENISKK;
(SEQ ID NO: 9)
VSEITEIKKTIEHIAKEMKEINKAIHTIRHALENIAKN;
or
(SEQ ID NO: 10)
VKEIKFLKNTAPQQLRELQNTNMALQDVRELLQQQSTL,

where one, some, or all the leucine residues of each sequence is replaced with a L_{TF} residue. The sequence may have 75 to 99% identity to any of the foregoing sequences. When the protein or peptide comprises a homologous sequence of any one of the foregoing sequences, at least one leucine residue is replaced with L_{TF}. In various examples, only one leucine residue is replaced with L_{TF}. In various examples, some, but not all, of the leucine residues are replaced with L_{TF}. In various examples, all the leucine residues are replaced with L_{TF}.

As described above a protein or peptide of the present disclosure may comprise MRGSHHHHHHGSIEGR (SEQ ID NO:2). MRGSHHHHHHGSIEGR (SEQ ID NO:2) may be from recombinant production of the protein or peptide. Examples of peptides/proteins including MRGSHHHHHHGSIEGR (SEQ ID NO:2), include, but are not limited to,

(SEQ ID NO: 11)
MRGSHHHHHHGSIEGRVKELLFLKKTAEQMLEELKETNKALHDVRHLLE
NQSKL;
(SEQ ID NO: 12)
MRGSHHHHHHGSIEGRVKEILFLKNTAYQMLLELKETNEALYDIRHLLQ
QQSKL;
(SEQ ID NO: 13)
MRGSHHHHHHGSIEGRVKEITFIKKTIEQIAEEMKEINKAIHDIRHALE
NISKK;
(SEQ ID NO: 14)
MRGSHHHHHHGSIEGRVKEQTFIKNTIEQMAEEIKEINKAIHDIRHQLE
NMSKQ;
(SEQ ID NO: 15)
MRGSHHHHHHGSIEGRVKEITFIKKQIEQIAEEMKEINKAIHDIRHALE
NISKK;
(SEQ ID NO: 16)
MRGSHHHHHHGSIEGRVKEMTFIKKTIEQQAEEMKEINKAIHDIRHALE
NISKK;
(SEQ ID NO: 17)
MRGSHHHHHHGSIEGRVSEITEIKKTIEHIAKEMKEINKAIHTIRHALE
NIAKN;
or
(SEQ ID NO: 18)
MRGSHHHHHHGSIEGRVKEIKFLKNTAPQQLRELQNTNMALQDVRELLQ
QQSTL,

where one, some, or all the leucine residues of each sequence is replaced with a L_{TF} residue. The sequence may have 75 to 99% identity to any of the foregoing sequences. When the protein or peptide comprises a homologous sequence of any one of the foregoing sequences, at least one leucine residue is replaced with L_{TF}. In various examples, only one leucine residue is replaced with L_{TF}. In various examples, some, but not all, of the leucine residues are replaced with L_{TF}. In various examples, all the leucine residues are replaced with L_{TF}.

In an aspect, the present disclosure provides protein fibers or peptide fibers and gels comprising those fibers. The protein fibers and peptide fibers may be referred to fibers. These fibers may comprise one or more protofibers. The one or more protofibers may comprise a plurality of proteins and/or peptides of the present disclosure.

The proteins and peptides of the present disclosure may have desirable properties. The proteins and peptides self-assemble/self-associate/aggregate to form protofibers. The assembled proteins and peptides are bound via non-covalent interactions (e.g., Coulombic interactions, hydrophobic interactions, π-π interactions, and the like, and combinations thereof) and van der Waals interactions. The protofibers may associate with other protofibers to form protein fibers or peptide fibers. The protofibers may have a diameter of less than 20 nm. The fibers may have a diameter of about 20 nm to about 2 pm, including all 0.1 nm values and ranges therebetween. The fiber has a coiled-coil morphology defining a 1-50 angstrom pore running along the length of fiber.

Without intending to be bound by any particular theory, it is considered that the peptides/proteins of the present disclosure favor longitudinal growth over lateral growth of their fibers. This may allow the fibers to achieve a higher crosslinking density within a hydrogel formed therefrom.

These protein fibers or peptide fibers may further aggregate to form an entangled network of fibers that restrict the flow of water to form a hydrogel. That is, a hydrogel of the present disclosure may comprise water and fibers of the present disclosure, where the fibers comprise protofibers comprising peptides/proteins of the present disclosure. In an embodiment, the hydrogel comprises crosslinked fibers. In an embodiment, the hydrogel comprises only non-covalently crosslinked fibers and no chemically crosslinked fibers. In an embodiment, the fibers non-covalently associate via one or more non-covalent interactions (e.g., hydrophobic interactions, π-π interactions, hydrogen bonds, and the like, and combinations thereof). Physical crosslinking is more likely to occur at low temperatures and above 1 mM protein concentrations.

A hydrogel can comprise various amounts of water. In various examples, a hydrogel comprises 80 to 99%, such as 85 to 99.9% by weight (based on the total weight of the composition) water. In an embodiment, the hydrogel comprises about 91 to 99.9% by weight water. In various embodiments, the hydrogel comprises 99 to 99.5% weight water.

A fiber may have various compounds bound thereto. A fiber may have one or more compounds bound thereto, which may be the same or different. The compounds may be therapeutic agents. In various examples, the compound are hydrophobic. In various examples, the compounds a non-hydrophobic. In various examples, the compounds have no net charge. Non-limiting examples of compounds include dyes, antibiotics, alkaloids, vitamins, lipids, fatty acids, sugars, amino acids, phenolic compounds, extracellular materials (e.g., proteins, cells, exosomes, and the like, and combinations thereof), metals, nucleic acids, and the like, and combinations thereof.

A gel may encapsulate various compounds. A gel may encapsulate one or more compounds, which may be the same or different. The compounds may be therapeutic agents. In various examples, the compound are hydrophobic. In various examples, the compounds a non-hydrophobic. In various examples, the compounds have no net charge. Non-limiting examples of compounds include dyes, antibiotics, alkaloids, vitamins, lipids, fatty acids, sugars, amino acids, phenolic compounds, extracellular materials (e.g., proteins, cells, exosomes, and the like, and combinations thereof), metals, nucleic acids, and the like, and combinations thereof.

In various examples, one or more chemotherapeutic agents may be bound to a fiber or encapsulated by a gel of the present disclosure. Any FDA approved chemotherapeutic agents (i.e., chemotherapy drugs) can be used. Combinations of chemotherapeutic agents can be used. Non-limiting examples of chemotherapeutic agents and combinations include abemaciclib, abiraterone acetate, ABITREXATE® (methotrexate), ABVD (doxorubicin, bleomycin, vinblastine, and dacarbazine), ABVE (doxorubicin, bleomycin, vincristine sulfate, etoposide phosphate), ABVE-PC (doxorubicin, bleomycin, vincristine sulfate, etoposide phosphate, prednisone, cyclophosphamide), AC (doxorubicin and cyclophosphamide), acalabrutinib, AC-T (doxorubicin, cyclophosphamide, paclitaxel), ADE (cytarabine, daunorubicin, etoposide), ADRIAMYCIN® (doxorubicin hydrochloride), afatinib dimaleate, AFINITOR® (everolimus), AKYNZEO® (netupitant and palonosetron hydrochloride), ALDARA® (imiquimod), aldesleukin, ALECENSA® (alectinib), alectinib, ALIMTA® (pemetrexed disodium), ALIQOPA® (copanlisib hydrochloride), ALKERAN® for injection (melphalan hydrochloride), ALKERAN® tablets (melphalan), ALOXI® (palonosetron hydrochloride), ALUNBRIG™ (brigatinib), ambochlorin (chlorambucil), amboclorin (chlorambucil), amifostine, aminolevulinic acid, anastrozole, aprepitant, AREDIA® (pamidronate disodium), ARIMIDEX® (anastrozole), AROMASIN® (exemestane), ARRANON® (nelarabine), arsenic trioxide, asparaginase Erwinia chrysanthemi, axicabtagene ciloleucel, axitinib, azacitidine, BEACOPP (bleomycin, etoposide, doxorubicin, cyclophosphamide, vincristine, procarbazine, prednisone), Becenum® (carmustine), Beleodaq® (belinostat), belinostat, bendamustine hydrochloride, BEP (bleomycin, etoposide, cisplatin), bexarotene, bicalutamide, BICNU® (carmustine), bleomycin, bortezomib, Bosulif® (bosutinib), bosutinib, brigatinib, BuMel (busulfan, melphalan hydrochloride), busulfan, BUSULFEX® (busulfan), cabazitaxel, CABOMETYX™ (cabozantinib-S-malate), cabozantinib-S-malate, CAF (cyclophosphamide, doxorubicin, 5-fluorouracil), CALQUENCE® (acalabrutinib), CAMPTOSAR® (irinotecan hydrochloride), capecitabine, CAPOX, CARAC™ (fluorouracil-topical), carboplatin, carboplatin-TAXOL®, carfilzomib, carmubris (carmustine), carmustine, carmustine implant, CASODEX® (bicalutamide), CEM (carboplatin, etoposide, melphalan), ceritinib, CERUBIDINE® (daunorubicin hydrochloride), CEV (carboplatin, etoposide phosphate, vincristine sulfate), chlorambucil, chlorambucil-prednisone, CHOP (cyclophosphamide, doxorubicin, vincristine, prednisone), cisplatin, cladribine, CLAFEN® (cyclophosphamide), clofarabine, CLOFAREX® (clofarabine), CLOLAR® (clofarabine), CMF (cyclophosphamide, methotrexate, fluorouracil), cobimetinib, COMETRIQ® (cabozantinib-S-malate), copanlisib hydrochloride, COPDAC (cyclophosphamide, vincristine sulfate, prednisone, dacarbazine), COPP (cyclophosphamide, vincristine, procarbazine, prednisone), COPP-ABV (cyclophosphamide, vincristine, procarbazine, prednisone, doxorubicin, bleomycin, vinblastine sulfate), COSMEGEN® (dactinomycin), COTELLIC® (cobimetinib), crizotinib, CVP (cyclophosphamide, vincristine, prednisolone), cyclophosphamide, CYFOS® (ifosfamide), cytarabine, cytarabine liposome, CYTOSAR-U® (cytarabine), CYTOXAN® (cyclophosphamide), dabrafenib, dacarbazine, DACOGEN® (decitabine), dactinomycin, dasatinib, daunorubicin hydrochloride, daunorubicin hydrochloride and cytarabine liposome, decitabine, defibrotide sodium, DEFITELIO® (defibrotide sodium), degarelix, denileukin diftitox, dexamethasone, dexrazoxane hydrochloride, docetaxel, doxorubicin, doxorubicin hydrochloride, doxorubicin hydrochloride liposome, DOX-SL® (doxorubicin hydrochloride liposome), DTIC-DOME® (dacarbazine), ELITEK® (rasburicase), ELLENCE® (epirubicin hydrochloride), ELOXATIN® (oxaliplatin), eltrombopag olamine, EMEND@ (aprepitant), enasidenib mesylate, enzalutamide, epirubicin hydrochloride, EPOCH (etoposide, prednisone, vincristine, cyclophosphamide, and doxorubicin hydrochloride), eribulin mesylate, ERIVEDGE® (vismodegib), erlotinib hydrochloride, ERWINAZE® (asparaginase Erwinia chrysanthemi), ETHYOL® (amifostine), ETOPOPHOS® (etoposide phosphate), etoposide, etoposide phosphate, everolimus, EVISTA® (raloxifene hydrochloride), EVOMELA® (melphalan hydrochloride), exemestane, 5-FU (fluorouracil), FARESTON® (toremifene), FARYDAK® (panobinostat), FASLODEX® (fulvestrant), FEC (5-fluorouracil, epirubicin, cyclophosphamide), FEMARA® (letrozole), filgrastim, FLUDARA® (fludarabine phosphate), fludarabine phosphate, flutamide, FOLEX® (methotrexate), FOLEX PFS® (methotrexate), FOLFIRI (leucovorin calcium, fluorouracil, irinotecan hydrochloride), FOLFIRINOX (leucovorin calcium, fluorouracil, irinotecan hydrochloride, oxaliplatin), FOLFOX (leucovorin calcium, fluorouracil, oxaliplatin), FOLOTYN® (pralatrexate), FU-LV (fluorouracil, leucovorin calcium), fulvestrant, gefitinib, gemcitabine hydrochloride, gemcitabine-cisplatin, gemcitabine-oxaliplatin, GEMZAR® (gemcitabine hydrochloride), GILOTRIF® (afatinib dimaleate), GLEEVEC® (imatinib mesylate), GLIADEL® (carmustine implant), goserelin acetate, HALAVEN® (eribulin mesylate), HEMANGEOL® (propranolol hydrochloride), Hycamtin® (topotecan hydrochloride), HYDREA® (hydroxyurea), hydroxyurea, Hyper-CVAD (course A: cyclophosphamide, vincristine, doxorubicin, dexamethasone, cytarabine, mesna, methotrexate; and course B: methotrexate, leucovorin, sodium bicarbonate, cytarabine), IBRANCE® (palbociclib), ibrutinib, ICE (ifosfamide, mesna, carboplatin, etoposide), ICLUSIG® (ponatinib hydrochloride), IDAMYCIN® (idarubicin hydrochloride), idarubicin hydrochloride, idelalisib, IDHIFA® (enasidenib mesylate), IFEX® (ifosfamide), ifosfamide, IFOSFAMIDUM™ (ifosfamide), imatinib mesylate, IMBRUVICA® (ibrutinib), imiquimod, IMLYGIC® (talimogene laherparepvec), INLYTA® (axitinib), IRESSA® (gefitinib), irinotecan, irinotecan hydrochloride, irinotecan hydrochloride liposome, ISTODAX® (romidepsin), ixabepilone, ixazomib citrate, IXEMPRA® (ixabepilone), JAKAFI® (ruxolitinib phosphate), JEB (carboplatin, etoposide phosphate, bleomycin), JEVTANA® (cabazitaxel), KEOXIFENE™ (raloxifene hydrochloride), KEPIVANCE® (palifermin), KISQALI® (ribociclib), KYMRIAH™ (tisagenlecleucel), KYPROLIS® (carfilzomib), lanreotide acetate, lapatinib ditosylate, lenalidomide, lenvatinib mesylate, LENVIMA® (lenvatinib mesylate), letrozole, leucovorin calcium, LEUKERAN® (chlorambucil), leuprolide acetate, LEUSTATIN® (cladribine), LEVULAN® (aminolevulinic acid), LINFOLIZIN™ (chlorambucil), lomustine, LONSURF® (trifluridine and tipiracil hydrochloride), LUPRON® (leuprolide acetate), LUPRON DEPOT@ (leuprolide acetate), LUPRON DEPOT-PED® (leuprolide acetate), LYNPARZA® (olaparib), MATULANE® (procarbazine hydrochloride), mechlorethamine hydrochloride, megestrol acetate, MEKINIST® (trametinib), melphalan, melphalan hydrochloride, mercaptopurine, mesna, MESNEX® (Mesna), METHAZOLASTONE™ (temozolomide), methotrexate, METHOTREXATE LPF™ (methotrexate), methylnaltrexone bromide, MEXATE® (methotrexate), MEXATE-AQ™ (methotrexate), midostaurin, mitomycin C, mitoxantrone hydrochloride, MITOZYTREX™ (mitomycin C), MOPP (mustargen, vincristine, procarbazine, prednisone), MOZOBIL™ (plerixafor), MUSTARGEN® (mechlorethamine hydrochloride), MUTAMYCIN™ (mitomycin C), MYLERAN® (busulfan), MYLOSAR® (azacitidine), NAVELBINE® (vinorelbine tartrate), nelarabine, NEOSAR® (cyclophosphamide), neratinib maleate, NERLYNX® (neratinib maleate), netupitant and palonosetron hydrochloride, NEULASTA® (pegfilgrastim), NEUPOGEN® (filgrastim), NEXAVAR® (sorafenib tosylate), NILANDRON® (nilutamide), nilotinib, nilutamide, NINLARO® (ixazomib citrate), niraparib tosylate monohydrate, NOLVADEX® (tamoxifen citrate), NPLATE® (romiplostim), ODOMZO® (sonidegib), OEPA (vincristine sulfate, etoposide phosphate, prednisone, doxorubicin hydrochloride), OFF (oxaliplatin, fluorouracil, leucovorin), olaparib, omacetaxine mepesuccinate, ondansetron hydrochloride, ONTAK® (denileukin diftitox), OPPA (vincristine sulfate, procarbazine hydrochloride, prednisone, doxorubicin hydrochloride), osimertinib, oxaliplatin, paclitaxel, PAD (bortezomib, doxorubicin hydrochloride, dexamethasone), palbociclib, palifermin, palonosetron hydrochloride, pamidronate disodium, panobinostat, paraplat (carboplatin), PARAPLATIN® (carboplatin), pazopanib hydrochloride, PCV (procarbazine hydrochloride, lomustine, vincristine sulfate), PEB (cisplatin, etoposide phosphate, bleomycin), pegfilgrastim, pemetrexed disodium, PLATINOL® (cisplatin), PLATINOL®-AQ (cisplatin), plerixafor, pomalidomide, POMALYST® (pomalidomide), ponatinib hydrochloride, pralatrexate, prednisone, procarbazine hydrochloride, PROMACTA® (eltrombopag olamine), propranolol hydrochloride, PURINETHOL® (mercaptopurine), PURIXAN® (mercaptopurine), radium 223 dichloride, raloxifene hydrochloride, rasburicase, regorafenib, RELISTOR® (methylnaltrexone bromide), REVLIMID® (lenalidomide), RHEUMATREX® (methotrexate), ribociclib, rolapitant hydrochloride, romidepsin, romiplostim, rubidomycin (daunorubicin hydrochloride), RUBRACA® (rucaparib camsylate), rucaparib camsylate, ruxolitinib phosphate, RYDAPT® (midostaurin), SCLEROSOL® Intrapleural Aerosol (Talc), sipuleucel-T, SOMATULINE® Depot (lanreotide acetate), sonidegib, sorafenib tosylate, SPRYCEL® (dasatinib), Stanford V (mechlorethamine hydrochloride, doxorubicin hydrochloride, vinblastine sulfate, vincristine sulfate, bleomycin, etoposide phosphate, prednisone), sterile talc powder (Talc), STERITALC® (Talc), STIVARGA® (regorafenib), sunitinib malate, SUTENT® (sunitinib malate), SYNRIBO™ (omacetaxine mepesuccinate), TABLOID@ (thioguanine), TAC (docetaxel, doxorubicin hydrochloride, cyclophosphamide), TAFINLAR® (dabrafenib), TAGRISSO® (osimertinib), Talc, tamoxifen citrate, TARABINE PFS® (cytarabine), TARCEVA® (erlotinib hydrochloride), TARGRETIN® (bexarotene), TASIGNA® (nilotinib), TAXOL® (Paclitaxel), TAXOTERE® (docetaxel), TEMODAR® (temozolomide), temozolomide, temsirolimus, thalidomide, THALOMID® (thalidomide), thioguanine, thiotepa, TOTECT® (dexrazoxane hydrochloride), TPF (docetaxel, cisplatin, fluorouracil), trabectedin, trametinib, TREANDA® (bendamustine hydrochloride), trifluridine and tipiracil hydrochloride, TRISENOX® (arsenic trioxide), TYKERB® (lapatinib ditosylate), uridine triacetate, VAC (vincristine sulfate, dactinomycin, cyclophosphamide), valrubicin, VALSTAR® (valrubicin), vandetanib, VAMP (vincristine sulfate, doxorubicin hydrochloride, methotrexate, prednisone), VARUBI® (rolapitant hydrochloride), VeIP (vinblastine sulfate, ifosfamide, cisplatin), VELBAN® (vinblastine sulfate), VELCADE® (bortezomib), VELSAR® (vinblastine sulfate), vemurafenib, VENCLEXTA™ (venetoclax), venetoclax, VERZENIO™ (abemaciclib), VIADUR® (leuprolide acetate), VIDAZA® (azacitidine), vinblastine sulfate, VINCASAR PFS® (vincristine sulfate), vincristine sulfate, vinorelbine tartrate, VIP (etoposide phosphate, ifosfamide, cisplatin), vismodegib, VISTOGARD® (uridine triacetate), vorinostat, VOTRIENT® (pazopanib hydrochloride), WELLCOVORIN® (leucovorin calcium), XALKORI® (crizotinib), XELODA® (capecitabine), XELIRI (capecitabine, irinotecan hydrochloride), XELOX (capecitabine, oxaliplatin), XOFIGO® (radium 223 dichloride), XTANDI® (enzalutamide), YESCARTA™ (axicabtagene ciloleucel), YONDELIS® (trabectedin), ZALTRAP® (ziv-aflibercept), ZARXIO® (filgrastim), ZEJULA® (niraparib tosylate monohydrate), ZELBORAF® (vemurafenib), ZINECARD® (dexrazoxane hydrochloride), ZOFRAN® (ondansetron hydrochloride), ZOLADEX® (goserelin acetate), zoledronic acid, ZOLINZA® (vorinostat), ZOMETA® (zoledronic acid), ZYDELIG® (idelalisib), ZYKADIA® (ceritinib), and ZYTIGA® (abiraterone acetate). In various examples, the chemotherapeutic agent is doxorubicin.

In an aspect, the present disclosure provides a composition. The composition comprises a protein or peptide designed using a method of the present disclosure. The composition may comprise a plurality of fibers comprising peptides or proteins of the present disclosure. The composition may be formulated into a gel and further comprise one or more compounds, which may be therapeutic agents.

A composition can comprise one or more proteins or peptides in a pharmaceutically acceptable carrier (e.g., carrier). The proteins or peptides may be in the form of fibers or protofibers. The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered, as well as by the particular method used to administer the composition. Accordingly, there are a wide variety of suitable formulations of pharmaceutical compositions of the present disclosure. Additional, non-limiting examples of carriers include solutions, suspensions, emulsions, solid injectable compositions that are dissolved or suspended in a solvent before use, and the like. Injections may be prepared by dissolving, suspending, or emulsifying one or more of active ingredients in a diluent. Examples of diluents, include, but are not limited to distilled water for injection, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. Parenteral administration may be prepared and include infusions such as, for example, intramuscular, intravenous, intraarterial, intraperitoneal, subcutaneous administration, and the like. For example, composition comprises vesicles of the present disclosure and a sterile, suitable carrier for administration to individuals including humans-such as a physiological buffer such as sucrose, dextrose, saline, pH buffering (such as from pH 5 to 9, from pH 7 to 8, from pH 7.2 to 7.6, (e.g., 7.4)) element such as histidine, citrate, or phosphate.

The pharmaceutical compositions or formulations of the application may further comprising one or more of the following excipients: antioxidant, buffering agent, bulking agent, non-aggregating agent, binding agent, filler, diluent (e.g., starches or partially gelatinized starches, sorbitol, mannitol, malitol, microcrystalline cellulose); disintegrant (e.g., sodium croscarmellose and sodium starch glycolate); plasticizers (e.g., glycerol, vitamin E TPGS, triacetin); anti-tacking agent (e.g., tricalcium phosphate, silicon dioxide, bentonite); wetting agent (sodium lauryl sulfate, sodium stearyl fumarate, polyoxyethylene 20 sorbitan mono-oleate (e.g., Tween™); sweetener (sucralose, sorbitol, and xylitol); colorant (FD&C Blue #1 Aluminum Lake, FD&C Blue #2, other FD&C Blue colors, titanium dioxide, iron oxide); flavorant (menthol, peppermint oil, almond oil); glidant (colloidal silica, precipitated silica, and talc); pH adjuster (arginine, tartaric acid, sodium hydrogen carbonate, adipic acid); or surfactant (ammonium lauryl sulfate, sodium lauryl sulfate (sodium dodecyl sulfate, SLS, or SDS), sodium laureth sulfate, sodium myreth sulfate, dioctyl sodium sulfosuccinate, fatty acid esters of glycerol, poloxamers).

Antioxidants, include, without limitation, hindered phenols (e.g., tetrakis [methylene (3,5-di-t-butyl-4-hydroxyhydrocinnamate)]methane), less-hindered phenols, and semi-hindered phenols; phosphates, phosphites, and phosphonites (e.g., tris (2,4-di-t-butylphenyl) phosphate); thio compounds (e.g., distearyl thiodipropionate, dilaurylthiodipropionate); various siloxanes; and various amines (e.g., polymerized 2,2,4-trimethyl-1,2-dihydroquinoline). In one embodiment, the antioxidant is selected from the group consisting of distearyl thiodipropionate, dilauryl thiodipropionate, octadecyl-3,5-di-t-butyl-4-hydroxyhydrocinnamate, benzenepropanoic acid, 3,5-bis (1,1-dimethylethyl)-4-hydroxy-thiodi-2,1-ehtanediyl ester, stearyl 3-(3,5-di-t-butyl-4-hydroxyphenyl) propionate, octadecyl-3-(3,5-di-tert-butyl-4-hydroxyphenyl)-propionate, 2,4-bis(dodecylthiomethyl)-6-methylphenol, 4,4′-thiobis(6-tert-butyl-m-cresol), 4,6-bis (octylthiomethyl)-o-cresol, 1,3,5-tris(4-tert-butyl-3-hydroxy-2,6-dimethyl benzyl)-1,3,5-triazine-2,4,6-(1H,3H,5H)-trione, pentaerythritol tetrakis (3-(3,5-di-t-butyl-4-hydroxyphenyl)propionate), 2′,3-bis[[3-[3,5-di-tert-butyl-4-hydroxyphenyl]propionyl]]propionohydrazide, and mixtures thereof. In one embodiment, the antioxidant is butylated hydroxyanisole, butylated hydroxytoluene (BHT), sodium metabisulfite, propyl gallate, cysteine, methionine, or ethylenediaminetetraacetic acid (EDTA).

Buffering agents are includes in the pharmaceutical formulations of the application in order to prevent or reduce a pH change in the dosage form after administration to a subject. Representative buffering agents include, without limitation, borates, borate-polyol complexes, succinate, phosphate buffering agents, citrate buffering agents, acetate buffering agents, carbonate buffering agents, organic buffering agents, amino acid buffering agents, or combinations thereof. In one embodiment, the buffering agent is citric acid, sodium phosphate, sodium citrate, sodium acetate, sodium hydroxide, acetic acid, potassium chloride, sodium chloride, sodium bicarbonate, L-arginine, a cholic acid derivative, or tris(hydroxymethyl)aminomethane (TRIS).

The pharmaceutical compositions or formulations of the application can be administered via a route selected from the group consisting of route selected from the group consisting of oral administration, nasal (intranasal) administration, administration by inhalation, rectal administration, intraperitoneal injection, intravascular injection, subcutaneous injection, transcutaneous administration, and intramuscular injection.

In an aspect, the present disclosure provides a method for detecting the location of a compound in an individual. The compound may be bound to a protein/peptide of the present disclosure or a fiber of the present disclosure and the protein/peptide or fiber is administered to an individual. The compound may be a therapeutic agent.

In various examples, a method of the present disclosure comprises administering to an individual a protein/peptide or fiber of the present disclosure, where the protein/peptide or fiber has a compound bound thereto or associated therewith (such as, for example, when the compound is encapsulated by a gel comprising a plurality of the fibers). After administration, a signal of the peptide/protein or fiber can be detected. In various examples, the signal can be detected via spectroscopy (e.g., magnetic resonance imaging (MRI)). The spectroscopy may be ¹⁹F MR spectroscopy or ¹H MR spectroscopy). In various examples, the signal can be detected ultrasound.

In various embodiments, the method may further comprise a treating step. The treating may be used to treat wounds (e.g., diabetic wounds) or to dose chemotherapeutic agents directly to a tumor site. The treatment step may comprise administering a therapeutic compound bound to the fiber or encapsulated by a gel comprising the fibers. In various examples, the tumor is a solid tumor. Any localized solid tumor may be suitable. This includes, but is not limited to, triple-negative breast cancers (TNBCs), brain cancer, head and neck cancer, and the like.

In an embodiment, the present disclosure provides compositions and methods for delivering therapeutic agents (e.g., compounds that act as wound healing agents) to wounds, including chronic wounds, such as chronic ulcers. The wounds maybe associated with diabetes, burns, venous disease, pressure ulcers, age-associated complications, or any disorder that compromises the barrier function and integrity of skin. High oxidative burden in the tissues of patients with diabetes is at the pathogenic roots of many complications secondary to diabetes, including poor wound healing and amputation. Oxidative stress is also implicated in pathologic healing of chronic and age-related contexts. A temporary increase in reactive oxygen species is expected and necessary to physiologic healing of cutaneous wounds. In patients without diabetes, endogenous antioxidant defense pathways, like nuclear factor erythroid 2-related factor 2 (Nrf2), transcriptionally upregulate genes that regenerate reducing equivalents, directly neutralize reactive oxygen species, and restores levels of metabolic enzymes. This system is unable to neutralize elevated oxidative loads characteristic of hyperglycemia secondary to diabetes. By exogenously enhancing this pathway, oxidative stress is reduced in wounded tissue of diabetic patients and the delay in closure in diabetic wounds is also reduced.

For example, when treating a wound (e.g., a diabetic wound), various therapeutic agents may be used. The method may comprise administering a therapeutic agent bound to the protein/peptide or fiber or encapsulated by a gel comprising a plurality of the fibers. The therapeutic agent may be any of the compounds described herein. In various examples, the compound is an extracellular material, such as, for example, an exosome. In various embodiments, the protein/peptide or fiber may be formulated as a gel. The amount of the therapeutic agent may be a therapeutically effective amount as described herein.

Various methods known to those skilled in the art can be used to introduce the proteins/peptides and/or compositions of the present disclosure to an individual. These methods include, but are not limited to, intravenous, intramuscular, intracranial, intrathecal, intradermal, subcutaneous, oral routes, and the like, and combinations thereof. The dose of the composition comprising a compound and a pharmaceutical agent will necessarily be dependent upon the needs of the individual to whom the composition is to be administered. These factors include, but are not necessarily limited to, the weight, age, sex, medical history, and nature and stage of the disease for which a therapeutic effect is desired, and wherein inhibiting coagulation is desired.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

In an aspect, the present disclosure provides machine learning models using the electrostatic potential energy of atoms responsible for the fibril assembly of a coiled-coil domain to quantify the likelihood of self-assembly and the subsequent use of the parameter for search and prediction of fiber size and mechanical properties of resulting hydrogels and the proteins that result from these methods.

These models and search algorithms using the outlined parameters serve to create coiled-coil domain fibers and hydrogels of predictable and targeted sizes and mechanical properties (i.e. crosslinking density, storage modulus, and critical gelation time).

The present disclosure provides any and all models used for the prediction of coiled-coil protein function based on the ΔEE_{bcf} parameter or EE_{bcf} of the N- and C-terminus depending on their relationship to the N- or C-terminal ends of the coiled-coil protein and any proteins developed from these models or the use of the EE_{bcf} of the N- and C-terminus depending on their relationship to the N- or C-terminal ends of the coiled-coil protein. The parameter may be determined using the following equation:

$$Δ E {E}_{bcf} = {\sum }_{n = l / 2}^{l} E {E}_{bcf} - {\sum }_{n = 1 + ( tag length )}^{\frac{l}{2} - 1} {EE}_{bcf} ,$$

where n is the sequence position number, l is the length of the sequence, and EE_{bcf} is the electrostatic potential energy of a residue in the b, c, or f helical wheel position.

The ability to engineer the solvent-exposed surface of self-assembling coiled-coils allows one to achieve higher order hierarchical assembly such as nano- or microfibers as well as hydrogels. Currently these materials are being developed for a range of biomedical applications, including drug delivery systems, however ways to mechanistically optimize the coiled-coil structure for different desired fiber sizes, hydrogel mechanical strengths, and hydrogel gelation rates has not been made clear. Protein-engineered biomaterials have become an increasingly popular choice for a variety of biomedical and materials-based applications including therapeutics and tissue-engineered scaffolds. An important subset of these materials includes those that have the ability to self-assemble. In particular, self-assembling protein biomaterials possess ideal drug delivery potential as they are both inherently biodegradable and can provide effective encapsulation of a variety of drugs and can easily be modified for targeted drug release. One such protein biomaterial that has garnered increasing interest are those that self-assemble into fibers and hydrogels. Formation of fibers and or hydrogels is often the result of a variety of stimuli such as pH, temperature, concentration, ionic strength, and sequence; thus their formation can be readily tuned.

The present disclosure also provides method to quickly and simply predict the fiber diameter and hydrogel mechanical properties (i.e., storage modulus, crosslinking density, critical time to gelation) based on machine learning models using the ΔEE_{bcf} parameter or EE_{bcf} of the N- and C-terminus depending on their relationship to the N- or C-terminal ends of the protein.

New protein sequences that have a variety of fiber diameters and gelation rates that may be used for various biomedical applications depending on the required mechanical properties. For instance, faster gelling hydrogels are considered to be clinically useful for in situ gelation for drug and or gene delivery, while slower gelling systems have been shown to be applicable in tissue engineering due to their potential to maintain cell viability and homogeneity throughout the matrix.

A subset of coiled-coil fibers was used to develop a linear relationship to the electrostatic potential of different termini depending on their relationship to the N- or C-terminal ends of the protein, additional variants will establish both an improved machine learning model for prediction of fiber diameter and hydrogel mechanical properties (i.e. storage modulus, crosslinking density, critical time to gelation) as well as the use of other artificial computational inputs such as Rosetta Score, hydrophobicity scores and others, to amplify the models that result from the use of the ΔEE_{bcf} parameter.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various embodiments of the present disclosure.
Statement 1. A protein or peptide having or comprising the following sequence:

(SEQ ID NO: 1)
VX₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X
₂₄X₂₅X₂₆X₂₇X₂₈X₂₉X₃₀X₃₁X₃₂X₃₃X₃₄X₃₅X₃₆X₃₇,

where
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R, or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₆ is A, E, or Q;
X₁₇ is A, L, M, I, V, or L_{TF};
X₁₈ is A, K, E, D, K, R, H, N, or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇, or any L is replaced with a trifluoroleucine residue.
Statement 2. A protein or peptide according to Statement 1, where one, some, or all L residues and/or X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ residues are replaced with a trifluoroleucine residue.
Statement 3. A protein or peptide according to Statement 1 or Statement 2, wherein the sequence is or comprises:

(SEQ ID NO: 3)
VKELLFLKKTAEQMLEELKETNKALHDVRHLLENQSKL;
(SEQ ID NO: 4)
VKEILFLKNTAYQMLLELKETNEALYDIRHLLQQQSKL;
(SEQ ID NO: 5)
VKEITFIKKTIEQIAEEMKEINKAIHDIRHALENISKK;
(SEQ ID NO: 6)
VKEQTFIKNTIEQMAEEIKEINKAIHDIRHQLENMSKQ;
(SEQ ID NO: 7)
VKEITFIKKQIEQIAEEMKEINKAIHDIRHALENISKK;
(SEQ ID NO: 8)
VKEMTFIKKTIEQQAEEMKEINKAIHDIRHALENISKK;
(SEQ ID NO: 9)
VSEITEIKKTIEHIAKEMKEINKAIHTIRHALENIAKN;
or
(SEQ ID NO: 10)
VKEIKFLKNTAPQQLRELQNTNMALQDVRELLQQQSTL,

or a sequence having at least 75-99% identity to any one of the foregoing sequences, where one, some, or all the leucine residues of each sequence is replaced with a trifluoroleucine residue.
Statement 4. A protein or peptide according to any one of the preceding Statements, where the protein or peptide has the following sequence:

(SEQ ID NO: 3)
VKELLFLKKTAEQMLEELKETNKALHDVRHLLENQSKL;
(SEQ ID NO: 4)
VKEILFLKNTAYQMLLELKETNEALYDIRHLLQQQSKL;
(SEQ ID NO: 5)
VKEITFIKKTIEQIAEEMKEINKAIHDIRHALENISKK;
(SEQ ID NO: 6)
VKEQTFIKNTIEQMAEEIKEINKAIHDIRHQLENMSKQ;
(SEQ ID NO: 7)
VKEITFIKKQIEQIAEEMKEINKAIHDIRHALENISKK;
(SEQ ID NO: 8)
VKEMTFIKKTIEQQAEEMKEINKAIHDIRHALENISKK;
(SEQ ID NO: 9)
VSEITEIKKTIEHIAKEMKEINKAIHTIRHALENIAKN;
or
(SEQ ID NO: 10)
VKEIKFLKNTAPQQLRELQNTNMALQDVRELLQQQSTL,

wherein at least one leucine residue is replaced with a trifluoroleucine residue.
Statement 5. A protein or peptide according to any one of Statements 1-4, where the protein or peptide has the following structure:
VKELLFLKKTAEQMLEELKETNKALHDVRHLLENQSKL (SEQ ID NO:3), wherein 2, 3, 4, 5, 6, 7, 8, or 9 of the leucine residues are replaced with trifluoroleucine residues.
Statement 6. A protein or peptide according to any one of Statements 1-4, wherein the protein or peptide has the following structure:
VKEILFLKNTAYQMLLELKETNEALYDIRHLLQQQSKL (SEQ ID NO:4), wherein 2, 3, 4, 5, 6, 7, or 8 of the leucine residues are replaced with trifluoroleucine residues.
Statement 7. A protein or peptide according to any one of Statements 1-4, wherein the protein or peptide has the following structure:
VKEITFIKKTIEQIAEEMKEINKAIHDIRHALENISKK (SEQ ID NO:5), wherein the leucine residue is replaced with a trifluoroleucine residue.
Statement 8. A protein or peptide according to any one of Statements 1-4, wherein the protein or peptide has the following structure:
VKEQTFIKNTIEQMAEEIKEINKAIHDIRHQLENMSKQ (SEQ ID NO:6), wherein the leucine residue is replaced with a trifluoroleucine residue.
Statement 9. A protein or peptide according to any one of Statements 1-4, wherein the protein or peptide has the following structure:
VKEITFIKKQIEQIAEEMKEINKAIHDIRHALENISKK (SEQ ID NO:7), wherein the leucine residue is replaced with a trifluoroleucine residue.
Statement 10. A protein or peptide according to any one of Statements 1-4, wherein the protein or peptide has the following structure:
VKEMTFIKKTIEQQAEEMKEINKAIHDIRHALENISKK SEQ ID NO:8), wherein the leucine residue is replaced with a trifluoroleucine residue.
Statement 11. A protein or peptide according to any one of Statements 1-4, wherein the protein or peptide has the following structure:
VSEITEIKKTIEHIAKEMKEINKAIHTIRHALENIAKN (SEQ ID NO:9), wherein the leucine residue is replaced with a trifluoroleucine residue.
Statement 12. A protein or peptide according to any one of Statements 1-4, wherein the protein or peptide has the following structure:
VKEIKFLKNTAPQQLRELQNTNMALQDVRELLQQQSTL (SEQ ID NO:10), wherein 2, 3, 4, 5, 6, or 7 of the leucine residues are replaced with trifluoroleucine residues.
Statement 13. A protein or peptide according to any one the preceding Statements, wherein all the leucine residues of the protein or peptide is replaced with trifluoroleucine.
Statement 14. A protein or peptide having or comprising the following sequence:

(SEQ ID NO: 26)
MRGSHHHHHHGSIEGRVX₁X₂X₃X₄X₅X₆X₇X₈X₉X₁₀X₁₁X₁₂X₁₃X₁₄X₁₅X₁₆
X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇

where
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R, or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₆ is A, E, or Q;
X₁₇ is A, L, M, I, V, or L_{TF};
X₁₈ is A, K, E, D, K, R, H, N, or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
where at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ or any L is replaced with a trifluoroleucine residue.
Statement 15. A protein or peptide according to any one of Statements 1-3, wherein the protein or peptide has the following sequence or comprises the following sequence:

(SEQ ID NO: 11)
MRGSHHHHHHGSIEGRVKELLFLKKTAEQMLEELKETNKALHDVRHLLE
NQSKL;
(SEQ ID NO: 12)
MRGSHHHHHHGSIEGRVKEILFLKNTAYQMLLELKETNEALYDIRHLLQ
QQSKL;
(SEQ ID NO: 13)
MRGSHHHHHHGSIEGRVKEITFIKKTIEQIAEEMKEINKAIHDIRHALE
NISKK;
(SEQ ID NO: 14)
MRGSHHHHHHGSIEGRVKEQTFIKNTIEQMAEEIKEINKAIHDIRHQLE
NMSKQ;
(SEQ ID NO: 15)
MRGSHHHHHHGSIEGRVKEITFIKKQIEQIAEEMKEINKAIHDIRHALE
NISKK;
(SEQ ID NO: 16)
MRGSHHHHHHGSIEGRVKEMTFIKKTIEQQAEEMKEINKAIHDIRHALE
NISKK;
(SEQ ID NO: 17)
MRGSHHHHHHGSIEGRVSEITEIKKTIEHIAKEMKEINKAIHTIRHALE
NIAKN;
or
(SEQ ID NO: 18)
MRGSHHHHHHGSIEGRVKEIKFLKNTAPQQLRELQNTNMALQDVRELLQ
QQSTL,

or a sequence having at least 75-99% identity to any one of the foregoing sequences, where one, some, or all the leucine residues of each sequence is replaced with a trifluoroleucine residue.
Statement 16. A protein or peptide according to any one of Statements 1-3 or 15, wherein the protein or peptide has the following structure:
MRGSHHHHHHGSIEGRVKELLFLKKTAEQMLEELKETNKALHDVRHLLENQSKL (SEQ ID NO:11), wherein 2, 3, 4, 5, 6, 7, 8, or 9 of the leucine residues are replaced with trifluoroleucine residues.
Statement 17. A protein or peptide according to any one of Statements 1-3 or 15, wherein the protein or peptide has the following structure:
MRGSHHHHHHGSIEGRVKEILFLKNTAYQMLLELKETNEALYDIRHLLQQQSKL (SEQ ID NO:12), wherein 2, 3, 4, 5, 6, 7, 8, or 9 of the leucine residues are replaced with trifluoroleucine residues.
Statement 18. A protein or peptide according to any one of Statements 1-3 or 15, wherein the protein or peptide has the following structure:
MRGSHHHHHHGSIEGRVKEITFIKKTIEQIAEEMKEINKAIHDIRHALENISKK (SEQ ID NO:13), wherein the leucine residue is replaced with a trifluoroleucine residue.
Statement 19. A protein or peptide according to any one of Statements 1-3 or 15, wherein the protein or peptide has the following structure:
MRGSHHHHHHGSIEGRVKEQTFIKNTIEQMAEEIKEINKAIHDIRHQLENMSKQ (SEQ ID NO:14), wherein the leucine residue is replaced with a trifluoroleucine residue.
Statement 20. A protein or peptide according to any one of Statements 1-3 or 15, wherein the protein or peptide has the following structure:
MRGSHHHHHHGSIEGRVKEITFIKKQIEQIAEEMKEINKAIHDIRHALENISKK (SEQ ID NO:15), wherein the leucine residue is replaced with a trifluoroleucine residue.
Statement 21. A protein or peptide according to any one of Statements 1-3 or 15, wherein the protein or peptide has the following structure:
MRGSHHHHHHGSIEGRVKEMTFIKKTIEQQAEEMKEINKAIHDIRHALENISKK (SEQ ID NO:16), wherein the leucine residue is replaced with a trifluoroleucine residue.
Statement 22. A protein or peptide according to any one of Statements 1-3 or 15, wherein the protein or peptide has the following structure:
MRGSHHHHHHGSIEGRVSEITEIKKTIEHIAKEMKEINKAIHTIRHALENIAKN (SEQ ID NO:17), wherein the leucine residue is replaced with a trifluoroleucine residue.
Statement 23. A protein or peptide according to any one of Statements 1-3 or 15, wherein the protein or peptide has the following structure:
MRGSHHHHHHGSIEGRVKEIKFLKNTAPQQLRELQNTNMALQDVRELLQQQSTL (SEQ ID NO:18), wherein 2, 3, 4, 5, 6, 7, 8, or 9 of the leucine residues are replaced with trifluoroleucine residues.
Statement 24. A protein or peptide fiber comprising one or more protofibers comprising one or more proteins or peptides according to any one of the preceding Statements.
Statement 25. A protein or peptide fiber according to Statement 24, wherein one or more compounds are bound to the protein or peptide fiber.
Statement 26. A protein or peptide fiber according to Statement 24, wherein the one or more compounds are hydrophobic.
Statement 27. A protein or peptide fiber according to Statement 25 or Statement 26, wherein the one or more compounds are dyes, antibiotics, alkaloids, lipids, fatty acids, sugars, amino acids, phenolic compounds, extracellular materials (e.g., proteins, cells, exosomes, and the like, and combinations thereof), metals, nucleic acids, and the like, and combinations thereof.
Statement 28. A protein or peptide fiber according to any one of Statements 24-27, wherein the protein or peptide fiber has a fiber diameter of about 20 nm to about 2 μm.
Statement 29. A protein or peptide fiber according to any one of Statements 24-28, wherein the protein or peptide fiber has a coiled-coil morphology defining a pore along the length of the protein or peptide fiber.
Statement 30. A composition comprising a plurality of protein or peptide fibers according to any one of Statements 24-29.
Statement 31. A composition according to Statement 30, wherein the composition is formulated as a gel.
Statement 32. A method for detecting the location of a compound in an individual, wherein the individual has been administered one or more of the compounds bound to one or more protein or peptide fibers according to any one of Statements 24-29 or a composition of
Statement 31 or Statement 32, wherein the composition comprises the compound, comprising detecting a signal of the protein or peptide fibers, wherein the location of the compound is determined by the detection of the signal of the one or more protein or peptide fibers.
Statement 33. A method according to Statement 32, wherein the signal is detected via MRI.
Statement 34. A method according to Statement 33, wherein the MRI is ¹⁹F MR spectroscopy.
Statement 35. A method according to Statement 33, wherein the MRI is ¹H MR spectroscopy.
Statement 36. A method according to Statement 32, wherein the signal is detected via ultrasound.
Statement 37. A method according to Statement 36, wherein ultrasound is ¹⁹F MR spectroscopy.
Statement 38. A method according to any one of Statements 32-37, wherein the compound is a therapeutic agent.
Statement 39. A method according to Statement 38, wherein the therapeutic agent is an exosome.
Statement 40. A method according to any one of Statements 32-39, wherein the individual has a wound associated with diabetes, burns, venous disease, pressure ulcers, age-associated complications, or any disorder that compromises the barrier function and integrity of skin.
Statement 41. A method according to any one of Statements 32-37, wherein the individual has cancer.
Statement 42. A method for determining an optimal sequence of a protein to achieve a desired diameter of a fiber formed by the self-assembled protein, comprising:
   determining ΔEE_{bcf} of a primary structure of a protein;
   determining a stability score of the primary structure; and
   using a computational algorithm to optimize a variant structure of the protein by substituting one or more of the solvent-exposed residues of the primary structure to achieve a desired ΔEE_{bcf}, while maintaining or increasing the stability score for the varied structure.
Statement 43. A method according to Statement 42, wherein the computational algorithm is a machine learning algorithm.
Statement 44. A method according to Statement 42, wherein the computational algorithm is a Monte Carlo simulation.
Statement 45. A method according to any one of Statements 42-44, wherein ΔEE_{bcf} is determined by:

$$Δ E {E}_{bcf} = {\sum }_{n = l / 2}^{l} E {E}_{bcf} - {\sum }_{n = 1 + ( tag length )}^{\frac{l}{2} - 1} {EE}_{bcf} ,$$

where n is the sequence position number, l is the length of the sequence, and EE_{bcf} is the electrostatic potential energy of a residue in the b, c, or f helical wheel position.
Statement 46. A method according to any one of Statements 42-45, wherein the desired ΔEE_{bcf} is a maximum ΔEE_{bcf} (i.e., maximizing ΔEE_{bcf}).
Statement 47. A method according to any one of Statements 42-46, wherein the desired ΔEE_{bcf} is a minimum ΔEE_{bcf} (i.e., minimizing ΔEE_{bcf}).
Statement 48. A method according to any one of Statements 42-47, wherein the desired ΔEE_{bcf} is determined based on the determined ΔEE_{bcf} of the primary structure and a desired diameter of a fiber formed by the self-assembled protein.
Statement 49. A protein or peptide having a sequence determined by a method according to any one of Statements 42-47.
Statement 50. A protein or peptide having a sequence or comprising a sequence of the present disclosure or a protein or peptide having at least 75% (e.g., at least 80%, at least 85%, at least 90, or 95%) homology thereto.
Statement 51. A composition comprising a protein or peptide according to Statement 49 or
Statement 50.
Statement 52. A composition according to Statement 51, wherein the composition comprises a supramolecular structure comprising the protein or peptide according to Statement 49 or
Statement 50.
Statement 53. A composition according to Statement 52, wherein the supramolecular structure is a fiber.
Statement 54. A composition according to any one of Statements 51-53, wherein the supramolecular structure further comprises a therapeutic (e.g., a drug, such as, for example, a small molecule drug).
Statement 55. A gel comprising a protein or peptide of the present disclosure.
Statement 56. A gel comprising a protein or peptide having a sequence determined by a method according to any one of Statements 42-47.
Statement 57. A method for determining an optimal sequence of a protein to achieve a desired diameter of a fiber formed by the self-assembled protein, comprising:
   determining ΔEE_{bcf} of a primary structure of a protein;
   determining a stability score of the primary structure; and
   substituting one or more solvent-exposed residues of the primary structure to achieve a desired ΔEE_{bcf}, while maintaining or increasing the stability score for the varied structure, to optimize a variant structure of the protein.
Statement 58. A method according to Statement 57, wherein substituting one or more solvent-exposed residues of the primary structure to achieve a desired ΔEE_{bcf}, while maintaining or increasing the stability score for the varied structure, is performed using machine learning algorithm.
Statement 59. A method according to Statement 57, wherein substituting one or more solvent-exposed residues of the primary structure to achieve a desired ΔEE_{bcf}, while maintaining or increasing the stability score for the varied structure, is performed using a Monte Carlo simulation.
Statement 60. A method according to any one of Statements 57-59, wherein ΔEE_{bcf} is determined by:

$$Δ E {E}_{bcf} = {\sum }_{n = l / 2}^{l} E {E}_{bcf} - {\sum }_{n = 1 + ( tag length )}^{\frac{l}{2} - 1} {EE}_{bcf} ,$$

where n is the sequence position number, l is the length of the sequence, and EE_{bcf} is the electrostatic potential energy of a residue in the b, c, or f helical wheel position.
Statement 61. A method according to any one of Statements 57-60, wherein the desired ΔEE_{bcf} is a maximum ΔEE_{bcf} (i.e., maximizing ΔEE_{bcf}).
Statement 62. A method according to any one of Statements 57-61, wherein the desired ΔEE_{bcf} is a minimum ΔEE_{bcf} (i.e., minimizing ΔEE_{bcf}).
Statement 63. A method according to any one of Statements 57-62, wherein the desired ΔEE_{bcf} is determined based on the determined ΔEE_{bcf} of the primary structure and a desired diameter of a fiber formed by the self-assembled protein.
Statement 64. A protein or peptide having a sequence determined by a method according to any one of Statements 57-63.

The following examples are presented to illustrate the present disclosure. They are not intended to be limiting in any matter.

### Example 1

This example provides a description of the methods and peptides/proteins of the present disclosure.

A parameter for electrostatic potential energy difference between the termini has been designed and is described herein. The electrostatic potential energy difference between the termini was calculated using Equation 1, where n is the sequence position number, l is the length of the sequence, EE_{bcf} is the electrostatic potential energy of a residue if it is in the b, c, or f helical wheel position.

$$Δ {EE}_{bcf} = {\sum }_{n = l / 2}^{l} E {E}_{bcf} - {\sum }_{n = 17}^{\frac{l}{2} - 1} E {E}_{bcf} \qquad ( Equation 1 )$$

In the case of coiled coils, a 16 residue tag begins the sequence including a hexahistidine sequence for purification. Thus, this is considered a constant in the coiled-coil relationship that may or may not exist in other sequences depending on those residues that would exist in the coiled-coil domain. Thus, residues existing in the b, c, and f helical wheel positions were counted starting at residue 17. This may be different for different coiled-coiled sequences depending on the composition or absence of a tag. The same logic applies to tags on the C-terminus of the protein. The electrostatic potential of an atom in a residue is then summed together with all other residues existing in the b, c, and f helical wheel positions up to the halfway point of the coiled-coil domain. This is repeated for the latter half of the coiled-coil domain where the electrostatic potential difference between the termini is calculated as the difference of these electrostatic potential sums.

This relationship was validated by synthesizing all protein under the same conditions. Protein fibers are purified under denaturing conditions using 50 mM tris hydrochloride, pH 8, and 500 mM NaCl, 6 M urea and then dialyzed into 5 L buckets of 50 mM NaH₂PO₄ pH 4.0 buffer with 3M urea, 1.5 M urea, 0.75 M urea, and then six 0 M urea. Protein hydrogels are purified under native conditions using 50 mM tris hydrochloride, pH 8, and 500 mM NaCl, and then dialyzed into six 5 L buckets of 50 mM tris hydrochloride, pH 8, and 500 mM NaCl. Protein purified for gelation is concentrated in 3 kDa centrifugal filters to a concentration of 2 mM, where the protein self-assembles to form a hydrogel at 4° C. Interestingly, these hydrogels demonstrate a UCST (upper critical solution temperature) type behavior, exhibiting gel-sol transition above a UCST (˜16° C. for Q).

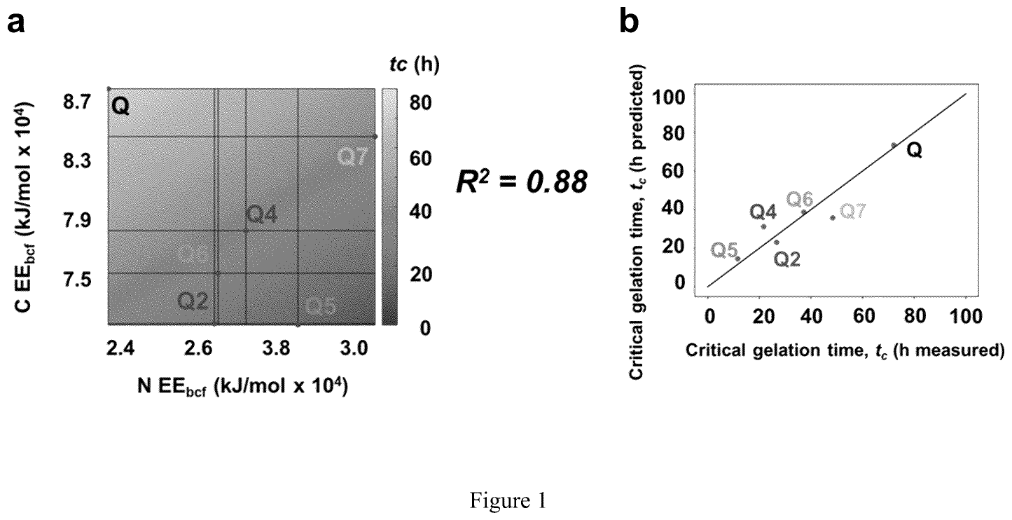

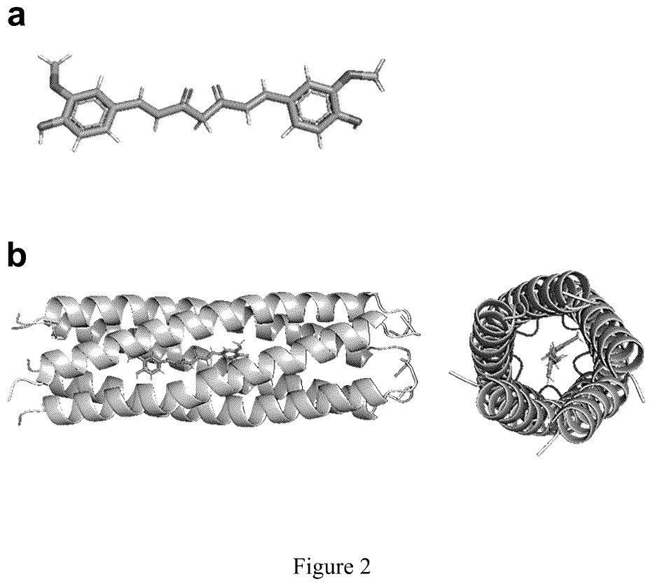

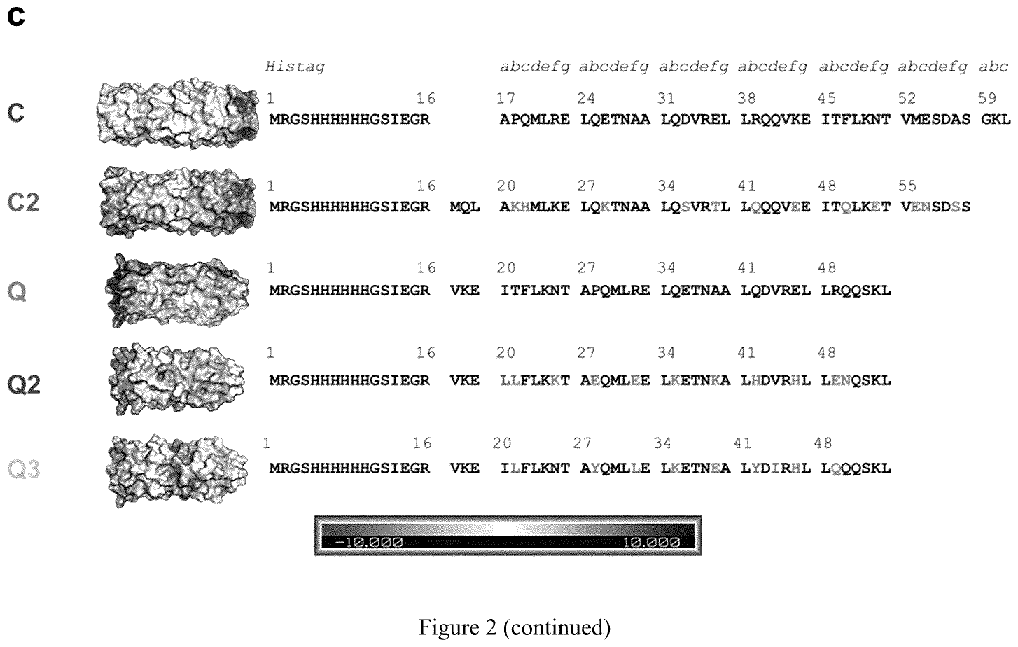

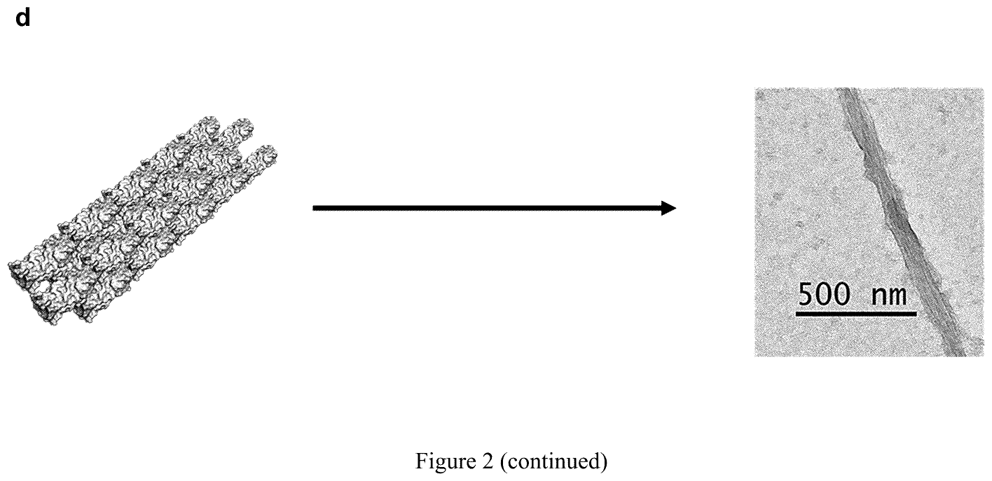

A mini-library was designed with a breadth of electrostatic distributions while retaining a positively and negatively charged patch on the N- and C-termini, respectively. Previously studied protein sequences C and Q were also used in the library (FIG. 1).

First Q2 was designed to undergo reduced lateral assembly. The protonation and deprotonation of solvent-exposed residues is believed to be a key factor affecting the self-assembly of Q into fibers. Using Rosetta, charge in the solvent exposed b, c, and f positions was modified to: 1) redistribute the surface to be more negative and lower the pI; 2) lower the overall electrostatic potential energy (EE); and 3) improve the Rosetta stability score. A more evenly distributed negative surface on the C-terminus was hypothesized to push the pI to be more neutral and balance the positive and negative surface patches resulting from the largely positive patch constituted by the N-terminus and His tag. Recent studies on electrostatically interacting coiled-coils have shown a strong correlation between the gelation kinetics and overall charge of the protein where faster gelation and an increased storage modulus is seen at pH near the pI of the protein. A lower overall U_{E} is hypothesized to preferentially decrease the lateral interaction of neighboring coiled-coils since end-to-end protofibril stacking is conserved from C-derived coiled-coil fibers. Mutations were rationally chosen and iteratively checked using the Rosetta relax score functions and APBS electrostatic potential energy (U_{E}) until these goals are achieved. Complementary to redistribution of the surface charge, hydrophobic residues necessary for helix and coiled-coil formation are maintained throughout the surface charge optimization. A single point mutation, I20L, is then made in the hydrophobic pore to further stabilize it from changes in temperature for future studies. A final design of Q2 exhibits a more neutral pI of 8.2 compared to 9.7 for Q.

Two additional sequences have been conceived to elucidate the relationship to electrostatic, self-assembly, and CCM binding studied here—one based on the Q domain, and another based on the C domain (to establish that redistribution and self-assembly might be controlled for pentamers not based on the Q domain). Similar criteria to the design of Q2 has been used to generate sequences with electrostatic diversity on the surface where mutations are constricted to the solvent exposed b, c, and f helical wheel positions using Rosetta. C2 is generated by redistribution of the electrostatic patches of C such that a more negatively charged patch is represented on the surface of the C-terminus in the electrostatic map and a more positively charged patch is represented on the surface of the N-terminus in the electrostatic map. Similarly, Q3 is designed to redistribute the electrostatic potential into a unique electrostatic potential map and this time with a preferred hydrophobic pore mutation V44I as determined by Rosetta. Both variants have been designed by iterative mutations that were rationally chosen and checked for an equal or improved stability using the Rosetta relax, score functions and APBS electrostatic potential maps. The resulting Rosetta scores are −760 kcal/mol for C, −777 kcal/mol for C2, −610 kcal/mol for Q, −637 kcal/mol for Q2, and −624 kcal/mol for Q3.

We demonstrate that these protein variants of Q (or COMPcc) form fibers of various sizes or hydrogels with various mechanical properties (with critical gelation time being specifically explored here).

It was shown that with increasing ΔEE_{bcf} the fibers exhibit increasing diameters. Using this relationship under a multi-state Monte-Carlo simulation with the goal to increase ΔEE_{bcf} while maintaining or increasing Rosetta score using the Rosetta macromolecular modeling suite to provide mutations to the Q domain, the output sequence, Q9, was created that we subsequently synthesized using the same method for the previous fibers. Electron micrographs reveal that Q9 possessed fiber diameters averaging 760±170 nm with the largest population of sizes averaging 1280±330 nm. It is believed that this proves that a Monte-Carlo search incorporating the use of ΔEE_{bcf} provides as a method for designing and searching a coiled-coil sequence space for different desired fiber diameters. The use of the ΔEE_{bcf} as a parameter to optimize these coiled-coils towards larger fiber diameter was validated with the design of Q9.

Two additional designs were characterized for gelation by computationally altering residues in the a, d, e, and g helical wheel positions of Q2 so as to maintain its solvent exposed surface and assess the impact of the hydrophobic pore on gelation and determine the difference in gelation properties of sequences with the same solvent exposed residues. These designs were mutated using a Monte-Carlo search in Rosetta for 1000 iterations each where the sequence with the highest Rosetta score was selected. This simulation was run sixty times and evaluated for the highest probability of each residue in each position. This method was performed twice to the result of two new sequences, Q5 and Q6. Q based sequences were then evaluated under native purification conditions for gelation. Using multivariate linear regression of the N- and C-terminal EE_{bcf} a clear optimum for improved gelation is exhibited (FIG. 1) providing a strong R²=0.72 relationship. The resulting model can be used to design sequences with specific rates of gelation based on any coiled-coil protein sequence dependent on the ΔEE_{bcf} (FIG. 1). This model may also be established for hydrogel crosslinking density, storage modulus, and other correlated self-assembly properties.

### Example 2

The following provides examples of peptides of the present disclosure.

The ability to engineer the solvent-exposed surface of self-assembling coiled-coils allows one to achieve higher order hierarchical assembly such as nano- or microfibers. Currently these materials are being developed for a range of biomedical applications, including drug delivery systems, however ways to mechanistically optimize the coiled-coil structure for drug binding has yet to be explored. The functional properties of the naturally occurring cartilage oligomeric matrix protein coiled-coil (C) have been leveraged for its favorable motif and for the presence of a hydrophobic pore to allow for small molecule binding. This includes the development of Q, a rationally designed pentameric coiled-coil derived from C. Described herein is a small library of protein microfibers derived from the parent sequences of C and Q bearing various electrostatic potentials, with the aim to investigate the influence of higher order assembly and encapsulation of candidate small molecule, curcumin. The supramolecular fiber size appears to be well-controlled by sequence imbued electrostatic surface potential and protein stability upon curcumin binding is well correlated to relative structure loss, which can be predicted by in silico docking.

A subset of three new coiled-coil protein variants were defined for the study of protein nanofiber assemblies. The new protein variants were compared to C and Q for their ability to self-assemble. The relationship between the protein electrostatic patches of the coiled-coils while maintaining the ability to bind curcumin and undergo higher order assembly into the mesoscale were elucidated. It was found that the electrostatic potential of surface-facing residues in the coiled-coils have a direct correlation to the diameters of the supramolecular assembling fibers. Upon binding to curcumin, these protein fibers increase in diameter proportionally to their original fiber diameters without curcumin—becoming microns in diameters—and increase in thermostability proportional to their relative loss of structure. Utilizing a combination of computational protein modeling and structural characterization we further define the relationship between coiled-coil electrostatics and supramolecular assembly.

Materials. Chemically competent M15MA E. coli cells were gifted from David Tirrell at California Institute of Technology. Bacto-tryptone, sodium chloride (NaCl), yeast extract, tryptic soy agar (TSA), ampicillin sodium salt, sodium phosphate dibasic anhydrous (Na₂HPO₄), sodium hydroxide (NaOH), urea, dextrose monohydrate (_{D}-glucose), magnesium sulfate (MgSO₄), calcium chloride (CaCl₂), manganese chloride tetrahydrate (MnCl₂·4H₂O), cobaltous chloride hexahydrate (CoCl₂·6H₂O), isopropyl R-_{D}-1-thiogalactopyranoside (IPTG), Pierce bicinchoninic acid (BCA) assay kit, Pierce snakeskin dialysis tubing 3.5 K molecular weight cutoff (MWCO), sodium dodecyl sulfate (SDS), Nunc ninety-six well plates, and BD Clay Adams glass microscopy slides were acquired from Thermo Fisher Scientific. The twenty naturally occurring amino acids, dimethylsulfoxide (DMSO), nickel (III) chloride hexahydrate (NiCl₂·6H₂O), sodium molybdate dihydrate (Na₂MoO₄·2H₂O), iron (III) chloride (FeCl₃), iron (II) chloride tetrahydrate (FeCl₂-4H₂O), thiamine hydrochloride (vitamin B), curcumin, and copper (II) sulfate pentahydrate (CuSO₄·5H₂O) were purchased from Sigma Aldrich. Hydrochloric acid (HCl), Coomassie® Brilliant Blue G-250 were purchased from VWR. HiTrap FF 5 mL columns for protein purification were purchased from Cytiva Life Sciences. Macrosep and Microsep Advance Centrifugal Devices 3K MWCO and 0.2 μm syringe filters were purchased from PALL. Acrylamide/bis solution (30%) 29:1, and natural polypeptide sodium dodecyl sulfate-polyacrylamide gel electrophoresis (SDS-PAGE) standard were purchased from Bio-Rad. Copper (II) chloride anhydrous (CuCl₂), sodium selenite (Na₂SeO₃), and imidazole were purchased from Acros Organics. Formvar/carbon-coated copper grids (FCF400-Cu) and 1% uranyl acetate for transmission electron microscopy were purchased from Electron Microscopy Sciences.

Computational Modeling and Docking of Protein and Curcumin. To assess the iterative effect of mutations on the stability of a coiled-coil protein variant, and perform small-molecule docking simulations, Rosetta suite of macromolecular modeling tools (Version 3.5) was used. Rosetta Relax protocol was used on protein sequences using the symmetry of COMPcc (PDB: 3V2P) with the all-atom energy score function. To dock curcumin (CCM) into the pore of a protein fiber variant, CCM conformer libraries were generated using the BioChemical Library BCL::Conformer Generation application. Docking was performed in Rosetta using the GALigand Docking protocol in virtual high-screening mode (VSH) to allow sampling of both the receptor and ligand conformational space. To accommodate the N- and C-terminal possible sites of the long and narrow cavity of the protein coiled-coil, CCM was docked with starting positions inside the first N-terminal half of the coiled-coil and in the second C-terminal half of the coiled-coil. 500 models were generated from each starting conformation for a total of 1000 models, the best of which was used for analysis. Protein structures were visualized using PyMOL. Spatial aggregation propensity (Sap) score was calculated in Rosetta with SapScoreMetric.

Computational Electrostatics Calculation. PDB2PQR software (version 3.1.0) was used to set up titration states at room temperature with the amber forcefield and propka pH calculation method. Electrostatic maps and electrostatic potential energies were calculated using Adaptive Poisson-Boltzmann Solver (APBS) (Version 3.0.0) from subsequent PDB2PQR input files. In calculation of Rosetta score and electrostatic potential energy, the conserved His tag (sequence position 1-17) is negated due to Rosetta's inability to define a consistent minimum energy state. The electrostatic potential energy difference between the termini was calculated using Equation 2, where n is the sequence position number, l is the length of the sequence, EE_{bcf} is the electrostatic potential energy of a residue if it is in the b, c, or f helical wheel position.

$$Δ {EE}_{bcf} = {\sum }_{n = l / 2}^{l} E {E}_{bcf} - {\sum }_{n = 17}^{\frac{l}{2} - 1} E {E}_{bcf} \qquad ( Equation 2 )$$

Expression and Purification. C, C2, Q, Q2, and Q3 were all expressed and purified using the same protocol as follows. New variants plasmids (C2, Q2, and Q3) were cloned and purchased from Genscript in PQE60 vector. Cloned plasmids were transformed into chemically competent methionine auxotrophic M15MA E. coli cells using heat shock. The transformed cells were grown for 16 hrs at 37° C. on TSA plates supplemented with 200 g/mL ampicillin and 35 μg/mL kanamycin. Single colonies from the plate were used to inoculate 16 mL of supplemented M9 minimal media (0.5 M Na₂HPO₄, 0.22 M KH₂PO₄, 0.08 M NaCl, and 0.18 M NH₄Cl) containing all 20 natural amino acids (100 μg/mL), ampicillin (200 μg/mL), kanamyacin (35 μg/mL), vitamin B (35 μg/mL), D-glucose (100 μg/mL), magnesium sulfate (1 mM), calcium chloride (0.1 mM). Starter cultures were incubated at 37° C. and 350 rpm for 16 hours. All 16 mL of starter culture was used to inoculate 400 mL of supplemented M9 minimal media, which was then incubated at 37° C. and 350 rpm. Expression of each protein was induced with addition of 200 μg/mL IPTG once the optical density (OD₆₀₀) reached ˜0.8. Following 3 hrs of expression, cells were harvested by centrifuging for 30 min at 4000×g at 4° C. in an Avanti J-25 centrifuge (Beckman Coulter) where resulting pellets were stored at −20° C. until purification.

All proteins were purified under denaturing conditions and pellets were resuspended in buffer A (50 mM NaH₂PO₄, 6 M urea, pH 8.0). Lysis was performed using a Q500 sonicator at 50% amplitude and 5 seconds (s) on and 5 s off pulse sequence for a total time of 2.5 min. Cell lysates were then centrifuged at 14,000×g for 50 min at 4° C. to remove cell debris. The supernatant was purified using a syringe-pump driven IMAC Sepharose high performance 5 mL column (HiTrap Q HP 5, Cytvia) charge with CoCl₂. The protein was eluted using an increasing concentration of buffer B (50 mM NaH₂PO₄, 6 M urea, 500 mM imidazole, pH 8.0) with buffer A ranging from 0 to 100% v/v. Elutions were collected and run on a 12% SDS-PAGE. Pure elutions were removed and dialyzed using 3.5 kDa MWCO tubing at 4° C. in κ L volumes using stepwise concentrations of 3 M, 1.5 M, and 0.75 M urea in 50 mM NaH₂PO₄ at pH 4.0 followed by six 5 L volumes of buffer composed of 50 mM NaH₂PO₄ at pH 4.0. Protein was then concentrated to approximately 1.5 mL using 3 kDa MWCO Macrosep and Microsep Advance centrifugal devices (Pall Corporation) at 3,000×g. Protein concentration was then determined by a bicinchoninic acid (BCA) assay with a standard curve based on bovine serum albumin concentrations.

Circular Dichroism Spectroscopy. Secondary structure was first assessed using the Jasco J-815 CD spectrometer with a PTC-423S single position Peltier temperature control system. Wavelength scans were performed from 190 to 250 nm using 15 μM samples at 1 nm step sizes at 25° C. Temperature scans were performed at α-helical minimum 222 nm from 20 to 85° C. The mean residue ellipticity (MRE) was calculated as described previously. MRE at 222 nm and 208 nm and their ratios were used to assess relative helicity.

Attenuated Total Reflectance-Fourier Transform Infrared Spectroscopy. Secondary structure of protein fiber variants was confirmed using attenuated total reflectance-Fourier transform infrared (ATR-FTIR) spectroscopy using the Nicolet 6700 Fourier Transform Infrared Spectrometer equipped with a diamond ATR accessory and a mercury cadmium telluride (MCT)-A detector. Spectra were collected for 5 μL of 500 μM protein from 4000-400 cm⁻¹ with 0.5 cm⁻¹ increments at room temperature (RT). For CCM bound protein, protein was bound at a final concentration of 500 μM and saturated with CCM as determined by fluorescent spectroscopic drug binding with a final 1% v/v DMSO. Sample spectra were normalized using buffer background and analyzed from 1700-1600 cm⁻¹ corresponding to the amide I region. Peaks were deconvoluted using Gaussian functions in PeakFit software until the goodness of fit reached r²≥0.99. Peaks were associated with secondary structure were determined by methods known in the art.

Transmission Electron Microscopy. Transmission electron microscopy (TEM) images were taken with a FEI Talos L120C transmission electron microscope. Protein fiber samples were diluted to 50 μM and 3 μL was spotted on Formvar/carbon-coated copper grids followed by 5 μL wash with water, and 3 μL staining with 1% v/v uranyl acetate solution each with incubation times of 1 min at RT. Fibrils were sized in ImageJ software (Version 1.52q).

Drug Binding. Protein variants were bound to curcumin (CCM) in increasing CCM:protein ratios at 1:1 to 10:1. Protein was diluted to a final concentration of 15 μM before CCM solubilized in DMSO was added to the protein sample at a final 1% v/v DMSO. Samples were incubated on a thermomixer for 30 min at 300 rpm in the dark at 4° C. Samples were then removed and 300 μL of each sample was added to a 96-well solid black plate. Protein variants were excited at 420 nm and emission was read at 520 nm using a BioTek Synergy H1 microplate reader at RT. The baseline spectra of CCM, at 15-150 μM in 50 mM PB pH 4.0 containing 1% v/v DMSO, was subtracted from the fluorescence intensities of the protein-CCM at corresponding concentrations. Resulting normalized relative fluorescence intensities were analyzed using Specific binding kinetics in GraphPad Prism (GraphPad Software).

Confocal Microscopy. Protein bound to CCM was studied by measuring 50 μM protein samples saturated with CCM as determined by fluorescent spectroscopic drug binding with a final 1% v/v DMSO (or 2×K_{d}). 10 μL of protein was added to a micro slide and a 22×22 mm #1 microscope cover glass. Slides were inverted and imaged using a Leica TCS SP8 X Laser Confocal Microscope using a dry 10× objective at RT. CCM was excited using a 460 nm line of the laser, and images were taken using a 470 to 550 nm detection window.

Statistical Analysis. GraphPad Prism (GraphPad Software) was employed for statistical analysis using student's t-test.

Coiled-coil mini-library design. A mini-library was designed with a breadth of electrostatic distributions while retaining a positively and negatively charged patch on the N- and C-termini, respectively. Previously studied protein sequences C and Q were also used in the library.

First Q2 was designed to undergo reduced lateral assembly. The protonation and deprotonation of solvent-exposed residues is believed to be a key factor affecting the self-assembly of Q into fibers. Using Rosetta, charge in the solvent exposed b, c, and f positions was modified to: 1) redistribute the surface to be more negative and lower the pI; 2) lower the overall electrostatic potential energy (U_{E}); and 3) improve the Rosetta stability score. A more evenly distributed negative surface on the C-terminus was hypothesized to push the pI to be more neutral and balance the positive and negative surface patches resulting from the largely positive patch constituted by the N-terminus and His tag. Recent studies on electrostatically interacting coiled-coils have shown a strong correlation between the gelation kinetics and overall charge of the protein where faster gelation and an increased storage modulus is seen at pH near the pI of the protein. A lower overall U_{E} is hypothesized to preferentially decrease the lateral interaction of neighboring coiled-coils since end-to-end protofibril stacking is conserved from C-derived coiled-coil fibers. Mutations are rationally chosen and iteratively checked using the Rosetta relax score functions and APBS electrostatic potential energy (U_{E}) until these goals are achieved. Complementary to redistribution of the surface charge, hydrophobic residues necessary for helix and coiled-coil formation are maintained throughout the surface charge optimization. A single point mutation, I20L, is then made in the hydrophobic pore to further stabilize it from changes in temperature for future studies. A final design of Q2 exhibits a more neutral pI of 8.2 compared to 9.7 for Q.

Two additional sequences have been conceived to elucidate the relationship to electrostatic, self-assembly, and CCM binding studied here—one based on the Q domain, and another based on the C domain (to establish that redistribution and self-assembly might be controlled for pentamers not based on the Q domain). Similar criteria to the design of Q2 has been used to generate sequences with electrostatic diversity on the surface where mutations are constricted to the solvent exposed b, c, and f helical wheel positions using Rosetta. C2 is generated by redistribution of the electrostatic patches of C such that a more negatively charged patch is represented on the surface of the C-terminus in the electrostatic map and a more positively charged patch is represented on the surface of the N-terminus in the electrostatic map. Similarly, Q3 is designed to redistribute the electrostatic potential into a unique electrostatic potential map and this time with a preferred hydrophobic pore mutation V44I as determined by Rosetta. Both variants have been designed by iterative mutations that were rationally chosen and checked for an equal or improved stability using the Rosetta relax, score functions and APBS electrostatic potential maps. The resulting Rosetta scores are −760 kcal/mol for C, −777 kcal/mol for C2, −610 kcal/mol for Q, −637 kcal/mol for Q2, and −624 kcal/mol for Q3.

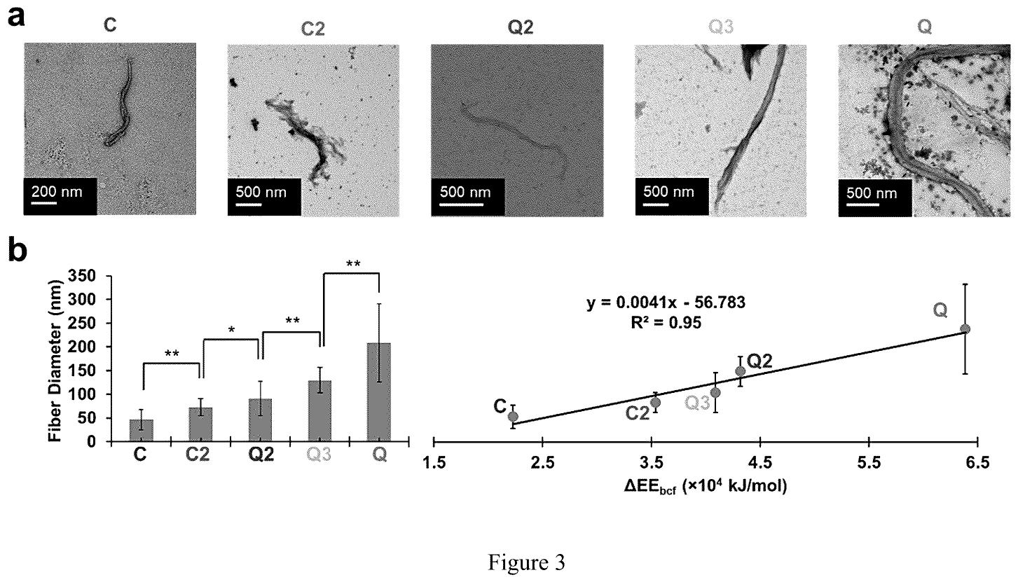

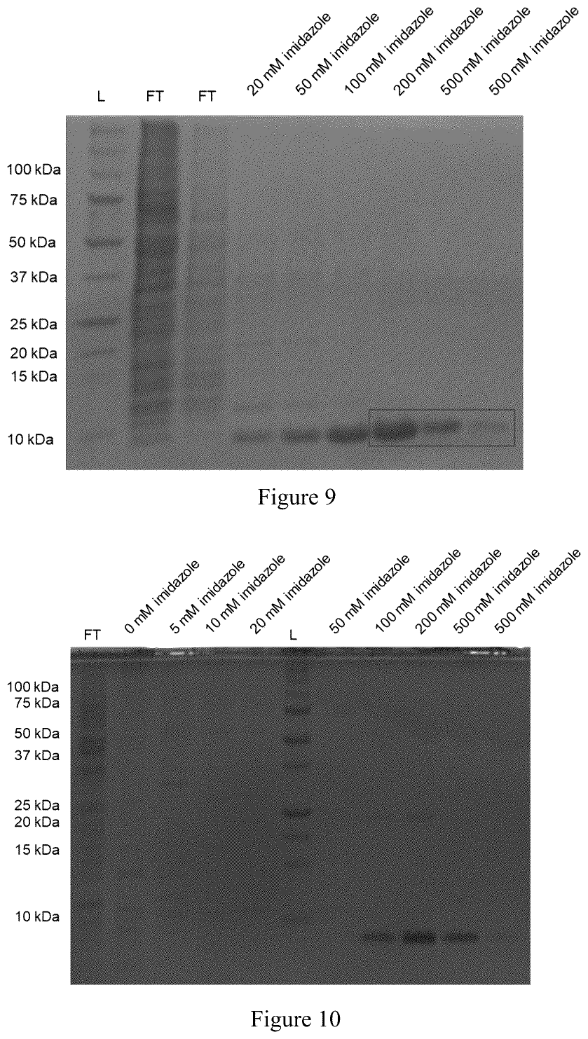

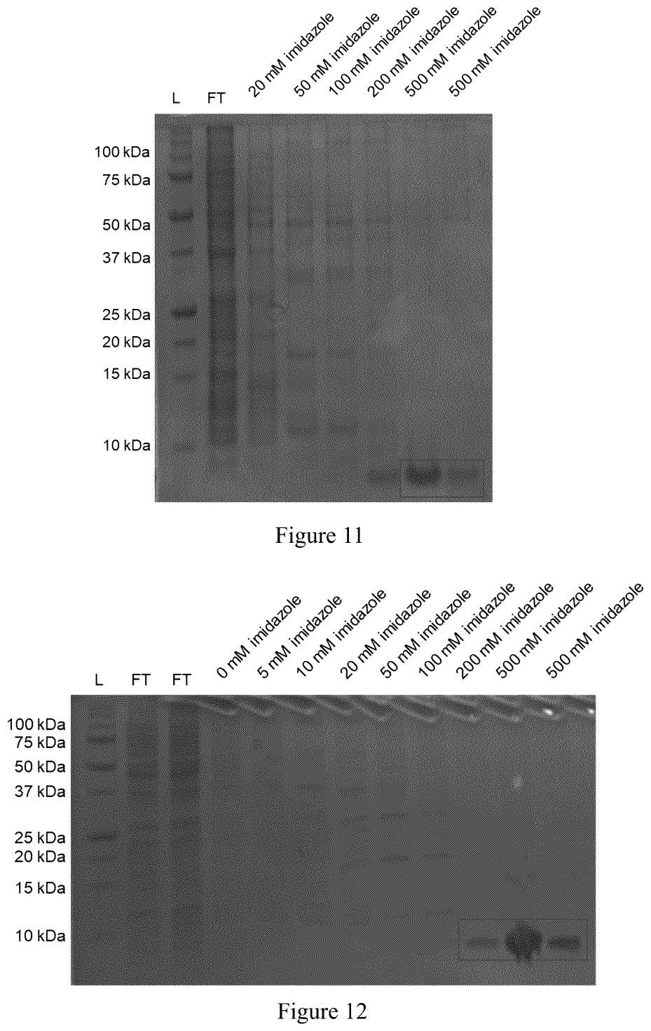

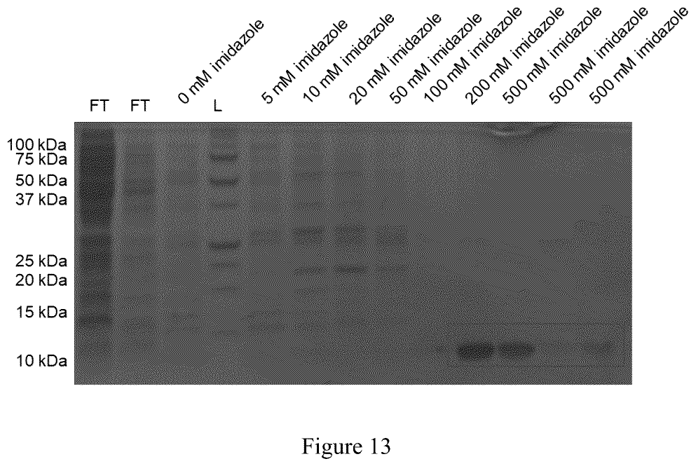

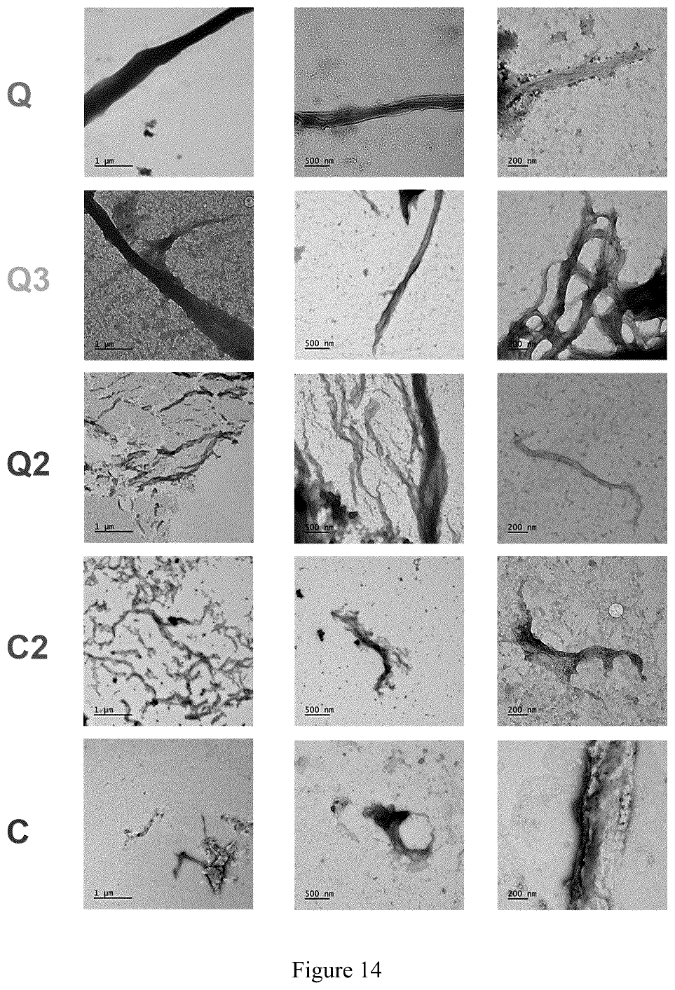

Electrostatics and fiber assembly. Protein variants were successfully purified (FIG. 9-13) and concentrated to approximately 1 mM in 50 mM NaH₂PO₄ buffer at pH 4.0. Proteins were first confirmed to maintain its ability to undergo supramolecular assembly by TEM (FIG. 3a and FIG. 14). All variants studied demonstrated the formation of nanoscale fibers. Average protein fiber diameters were ranked from smallest to largest with 47±22 nm for C, 73±18 nm for C2, 91±37 nm for Q2, 130±27 nm for Q3, and 208±82 nm for Q (FIG. 3b). By conventional criteria (p-value <0.05), protein fiber diameters were all calculated to be significantly different from each other by an unpaired t-test.

Because the large fiber assemblies seen previously in Q are the result of electrostatic coupling and the end-to-end alignment of coiled-coils, it was hypothesized that quantification of the electrostatic patches that comprise these fibers might be correlated to the ability to undergo supramolecular fiber assembly. Using Poisson-Boltzmann electrostatic potential calculated from APBS, the cumulative electrostatic potential energy difference was assessed of each terminus (split halfway in the sequence) calculated from the solvent exposed residues in the b, c, and f positions (ΔEE_{bcf}) as seen in Equation 2. The growth in diameter appears to also be increasing in a near exponential relationship. This difference is strongly correlated by a linear relationship (R²=0.95) with the average observed fiber diameters by TEM (FIG. 3b).

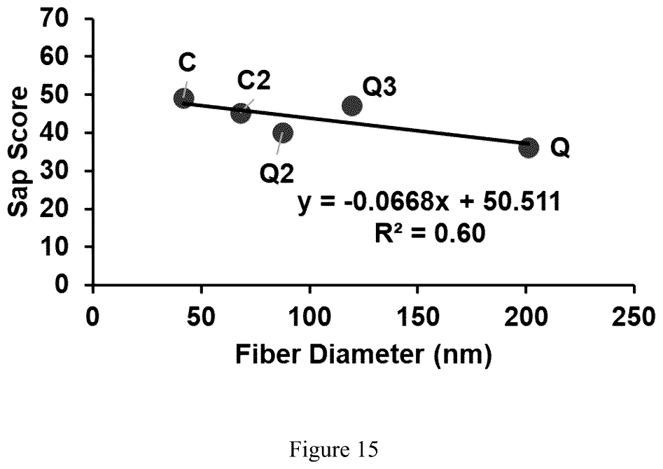

To confirm that the change in fiber diameter is not the result of a change in surface hydrophobicity, the Spatial aggregation propensity (Sap) score was used to calculate the relative aggregation between protein fiber variants of residues in the b, c, and f helical wheel positions. While the scores presented a slight negative correlation with fiber diameter (R²=0.60), it was significantly weaker than that from the ΔEE_{bcf} calculation noted above (FIG. 15), and the correlation is strongly influenced by the much lower Sap score exhibited by Q. Additionally, a higher Sap score is expected to correlate to increased aggregation contrary to the correlation. While increased fiber growth is thought to occur by electrostatic coupling, the reduction of hydrophobic coupling may also assist or allow for increased fiber growth. Overall, these results strengthen the conclusion that the supramolecular assemblies of the coiled-coil proteins of the present disclosure are driven by the electrostatic potential of positively and negatively charged patches in the fiber. Moreover, this shows that the morphology of the protein assemblies may be tuned by selective mutations to the solvent exposed surface of a coiled-coil.

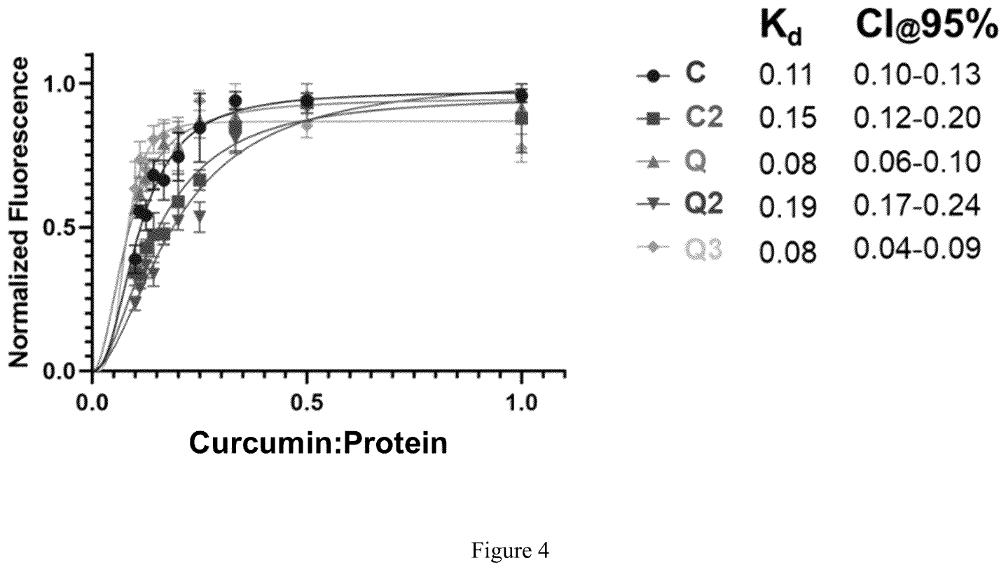

Curcumin binding and fiber thickening. Previously, C and Q have exhibited a promiscuous binding site capable of binding both hydrophilic and hydrophobic small molecules. Small molecule curcumin (CCM) to was bound to Q as a candidate molecule for drug delivery previously since the compound has been used therapeutically due to its antiproliferative, antibacterial, and anti-inflammatory properties. Conservation of this binding pocket in these protein fiber variants was confirmed by fluorescence spectroscopy (FIG. 4) where binding behavior are similar across all variants. Specific binding kinetics have been used to calculate the K_{d} of the fluorescence curves. Using 2×K_{d} to calculate the saturation of CCM binding, it was inferred that one CCM molecule binds approximately 3-5 protein monomers on average.

Curcumin has also previously been known to induce fiber thickening of protein fibers including collagen and Q using CD, surface tension and viscosity. The fiber thickening by CCM is driven by the interaction of negatively charged residues on the surface of the protein with CCM while it is fully protonated in a low pH system. It was previously noted that this interaction assists in solvating polar groups on the surface and burying hydrophobic residues. Furthermore, it was noted that CCM causes a local restructuring of water molecules leading to increased protein surface activity and exposure of nonpolar groups, which creates the driving force for protein-protein interaction or induced thickening of α-helical protein fibers in an effort to reduce surface energy. In studies of Q using increased concentrations of CCM, CD measurements show that helical packing is unaffected and confirming protein conformation is maintained.

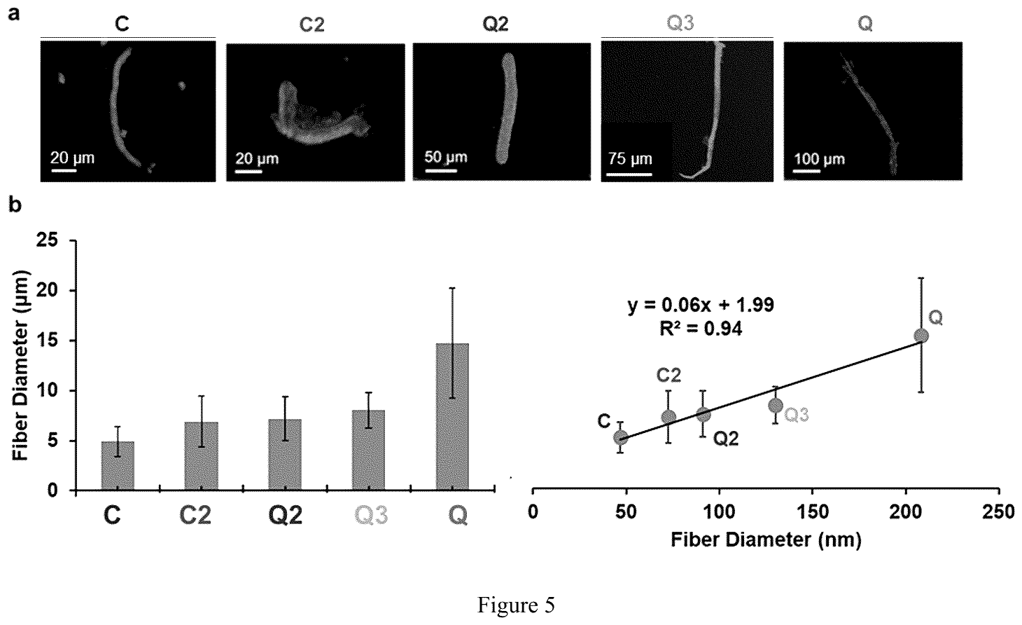

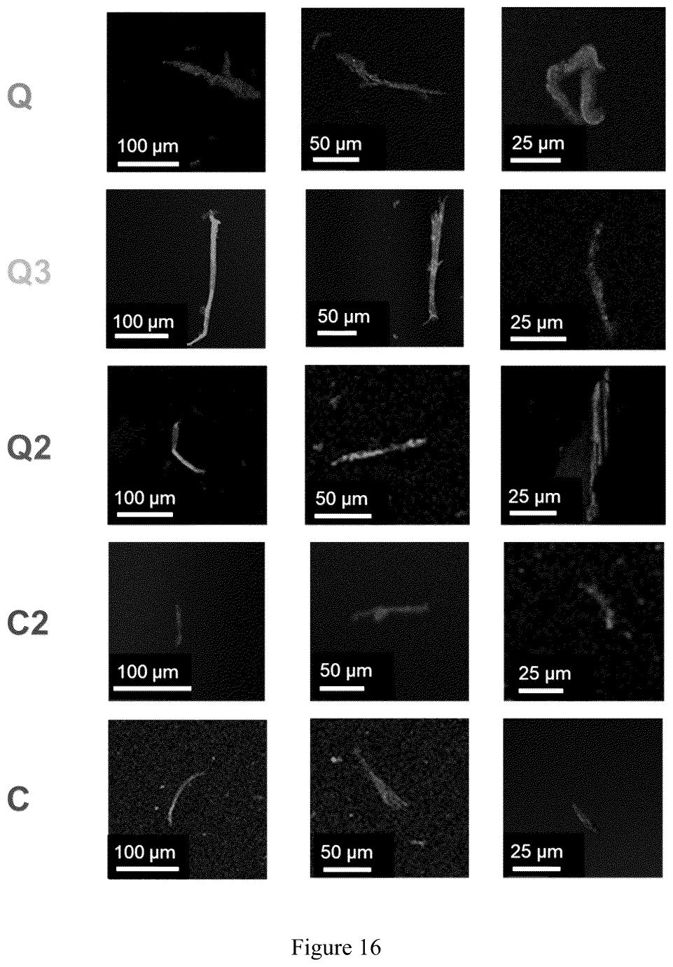

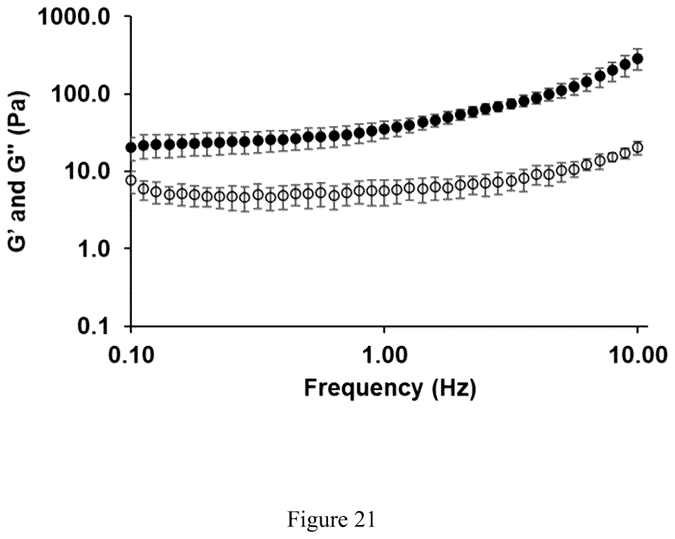

Using confocal microscopy, we test the relative thickening upon CCM binding in the coiled-coil variants (FIG. 5a and FIG. 16) was tested. In case of Q, the addition of CCM increased the diameter of the fiber to 14.74±5.501 μm, consistent with prior studies. The protein variants further assemble into mesoscale fibers with diameters of 8.04±1.78 μm for Q3, 7.17±2.19 μm for Q2, 6.89±2.54 μm for C2, and 4.91±1.49 μm for C (FIG. 21b). Notably, the fibers appear to thicken proportionally to their original fiber assemblies shown in FIG. 3. This correlation was confirmed by comparing the fiber diameters (by TEM) to the protein-CCM fiber diameters (by confocal microscopy) (FIG. 5b) with a directly proportional relationship possessing an R²=0.94; fiber thickening is not only present in all protein variants tested, but is also dependent on the original self-assembly properties (and thus electrostatics) of the fibers, independent of CCM.

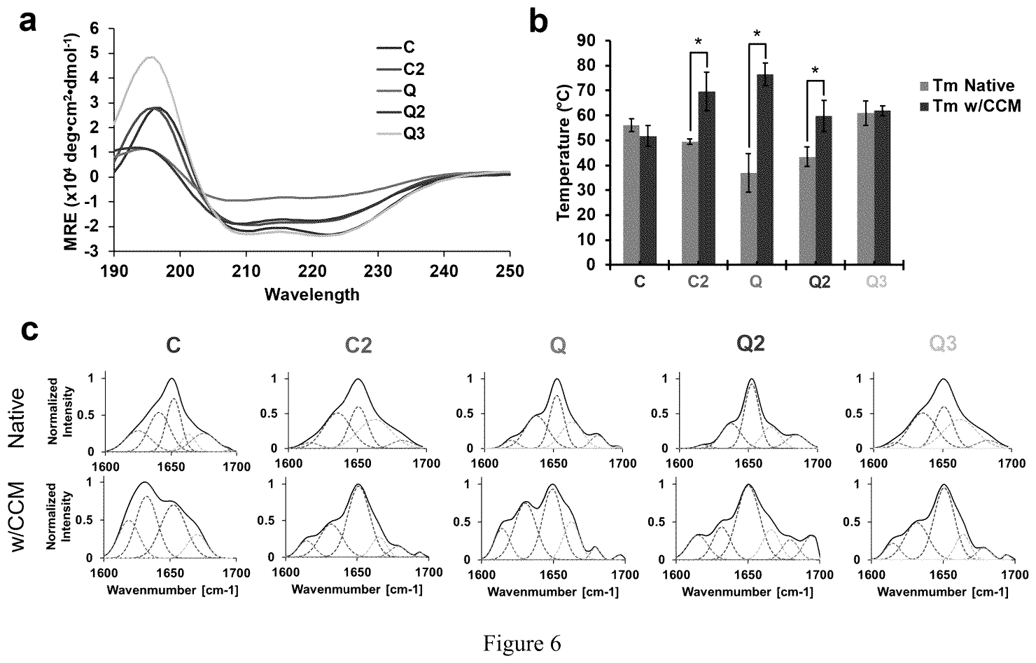

Structure and thermostability. To assess the secondary structure of the proteins, CD and ATR-FTIR measurements were performed (FIG. 6). CD wavelength scans were unable to be performed on protein after CCM binding since binding experiments used DMSO to solubilize CCM, which interferes with the measurement through strong absorbance of far UV light. CD spectra of all protein variants exhibited a double minimum at 222 nm and 208 nm, indicative of α-helical content (FIG. 6a and Table 1). Q displayed the lowest amount of helical content with minima −8,000±3,000 deg·cm²·dmol⁻¹ and −10,000±6,000 deg·cm²·dmol⁻¹ at 222 nm and 208 nm, respectively. Previous studies of Q agreed with a dampened signal in CD measurements, which was proposed to be due to reordering of the heptads such that residue P28 resided towards the center of Q. Variants Q2 and Q3 possessing mutations P28E and P28Y, respectively and possessing similar helical signal intensity to C and C2 confirmed the negative impact of the P28 mutation on Q α-helicity. Remaining coiled-coil proteins displayed an average double minima of −20,000±5,000 deg·cm²·dmol⁻¹ at 208 nm and −19,000±6,000 deg·cm²·dmol⁻¹ at 222 nm. Similarly, all proteins exhibited strong coiled-coil content in the range of 0.91-1.11 (Table 1) where helical systems with a 222/208 ratio >1 was indicative of the α-helix being found within a coiled-coil structure rather than in isolation.

TABLE 1
Mean residue ellipticity (MRE) of minima at 222 nm
and 208 nm from circular dichroism spectra. Results
represent the average of three independent trials.

| Pro- | θ₂₂₂ | θ₂₀₈ | |
|---|---|---|---|
| tein | (mdeg · cm² · dmol⁻¹) | (mdeg · cm² · dmol⁻¹) | θ₂₂₂/θ₂₀₈ |
| C1 | −23,000 ± 5,000 | −21,000 ± 5,000 | 1.11 ± 0.06 |
| C2 | −18,000 ± 600 | −19,000 ± 1,000 | 0.97 ± 0.02 |
| Q1 | −8,000 ± 3,000 | −10,000 ± 6,000 | 1.04 ± 0.57 |
| Q2 | −17,000 ± 2,000 | −19,000 ± 2,000 | 0.91 ± 0.05 |
| Q3 | −20,000 ± 2,000 | −19,000 ± 3,000 | 1.08 ± 0.08 |

ATR-FTIR measurements were used to assess secondary structure and compare proteins before and after binding of CCM (Table 2-6). Prior to CCM binding (native), protein variants showed a high order of structured content with a strong presence of α-helicity in agreement with CD measurements. Q exhibited the weakest helical composition with a value of 32.8±4.2% and highest percentage of random content with a value of 25.1±10.3% (Table 2-6). In contrast, Q2 demonstrated the strongest helical percent composition of 50.8±6.0% and C2 demonstrated the weakest random percent composition of 9.4±7.2% (Tables 2-6). In comparison, after binding CCM, the proteins all experienced a consistent loss in structured content noted by the broadening of the amide II bond region curves comprised of random coil content signals in the ranges 1610-1628 cm⁻¹ and 1660-1670 cm⁻¹, respectively (FIG. 6c).

TABLE 2
Peak deconvolution using PeakFit software from ATR-FTIR spectra
for C. Results represent the average of three independent trials.

| | C | | | | | |
|---|---|---|---|---|---|---|
| | antiparallel β-sheet/ | | | 3-10 | aggregated | |
| | aggregated strands | β-sheet | α-helix | Helix | strands | Unordered |
| Secondary Structure | β-sheet | | α-helix | Turns/Coils | | |
| % Compositions | 11.5 ± | 25.1 ± | 42.7 ± | 10.6 ± | 10.0 ± | 0.0 ± |
| native | 5.0% | 3.6% | 6.4% | 9.2% | 7.7% | 0.0% |
| | 36.5 ± 6.2% | | 42.7 ± | 20.6 ± 12.0% | | |
| | | | 6.4% | | | |
| % Compositions | 0.0 ± | 33.5 ± | 26.6 ± | 19.1 ± | 20.8 ± | 0.0 ± |
| w/CCM | 0.0% | 37.9% | 16.2% | 17.8% | 10.3% | 0.0% |

| | | |
|---|---|---|
| 33.5 ± 37.9% | 26.6 ± | 39.9 ± 20.6% |

16.2%

TABLE 3
Peak deconvolution using PeakFit software from ATR-FTIR spectra for
C2. Results represent the average of three independent trials.

| | C2 | | | | | |
|---|---|---|---|---|---|---|
| | antiparallel β-sheet/ | | | 3-10 | aggregated | |
| | aggregated strands | β-sheet | α-helix | Helix | strands | Unordered |
| Secondary Structure | β-sheet | | α-helix | Turns/Coils | | |
| % Compositions | 15.0 ± | 37.2 ± | 38.4 ± | 4.1 ± | 5.3 ± | 0.0 ± |
| native | 3.2% | 1.8% | 0.3% | 7.2% | 0.6% | 0.0% |
| | 52.2 ± 3.6% | | 38.4 ± | 9.4 ± 7.2% | | |
| | | | 0.3% | | | |
| % Compositions | 4.8 ± | 23.9 ± | 50.0 ± | 12.3 ± | 9.0 ± | 0.0 ± |
| w/CCM | 2.0% | 5.5% | 10.1% | 6.4% | 2.1% | 0.0% |

| | | |
|---|---|---|
| 28.7 ± 37.9% | 50.0 ± | 21.2 ± 6.7% |

10.1%

TABLE 4
Peak deconvolution using PeakFit software from ATR-FTIR spectra
for Q. Results represent the average of three independent trials.

| | Q | | | | | |
|---|---|---|---|---|---|---|
| | antiparallel β-sheet/ | | | | aggregated | |
| | aggregated strands | β-sheet | α-helix | 3-10 | strands | Unordered |
| Secondary Structure | β-sheet | | α-helix | Helix | Turns/Coils | |
| % Compositions | 13.0 ± | 29.2 ± | 32.8 ± | 19.1 ± | 6.0 ± | 0.0 ± |
| native | 4.1% | 2.7% | 4.2% | 9.2% | 4.8% | 0.0% |
| | 42.4 ± 4.9% | | 32.8 ± | 25.1 ± 10.3% | | |
| | | | 4.2% | | | |
| % Compositions | 7.8 ± | 32.5 ± | 32.9 ± | 16.3 ± | 15.4 ± 3.8% | 0.0 ± |
| w/CCM | 8.3% | 5.1% | 3.3% | 0.7% | | 0.0% |

| | | |
|---|---|---|
| 40.3 ± 9.7% | 32.9 ± | 31.6 ± 3.8% |

3.3%

TABLE 5
Peak deconvolution using PeakFit software from ATR-FTIR spectra for
Q2. Results represent the average of three independent trials.

| | Q2 | | | | | |
|---|---|---|---|---|---|---|
| | antiparallel β-sheet/ | | | 3-10 | aggregated | |
| | aggregated strands | β-sheet | α-helix | Helix | strands | Unordered |
| Secondary Structure | β-sheet | | α-helix | Turns/Coils | | |
| % Compositions | 20.6 ± | 16.3 ± | 50.8 ± | 8.4 ± | 3.9 ± | 0.0 ± |
| native | 13.8% | 8.9% | 6.0% | 9.3% | 2.4% | 0.0% |
| | 36.5 ± 6.2% | | 50.8 ± | 23.7 ± 9.6% | | |
| | | | 6.0% | | | |
| % Compositions | 0.0 ± | 33.5 ± | 46.9 ± | 16.9 ± | 10.6 ± | 0.0 ± |
| w/CCM | 0.0% | 37.9% | 7.0% | 2.3% | 3.6% | 0.0% |

| | | |
|---|---|---|
| 28.9 ± 13.6% | 46.9 ± | 26.7 ± 1.7% |

7.0%

TABLE 6
Peak deconvolution using PeakFit software from ATR-FTIR spectra for
Q3. Results represent the average of three independent trials.

| | Q3 | | | | | |
|---|---|---|---|---|---|---|
| | antiparallel β-sheet/ | | | 3-10 | aggregated | |
| | aggregated strands | β-sheet | α-helix | Helix | strands | Unordered |
| Secondary Structure | β-sheet | | α-helix | Turns/Coils | | |
| % Compositions | 6.8 ± | 33.4 ± | 41.1 ± | 13.6 ± | 5.2 ± | 0.0 ± |
| native | 5.9% | 1.1% | 1.1% | 5.3% | 2.7% | 0.0% |
| | 40.1 ± 5.9% | | 41.1 ± | 18.8 ± 5.9% | | |
| | | | 1.1% | | | |
| % Compositions | 7.6 ± | 22.0 ± | 34.3 ± | 18.3 ± | 17.8 ± | 0.0 ± |
| w/CCM | 6.5% | 9.2% | 10.7% | 9.4% | 14.8% | 0.0% |

| | | |
|---|---|---|
| 29.6 ± 11.3% | 34.3 ± | 36.1 ± 17.5% |

10.7%

Melting temperatures of the proteins as measured by CD also experienced a shift as a result of curcumin binding. Melting temperatures for C and Q were 56.0±2.6° C. and 37.0±7.8° C., respectively at pH 4.0. Previously, C resulted in a melting temperature of 41-42° C. in Na₂HPO₄ pH 8.0. Q possessed a melting temperature of 55.3° C., 63.5° C., and 46.4° C. in 50 mM Na₂HPO₄ at pH 4.0, 8.0, and 10.0, respectively, and 39° C. in 50 mM Na₂HPO₄ at pH 8.0 in previous studies. Notably, melting temperature of Q was sensitive to changes in buffer composition and pH. In comparison here, proteins were measured in 50 mM NaH₂PO₄ at pH 4.0, which would provide a lower ionic strength buffer than used in previous studies. Melting temperatures of the protein variants were measured as 49.4±1.1° C. for C2, 43.4±4.0° C. for Q2, and 61.0±4.9° C. for Q3 (FIG. 6b). After binding CCM, C2, Q1, and Q2 all experienced a significant change in melting temperature compared to its unbound state with p-values of 0.012, <0.001, 0.019 by an unpaired t-test, respectively. All CCM-bound proteins resulted in melting temperatures greater than 50° C. with melting temperatures of 51.8±4.3° C. for C, 69.6±7.8° C. for C2, 76.4±4.6° C. for Q, 59.9±6.3° C. for Q2, and 61.9±2.0° C. for Q3 (FIG. 6b).

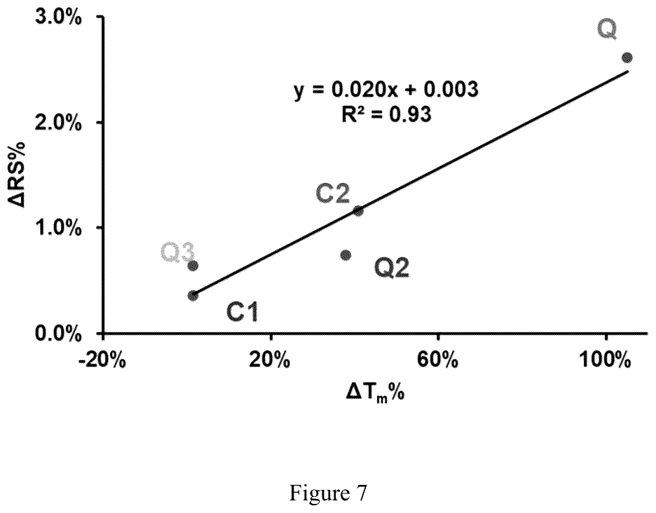

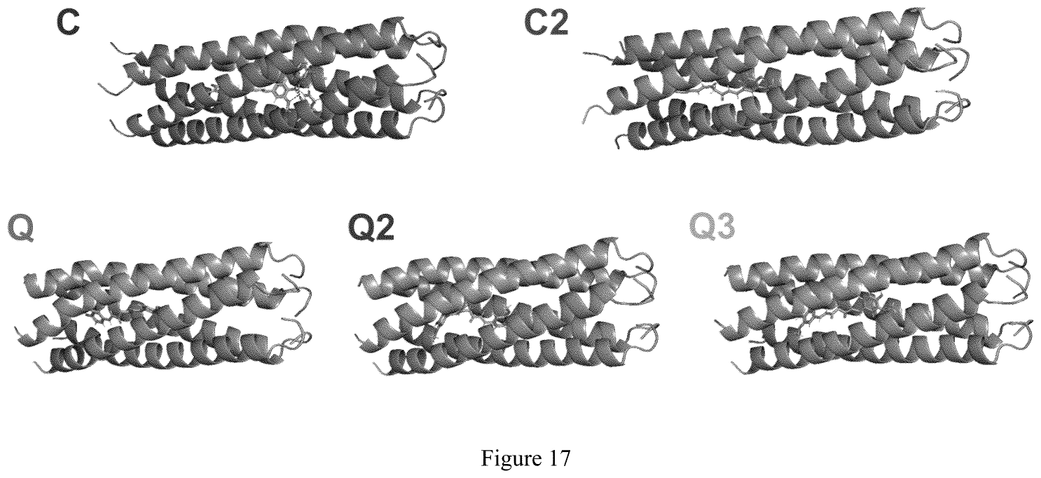

To help decipher the impact of CCM binding on the protein, Rosetta macromolecular suite was used to dock CCM and score the protein before and after docking. The best scoring complexes were used to extract the Rosetta score of the protein (with and without the CCM ligand). Protein variants exhibited interface scores of −45.4 kcal/mol for C, −20.6 kcal/mol for C2, −46.2 kcal/mol for Q, −46.3 kcal/mol for Q2, and −45.2 kcal/mol for Q3, affirming promiscuity of the C and Q binding pockets. The lesser interface score exhibited by C2 as compared to its parent, C, can be explained by the absence of hydrogen bonding with the glutamic acid ring at sequence position 54 (FIG. 17) due to a decrease in distance (0.3 Å) between chains at the C-terminus measured by PyMOL of the lowest-scoring poses preventing C-terminal encapsulation. When comparing the percent change of the protein Rosetta score before and after docking, a strong correlation (R²=0.93) was found with the percent difference of the change in melting temperature (ΔTₘ%) (FIG. 7). The binding of curcumin positively impacted the stability of the fiber pentamer.

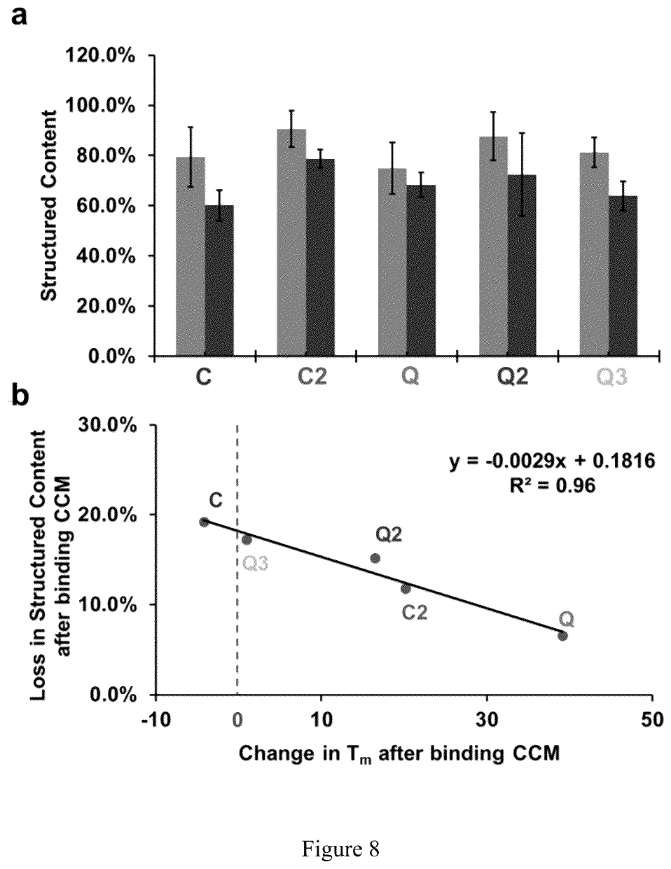

At the same time, a loss of structure is observed by ATR-FTIR measurements (FIG. 8a). To elucidate this interplay of structure and stability, a relationship with the change in stability and the change in structure was defined. It is noted that upon CCM binding, a greater increase in stability linearly correlates to a smaller loss in structured content (denoted by secondary structure not associated with random coil content in ATR-FTIR) with an R²=0.96 (FIG. 8, Table 2-6). Thus, while CCM binding imposes a negative impact on the ordered structure of the protein fibers based on the ATR-FTIR, CCM imparts a positive interaction in the pore of the coiled-coil. Based on the y-intercept of the linear relationship denoted in FIG. 8b, this interaction appears to stabilize the pentameric interface and increases thermostability when the loss of structure is less than 18.2%. When the loss of structure is greater than 18.2% as in the case of C, the difference in Tₘ of the native and CCM-bound protein fibers (FIG. 8b) indicates a continuation of this trend where a greater loss in structure will begin to cause a decrease in thermostability of the protein.

A mini-library of coiled-coil proteins with mutations to create a diverse set electrostatic distributions on the surface of the coiled-coil surface was designed and synthesized. The ability of these coiled-coils to undergo supramolecular assembly into nanoscale fibers of different degrees and correlation of this relationship to the magnitude of the electrostatic potential difference of the surface was assessed. Furthermore, all fiber variants can bind the candidate small hydrophobic molecule, curcumin, which induces fiber assembly into the mesoscale. The relationship of electrostatic potential of the surface on the resulting diameters after curcumin binding is maintained. While curcumin binding consistently induces a loss of structure, the binding of the small molecule appears to have a positive interaction in the hydrophobic pore that competes to affect an improved thermostability.

### Example 3

The following provides examples of peptides of the present disclosure.

As previously described, Q is a thermoresponsive coiled-coil protein capable of higher-order supramolecular assembly into fibers and hydrogels with upper critical solution temperature (UCST) behavior. Described in this example is a new coiled-coil protein, Q2, that is redesigned to favor longitudinal growth over lateral growth of its fibers and thus achieve a higher crosslinking density within the formed hydrogel. A favorable hydrophobic mutation was introduced to the pore of the coiled-coil domain for increased thermostability of the protein. It is noted that an increase in storage modulus of the hydrogel and crosslinking density is coupled with a decrease in fiber diameter. The α-helical coiled-coil Q2 hydrogel was characterized for its structure, nano-assembly, and rheology relative to our previous single domain protein, Q, over the time of its gelation demonstrating the nature of our hydrogel self-assembly system. The design parameters here not only show the importance of electrostatic potential in self-assembly but provide a step towards predictable design of electrostatic protein interactions.

The coiled-coil hydrogel, Q, was originally prepared by domain swapping its N- and C-terminus around the Q54 residue of C. The resulting Q possesses an electrostatic redistribution of surface charge such that the N-terminus is positively charged and the C-terminus is negatively charged. This leads to the protein undergoing electrostatically driven supramolecular assembly into a hydrogel at concentrations greater than 1 mM and at temperatures below its upper critical solution temperature (UCST) of 16.2° C., while maintaining its ability to encapsulate and release small hydrophobic molecules. It was previously shown Q to be stimuli-responsive towards changes in pH and ionic strength, allowing its phase transition, including its transition temperature and the gelation kinetics, to be tuned through alteration of the surrounding chemical environment.

The fiber growth of Q occurs predominantly through electrostatic coupling and the end-to-end alignment of coiled-coils (longitudinally). Less present, Q fiber diameters measure in tens of nanometer range signaling the presence of some side-to-side stacking of coiled-coils (laterally). To control the crosslinking density for our coiled-coil systems, Q was redesigned with the intent to tune its fiber geometry such that supramolecular assembly has an even greater preference for longitudinal over lateral growth. By manipulating the electrostatic distribution of the surface, it was hypothesized that the thinner protofibrils will possess increased solvent-exposed surface available for interaction and thus create increased physical crosslinking of the fibers. Described herein is a time-course structural and rheological characterization of the hydrogel, Q2, which has been rationally designed for reduced lateral growth.

Materials. Chemically competent M15MA E. coli cells were gifted from David Tirrell at California Institute of Technology. Bacto-tryptone, sodium chloride (NaCl), yeast extract, tryptic soy agar, ampicillin sodium salt, sodium phosphate dibasic anhydrous (Na₂HPO₄), sodium hydroxide (NaOH), dextrose monohydrate (_{D}-glucose), magnesium sulfate (MgSO₄), calcium chloride (CaCl₂), manganese chloride tetrahydrate (MnCl₂·4H₂O), cobaltous chloride hexahydrate (CoCl₂·6H₂O), isopropyl β-_{D}-1-thiogalactopyranoside (IPTG), Pierce bicinchoninic acid (BCA) assay kit, Pierce snakeskin dialysis tubing 3.5 K molecular weight cutoff (MWCO), sodium dodecyl sulfate (SDS), Nunc ninety-six well plates, Molecular Probes FluoSpheres (1.0 μm) and BD Clay Adams glass microscopy slides were acquired from Thermo Fisher Scientific. The twenty naturally occurring amino acids, dimethylsulfoxide (DMSO), 3,3′,5,5′-tetramethylbenzidine (TMB), nickel (III) chloride hexahydrate (NiCl₂·6H₂O), sodium molybdate dihydrate (Na₂MoO₄·2H₂O), iron (III) chloride (FeCl₃), iron (II) chloride tetrahydrate (FeCl₂-4H₂O), thiamine hydrochloride (vitamin B), thioflavin T (ThT), and copper (II) sulfate pentahydrate (CuSO₄·5H₂O) were purchased from Sigma Aldrich. Hydrochloric acid (HCl), Coomassie® Brilliant Blue G-250 were purchased from VWR. HiTrap FF 5 mL columns for protein purification were purchased from Cytiva Life Sciences. Macrosep and Microsep Advance Centrifugal Devices 3K MWCO and 0.2 μm syringe filters were purchased from PALL. Acrylamide/bis solution (30%) 29:1, Mini Trans-Blot filter paper, Trans-Blot Transfer Medium (nitrocellulose membrane), and natural polypeptide sodium dodecyl sulfate-polyacrylamide gel electrophoresis (SDS-PAGE) standard were purchased from Bio-Rad. Copper (II) chloride anhydrous (CuCl₂), sodium selenite (Na₂SeO₃), and imidazole were purchased from Acros Organics. Formvar/carbon-coated copper grids (FCF400-Cu) and 1% uranyl acetate for transmission electron microscopy were purchased from Electron Microscopy Sciences. Borosilicate glass capillaries (0.2 mm×2 mm×75 mm) were purchased from VitroCom. Fast-curing two-component epoxy was acquired from JB Weld.

Computational Modeling and Design of Q2. To assess the iterative effect of mutations on the stability of a Q variant, ROSETTA suite of macromolecular modeling tools (Version 3.5) was used. ROSETTA Relax protocol was used on protein sequences using the symmetry of COMPcc (PDB: 3V2P) with the all-atom energy score function. To assess the isoelectric point (pI), PDB2PQR software (version 3.1.0) was used to set up titration states at room temperature with the amber forcefield and propka pH calculation method. Electrostatic maps and electrostatic potential energies were calculated using Adaptive Poisson-Boltzmann Solver (APBS) (Version 3.0.0) from subsequent PDB2PQR input files. In calculation of Rosetta score and electrostatic potential energy, the conserved His tag is negated due to Rosetta's inability define a consistent minimum energy state.

Expression and Purification. Q2 and Q protein were expressed as described previously. Briefly, Q2pqE30Q2 plasmid was cloned and purchased from Integrated DNA Technologies. Q and Q2 were expressed in methionine auxotrophic M15MA E. coli cells in supplemented M9 minimal media. Expression of each respective protein was induced through the addition of 200 μg/mL IPTG when the optical density at 600 nm (OD₆₀₀) reached ˜0.8. After incubation at 37° C. and 350 rpm for 3 hours, cells were harvested by centrifugation at 5000×g at 4° C. for 30 minutes in an Avanti J-25 centrifuge (Beckman Coulter) and stored at −20° C. until purification. 12% SDS-PAGE was used to confirm expression of the respective proteins. Q2 and Q were purified using affinity chromatography on a cobalt-charged HiTrap IMAC FF 5 mL column with Buffer A (50 mM Tris-HCl, 500 mM NaCl, pH 8.0). Protein was eluted using a gradient of Buffer B (50 mM Tris-HCl, 500 mM NaCl, 500 mM imidazole, pH 8.0) possessing an imidazole concentration range from 10-500 mM. Q2 Pure fractions were then dialyzed in six consecutive 5 L volumes of Buffer A and concentrated to 2 mM using 3 kDa Macrosep centrifugal filters (Pall). Protein purity was confirmed by 12% SDS-PAGE and concentration determined by BCA assay.

Tube Inversion Test. Immediately after concentration to 2 mM, the protein was incubated at 4° C. over the course of two weeks after aliquoting 150 μL into a 2 mL Eppendorf tube. The samples were visually assessed for gelation by inverting the tubes at 12-hour time intervals thereafter, with gelled samples not flowing upon inversion.

Microrheology. Protein was concentrated to 2 mM, as measured by BCA assay, and immediately aliquoted 27.7 μL into a 200 μL PCR tube. 1% v/v (or 0.3 μL) of 1 m diameter red polystyrene fluorometric beads (FluoSpheres) were then added to the sample. A glass capillary tube was then loaded with sample by capillary action and sealed to prevent evaporation while being affixed to a microscopy slide using two-component fast-curing epoxy (JB Weld). Samples were then imaged at 0 h as a starting measurement and then every 12 h using a Leica DMI4000b inverted microscope equipped with a Leica DFC310 FX 1.4 megapixel camera at 40× magnification with 2×2 binning. Between imaging, slides were incubated on a rotisserie at 8 rpm to prevent sedimentation of the fluorometric beads. Each image series was recorded for a total of 300 frames with a lag time (i) of 0.037 s between each frame. Relaxation exponents i.e. the logarithmic slopes of particle mean-squared displacements (MSDs) with respect to lag time as determined by multiple particle tracking (MPT) analysis were used to determine when equilibration of the hydrogel was complete and measurements were no longer recorded (60 h in this case). Images were stacked and converted to grayscale in MATLAB (Mathworks, R2021a) using code developed in-house, with MPT then being employed using MATLAB code developed and modified by Dufresene, Kilfoil, Blair, and O'Neill as described previously.

Rheology. Mechanical integrity of the Q2 hydrogel was assessed using a stress-controlled rheometer (Discovery Hybrid Rheometer 2, TA Instruments) equipped with a parallel plate geometry. After complete gelation of a 2 mM sample at 4° C., the sample was loaded onto the 8 mm diameter lower and upper plates with a 0.2 mm geometry gap. Strain and frequency settings were informed from previous Q hydrogel studies where the storage modulus (G′) and loss modulus (G″) were measured over the frequency range 0.1-10 Hz with a 5% oscillation strain.

Circular Dichroism Spectroscopy. Secondary structure of Q2 was measured using the Jasco J-815 CD spectrometer with a PTC-423S single position Peltier temperature control system. Wavelength scans (with 15 μM sample) were performed from 195 to 250 nm at 1 nm step sizes by diluting 2 mM of the protein into water in order to minimize the effects of sodium chloride. Scans were performed of Q2 before and after gelation at 4° C. The mean residue ellipticity (MRE) was calculated as described in previous studies.

Attenuated Total Reflectance-Fourier Transform Infrared Spectroscopy. Secondary structure of Q2 protein was confirmed using attenuated total reflectance-Fourier transform infrared (ATR-FTIR) spectroscopy using the Nicolet 6700 Fourier Transform Infrared Spectrometer equipped with a diamond ATR accessory and a mercury cadmium telluride (MCT)-A detector. Spectra were collected for 5 μL of 2 mM protein from 4000-400 cm⁻¹ with 0.5 cm⁻¹ increments. Sample spectra were normalized using buffer background and analyzed from 1700-1600 cm⁻¹ corresponding to the amide I region. Peaks were deconvoluted using Gaussian functions in PeakFit software until the goodness of fit reached r²>0.99.

Transmission Electron Microscopy. Transmission electron microscopy (TEM) images were taken with a FEI Talos L120C transmission electron microscope. The supramolecular assembly of Q2 and Q were monitored over the course of 60 and 144 hours, respectively (in correspondence with tube inversion and microrheology). TEM images were taken of the gel at times consistent with when microrheological measurements for Q2 for consistent fibril sizing. Samples were diluted to 50 μM and 3 μL was spotted on Formvar/carbon-coated copper grids followed by 5 μL wash with water, and 3 μL staining with 1% v/v uranyl acetate solution each with incubation times of 1 min. Following imaging, fibrils were sized in ImageJ software (Version 1.52q).

Statistical Analysis. GraphPad Prism (GraphPad Software) was employed for statistical analysis using student's t-test. Python sklearn module was employed for regression analysis of phase diagrams.

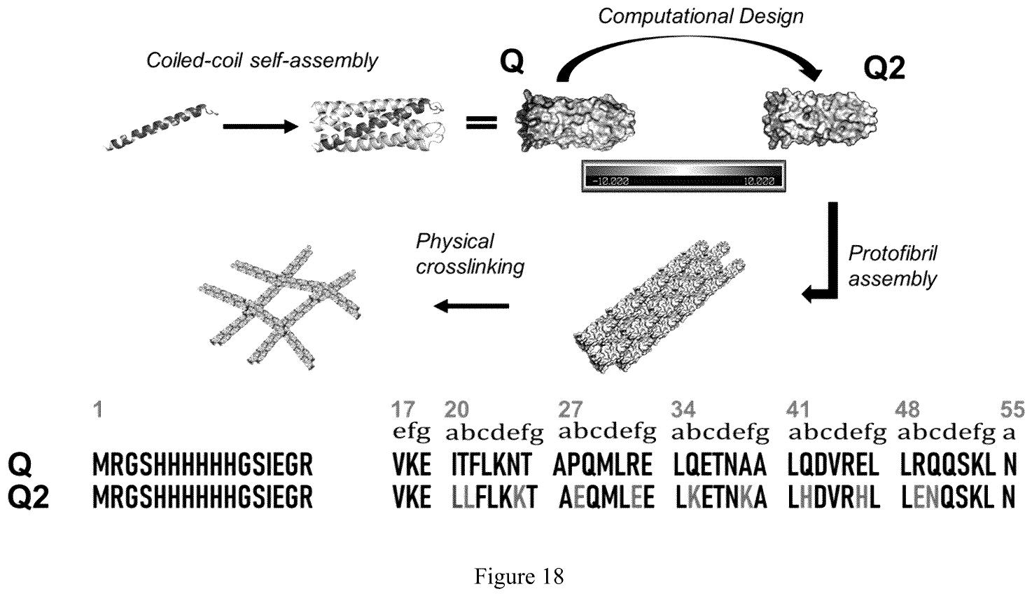

Design. Q2 was designed to undergo reduced lateral assembly (FIG. 18). The protonation and deprotonation of solvent-exposed residues is believed to be a key factor affecting the self-assembly of Q into fibers and further into a hydrogel. Using Rosetta, charge in the solvent exposed b, c, and f positions was modified to: 1) redistribute the surface to be more negative and lower the pI; 2) lower the overall electrostatic potential energy (U_{E}); and 3) improve the Rosetta stability score. A more evenly distributed negative surface on the C-terminus was hypothesized to push the pI to be more neutral and balance the positive and negative surface patches resulting from the largely positive patch constituted by the N-terminus and His tag. Recent studies on electrostatically interacting coiled-coils showed a strong correlation between the gelation kinetics and overall charge of the protein where faster gelation and an increased storage modulus was seen at pH near the pI of the protein. A lower overall U_{E} was hypothesized to preferentially decrease the lateral interaction of neighboring coiled-coils since end-to-end protofibril stacking was conserved from C-derived coiled-coil fibers. Mutations were rationally chosen and iteratively checked using the Rosetta relax and score functions and APBS electrostatic potential energy (U_{E}) until these goals were achieved. Complementary to redistribution of the surface charge, hydrophobic residues necessary for helix and coiled-coil formation were maintained throughout the surface charge optimization. A single point mutation, I20L, was then made in the hydrophobic pore to further stabilize it from changes in temperature. A final design (FIG. 18) was chosen, with a Rosetta score of −637 kcal/mol compared to the lower-magnitude Rosetta score of −610 kcal/mol for the parent Q protein, suggesting a higher stability for Q2. Additionally, Q2 exhibited a more neutral pI of 8.2 compared to 9.7 for Q. Q2 revealed a U_{E} of 1.29×10⁵ kJ/mol when compared to Q with 2.19×10⁵ kJ/mol.

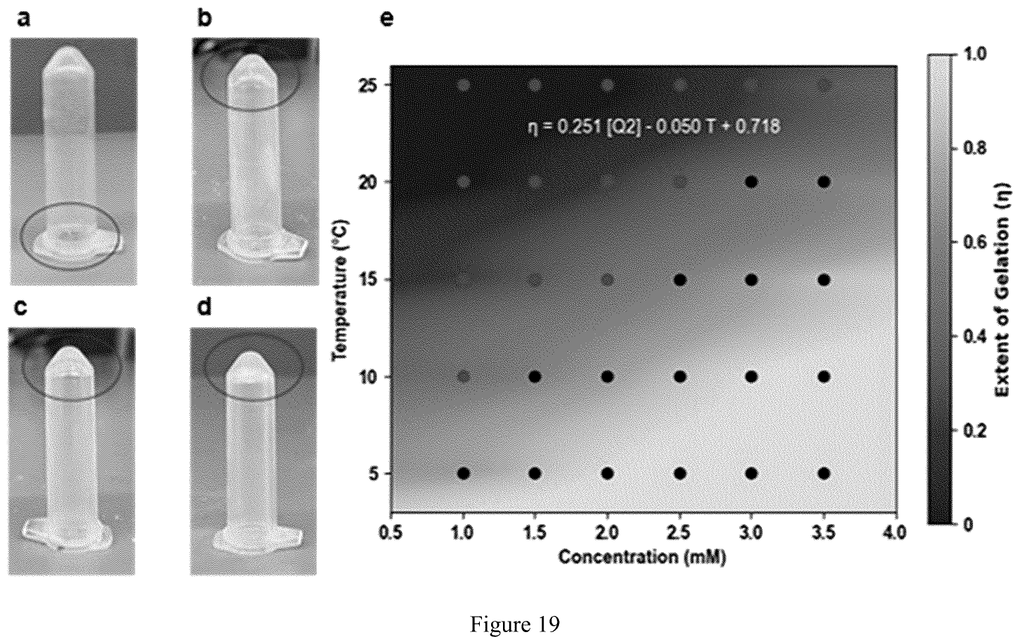

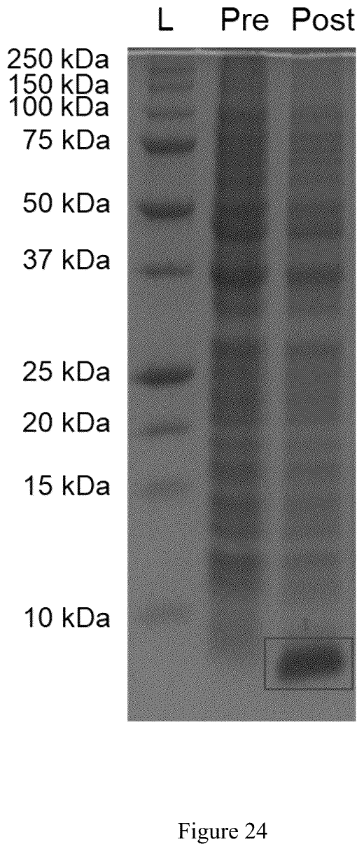

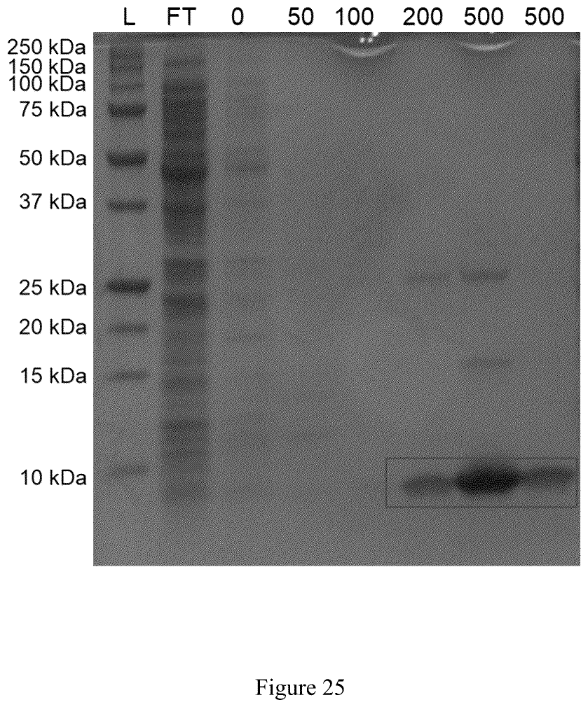

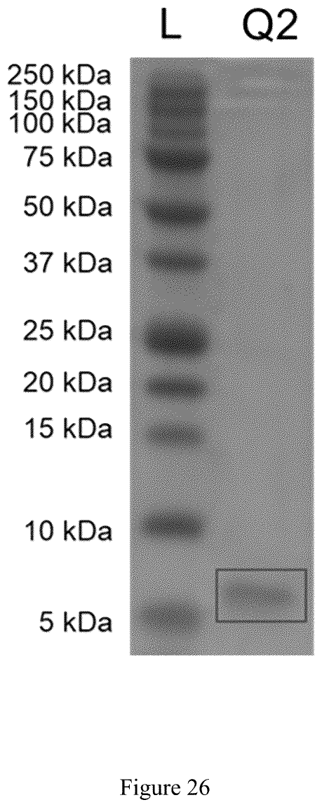

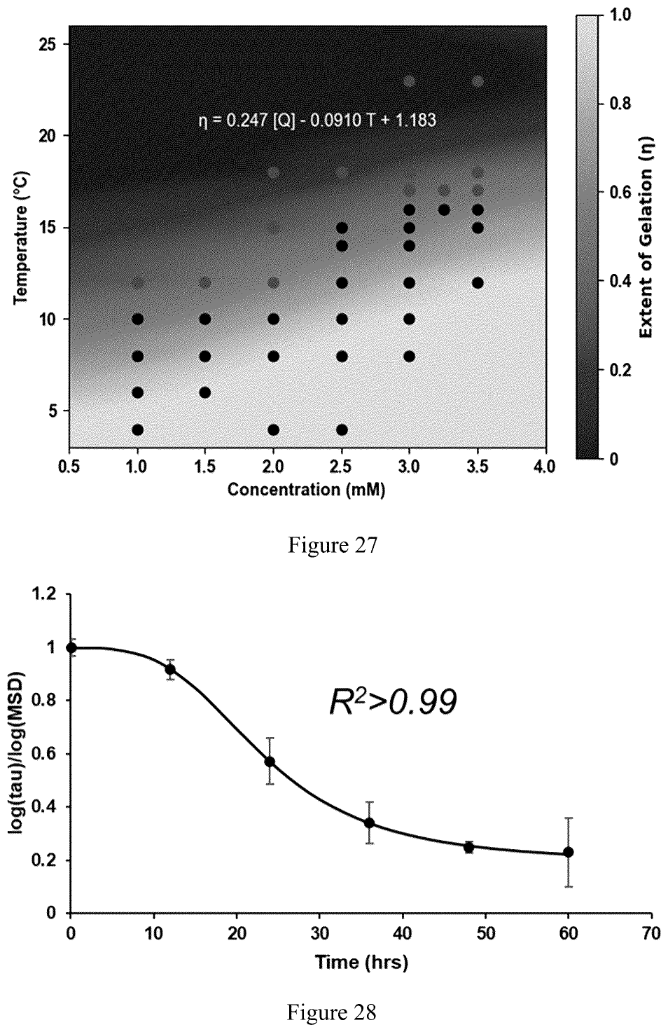

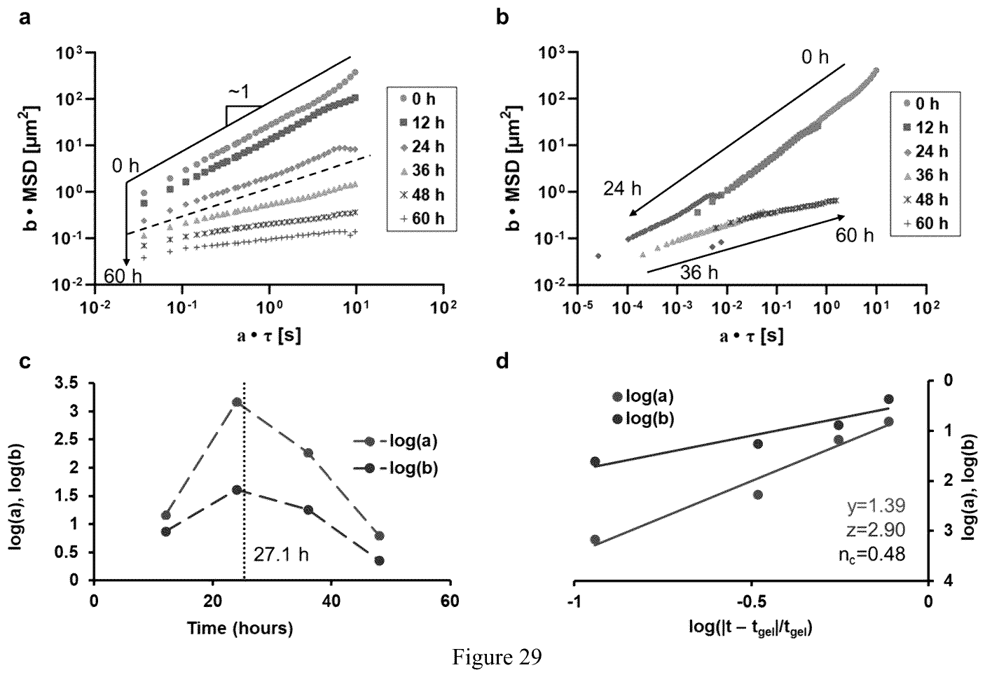

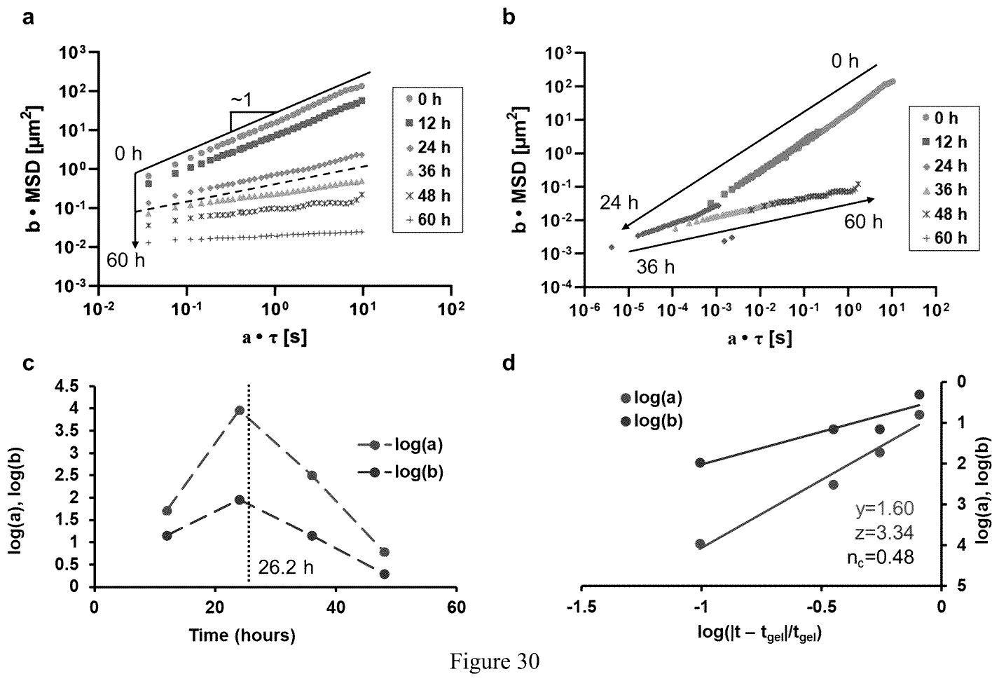

Gelation. To study the relative gelation of Q2 as compared to the previously reported coiled-coil hydrogel, Q, protein was successfully expressed (FIG. 24), purified (FIG. 25), and concentrated to 2 mM (1% w/v) in Tris buffer (50 mM Tris, 500 mM NaCl) at pH 8.0. Purity of the protein was measured to be >99% by SDS-PAGE (FIG. 26) and concentration was measured by BCA assay. Immediately following concentration, the gelation process of Q2 was studied using the microrheology assay described previously following incubation at 4° C. Separately, 150 μL samples were aliquoted for tube inversion tests performed after 2 weeks of incubation for visual assessment of gelation over time at 4° C. (FIG. 19a-d) and at concentrations from 1 mM to 3.5 mM with temperatures 5° C. to 25° C. using 0.5 mM and 5° C. step sizes, respectively, to create a matrix-based phase diagram (FIG. 19e). Temperature and concentration ranges were selected based on preliminary tube-inversion testing at high and low ends of the ranges. Protein concentrated past 3.5 mM was observed to precipitate out of solution at 25° C., indicating a solubility limit to the phase diagram, as also noted in the UCST of the parent protein, Q. In contrast, the UCST of Q2 was determined using sklearn linear processing in Python, where a bivariate linear relationship for the extent of gelation was established, assigning all tubes that passed for a gel by tube inversion (shown in red in FIG. 19e) a value of 1 and all tubes that failed to form a gel by tube inversion (shown in black in FIG. 19e) a value of 0. The relationship represented by the heatmap (FIG. 27) resolved a similar gelation system noted by a third-degree polynomial fit known in the art that revealed an apparent maximum gelation temperature near the end of the concentrations used. Alternatively, it was an established that an interpolated UCST of 22.0° C. at the end of the solubility limit, 3.5 mM; a linear relationship was used to solve for an extent of gelation, η, value of 0.5, representing the transition point of a material that is between a gel and solution. In comparison, the same analysis on the phase boundary points as known in the art represented a UCST of 17.0° C., at the end of the solubility limit for Q, 3.5 mM, resulting in an increase of 5° C. in the UCST (FIG. 19).

From this relationship, coefficients for the dependence of gelation on temperature and concentration, respectively, can be determined. These were used to compare the UCST dependence on concentration and temperature. In comparison, a concentration coefficient of 0.251 M⁻¹ and 0.247 mM⁻¹ for Q2 and Q, respectively, was noted; moreover, there is a temperature coefficient of −0.050° C.⁻¹ and −0.091° C.-1 for Q2 and Q, respectively. This translates to a 2% increase in concentration dependence and 45% decrease in temperature dependence from Q to Q2. Using this independent component analysis, Q2 shows both a stronger enthalpic drive to gelation, which can be attributed to its favorable stability due to sequence optimization (T21L, N25K, P28E, R32E, Q35K, Q42H, E46H, R49E, and Q50N) and introduction of the I20L mutation; this is supported by a higher Rosetta score, and entropic drive to gelation, which we attribute to improved surface charge and a pI closer to the buffer pH (8.0) used in the characterization of Q and Q2. In this regard, the increase in UCST of Q2 compared to that of Q, as well as rheological properties, is most dependent on its concentration.

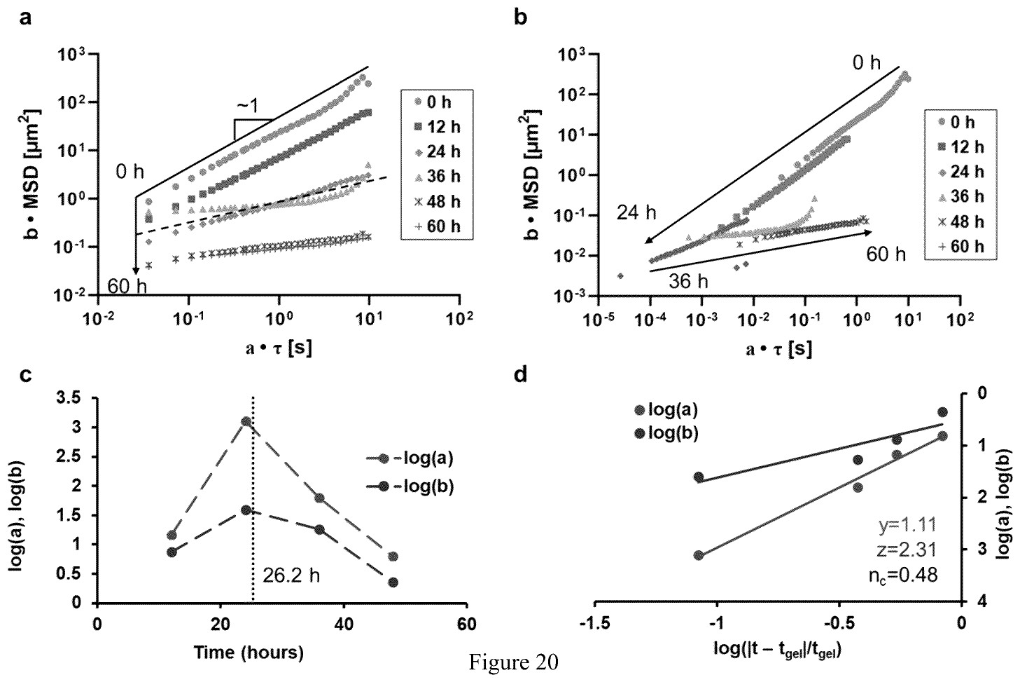

The solution-to-gel (sol-gel) transition with respect to time was quantified at 2 mM and 4° C. using passive microrheology. Multiple-particle tracking (MPT) analysis was employed to assess the movement of fluorescent tracer beads (FluoSphere), with bead trajectories tracked every 6 hours and their mean-square displacements (MSDs) analyzed using a best fit sigmoidal analysis (15) in MATLAB. Plotting the relaxation exponent with respect to time revealed a plateau, consistent with an equilibrating hydrogel, after 60 hours (FIG. 28). While the logarithmic slope of the particle MSDs began at 1.00±0.03 μm² s⁻¹ (consistent with Brownian motion), a gelation plateau was observed at 0.18±0.06 μm² s⁻¹ suggesting viscoelastic behavior of the final material (FIG. 20a). The respective MSD-τ curves for these relaxation exponents were selected as the master solution and master gelation curves, respectively, for time-cure superposition analysis, Q2 exhibited statistically insignificant differences compared to the master curves used for Q by an unpaired t-test (p-values 0.99 and 0.88, respectively) indicating similar gelation kinetics. Intermediate MSD-τ curves (between 0 h and 60 h) were superimposed onto the master solution or master gelation curve using horizontal and vertical shift factors, a and b, respectively (FIG. 20b). A divergence in the superposition onto the respective master curves was consistently seen between 24 h and 36 h, narrowing the time frame of the sol-gel transition to this window. As done previously, the asymptotic behavior of the shift factors as the critical extent of gelation is approached was used to determine the time to gelation, t_{gel} (FIG. 20c). Dynamic scaling factors, y and z, determined from the slopes of the horizontal and vertical shift factors, respectively, in relationship to the t_{gel} were used to calculate the critical relaxation exponent, ne, which is characteristic of the degree of crosslinking (FIG. 20d). Q2 possessed a t_{gel} of 26.6±0.5 h, revealing a large increase in the gelation kinetics compared to the Q hydrogel. A significant increase in crosslinking density of Q2 was marked by an n, of 0.48±0.001 as compared to Q (n, 0.53±0.03) by an unpaired t-test (p-value=0.045).

TABLE 7
Logarithmic slopes of the mean squared displacement (MSD)
of particles in the Q2 protein. Values are represented as
the average and standard deviation of the passive microrheology-
derived logarithmic slopes of the MSD for beads incubated
with Q2 at 4° C. and measured in 12 h time intervals.

| | Logarithmic Slope of MSD |
|---|---|
| Incubation Time (h) | (μm² s⁻¹) |
| 0 | 1.00 ± 0.03 |
| 12 | 0.92 ± 0.04 |
| 24 | 0.57 ± 0.09 |
| 36 | 0.34 ± 0.08 |
| 48 | 0.25 ± 0.02 |
| 60 | 0.18 ± 0.06 |

TABLE 8
Q2 storage (G′) and loss (G″) moduli from rheology
under 5% oscillatory strain at 4° C. Data is
represented as the average of three independent trials.

| Frequency (Hz) | G′ (Pa) | G″ (Pa) |
|---|---|---|
| 0.10 | 20.6 | 7.7 |
| 0.11 | 21.9 | 5.9 |
| 0.13 | 22.4 | 5.5 |
| 0.14 | 22.4 | 5.0 |
| 0.16 | 22.6 | 5.2 |
| 0.18 | 23.2 | 5.0 |
| 0.20 | 23.6 | 4.8 |
| 0.22 | 23.9 | 4.8 |
| 0.25 | 24.1 | 4.8 |
| 0.28 | 24.7 | 4.6 |
| 0.32 | 25.1 | 5.1 |
| 0.35 | 25.5 | 4.6 |
| 0.40 | 25.9 | 4.9 |
| 0.45 | 26.7 | 5.1 |
| 0.50 | 27.7 | 5.1 |
| 0.56 | 28.2 | 5.3 |
| 0.63 | 28.9 | 4.9 |
| 0.71 | 30.1 | 5.3 |
| 0.79 | 31.6 | 5.6 |
| 0.89 | 33.0 | .7 |
| 1.00 | 35.4 | 5.7 |
| 1.12 | 37.3 | 5.7 |
| 1.26 | 39.8 | 6.1 |
| 1.41 | 42.9 | 6.0 |
| 1.58 | 45.9 | 6.4 |
| 1.78 | 49.7 | 6.2 |
| 2.00 | 54.1 | 6.7 |
| 2.24 | 58.6 | 6.9 |
| 2.51 | 64.0 | 7.0 |
| 2.82 | 69.2 | 7.4 |
| 3.16 | 75.2 | 7.6 |
| 3.55 | 81.9 | 8.1 |
| 3.98 | 89.2 | 9.2 |
| 4.47 | 99.5 | 9.3 |
| 5.01 | 110.9 | 10.2 |
| 5.62 | 125.7 | 10.7 |
| 6.31 | 144.5 | 12.4 |
| 7.08 | 168.8 | 13.6 |
| 7.94 | 200.0 | 15.4 |
| 8.91 | 239.9 | 17.2 |
| 10.00 | 289.6 | 20.3 |

TABLE 9
ATR-FTIR compositional analysis from Q2 protein in solution
and gel states. Summary of secondary structure content uses
the average and standard deviation of the integrated area
of deconvoluted peaks of three independent trials.

| | % composition | | | | | |
|---|---|---|---|---|---|---|
| | α- | β- | Antiparallel | 3-10 | Unor- | Aggregated |
| | helix | sheet | β-sheet | helix | dered | Strands |
| Solution | 38.7 ± | 18.7 ± | 7.6 ± | 8.8 ± | 24.1 ± | 2.1 ± |
| | 0.3% | 0.2% | 0.8% | 0.1% | 0.2% | 0.0% |
| Gel | 51.5 ± | 29.5 ± | 11.3 ± | 5.4 ± | 0.0 ± | 2.3 ± |
| | 10.5% | 9.6% | 0.8% | 5.1% | 0.0% | 4.0% |

After gelation, rheological analysis of the storage and loss moduli was performed to assess the macroscopic mechanical integrity of Q2 (FIG. 21). As done previously for Q, a frequency sweep from 0.1 Hz to 10 Hz was performed at an oscillation strain of 5%. Q2 showed a strong improvement in mechanical integrity with a near 6-fold improvement in storage modulus reported at 10 Hz with a G′ of 289.6±86.9 Pa compared to 50.4 Pa for Q. In a physically crosslinked system, a higher G′ is associated with a higher degree of crosslinking, which is consistent with the lower critical relaxation exponent determined through time-cure superposition.

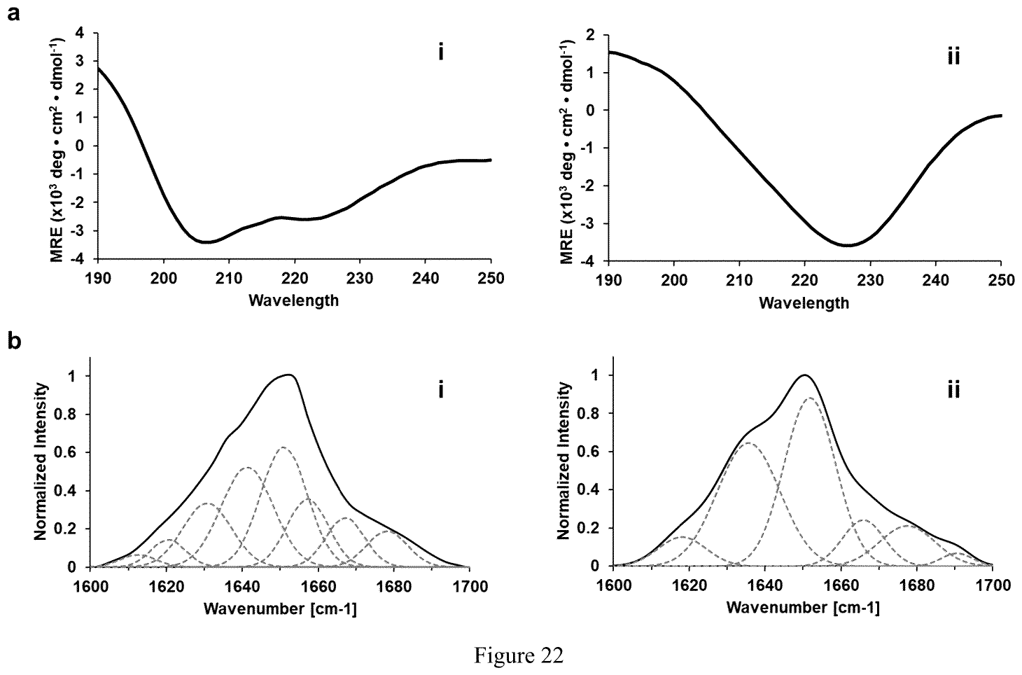

Secondary Structural Changes Upon Gelation. ATR-FTIR and CD spectroscopy were used to analyze protein secondary structure before and after gelation. In solution, immediately following concentration to 2 mM, Q2 revealed a typical α-helical protein secondary structure noted by CD wavelength scans performed at 4° C., showing a double-minima of −3,400±1,000 deg·cm²·dmol⁻¹ at 208 nm and −2,600±600 deg·cm²·dmol⁻¹ at 222 nm, indicative of helical conformation (FIG. 22a). The wavelength signature was similar to that of Q. Moreover, ATR-FTIR revealed helical conformation for Q2, typical of previous results for Q prior to gelation (FIG. 22a, b). Following incubation at 4° C. for one week and confirmation of gelation, secondary structure of Q2 in the hydrogel state was assessed (FIG. 22aii, bii). Both CD and FTIR spectra reveal a substantial increase in structured content. Notably, the CD spectrum of Q2 post-gelation exhibited a single minimum at 227 nm of −3,600±800 deg·cm²·dmol¹, which may be characteristic of a linear combination of α-helix and β-sheet conformation. Significantly, the Q2 spectra post-gelation exhibited a 222/208 ratio of 3.35±0.37 where helical systems with 222/208 ratios >1 have been used to indicate the likelihood of the α-helix being found within a coiled-coil structure rather than in isolation. Deconvolution of FTIR spectra of Q2 reveals an increase in α-helical content (+12.8%) and β-sheet (+14.5%) conformation at the expense of random coil conformation (−27.3%), indicating that the gels shifts to primarily α-helical secondary structure (51.5%) upon gelation (Table 9) similar to Q (52%).

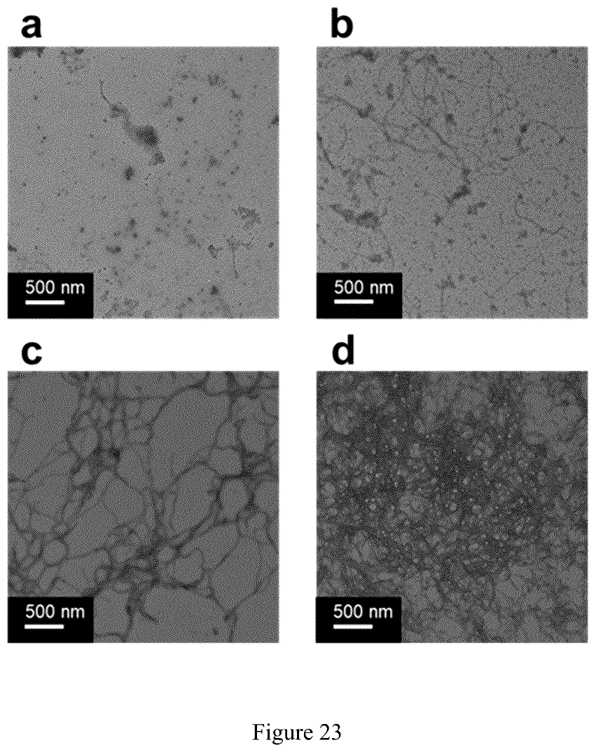

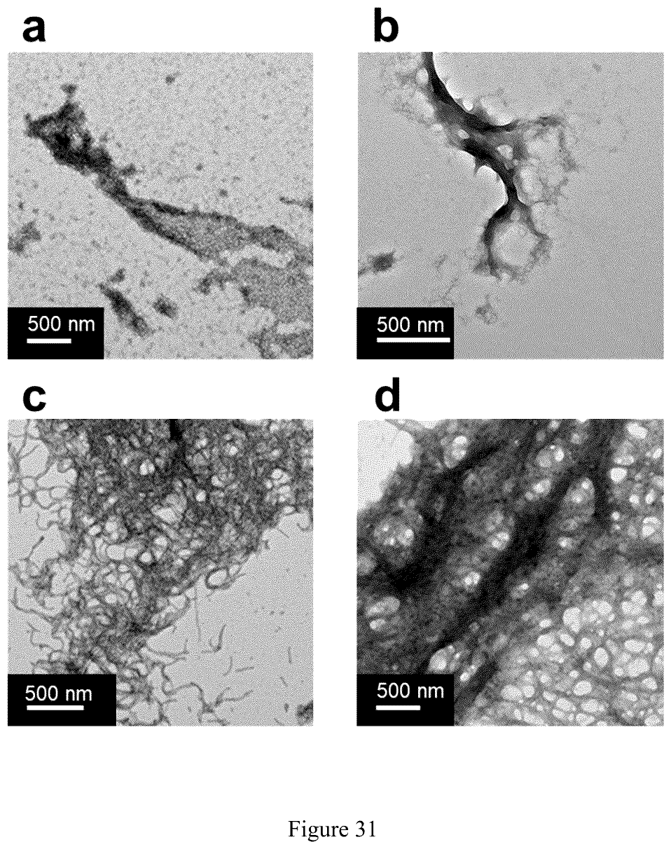

Nanoscale Assembly and Hydrogel Microstructure. To assess the morphology and structure of the Q2 hydrogel over time, samples were imaged through TEM at 0 h, 12 h, 36 h, and 60 h. Whereas at 0 h, Q2 illustrated unorganized protein deposits or aggregates, the formation of nanometer-scaled fibrous structures was observed at 12 h. By 36 h, Q2 demonstrated a high degree of physical crosslinking characteristic of a networked hydrogel. At 60 h where equilibrated gelation was observed by microrheology, the hydrogel network became more densely crosslinked, which was consistent with rheological measurements of the hydrogel. To investigate the effect of lateral assembly, micrograph images of Q2 (FIG. 23a-d) and Q (FIG. 31a-d) were used to assess the physically crosslinked hydrogel microstructure. In the final hydrogel structures, Q2 displayed interconnected fibril diameters of 28.9±8.6 nm, which was significantly less than the microstructures resolved by Q's hydrogel network with average diameters of 40.5±25.2 nm (p-value=0.0001) indicating a decrease in the lateral assembly of protofibrils. At this point, only organized fibril networks were visible with no noticeable aggregates. Coupled with rheological measurements, protofibril formation of Q2 disfavored lateral assembly while conserving longitudinal assembly, resulting in thinner fibers that yielded more densely crosslinked networks.

A new single domain coiled-coil hydrogel was described herein to increase its UCST, mechanical integrity, and gelation kinetics through iterative rational design using protein stability and surface charge to favor longitudinal over lateral fiber assembly. To fully assess the impact of these changes, a time-course study on structure and gelation properties of Q2 compared to Q was performed. The results demonstrate a higher density of crosslinking as a result of its design. The favorability of these design choices for reduced lateral assembly can potentially be used to tune the gelation properties of physically crosslinked hydrogels and supramolecular assemblies. This bottom-up approach may inform design choices to tune hydrogel gelation properties for various biomedical applications and understand the change in morphology and secondary structure over time.

### Example 4

The following provides examples of peptides and methods of the present disclosure.

Theranostic materials research is experiencing rapid growth driven by the interest in integrating both therapeutic and diagnostic modalities. These materials offer the unique capability to not only provide treatment but also track the progression of a disease. However, to create an ideal theranostic biomaterial, without compromising drug encapsulation, diagnostic imaging must be optimized for improved sensitivity and spatial localization. Described herein is a protein-engineered fluorinated coiled-coil fiber, Q2_{LTF}, capable of improved sensitivity to ¹⁹F magnetic resonance spectroscopy (MRS) detection. Leveraging residue-specific non-canonical amino acid incorporation of trifluoroleucine (L_{TF}) into the coiled-coil, Q2, which self-assembles into nanofibers, Q2_{LTF} was generated. Demonstrated herein is that fluorination results in a greater increase in thermostability and ¹⁹F magnetic resonance detection compared to the non-fluorinated parent, Q2. Q2_{LTF} also exhibits ¹⁹F MRS thermoresponsiveness allowing it to act as a temperature probe. Furthermore, the ability of Q2_{LTF} to encapsulate the anti-inflammatory small molecule, curcumin (CCM), and its impact on coiled-coil structure, was explored. Q2_{LTF} also provides hyposignal contrast in ¹H MRI, echogenic signal with high-frequency ultrasound and sensitive detection by ¹⁹F MRS in vivo illustrating fluorination of coiled-coils for supramolecular assembly and their use as multi-modal theranostic agents.

Described herein is a protein-based fluorinated self-assembling fiber, Q2_{LTF} as a theranostic agent capable of ¹⁹F MRS. It is also demonstrated herein that Q2_{LTF} has increased sensitivity for ¹⁹F MRS, and increased thermostability compared to previous constructs and can encapsulate the hydrophobic small molecule, curcumin (CCM), which provides further stabilization. Furthermore, we show that Q2_{LTF} may be used in vivo as a visible fiber assembly via ¹H MRI and high-frequency ultrasound as well as a sensitive biomaterial using ¹⁹F MRS. It was also shown that Q2_{LTF} possesses a ¹⁹F MRS signal proportional to its protein structure and environmental temperature indicating its potential as a multifunctional in vivo probe.

Materials. Electrically competent LAM1000 E. coli cells were gifted from David Tirrell at California Institute of Technology. Bacto-tryptone, sodium chloride (NaCl), yeast extract, tryptic soy agar, ampicillin sodium salt, sodium phosphate dibasic anhydrous (Na₂HPO₄), sodium hydroxide (NaOH), dextrose monohydrate (_{D}-glucose), magnesium sulfate (MgSO₄), calcium chloride (CaCl₂), manganese chloride tetrahydrate (MnCl₂·4H₂O), cobaltous chloride hexahydrate (CoCl₂·6H₂O), isopropyl β-_{D}-1-thiogalactopyranoside (IPTG), Pierce bicinchoninic acid (BCA) assay kit, Pierce snakeskin dialysis tubing 3.5 K molecular weight cutoff (MWCO), sodium dodecyl sulfate (SDS), Nunc ninety-six well plates, BD Clay Adams glass microscopy slides, Pierce C18 tips, and 5,5,5-Trifluoroleucine were acquired from Thermo Fisher Scientific. The twenty naturally occurring amino acids, dimethylsulfoxide (DMSO), nickel (III) chloride hexahydrate (NiCl₂·6H₂O), sodium molybdate dihydrate (Na₂MoO₄·2H₂O), iron (III) chloride (FeCl₃), iron (II) chloride tetrahydrate (FeCl₂-4H₂O), thiamine hydrochloride (vitamin B), thioflavin T (ThT), curcumin (CCM), trifluoroacetic acid (TFA), ProteoMass peptide and protein MALDI-MS calibration kit containing sinnapinic acid, D₂O, and copper (II) sulfate pentahydrate (CuSO₄·5H₂O) were purchased from Sigma Aldrich. Hydrochloric acid (HCl), Coomassie® Brilliant Blue G-250 were purchased from VWR. HiTrap FF 5 mL columns for protein purification were purchased from Cytiva Life Sciences. Macrosep and Microsep Advance Centrifugal Devices 3K MWCO and 0.2 μm syringe filters were purchased from PALL. Acrylamide/bis solution (30%) 29:1, and natural polypeptide sodium dodecyl sulfate-polyacrylamide gel electrophoresis (SDS-PAGE) standard were purchased from Bio-Rad. Copper (II) chloride anhydrous (CuCl₂), sodium selenite (Na₂SeO₃), and imidazole were purchased from Acros Organics. Formvar/carbon-coated copper grids (FCF400-Cu) and 1% uranyl acetate for transmission electron microscopy were purchased from Electron Microscopy Sciences. Borosilicate glass capillaries (0.2 mm×2 mm×75 mm) were purchased from VitroCom.

Expression and Purification. Q2_{LTF} and Q_{LTF} proteins were expressed as described previously. While and pQE30/Q was used from prior studies, pQE60/Q2 plasmid was cloned and purchased from Genscript and Integrated DNA Technologies respectively. Q and Q2 were expressed in leucine auxotrophic LAM1000 E. coli cells in supplemented M9 minimal media. Prior to induction, expression media was allowed to grow to an optical density at 600 nm (OD₆₀₀) of 0.8-1.0 before pelleting at 5000×g at 4° C. for 30 minutes in an Avanti J-25 centrifuge (Beckman Coulter). Cells were washed a total of three times by resuspending in 0.9% NaCl previously stored at 4° C. overnight, centrifuging to re-pellet the cells in between washes. Following the final wash and centrifugation cycle, the cell pellet was resuspended in M9 media supplemented instead with 19 amino acids (minus leucine) and containing all other media chemicals. The expression culture was then incubated for 15 min at 37° C. and 350 rpm allowing for recovery while starving of leucine before addition of 555 μg/mL of L_{TF} and 200 μg/mL of IPTG to induce expression. After incubation at 37° C. and 350 rpm for 3 hours, cells were harvested by centrifugation at 5000×g at 4° C. for 30 minutes in an Avanti J-25 centrifuge (Beckman Coulter) and stored at −20° C. until purification. 12% SDS-PAGE was used to confirm expression of Q_{LTF} and Q2_{LTF}. Protein was purified using affinity chromatography on a cobalt-charged HiTrap IMAC FF 5 mL column with Buffer A (50 mM Tris-HCl, 500 mM NaCl, pH 8.0). Protein was eluted using a gradient of Buffer B (50 mM Tris-HCl, 500 mM NaCl, 500 mM imidazole, pH 8.0) possessing an imidazole concentration range from 10-500 mM. Pure fractions were then dialyzed in six consecutive 5 L volumes of Buffer A and concentrated to approximately 2 mM using 3 kDa Macrosep centrifugal filters (Pall). Protein purity was confirmed by 12% SDS-PAGE and concentration determined by BCA assay.

Assessment of Trifluoroleucine Incorporation. Trifluoroleucine (L_{TF}) was assessed by matrix-assisted laser desorption/ionization-time of flight mass spectrometry (MALDI-TOF MS) using a Bruker UltrafleXtreme MALDI-TOF/TOF. Protein was diluted 1:50 in water before being mixed in equal parts diluted sample to sinnapinic acid matrix. Protein sample was spotted onto a Bruker MTP 384 steel target plate and vacuum dried in a desiccator. Using the same protocol, Sigma Aldrich peptide standards were also spotted onto the target plate. The spectra were then deconvoluted to Gaussian functions in PeakFit software to its maximum goodness of fit by R² value using one peak to represent full incorporation, and >1 peak to represent masses less than full incorporation. The relative percent area of the incorporated Gaussian peak was used to determine the incorporation based on n number of peaks deconvoluted and if the Gaussian fit peak of the expected L_{TF} peak was less than the expected m/z, the % difference was incorporated into the assessment (Equation 3).

$$TFl incorporation ( \% ) = 100 \times \frac{Integrated of area of TFl peak}{{\sum }_{1}^{n} Integrated area of peak} \times \frac{1 - ( Measured TFl peak m / z - Expected TFl peak m / z )}{Expected TFl peak m / z} \qquad ( 3 )$$

Circular Dichroism Spectroscopy. The secondary structure of Q2_{LTF} and Q_{LTF} was assessed using a Jasco J-815 circular dischroism (CD) spectrometer with a PTC-423S single position Peltier temperature control system. Wavelength scans were performed from 195 to 250 nm at 1 nm step sizes by diluting the protein into water (at approximately 10 μM) in order to minimize the effects of sodium chloride. Temperature scans were performed from 20° C. to 85° C. in water and in phosphate buffer (50 mM Na₂HPO₄, pH 8.0) and in phosphate buffer in the presence of saturated CCM (as determined by binding data) at 1° C./min. The mean residue ellipticity (MRE) was calculated as described.

Attenuated Total Reflectance-Fourier Transform Infrared Spectroscopy. Secondary structure of Q2_{LTF} and Q_{LTF} protein was confirmed using attenuated total reflectance-Fourier transform infrared (ATR-FTIR) spectroscopy using a Nicolet 6700 Fourier Transform Infrared Spectrometer equipped with a diamond ATR accessory and a mercury cadmium telluride (MCT)-A detector. Spectra were collected for 5 μL of 1 mM protein from 4000-400 cm⁻¹ with 4.0 cm⁻¹ increments. Sample spectra were normalized using buffer background and analyzed from 1700-1600 cm⁻¹ corresponding to the amide I region. Peaks were deconvoluted using Gaussian functions in PeakFit software until the goodness of fit reached r²≥0.99.

Curcumin Binding. CCM was bound to Q2_{LTF}. Briefly, increasing ratios of CCM:Q2_{LTF} were made at final volumes of 1 mL with final concentrations of Q2_{LTF} at 15 μM and a final concentration of 1% v/v DMSO. Samples were loaded onto a 96-well black plate and excited at 420 nm and emission was read at 520 nm using a BioTek Synergy H1 microplate reader at room temperature (RT). Normalized relative fluorescence intensities were calculated and analyzed in Graphpad Prism (GraphPad Software). Binding affinity was calculated using the specific binding kinetics equation.

Transmission Electron Microscopy. Transmission electron microscopy (TEM) images were taken with a FEI Talos L120C transmission electron microscope. Samples were diluted to 50 μM and 3 μL was spotted on Formvar/carbon-coated copper grids followed by a 5 μL wash with water, and 3 μL staining with 1% v/v uranyl acetate solution each with incubation times of 1 min. Between steps, filter paper was used to wick the grids. Following imaging, fibrils were sized in ImageJ software (Version 1.52q).

Confocal Microscopy. Q2_{LTF} was diluted to 50 μM and saturated with 40 μM of curcumin (solubilized in DMSO) as determined by the binding affinity in drug-binding experiments. The final concentration of samples for confocal microscopy possessed 1% v/v DMSO. 5 μL of sample was deposited onto a microslide and covered with a 22×22 mm #1 microscope cover glass. Images were taken with a Leica TCS SP8 X laser confocal microscope using a dry 10× objective at RT. Samples were excited at 460 nm and images were taken with a 470-550 nm detection window.

¹⁹F Nuclear Magnetic Resonance. ¹⁹F detection was studied using a Bruker AVIII-500 (11.7 T) nuclear magnetic resonance (NMR) instrument equipped with a BB(F)O CryoProbe. ¹⁹F one-pulse sequence was performed with the zgig single pulse program, spectral width 241.5 ppm and 113636.4 Hz, 0.577 s acquisition time, and 254 scans. 1D ¹⁹F NMR spectra of Q_{LTF} and Q2_{LTF} in 10% v/v D₂O were collected in the approximate range of 0.25-2.0 mM based on concentrations measured by BCA assay following dilution in 10% v/v D₂O spiked buffer (50 mM Tris, 500 mM HCl, pH=8.0). 90% TFA/10% D₂O was acquired with the same sequence for comparison. Topspin 3.2 software was used to visualize spectra and quantify the signal-to-noise ratio (SNR) using the Bruker SINO command by calculating the ratio of the peak amplitude (signal) to the standard deviation of the noise level in the spectrum. To compare SNR signals between Q2_{LTF} and F-TRAP, SNR for F-TRAP at 11.7 T magnetic field was estimated using Equation 4.

SNR˜ηγₑ\sqrt{γ_{d}³Bₒ³t} (4)

Where η is the number of nuclear spins being observed, γₑ is the gyromagnetic ratio of the spin being excited, γ_{d} is the gyromagnetic ratio of the spin being detected, Bₒ is the magnetic field strength, and t is the experiment acquisition time.

T₁ and T₂ relaxation time of the fluorine nuclei in Q2_{LTF} was examined using the inversion recovery and Carr-Purcell-Meiboom-Gill sequence, respectively. The T₁ measurement was performed with a 4 s repetition time (TR) and 200 scans. Variable inversion times (TI) of 0.001, 0.05, 0.1, 0.25, 0.8, 1.5, 3.0, 5.0 s were used. The T₂ measurement was conducted using 4 s TR and 512 scans. Variable echo times (TE) of 0.002, 0.02, 0.05, 0.1, 0.2, 0.4, 0.6, 0.8, 1, 1.4, 1.6, 1.8, 2.5, 5, 10, 20 ms were employed. T₁ relaxation time was calculated using one-phase decay fitting analysis using Graphpad Prism software.

Phantom and In Vivo Magnetic Resonance Imaging. Magnetic resonance imaging (MRI) and spectroscopy (MRS) were performed on a Biospec 70/30 micro-MRI system (Bruker—Billerica MA, USA) equipped with zero helium boil-off 300 mm horizontal bore 7-Tesla (7-T) superconducting magnet (300 MHz) based on ultra-shield refrigerated magnet technology (USR). The magnet is interfaced to an actively shielded gradient coil insert (Bruker BGA-12S-HP; OD=198-mm, ID=114-mm, 660-mT/m gradient strength, 130-s rise time) and powered by high-performance gradient amplifier (IECO, Helsinki—Finland) operating at 300 A/500V. This installation is controlled by an Avance-3HD console operated under Paravision 6.1 and TopSpin 3.1. The MR imaging and spectroscopy setup utilized in this study involved in-house design of two distinct radiofrequency (rf) resonators for scanning a mouse body. The first coil was a volume transmit-receive linear birdcage rf coil with 16 rungs, possessing an outer diameter (OD) of 72 mm, an inner diameter (ID) of 42 mm, and a length (L) of 64 mm (FIG. 38a). This rf coil was tuned to 300.16 MHz, corresponding to the ¹H proton Larmor frequency. It served the purpose of transmitting and receiving signals during the imaging process, providing rf coverage for the mouse body. A rectangular flexible rf coil was also designed to enable specific detection of the fluorine (¹⁹F) nuclei (280 MHz) at 7-T. This flexible surface coil was fabricated by attaching adhesive flat copper tape circuitry affixed to a sheet transparency film. The coil had dimensions of L=10 mm and a width (W) of 30 mm (FIG. 38b). The coil incorporated four distributed fixed ceramic capacitors (Kyocera Co Ltd, Kyoto, JP), which facilitated tuning to a frequency near 280 MHz, corresponding to the ¹⁹F Larmor frequency.

The flexible rf coil was skillfully wrapped into the inner part of the cylindrical birdcage rf coil and positioned to optimize inductive coupling and radiofrequency (rf) coverage (FIG. 38c). This configuration enabled the achievement of dual resonance for both ¹H and ¹⁹F nuclei utilizing a single port. The single port was connected to a tune/match box, which served as an interface between the coil and the spectrometer (FIG. 38d). The utilization of this dual-tune setup facilitated the acquisition of imaging and spectroscopy data for both proton (¹H) and fluorine (¹⁹F) signals, allowing for comprehensive analysis and investigation described herein. A set of ¹⁹F magnetic resonance spectra were acquired for the calibration and characterization of the custom-designed RF coils setup. The acquisition parameters included a TR=5 s to enable full magnetization recovery, a number of averages (Nav)=1, and 2048 points for the acquisition with a spectral width (SW)=85.227 kHz resulting in spectral resolution of 21 Hz/pt.

A set of water phantoms doped with copper sulfate and NMR tubes filled individually with 100 μL of 100% water, 13 mM trifluoroacetic acid (TFA, 100%) and 1 mM Q2_{LTF} were used for characterizing ¹H/¹⁹F rf coil set sensitivity and performance. After conducting rf power and shim calibrations using the ¹H signal, serial dilutions of TFA NMR tubes were utilized as a reference to evaluate the limit of detection (LOD) for the ¹⁹F signal in our experimental setup. The LOD was established by determining the concentration that achieved a SNR above 3 standard deviations of the noise floor. The ¹⁹F signal optimization of Q2_{LTF} was subsequently carried out. To achieve a constant scan time of 4 minutes, TR was incrementally increased from 50 ms to 1000 ms by adjusting the number of averages. The objective of this optimization was to determine the best combination of TR and Nav to acquire Q2_{LTF} spectra with maximum sensitivity under 4 minutes. Additionally, the same optimization process was repeated at a reduced acquisition time of 1 minute to evaluate the impact of improved temporal resolution on SNR. The SNR values were calculated using the “sino” command in Bruker Topspin software. Specifically, the ¹⁹F signal interval was defined between −50 ppm and −100 ppm, while the background noise region was selected within the 0 ppm to 50 ppm chemical shift range. The spectra were set to a line broadening (LB) value of 30 for display purposes only. By following this experimental protocol, the calibration of rf power, shim adjustments, and optimization of TR were achieved, ensuring accurate NMR spectral acquisition and analysis for the Q2_{LTF} samples.

To perform in vivo MRI scans, the lower body of the mouse was positioned within the isocenter of the magnet within the rf coil to ensure comprehensive anatomical coverage with the knee closely fitting the rectangular surface coil. To provide anatomical context, a ¹H MRI scan was performed using a 3D gradient echo (3D-GE) Flash sequence. The scan parameters were set as follows: TR=40 ms, echo time (TE)=2.1 ms, flip angle (FA)=30 degrees, matrix size (Mx)=256×128×128, field of view (FOV)=51.2×25.6×25.6 mm, N_{AV}=2. The acquisition time for this scan was less than 22 minutes, resulting in an isotropic image resolution of 200 μm. The primary objective of this scan was to accurately visualize the intra-articular location of Q2_{LTF} injected at 1 mM (50 μl) within the mouse.

For ¹⁹F scans, the phantom MRI settings were utilized as a reference, with an acquisition time of 10 minutes (TR=100 ms, N_{AV}=6000). This setup ensured consistent imaging conditions for the ¹⁹F scans, enabling accurate comparison and analysis of the Q2_{LTF} biomaterial.

To compare the ¹⁹F MRS sensitivity of the current Q2_{LTF} biomaterial with a previously studied L_{TF}-incorporated construct called F-TRAP, the scan time was adjusted to 6 min. 40 sec (TR=100 ms, N_{AV}=4,000). This adjustment was made while keeping the MRI settings optimized for Q2_{LTF} and utilizing the same coil setup employed in this study. Consequently, the following parameters were employed: 2048 points and SW=85.227 kHz, resulting in a spectral resolution of 21 Hz per data point.

Ultrasound guided injection. The image-guided intra-articular injection of the Q2_{LTF} was performed using a Vevo 3100 high-frequency ultrasound (US) system (Visualsonics/Fujifilm, Toronto ON, CA). The system was equipped with an adjustable rail system designed for small animal handling, precise positioning, and optimization. This setup allowed for noninvasive in vivo imaging under accurate physiological conditions, which included a temperature-controlled heated stage, gas anesthesia, and a syringe injection system for simultaneous compound administration.

A 50 MHz high-frequency US transducer (MX700 D) was utilized, providing axial resolution of 30 μm and enabling real-time imaging at a rate of up to 300 frames per second. To ensure optimal imaging conditions, mice were positioned in a dorsal recumbent posture on the US heated stage. The hind limbs were flexed and externally rotated approximately 450 while applying a surgical tape to immobilize the limbs and facilitate access to the knee joint.

Prior to the injection, sterile US gel was applied over the joint area to enhance visualization and guidance during the injection process. The US transducer was positioned parallel to the femur, allowing for clear visualization of the patellar ligament, which appeared as a dark band in the ultrasound image.

For the injection itself, a needle was carefully inserted laterally to the patellar tendon, within the joint capsule. The Q2_{LTF} (1 mM, 50 μl) solution was slowly infused through the needle, while the intra-articular release was continuously monitored using ultrasound imaging.

By employing this technique, the image-guided intra-articular injection of the Q_{2LTF} was successfully performed, ensuring reproducible targeting and delivery of the compound within the joint space while allowing for real-time monitoring of the injection process.

Statistical Analysis. GraphPad Prism (GraphPad Software) was employed for statistical analysis using student's t-test.

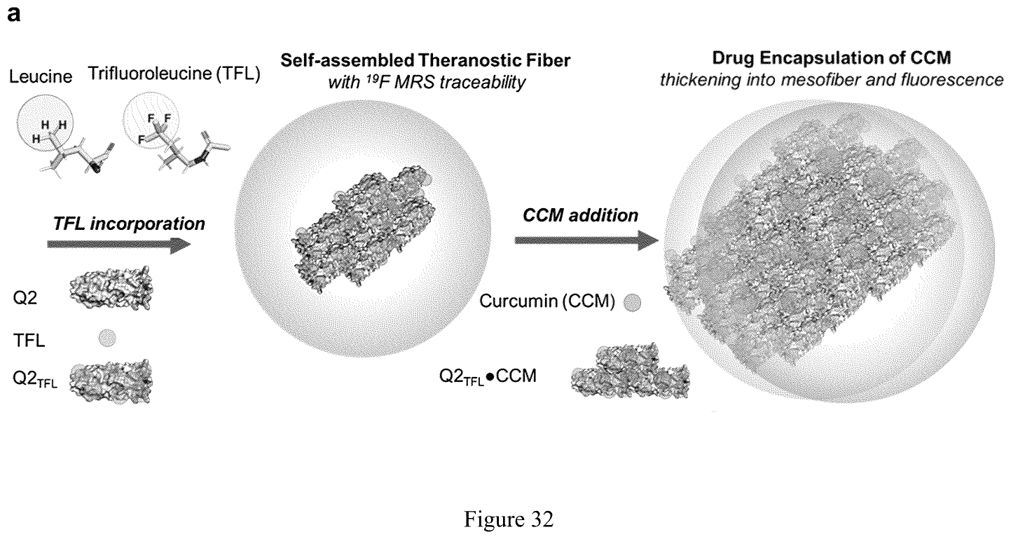

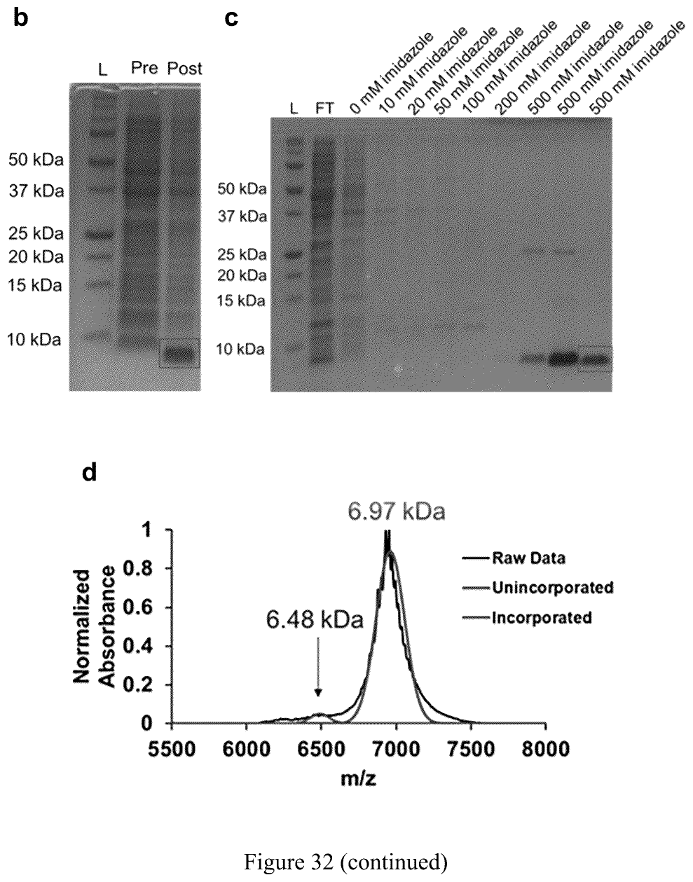

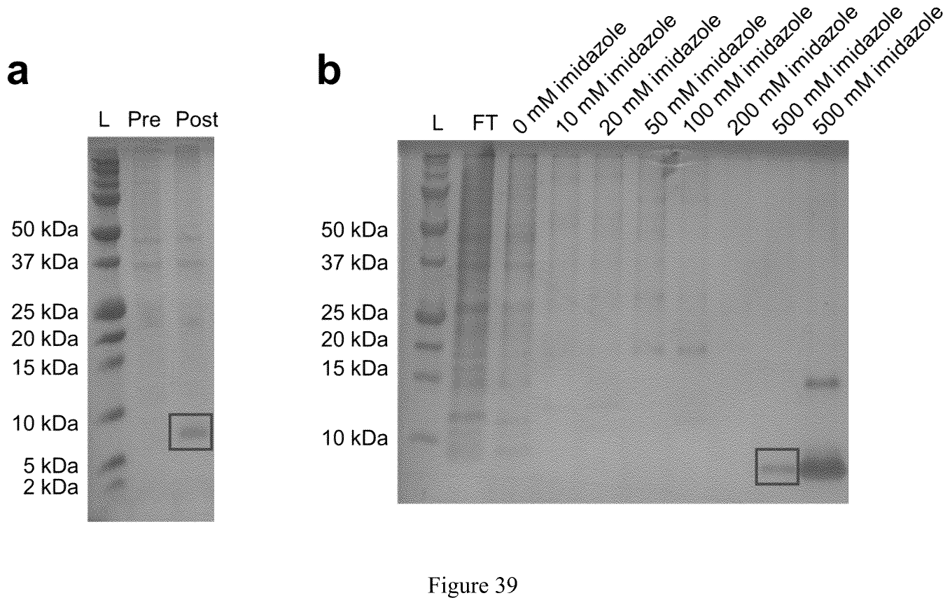

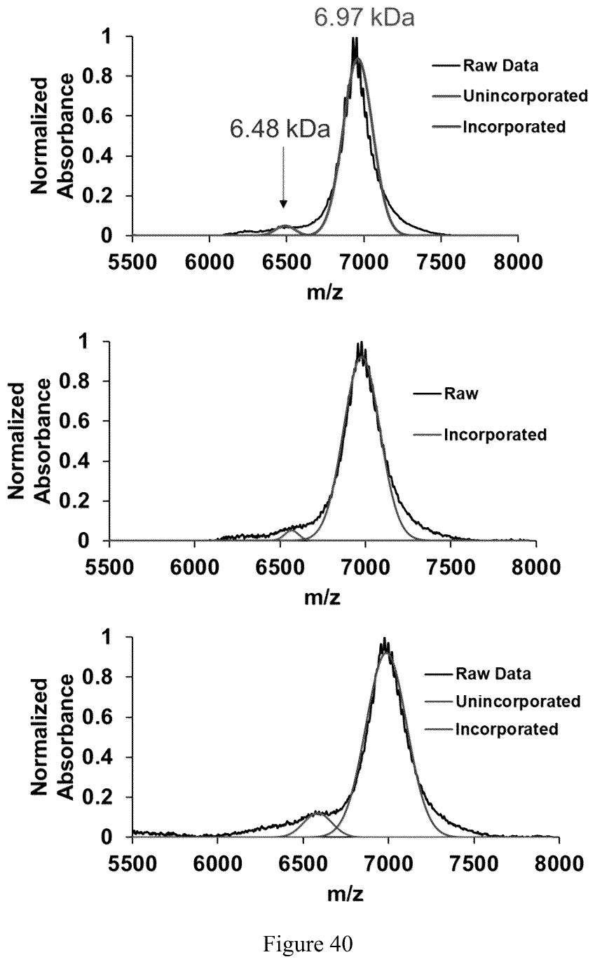

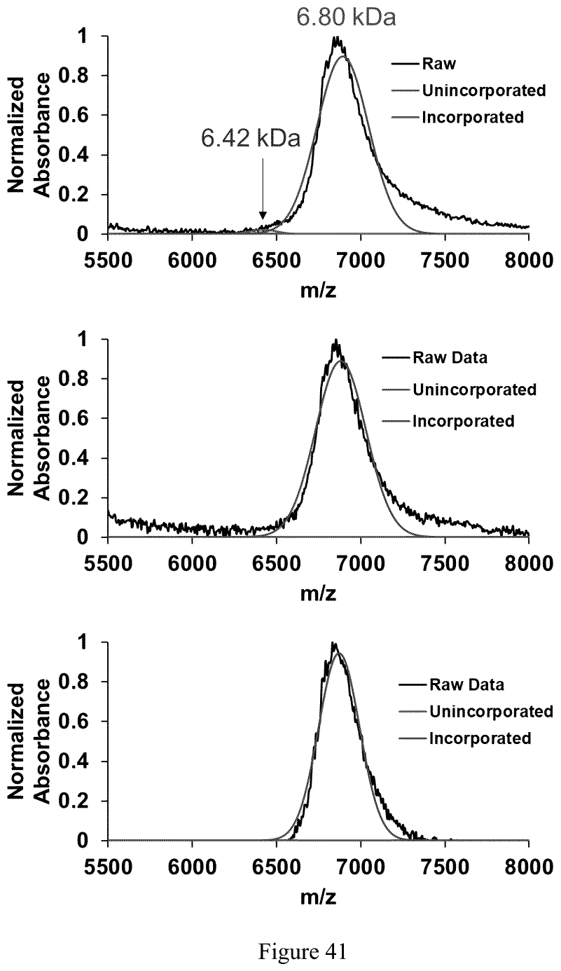

Rationale and Protein Synthesis. Q2_{LTF} was designed to for greater thermostability possessing 9 leucines, compared to 7 in Q_{LTF}, which was confirmed by a higher Rosetta Score with the aim of creating a fluorinated fiber capable of curcumin (CCM) encapsulation (FIG. 32a). Q2_{LTF} was generated by residue-specific non-canonical amino acid incorporation of trifluoroleucine (L_{TF}) using leucine auxotrophic LAM1000 E. coli cells. Protein expression (FIG. 32b,) and purification (FIG. 32c) were assessed by 12% SDS-PAGE gels showing protein bands at molecular weight of 6.97 kDa for Q2_{LTF}. Percent of L_{TF} incorporation was assessed using MALDI-TOF based on the molecular weight of Q2 (6.48 kDa) (Table 10). Q2_{LTF} showed an expected increase in molecular weight upon incorporation of L_{TF} of 0.49 kDa based on the difference in molecular weight of L_{TF} (185.14 Da) and leucine (131.17 Da) and the number of leucines. Using best-fit Gaussian peaks based on the expected molecular weight of incorporated and unincorporated proteins, Q2_{LTF} was determined to have an average incorporation of 95.0±2.3% (FIG. 32d, 41, Table 11) with this value near the expected range based on previous incorporation levels for L_{TF} in coiled-coils from previous studies, which ranged from 90.7%-95.1%. As a control, Q_{LTF} was biosynthesized, purified and confirmed for L_{TF} incorporation as previously described (FIG. 39, 42, Table 10.

TABLE 10
Sequences for fluorinated and non-fluorinated Q and Q2 constructs
and calculated molecular weights, where X is L or L_{TF}

| Protein | Sequence | MW (kDa) |
|---|---|---|
| Q/Q_{LTF} | MRGSHHHHHHGSIEGR VKE ITFXKNT | 6.42/6.80 |
| | APOMXRE XQETNAA XQDVREX XRQQSKX (SEQ ID | |
| | NO: 19) | |
| Q2/Q2_{LTF} | MRGSHHHHHHGSIEGR VKE XXFXKKT AEQMXEE | 6.48/6.97 |
| | XKETNKA XHDVRHX XENQSKX (SEQ ID NO: 20) | |

TABLE 11
Calculation results for L_{TF} incorporation of
Q2_{LTF}. Color-coded to calculation steps in Equation 3.

| Incorporation | Sample 1 | Sample 2 | Sample 3 | Average |
|---|---|---|---|---|
| % integrated area | 96.5% | 92.5% | 91.7% | 93.6 ± 0.6% |
| of incorporated | | | | |
| peak | | | | |
| % distance to total | 100% | 99.8% | 99.7% | 99.9 ± 0.2% |
| incorporated peak | | | | |
| (6.97 kDa) | | | | |
| Product of % | 96.5% | 92.4% | 96.0% | 95.0 ± 2.3% |
| incorporation | | | | |

TABLE 12
Calculation results for L_{TF} incorporation of Q_{LTFl}
corresponding to calculation steps in Equation 1.

| Incorporation | Sample 1 | Sample 2 | Sample 3 | Average |
|---|---|---|---|---|
| % integrated area | 100.0% | 98.8% | 100.0% | 99.5 ± 0.8% |
| of incorporated | | | | |
| peak | | | | |
| % distance to total | 100.0% | 98.6% | 100.0% | 99.6 ± 0.9% |
| incorporated peak | | | | |
| (6.80 kDa) | | | | |
| Product of % | 100.0% | 96.0% | 100.0% | 98.7 ± 2.3% |
| incorporation | | | | |

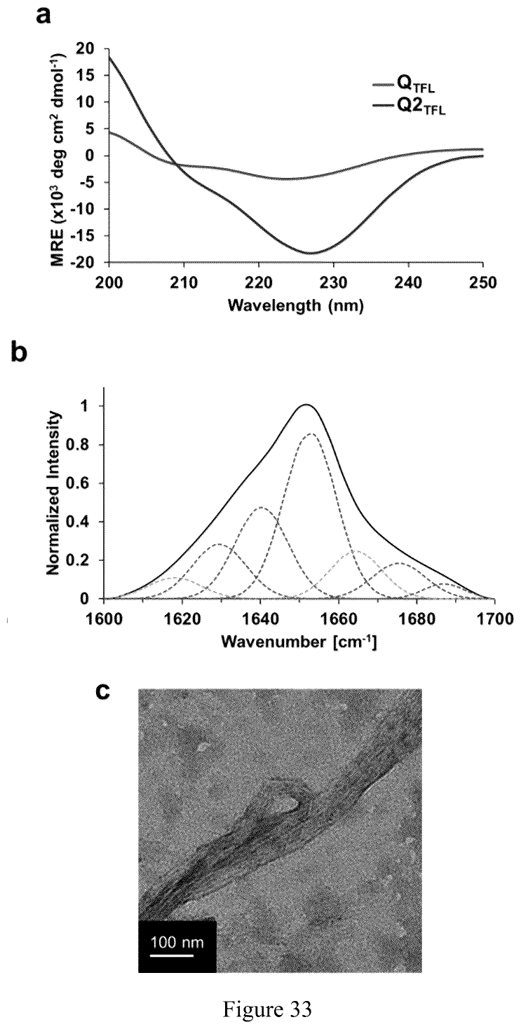

Fluorinated coiled-coil structure. The secondary structure of Q2_{LTF} was assessed using CD spectroscopy. Q2_{LTF} exhibited a characteristic α-helical spectrum with a double minimum of −100 deg·cm²·dmol⁻¹ and −15,000 deg·cm²·dmol⁻¹ at 208 nm and 222 nm, respectively (FIG. 33a, Table 13). Additionally, Q2_{LTF} possessed a 222/208 ratio of 150. The large magnitude of the 222/208 ratio suggests that ax-helices were found in proximity of other α-helices reflecting the coiled-coil and fibrous nature of Q2_{LTF}. To further explore the impact of the higher L_{TF} content in Q2_{LTF}, we compared the data with the previously fluorinated fiber, Q_{LTF}. The parent Q_{LTF} exhibited a double minimum of −500 deg·cm²·dmol⁻¹ and −4,300 deg·cm²·dmol⁻¹ at 208 and 222 nm, respectively and a 222/208 ratio of 8.6 (FIG. 33a, Table 13). Q2_{LTF} demonstrated a much stronger coiled-coil structure and α-helical characteristic minimum at 222 nm, in agreement with previous studies of fluorination on coiled-coil structure.

TABLE 13
MRE values calculated from CD measurements at 208 nm and 222
nm. Standard error is the result of three independent trials.

| | θ₂₀₈ (deg · cm² · dmol⁻¹) | θ₂₂₂ (deg · cm² · dmol⁻¹) |
|---|---|---|
| Q_{LTF} | −500 ± 800 | −4,300 ± 300 |
| Q2_{LTF} | −100 ± 1,800 | −15,000 ± 2,000 |

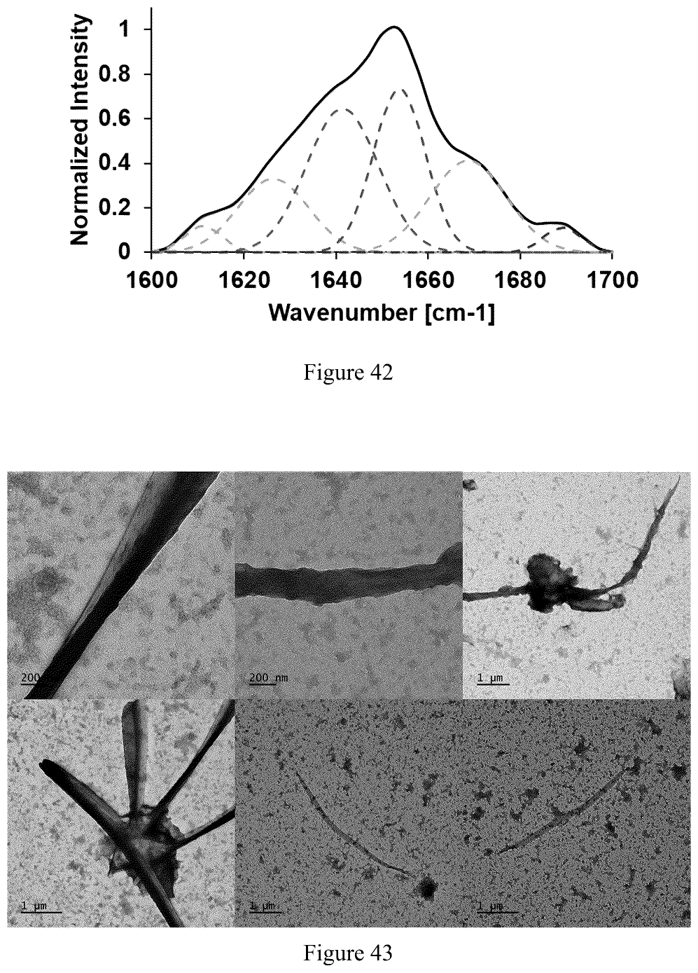

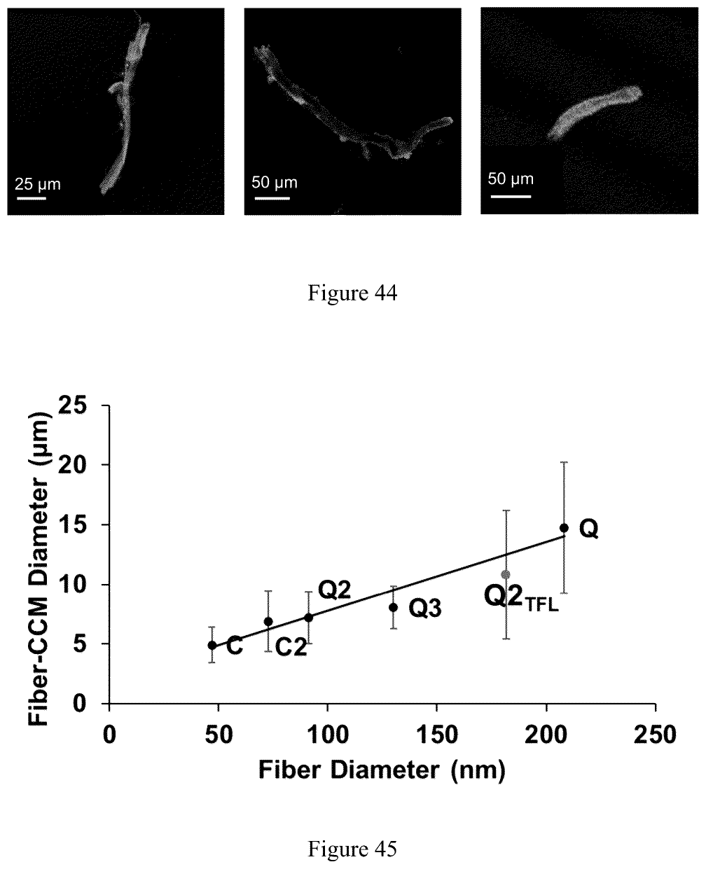

In addition, Q2_{LTF} secondary structure in its native buffer conditions was assessed using ATR-FTIR of the samples at 2 mM (FIG. 33b). In agreement with CD results, Q2_{LTF} revealed a helical content of 38.4% after deconvolution (Table 14), which was 13.6% higher than the parent Q_{LTF} (FIG. 42, Table 14), indicating the positive effect of additional L_{TF}'s on coiled-coil structure.

TABLE 14
ATR-FTIR compositional analysis from Q_{LTF}, Q2_{LTF}, and
Q2_{LTF}-CCM. Summary of secondary structure content uses
the average and standard deviation of the integrated
area of deconvoluted peaks of three independent trials.

| | % composition | | | | | |
|---|---|---|---|---|---|---|
| | α- | β- | Antiparallel | 3-10 | Unor- | Aggregated |
| | helix | sheet | β-sheet | helix | dered | Strands |
| Q_{LTF} | 24.8 ± | 30.6 ± | 8.0 ± | 18.6 ± | 0.0 ± | 17.4 ± |
| | 6.8 | 10.2 | 8.8 | 3.6 | 0.0 | 4.6 |
| Q2_{LTF} | 38.4 ± | 28.7 ± | 13.8 ± | 12.4 ± | 0.0 ± | 6.8 ± |
| | 14.0 | 6.6 | 5.5 | 7.7 | 0.0 | 5.9 |
| Q2_{LTF}- | 30.8 ± | 21.7 ± | 0.0 ± | 23.5 ± | 0.0 ± | 9.8 ± |
| CCM | 6.9 | 9.9 | 0.0 | 6.1 | 0.0 | 5.6 |

Supramolecular assembly and microstructure. Given the nature of the Q proteins to undergo supramolecular assembly at low temperatures, Q2_{LTF} was incubated at 4° C. after concentration to 2 mM in 50 mM Tris, 500 mM NaCl, pH 8.0 buffer. Q2_{LTF} underwent supramolecular assembly into nanofibers. Lower resolution micrographs showed Q2_{LTF} fiber morphology to appear similar to those found for Q_{LTF} containing large diameter and sheet-like structures. Higher resolution micrograph images revealed a fibrous structure composed of striations measuring 3.6±0.8 nm in size (n=20) (FIG. 33c, 43), approximately the diameter of a single coiled-coil domain and in agreement with the 3.5±0.5 nm protofibril diameters measured in Q previously and suggesting a similar end-to-end stacking mechanism.

Overall, fiber assemblies are measured to be 215.8±38.6 nm (n=20) in size by TEM (FIG. 33c, 43). The large standard error is explained by the presence of large fiber aggregates as big as 840 nm in diameter. As a result, the median diameter, 181.7 nm, was viewed as a better representation of typical fiber diameter was viewed as the median diameter. Previously, nanofibril diameter size has been associated with the electrostatic potential of protofibril termini, the increase in diameter of Q2_{LTF} fibrils suggested the size can also be modulated by hydrophobicity, namely by fluorinating or modifying the number of hydrophobic residues lining the coiled-coil pore. To this extent, this agrees with phenomena associated with fiber thickening upon introduction of hydrophobic small molecule curcumin (CCM) in fibers described herein. Strong interaction of CCM in the hydrophobic pore and in between fibers causes hydrophobic residues to be further buried and increases the exposure of nonpolar residue groups and thus increases protein surface activity. Without intending to be bound by any particular theory, the introduction of hydrophobic residues is associated with increased hydrophobic residue packing and surface activity, which in turn, increases protofibril interaction resulting in a population of larger fiber diameters.

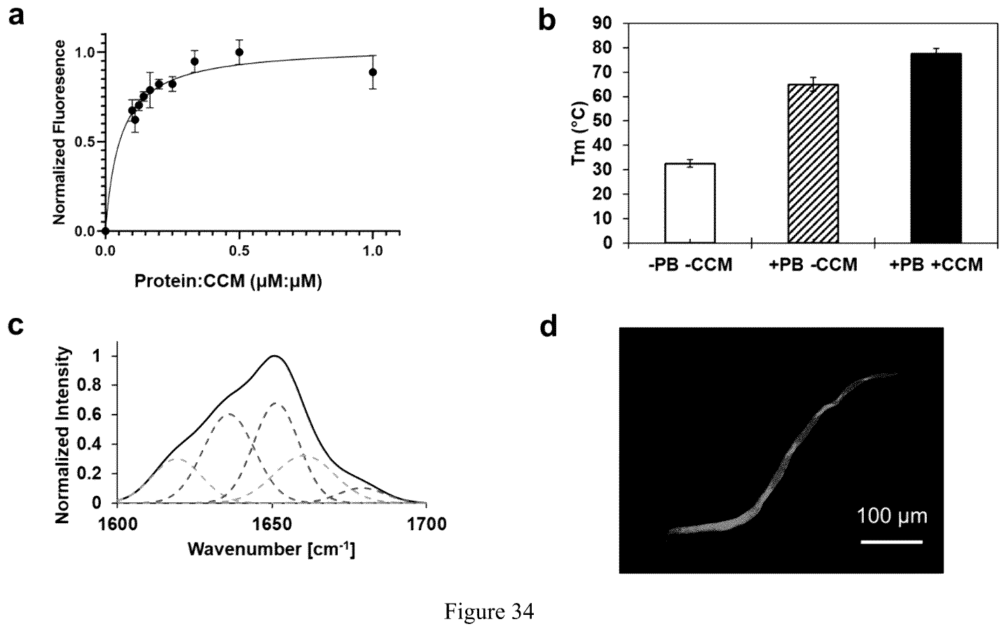

Q2_{LTF} thermostability was measured by CD temperature scans from 20° C. to 85° C. In only water, in the absence of salts or buffers, Q2_{LTF} exhibited a melting temperature of 32.6±1.6° C. (FIG. 34a). However, under physiologically relevant buffer conditions such as the phosphate buffer used herein, Q2_{LTF} possessed a melting temperature of 65.0±2.9° C. This range spans physiological temperature where Q2_{LTF} meets the criteria of an ionic strength-responsive protein biomaterial for controlled drug release. In previous studies, it was observed that Q_{LTF} exhibited an increase in melting temperature, rising from 39° C. to 52° C. This substantial enhancement in thermostability aligns with previous research indicating that fluorinated coiled-coils tend to improve stability. Notably, a higher content of L_{TF} resulted in a more significant increase in stability, highlighting the relationship between L_{TF} concentration and improved stability.

Curcumin binding. Coiled-coil proteins have traditionally been studied for their hydrophobic small molecule-binding ability due to the presence of a hydrophobic pore. In particular, curcumin (CCM) has been used as a model candidate drug due to its therapeutic use as an antiproliferative, antibacterial, and anti-inflammatory agent. The ability of Q2_{LTF} to bind CCM (FIG. 34b) and the impact of encapsulation on Q2_{LTF} structure and stability was assessed, where Q2_{LTF} exhibits a K_{d} of 0.06 μM:μM [protein:CCM] (0.03-0.09 μM:μM @95% CI), which translates to an 8:1 ratio of monomer to CCM, a significant increase compared to Q2. 2×K_{d} or a ratio of 0.12 was used to mark saturation of CCM binding and where a negligible increase in fluorescence is seen. Moreover, CCM-bound Q2_{LTF} (Q2_{LTF}·CCM) exhibits a 12.6° C. increase in melting temperature to 77.6±2.0° C. (FIG. 34b) via CD, which is consistent with previously reported increases upon CCM binding.

ATR-FTIR was used to decipher secondary structure of Q2_{LTF} (FIG. 33b) and compared to Q2_{LTF}·CCM (FIG. 34c). After deconvolution of spectra, Q2_{LTF} exhibited 42.4±8.6% α-helical content, 38.4±14.0% β-sheet content, and 19.2±9.7% random coil content (Table 14). Upon binding to CCM, noted by broadening of the ATR-FTIR spectra, Q2_{LTF}·CCM possesses 30.8±6.9% α-helical content, 33.0±130.7% β-sheet content, 33.3±8.3% random coil content (Table 14) exhibiting a 14.1% loss in ordered structure—a behavior consistent with previous fiber·CCM binding studies.

A linear model was previously established correlating the increase in thermostability upon binding CCM, and the loss of ordered structure as measured by ATR-FTIR. Based on this model, a 14.5% loss of structure is predicted, which translates to an error of just 0.4% from a measured structure loss of 14.1%, within the root mean squared error (RMSE) of the model, which is calculated here to be 0.9%. These results both validate the linear model and strengthen the conclusion that Q2_{LTF} possesses similar binding behavior to non-fluorinated fibers previously studied. While CCM-binding imposes a negative impact on the ordered structure of the coiled-coil, a loss of secondary structure measured by ATR-FTIR has been associated with a positive interaction of CCM in the hydrophobic pore, which helps stabilize the protein and increase thermostability. Thus, the fluorination of Q2_{LTF} results in a strengthened interaction with CCM.

Binding of CCM causes fiber thickening of Q2_{LTF} (FIG. 34d, 44), consistent with recent analysis of supramolecular coiled-coil fibers. Fiber-thickening by CCM has been established for collagen activity as well as coiled-coil fibers and is explained by increased solvation of polar groups and burying of the hydrophobic residues leading to increased surface activity. The average fiber diameter of 10.8±5.4 μm is similar to the median fiber diameter predicted by a recently established relationship between nanofiber and CCM-thickened fiber diameters. While this relationship was assessed for non-fluorinated CCM fibers, the predicted fiber diameter is 12.9 μm based on a 181.7 nm Q2_{LTF} fiber diameter translating to an error of 2.1 μm, just outside our model's root mean squared error (RMSE) of 0.8 μm (FIG. 45). These results suggest that Q2_{LTF} supramolecular fiber assembly upon CCM-binding remains similar to previous non-fluorinated constructs.

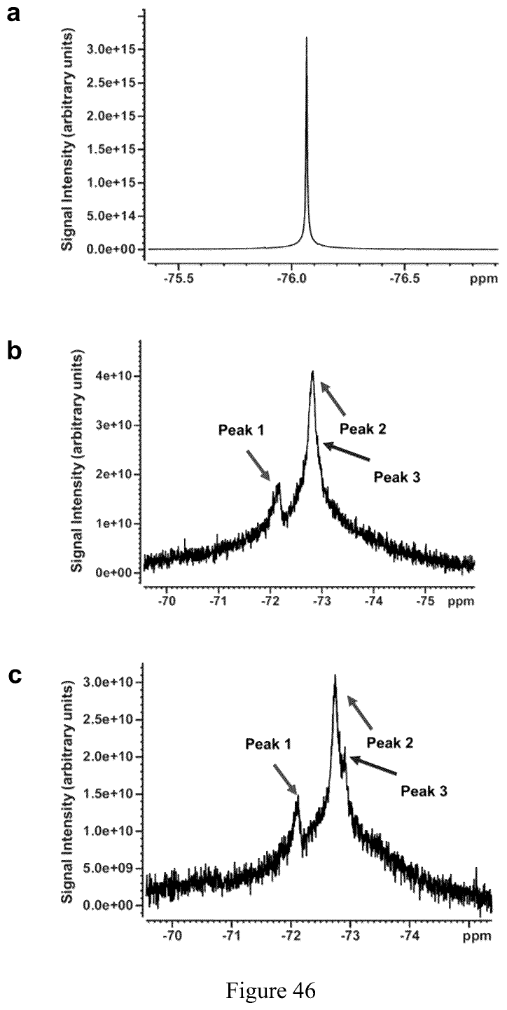

¹⁹F Nuclear Magnetic Resonance. To determine the potential for Q2_{LTF} as a non-invasive ¹⁹F MR dynamic probe, initial ¹⁹F NMR was performed on a 500 MHz spectrometer. Q_{LTF} and Q2_{LTF} exhibited triplet NMR peaks (FIG. 46a-b) consistent with the triple fluorinated residue motif. Due to the presence of peak overlap in the spectrum of FIG. 46a-b, the accurate identification and distinction of individual peaks becomes challenging. Specifically, peak 3, characterized by the largest chemical shift, overlaps with peak 2, making it difficult to reliably detect and distinguish them. This overlap and reduced clarity was attributed to protein conformational heterogeneity, which can result in line broadening. This overlap hinders the clear resolution of the individual contributions of these peaks, potentially complicating their proper identification and quantification. Despite the challenges posed by the peak overlap, the overall T₁ and T₂ relaxation times of Q2_{LTF} in its ¹⁹F NMR spectrum were characterized. Q2_{LTF} demonstrated a ¹⁹F T₁ relaxation time of 329 ms and a T₂ relaxation time of 120 s in its ¹⁹F NMR spectrum at 25° C. and 5.6 mg/mL. In comparison, previous findings reported that F-TRAP at a concentration of 1 mg/mL and 22° C., exhibited ¹⁹F T₁ of 393 ms and a T₂ of 1.2 ms.

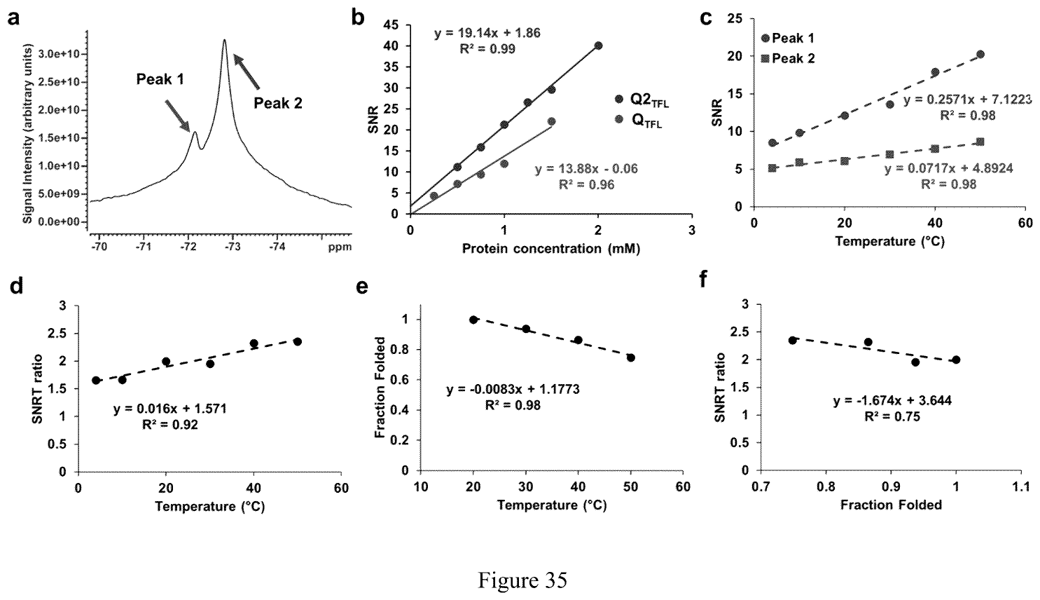

Q2_{LTF} and Q_{LTF} were measured at molar concentrations 0.25-2.0 mM (Table 15) and the signal-to-noise ratio (SNR) was measured for each spectrum using 50-100 ppm to represent all signal that appeared in the spectra and 0-50 ppm, where no signal appeared, to represent the noise. TFA exhibited a chemical shift of −76.1 ppm (FIG. 46a), consistent with reported values. Q2_{LTF} displayed a chemical shift of −72.8 ppm (FIG. 35a, 46b), whereas the parent Q_{LTF} exhibited a chemical shift of −72.6 (FIG. 46c). Additionally, Q2_{LTF} demonstrated a SNR dependence on ¹⁹F molar concentration of 19.14 mM⁻¹, while Q_{LTF} showed a relationship of 13.88 mM⁻¹ to SNR (FIG. 35b). Notably, the SNR efficiency with respect to ¹⁹F molar concentration of Q2_{LTF} was 1.38 times greater than that of Q_{LTF}, which is consistent with the expected increase based on the theoretical 9/7 LF ratio of Q2_{LTF}/Q_{LTF}.

TABLE 15
SNR values calculated from Q2_{LTF} and Q_{LTF} NMR
spectra at various molar concentrations.

| Concentration (mM) | Q2_{LTF} | Q_{LTF} |
|---|---|---|
| 2.0 | 40.20 | — |
| 1.5 | 29.70 | 1.32 |
| 1.25 | 26.67 | — |
| 1 | 21.31 | 0.88 |
| 0.75 | 15.98 | 0.66 |
| 0.5 | 11.25 | 0.44 |
| 0.25 | — | 0.22 |

In comparison, F-TRAP, exhibited an SNR efficiency with respect to ¹⁹F molar concentration of ˜13.6 mM⁻¹ at a magnetic field strength of 400 MHz. To account for the difference in magnetic field strength, an estimation of the SNR performance at a 500 MHz was made based on Equation 4, which states that the SNR is proportional to B₀^{3/2} (where B₀ is magnetic field strength). Using this equation, it can be estimated that F-TRAP would achieve an SNR performance of ˜19.0 mM⁻¹ at 500 MHz. This estimation suggests that Q2_{LTF}, as an improved protein-engineered drug delivery agent, generates a stronger ¹⁹F MR signal at equal molar concentrations compared to F-TRAP. Furthermore, Q2_{LTF} possesses 9 L_{TF} per monomer with a monomeric molecular weight of 6.97 kDa, whereas F-TRAP has 11 L_{TF} per monomer with a monomeric molecular weight of 16.74 kDa. This translates to a SNR slope of 2.74 mg/mL⁻¹ for Q2_{LTF}, which is 2.4 times higher than the 114 mg/mL⁻¹ SNR slope for F-TRAP. These results suggest that Q2_{LTF} is significantly more powerful by mass.

Finally, the Q2_{LTF} was assessed for temperature dependence by altering the environmental temperature in NMR. Q2_{LTF} exhibited an increase in SNR with an increase in temperature, dominated by peak 2 at all temperatures. SNR of each peak was assessed individually by acquiring 1 ppm breadths around peak 1 and peak 2 resulting in independent temperature coefficients (FIG. 35c). At constant concentration, the ratio of these slopes was used to determine an independent SNR-temperature coefficient for Q2_{LTF} dubbed SNRT ratio. As expected, linear temperature dependence was retained with the SNRT ratio (FIG. 35d), illustrating the ability to predict temperature using the ratio of peak 1 and peak 2. This suggested that it could serve as a valuable tool for temperature monitoring. Furthermore, Q2_{LTF} possessed a linear correlation at in vivo relevant temperatures with an R²=0.98 (FIG. 35e). Thus, SNRT ratio was correlated with fraction folded assessed by CD at in vivo relevant temperatures with an R²=0.75 (FIG. 35f), indicating a strong linear relationship and demonstrating the ability to predict relative structure from overall SNRT ratio alone. These preliminary results show promising potential for the applications of SNRT. Further investigations can explore its use as a valuable tool for in vivo monitoring of Q_{LTF} structure and temperature, particularly in areas such as hyperthermia and drug release control.

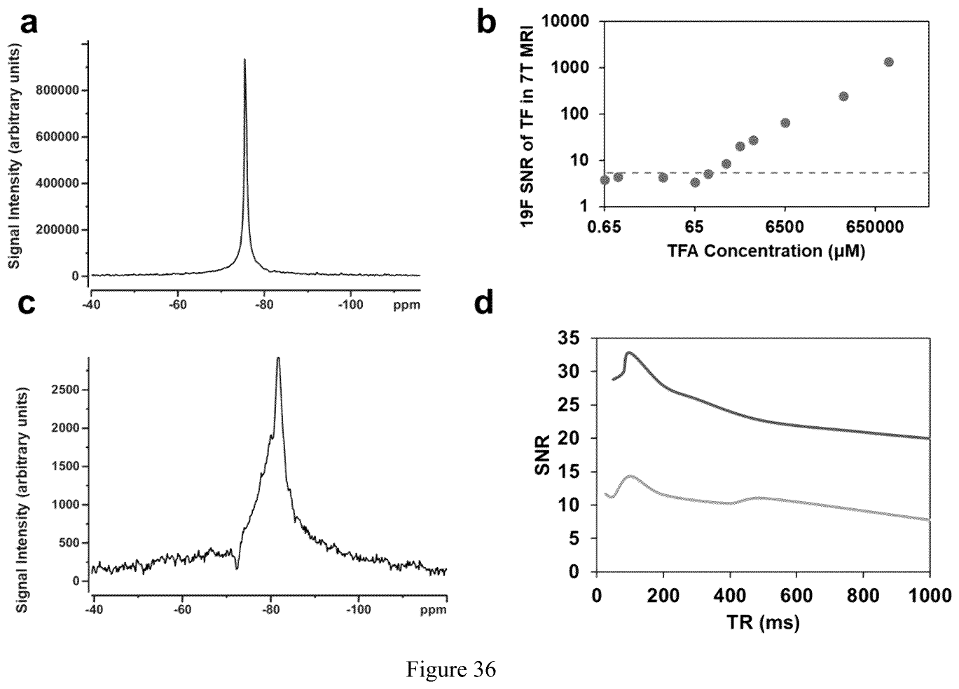

Phantom Magnetic Resonance Imaging. Q2_{LTF} was tested for its ability to act as a traceable drug delivery agent using an in vivo experimental setup with the custom rf coil designed for the 7-T Bruker 7030 Biospec β-MRI system. The MRI sequence parameters for the in-house customized dual-resonance rf probe were first optimized by phantom imaging of 100 μL of 100% TFA (13 mM) and 1 mM Q2_{LTF} samples. The limit of detection (LOD) (FIG. 36a) was assessed for ¹⁹F using TFA, which possessed chemical shift −75.4 ppm (FIG. 36b) with spectral resolution 41.6 Hz/pt. The LOD was calculated to be 5.3, which was reached by 130 μM TFA. Based on relative SNR of Q2_{LTF}, this suggests that the LOD would be reached by ˜100 μM of Q2_{LTF} using the 1.46 Q2_{LTF}:TFA (mM:mM) SNR ratio as determined by NMR. Phantom MRI of Q2_{LTF} signals measured by ¹⁹F MRS show a chemical shift of −79.8 ppm, similar to the difference to TFA assessed by NMR, confirming acquisition of Q2_{LTF} specific spectra.

Moreover, the in vivo study aimed to optimize signal maximization by varying the repetition time (TR) and scan times. The number of averages was adjusted to maintain a consistent scan time across different TR values (FIG. 36c, Table 16). Notably, Q2_{LTF} exhibited the highest signal performance at TR between 80 ms-100 ms. Longer scan time of 4 min. (FIG. 36d) showed a significant improvement in SNR, while shorter 1-minute scan times yielded an expected ˜2× reduction in SNR. Nevertheless, the sensitivity of the 1-minute remained above the LOD, allowing for the acquisition of Q2_{LTF} spectra with improved temporal resolution for traceability purposes.

TABLE 16
SNR values calculated from Q2_{LTF} at different TR and scan times
using number of averages to maintain scan time at different TR.

| TR (ms) | SNR (4 min.) | SNR (1 min.) |
|---|---|---|
| 1000 | 19.94 | 7.79 |
| 800 | 20.9 | — |
| 500 | 22.6 | 11.06 |
| 400 | — | — |
| 300 | 25.85 | 11.87 |
| 200 | 27.87 | 11.57 |
| 100 | 32.77 | 14.35 |
| 80 | 29.84 | — |
| 50 | 28.78 | 11.3 |
| 40 | — | — |
| 30 | 28.54 | — |
| 26 | — | 11.7 |

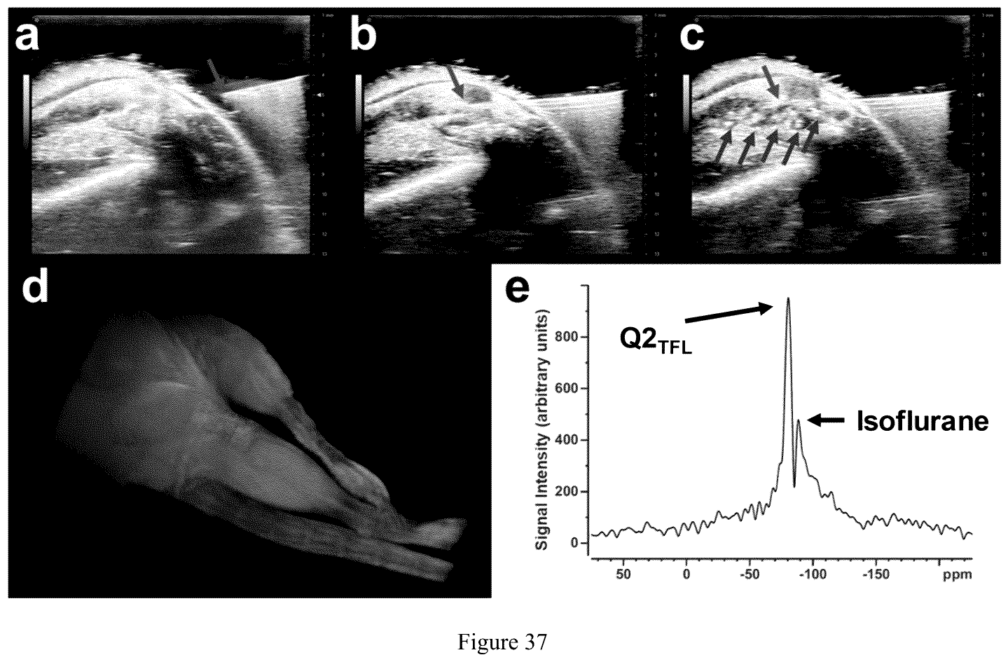

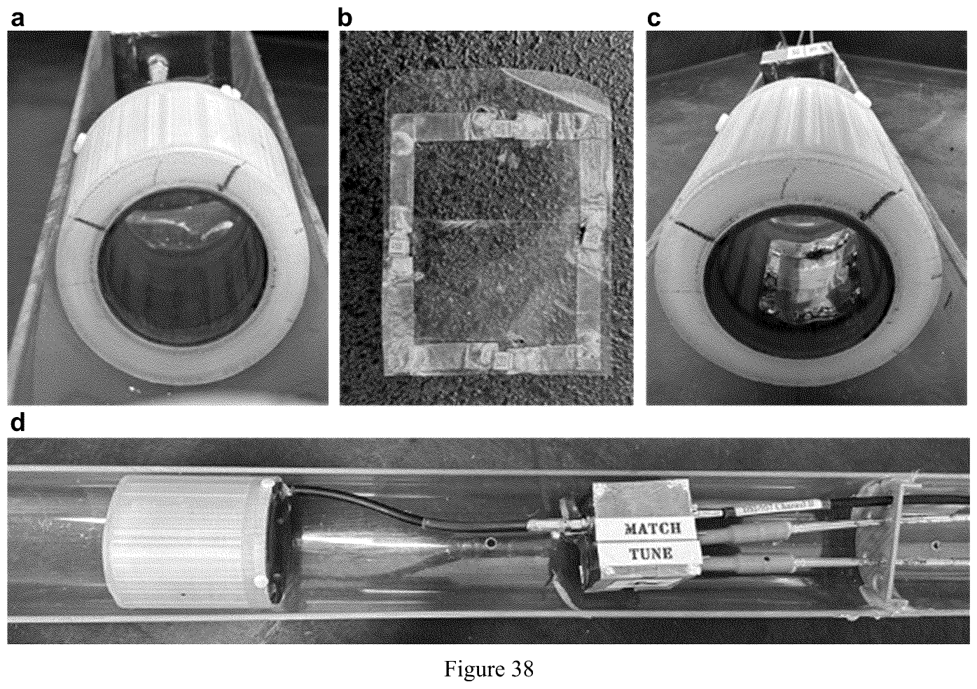

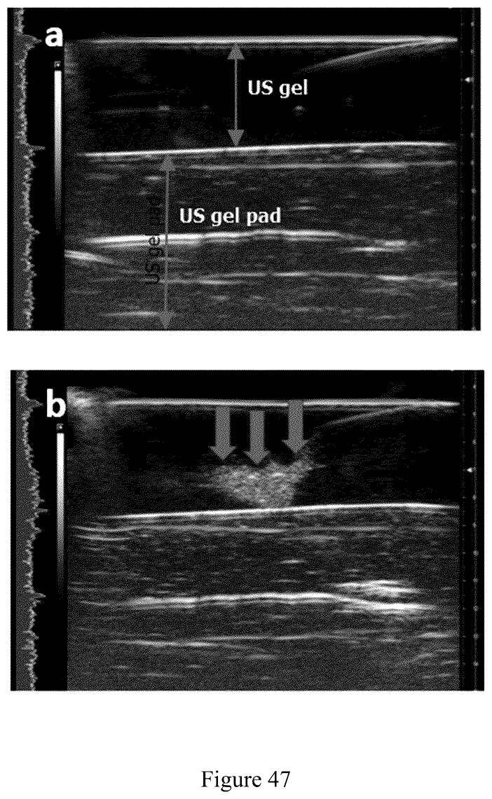

In vivo Magnetic Resonance Imaging. 4-to-6-week-old C57B16 mice were used to demonstrate the in vivo MRI traceability of ¹⁹F. Mice were intra-articularly injected with 50 μL volume of 1 mM Q2_{LTF} protein, guided by ultrasound (FIG. 37a-c). Throughout the imaging experiments, the Q2_{LTF} fibers appeared immobilized using both high frequency ultrasound and MRI. Notably, Q2_{LTF} revealed high frequency echogenic properties as shown using a phantom setup (FIG. 47) and in vivo experiments (FIG. 37a-c).

Three-dimensional gradient echo (3D-GE) imaging of mice hindlimbs was conducted under 200-μm isotropic resolution (FIG. 37d). The images revealed the presence of Q2_{LTF} in the injected joint, observed as a hypo-signal on MRI due to its short T₂ transverse relaxation time. T₂-shortening of Q2_{LTF} could be attributed to the semi-solid fibers, which provide rigidity and result in dipolar interactions within the protein. Additionally, the high protein concentration creates a hydrophobic environment, restricting water mobility and further contributing to the observed hypo-signal.

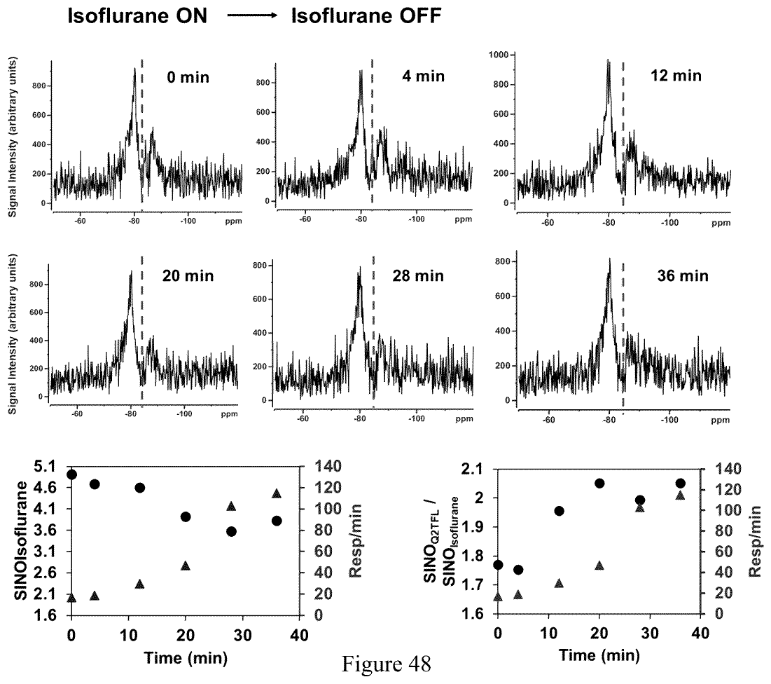

In vivo ¹⁹F MR spectroscopy showed a chemical shift of −81.5 ppm (FIG. 37e) corresponding to Q2_{LTF} with a SNR of 20.6. Interestingly, the spectra also revealed a neighboring peak with a chemical shift of −86.7 ppm, which is attributed to the use of isoflurane as an inhaled anesthetic during the in vivo mouse imaging. This was verified by turning off isoflurane while performing a series of 1-min scans over time. As respiration increased due to clearance of the anesthetic, the SNR of the peak at −86.7 ppm gradually decreased, while the Q2_{LTF} SNR remained stable (FIG. 48). This observation provides further evidence supporting the identification of the peak at −86.7 ppm as a result of isoflurane presence in the spectra.

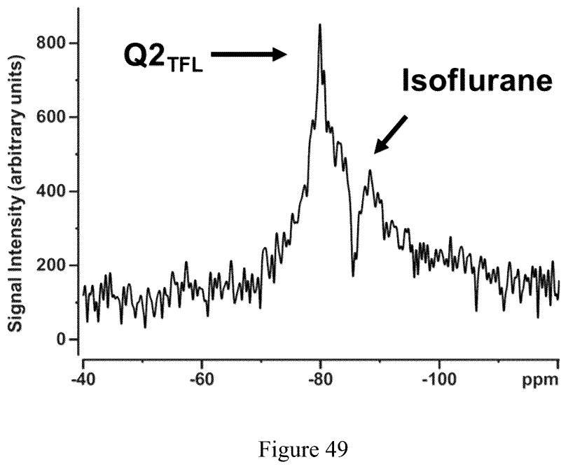

Finally, a comparative analysis was conducted to assess the relative SNR of Q2_{LTF} in vivo compared to F-TRAP. In vivo ¹⁹F MRS was performed using the same sequence timing as used for F-TRAP, while ensuring optimized conditions for Q2_{LTF} pulse sequence parameters (TE, TR, N_{AV}) and MRI coil. The scan consisted of 4000 averages with a TR of 100 ms, resulting in a total scan time of 6 minutes and 40 seconds. The obtained SNR for Q2_{LTF} was 11.45 using 7.0 mg/mL (corresponding to 1 mM Q2_{LTF} and 9 mM ¹⁹F) (FIG. 49). The results demonstrated a substantial improvement in the SNR of Q2_{LTF}, both in terms of weight (2.0×) and mM yield of ¹⁹F (2.5×), which can be attributed to a higher ¹⁹F-protein ratio and monomer density in the fiber morphology, leading to stronger ¹⁹F packing.

Q2_{LTF} forms fibers on the nano- to mesoscale and generates a larger increase in thermostability and SNR compared to a previously fluorinated fiber construct, Q_{LTF}, at the same concentration, demonstrating its ability for ¹⁹F magnetic resonance detection. Furthermore, Q2_{LTF}'s therapeutic potential in the form of drug delivery has been demonstrated by its ability to encapsulate CCM. Its ability to thicken and thermostabilize upon CCM binding was explored as well as its stimuli-responsiveness to ionic strength. Processing of L_{TF} triplet behavior in Q2_{LTF} allows potentially for additional function as a temperature probe and monitoring relative protein structure of the agent. Finally, the ability of Q2_{LTF} to provide multimodal contrast both in ¹H MRI and high frequency ultrasound with sensitive traceability by ¹⁹F MRS in vivo was demonstrated. The results here provide important criteria towards fluorination of coiled-coils for supramolecular assembly and design towards ¹⁹F MRS theranostic agents.

### Example 5

The following provides examples of gels and methods of the present disclosure.

Protein hydrogels represent an important and growing biomaterial for a multitude of applications including diagnostics and drug delivery. The ability to engineer thermoresponsive supramolecular assembling coiled-coil proteins into hydrogels with varying gelation properties was previously explored, where important parameters in coiled-coil hydrogel design were defined. Using Rosetta energy scores and Poisson-Boltzmann electrostatic energies, a computational design strategy was iterated to predict gelation of coiled-coil proteins while simultaneously exploring five new coiled-coil protein hydrogel sequences. Provided this library, the impact of in silico energies on structure and gelation kinetics was explored, where a range of blue autofluorescence that enables hydrogel disassembly and recovery was revealed. As a result of this library, we identify the new coiled-coil hydrogel sequence, Q5, capable of gelation within 24 h at 4° C., was identified. It has more than a 2-fold increase over Q2. The fast gelation time of Q5 enables the assessment of structural transition in real time using small angle X-ray scattering (SAXS) that is correlated to coarse-grained and atomistic molecular dynamics simulations revealing the supramolecular assembling behavior of coiled-coils towards nanofiber assembly and gelation. This work represents the first system of hydrogels with predictable self-assembly, autofluorescence capability, and a molecular model for coiled-coil fiber formation.

Herein, ΔEE_{bcf} is used to study fiber diameter and gelation as supramolecular assembly properties. Furthermore, ΔEE_{bcf} is used to expand the library of coiled-coil hydrogels by characterizing five newly generated hydrogel sequences. In doing so, an iterative workflow is used based on Rosetta energy scores and ΔEE_{bcf} to generate new sequences consecutively and to define regression models for prediction of coiled-coil gelation times. Strong relationships between Rosetta energy scores, thermoresponsiveness, fluorescent recovery after photobleaching (FRAP), and structural transitions during gelation are established. In the case of FRAP, the wide variety of fluorescence behavior and hydrogel recovery after induced fluorescence introduce a new inherent signal of coiled-coil hydrogels.

Among the coiled-coil hydrogel sequences studied, Q5 undergoes a complete sol-gel transition within 24 h, possessing a critical gelation time, t_{c}, of 11.5 h, a greater than 2-fold increase to previously reported coiled-coiled hydrogels. For fiber formation, transmission electron microscopy (TEM) suggests a mechanism predominantly dependent on end-to-end stacking. The fast gelation time allows the structural changes within a full 24 h window to be assessed using small angle X-ray scattering (SAXS) measurements to elucidate the interparticle (interchain) interaction and supramolecular assembly of the coiled-coil hydrogel. The interchain interactions and supramolecular assemblies are further studied using coarse-grained (CG) molecular dynamics (MD) simulations via measurements of radial density distributions and supramolecular structures across fibril sizes and effective electrostatic interaction strengths.

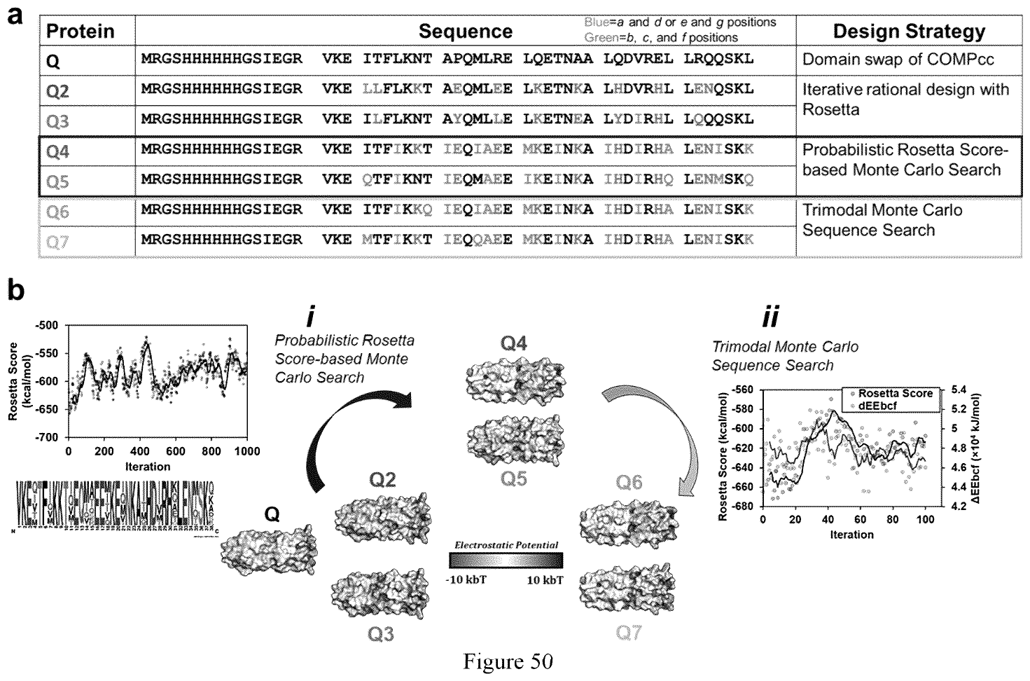

Hydrogel design. Hydrogel variants characterized herein are the result of previous rational design, (Q3), probabilistic Rosetta-based Monte Carlo searches (Q4 and Q5) and trimodal—Rosetta energy-based, NEE_{bcf}, and CEE_{bcf},—Monte Carlo searches (Q6 and Q7) (FIG. 50a) where numbering is the order of sequence design/characterization. Q and its sequel Q2 have been previously designed and characterized as hydrogel materials. We set out to automate the design process of coiled-coil hydrogels by using Monte Carlo search algorithms and incorporating ΔEE_{bcf}. With only three variants capable of gelation, of which only Q and Q2 were soluble at high concentrations (≥2 mM), a Monte Carlo search is employed to obtain variants with an improved Rosetta Score compared to Q, Q2, and Q3. In this search, the Q2 sequence is used as an input sequence and allowed to be mutated at positions in the a, d, e, and g positions; this is done to generate variants that possess similar surface charge distribution as well as solubility and thus an increased likelihood of gelation (FIG. 50bi). The resulting sequences, Q4 and Q5, are used to generate a linear model using ΔEE_{bcf} to predict critical gelation time, t_{c}. The model indicates a missing compositional space for hydrogels with t_{c} values between 30 and 70 h corresponding to ΔEE_{bcf} values between 45 and 64×10⁴ kJ/mol, respectively. It was sought to fill this compositional space and improve automation of design process using the ΔEE_{bcf} metric. Using a trimodal Monte Carlo search, N- and C-terminal EE_{bcf} values were targeted to generate Q6 and Q7 to fill this compositional space (FIG. 50bii).

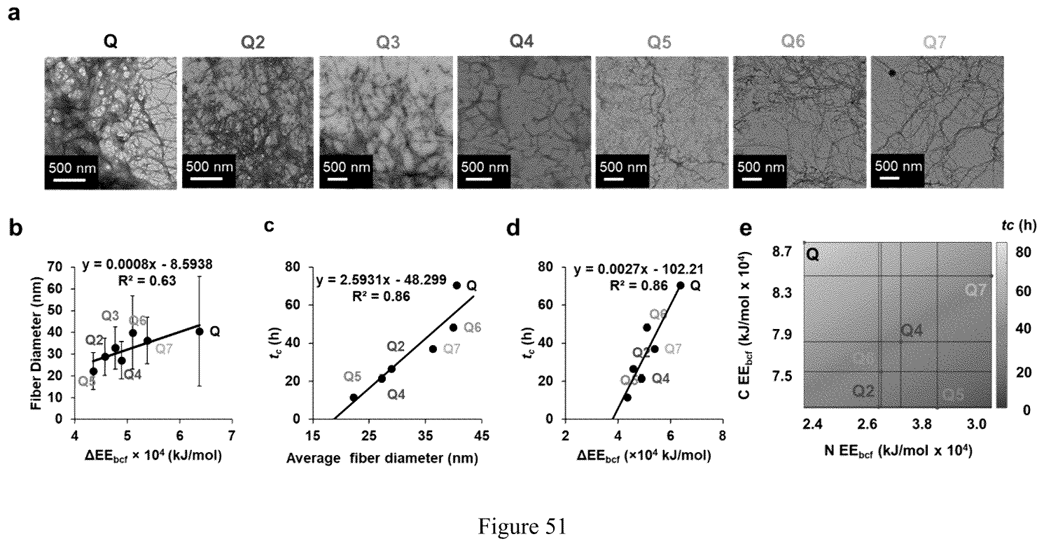

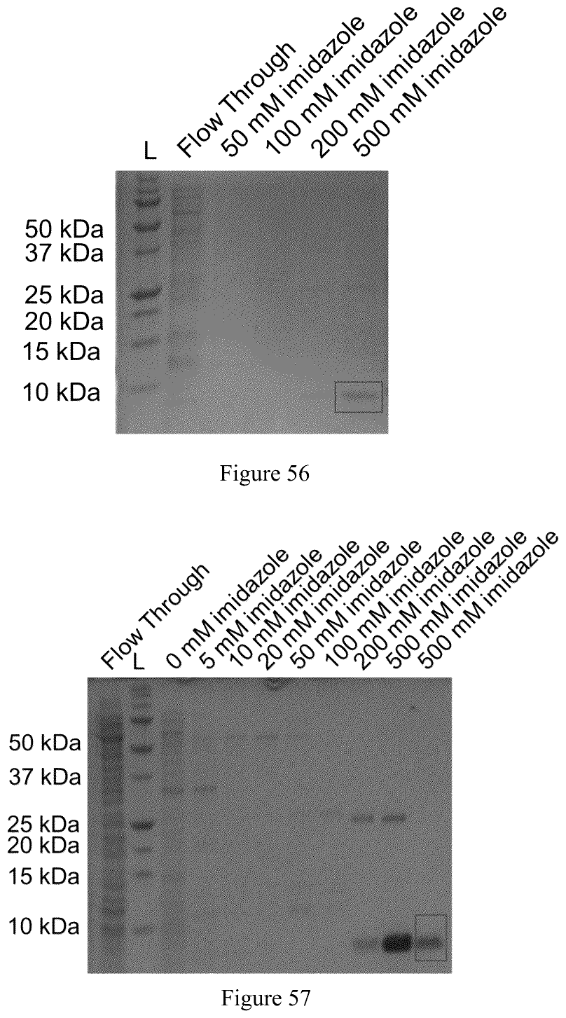

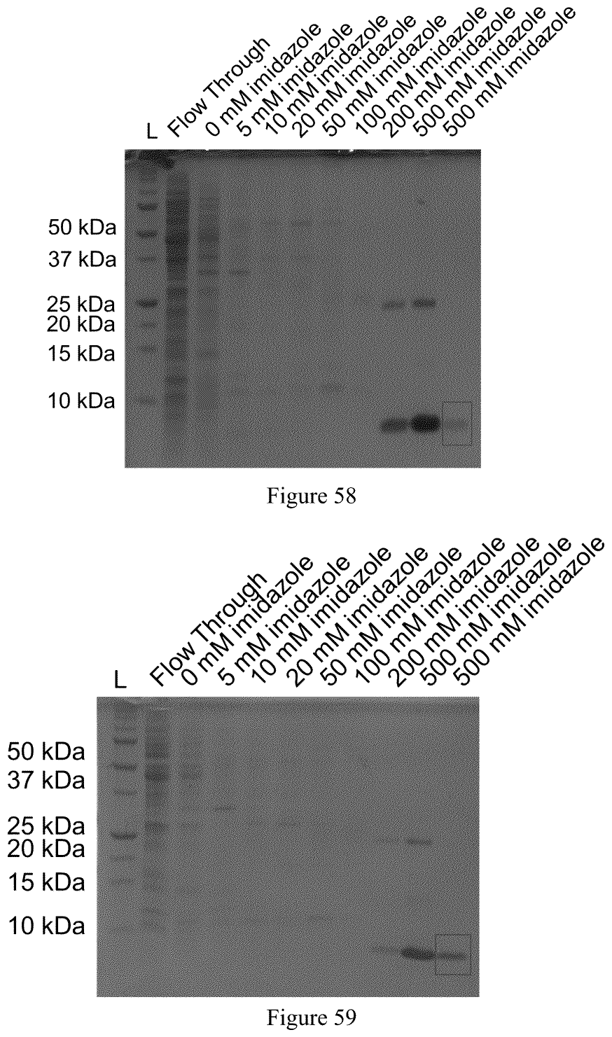

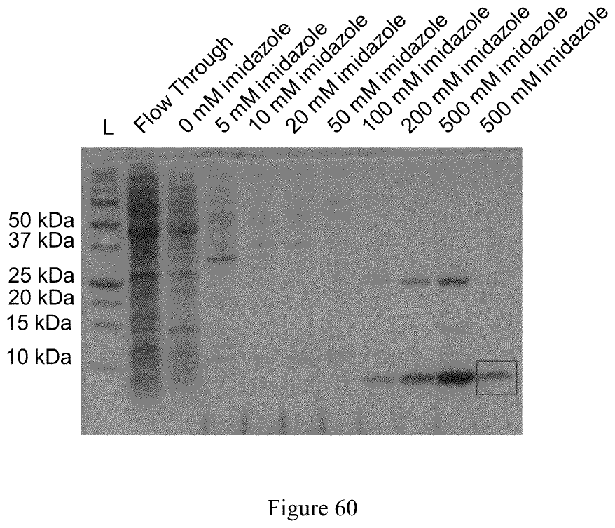

Hydrogel assembly and prediction. Q3, Q4, Q5, Q6, and Q7 were successfully expressed and purified (FIG. 56-60). Gelation was assessed by concentrating protein variants to 2 mM (1.3% w/v) in Tris buffer (50 mM Tris, 500 mM NaCl) at pH 8.0 within 8 h of starting concentration. Hydrogel assembly was first assessed using TEM images of the physically crosslinked nanofibers after incubation at 4° C. (FIG. 51a) and samples were visually inspected for gelation by tube inversion. Independent representative fibers (n=100) were identified and measured from images of the Q variants to determine average differences in lateral assembly, which were also compared to previously characterized Q and Q2 hydrogel nanofibers. The average protein fiber diameters were ranked from smallest to largest with 22.2±8.4 nm for Q5, 27.2±8.7 nm for Q4, 28.9±8.6 nm for Q2, 32.8±9.8 nm for Q3, 36.3±10.8 nm for Q7, 39.9±16.9 nm for Q6, and 40.5±25.2 nm for Q. By an unpaired t-test, all fiber populations were determined to be significantly different except for the following pairs and p-values: Q2/Q4: 0.1662, Q6/Q7: 0.0742, Q6/Q: 0.8434, Q7/Q: 0.1271. Consistent with previously measured differences of the nanofiber library, Q fibers were significantly larger than Q3 and Q2 fibers. Moreover, the growth in hydrogel fiber diameter also possesses a strong linear relationship with ΔEE_{bcf} (FIG. 51b). Notably, this relationship was weaker than the linear relationship for the large nanofibers synthesized under denaturing conditions found previously, which exhibited R²=0.95. This was partially attributed to the physical entanglement of hydrogels leading to the presence of isolated fibers in addition to fiber bundles resulting in proportionally higher standard deviations and lower ranges of hydrogel fiber diameters.

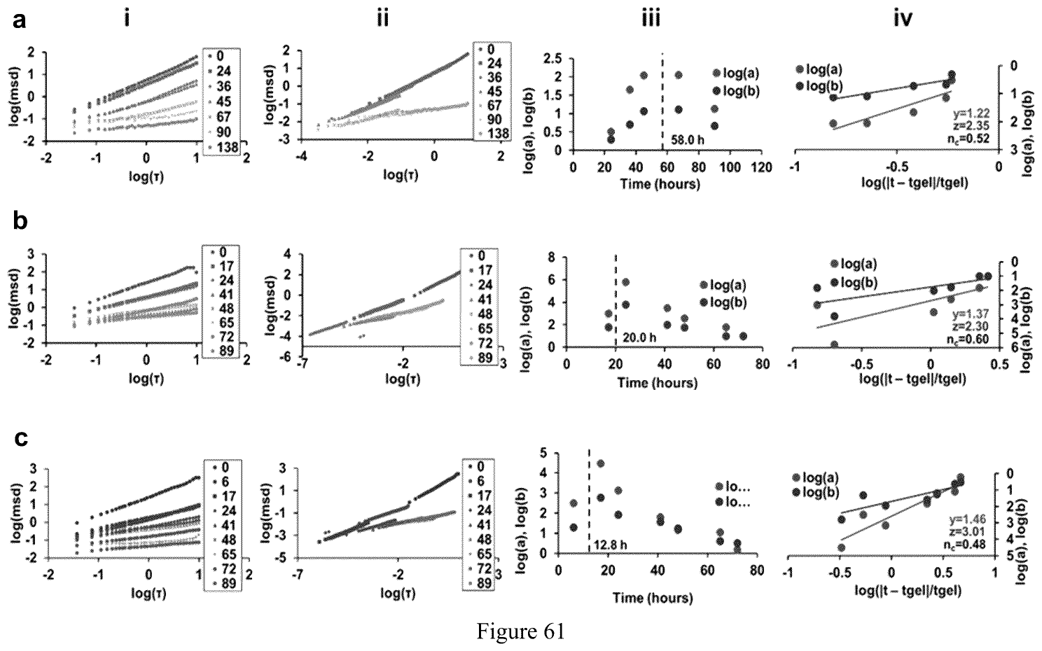

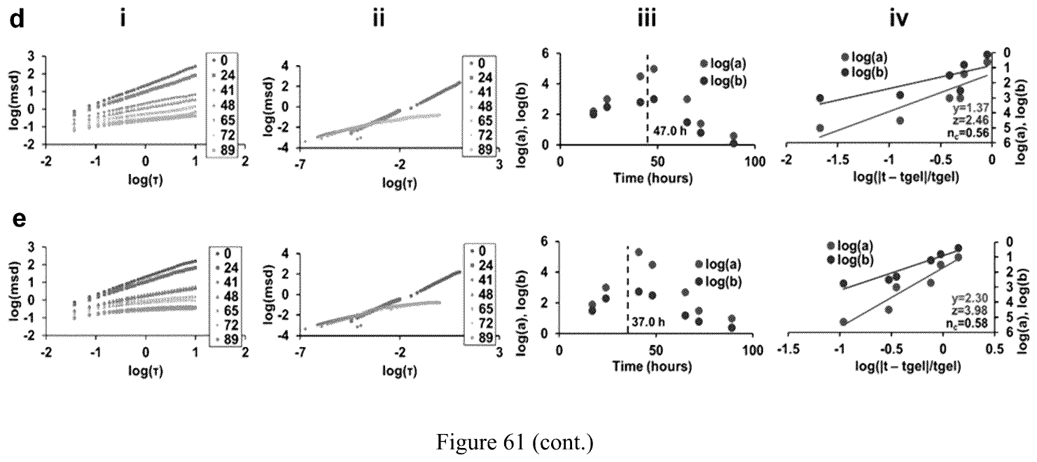

Gelation kinetics of the hydrogels were assessed using passive microrheology (FIG. 61a-e). Time to gelation was measured immediately after concentration to 2 mM at 4° C. by incubating with 1 μm fluorescent tracer beads added to the protein in solution state at a final concentration of 1% (v/v). Microrheology at 2 mM was performed for all variants with the exception of Q3, which possessed a solubility limit of approximately 1.5 mM, and was thus left out of analyses comparing gelation kinetics and mechanical strength. Mean square displacements (MSDs) of bead trajectories were analyzed periodically per the span of respective protein gelation times. An initial MSD-τ curve corresponding to a solution at 1.00 μm² s⁻¹ (consistent with Brownian motion) was used as the master solution curve and the final MSD-τ curve measured per protein gelation was selected as the master gel curve. Intermediate MSD-τ curves measured were superimposed onto either the master solution or master gel curve using horizontal and vertical shift factors, a and b, respectively. The window of sol-gel transition was determined between where a divergence in superposition occurred for each protein gelation.

The t_{c} is used to describe the time until the protein exhibits a majority gel behavior where tracer movement is confined rather than solution behavior where tracers exhibit Brownian motion. Both t_{c} and critical relaxation exponent, n_{c}, were determined (Table 17). The t_{c} ranked in increasing order is 11.5±1.5 h for Q5, 21.6±2 h for Q4, 26.6±0.5 h for Q2, 37.1±0.1 h for Q7, 48.3±1.7 h for Q6, 58±0.4 h for Q3, and 70.4±0.1 h for Q. The n_{c} is typically used to assess the degree of crosslinking. The n_{c} for the new protein hydrogels are ranked in increasing order with 0.56±0.03 for Q4, 0.49±0.02 for Q5, 0.60±0.03 for Q6, and 0.58±0.00 for Q7, suggesting the gels to be at the boundary of a loosely crosslinked hydrogel, similar to Q and Q2. t_{c} was employed to explore the relationship with fiber diameter as suggested by the faster gelation times of thinner Q2 fibers compared to Q. t_{c} exhibits a strongly positive linear relationship with average fiber diameter (FIG. 51c), which, in turn reveals a strong relationship with ΔEE_{bcf} (FIG. 51d), indicating the ability to predict gelation by linear regression. This relationship is also consistent with our previous finding that reduced lateral assembly of fibers may lead to a higher density of crosslinking and thus faster gelation kinetics.

TABLE 17
Critical gelation time, t_{c}, and critical gelation exponent,
nc calculated using MPT and superposition analysis and storage
modulus (G′) and loss modulus (G″) at 10 Hz calculated
by parallel plate rhinometry for Q3-7 hydrogel variants.

| Protein | t_{c} (h) | n_{c} | G′ (Pa) | G″ (Pa) |
|---|---|---|---|---|
| Q3 | 58 ± 0.4 | 0.52 ± 0.01 | 82.5 ± 35.4 | 12.3 ± 1.7 |
| Q4 | 21.6 ± 2.0 | 0.56 ± 0.03 | 69.9 ± 19.7 | 15.0 ± 3.2 |
| Q5 | 11.5 ± 1.5 | 0.49 ± 0.02 | 228.7 ± 52.2 | 11.8 ± 6.1 |
| Q6 | 48.3 ± 1.7 | 0.60 ± 0.03 | 252.9 ± 122.7 | 13.0 ± 5.7 |
| Q7 | 37.1 ± 0.1 | 0.58 ± 0.0 | 297.5 ± 61.7 | 20.6 ± 7.9 |

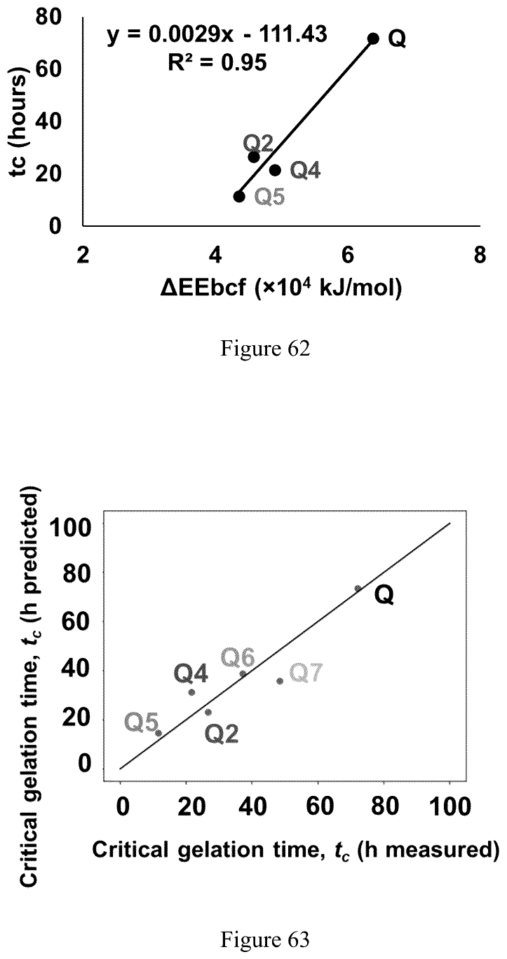

After design and characterization of Q4 and Q5 hydrogels, we used a preliminary linear relationship to correlate ΔEE_{bcf} to t_{c} (FIG. 62) as an early iteration to predict and design future variants. Gelation times of 36 h and 44 h were predicted for Q6 and Q7, respectively, with a root mean squared error (RMSE) of 5.5 h and R²=0.95 based on this linear relationship where Q6 and Q7 were measured to possess gelation times of ˜48 and ˜37 h, respectively. The final model (FIG. 51d) possessed an RMSE of 13.0 h and R²=0.86.

With the six variants measured for gelation time at 2 mM, this model was further expanded by delineating the weight of N- and C-terminal EE_{bcf} and EE_{bcf} (NEE_{bef} and CEE_{bef}, respectively) and generating a bivariate linear relationship (Equation 5, FIG. 51e).

t_{c}=−0.00391×NEE_{bcf}+0.00260×CEE_{bcf}−60.750 Equation 5

This relationship increases the correlation slightly from R²=0.86 to R²=0.88 and helps to explain the relative contribution of the N- and C-termini to t_{c}. The RMSE is also reduced to 6.9 h (FIG. 63). Contribution from the N-terminus shown here is due to 5 surface residues (AA17-28) in these protein variants whereas the C-terminus contribution is the result of 11 surface residues (AA29-54). As a result, the N-terminus is weighted more strongly in the gelation prediction and thus self-assembly of our Q hydrogel variants with a coefficient of −7.82×10⁻⁴ h mol k⁻¹ AA⁻¹ whereas the C-terminus coefficient is 2.36×10⁻⁴ h mol k⁻¹ AA⁻¹, approximately 3-fold greater. Thus, mutations of the N-terminus of the coiled-coil region have a relatively higher impact in the self-assembly and gelation of Q hydrogel variants.

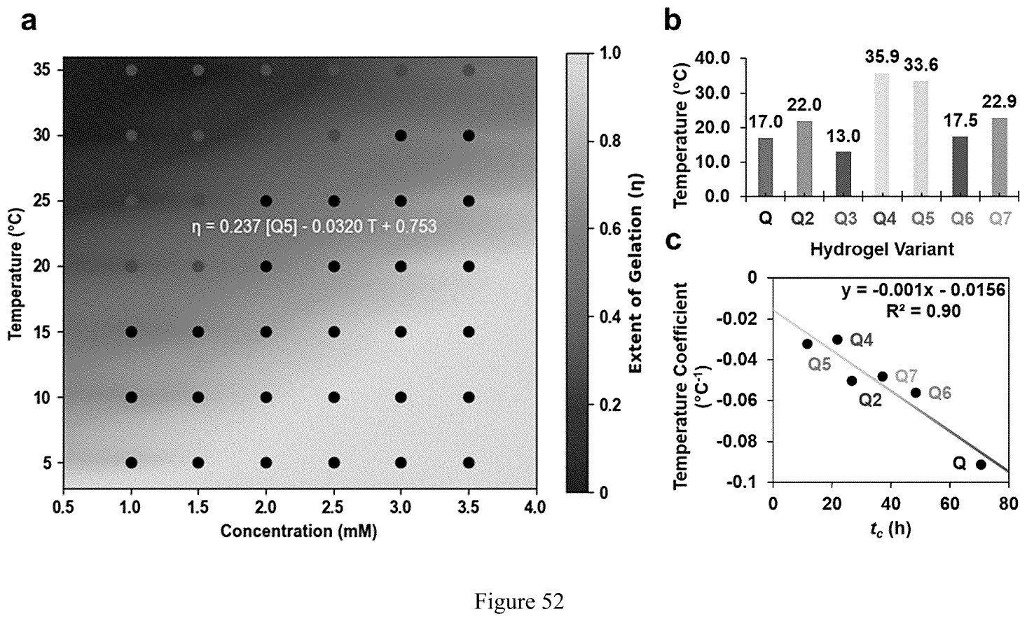

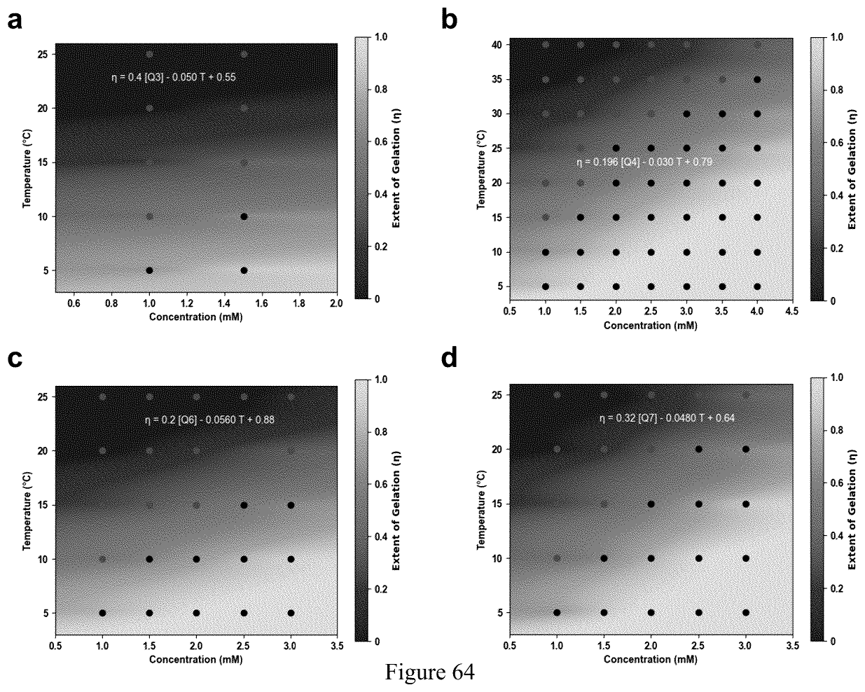

Thermoresponsiveness and material strength. UCST behavior of protein hydrogels was assessed by testing 100 mL samples via tube inversion in 2 mL microtubes after incubation for two weeks at various temperatures and concentrations. Temperature conditions were modulated in the range of 5-40° C. The concentration range was modified depending on if precipitation was visually noticeable in the microtube at an earlier concentration, indicating a solubility limit to our phase diagram, as has been done previously. These solubility limits for Q and Q2-7 were found to be 3.5 mM, 3.5 mM, 1.5 mM, 4.0 mM, 3.5 mM, 3.0 mM, and 3.0 mM, respectively. As done with Q2, the UCST of the hydrogels was determined using a bivariate linear relationship for the extent of gelation where tubes that passed gelation by tube inversion were assigned a value of 1 (shown in black in FIG. 52a and FIG. 64a-d) and tubes that failed gelation by tube inversion (shown in red in FIG. 52a and FIG. 64a-d) were assigned a value of 0. The heatmap in FIG. 52a and FIG. 64a-d was used to denote the range of extent of gelation where the equation was employed to solve for the UCST using the solubility limit and solving for an extent of gelation, η, value of 0.5. The UCSTs (FIG. 52b) for the hydrogels studied here were 13.0° C., 35.9° C., 33.6° C., 17.5° C., and 22.9° C. for Q3-7, respectively, as compared to 17.0° C. for Q and 22.0° C. for Q2 found previously. Bivariate regression allowed for solving the coefficients for the dependence of temperature and concentration on gelation. Interestingly, the dependence on temperature was strongly correlated (R²=0.90) with the critical gelation time, t_{c} (FIG. 52c), suggesting that the thermostability of the hydrogel was indicative of its gelation kinetics. Due to the significant time investment in periodic measurements during microrheology and large sample volumes required for full phase diagram measurements, this result suggested that carefully selected tube inversion testing of hydrogels for UCST may offer a less hands-on method for screening targeted coiled-coil gelation times, whereas microrheological assays may offer a lower production method to screen for targeted thermoresponsiveness.

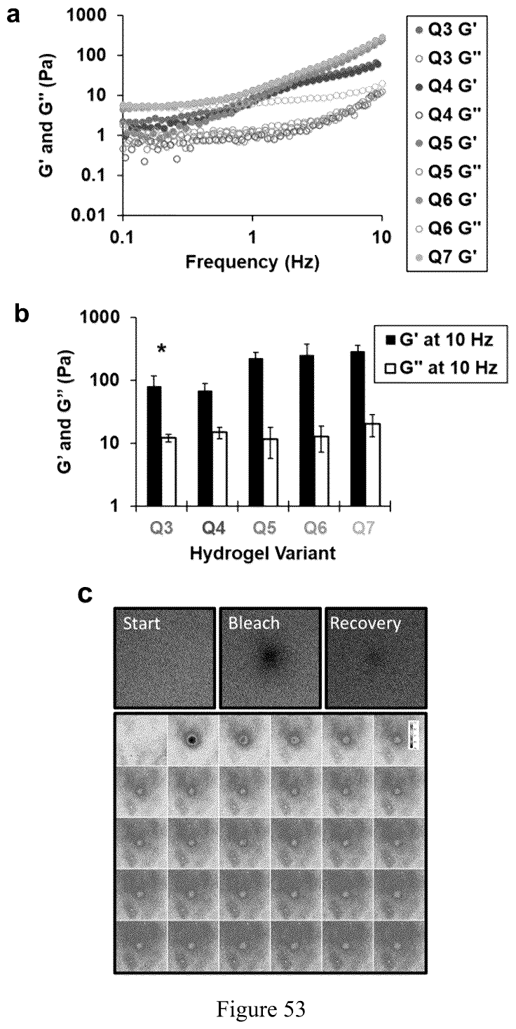

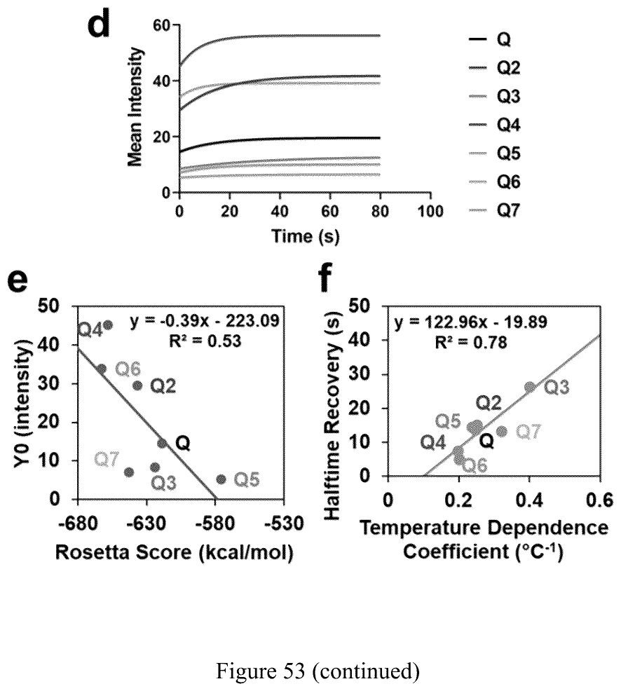

Following gelation, the storage (G′) and loss (G″) moduli of protein hydrogels were determined by rheological analysis (Table 17). Frequency sweeps were performed from 0.1 Hz to 10 Hz at an oscillation strain of 5% (FIG. 53a), with G′ and G″ at 10 Hz used to compare relative mechanical integrity of the variants (FIG. 53b). Important to note, all variants were assessed at 2 mM while Q3 was assessed at 1.5 mM due to its relatively lower solubility limit compared to the other variants. The storage moduli of the hydrogels at 10 Hz were measured to be 83±35 Pa for Q3, 70±20 Pa for Q4, 229±52 Pa for Q5, 253±123 Pa for Q6, and 298±62 Pa for Q7. Loss moduli at 10 Hz were measured to be 12±2 Pa for Q3, 19±4 Pa for Q4, 12±6 Pa for Q5, 13±6 Pa for Q6, and 21±8 Pa for Q7 indicating that G′>G″ for all hydrogel variants. All hydrogel variants assessed have a higher storage modulus than Q (50 Pa). Differences between the gels with the highest storage moduli (Q5, Q6, and Q7) were not statistically significant in comparison with the highest performer, Q2. Using this macroscopic assessment, Q2, Q5, Q6, and Q7 hydrogels were determined to have stronger networks than Q and Q4.

Protein fibrils are capable of blue autofluorescence as a result of protein self-assembly. Fluorescent recovery after photobleaching (FRAP) is measured by photobleaching using a 405 nm laser and measuring the spot intensity over time. This behavior was previously confirmed in the Q protein in a recent characterization. Using the correlation between autofluorescence and fibrilization, the relative autofluorescence of the coiled-coil protein hydrogels was measured. Interestingly, the fluorescence intensities of the hydrogels diminished nearest the center of laser, indicating a loss of fibrilization over time. To assess the relative laser response, hydrogels were subjected to FRAP imaging using two 100% intensity bleaches at 405 nm excitation followed by imaging at 2 second frame intervals (FIG. 53c).

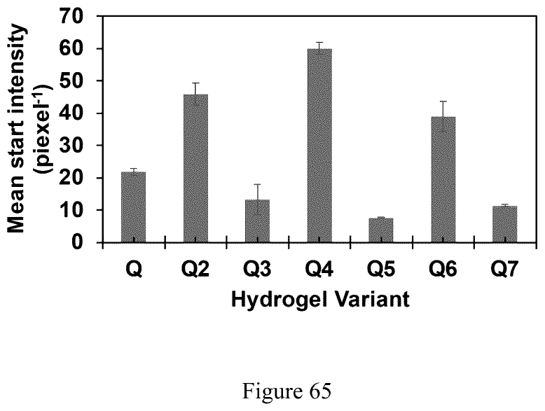

On average, hydrogels exhibited a loss of 67%, 64%, 64%, 75%, 68%, 87%, and 62% for Q-Q7, respectively indicating that Q4 and Q6 experienced more resilience to bleaching. Recovery of the hydrogels over time was fit to a one-phase association equation in GraphPad Prism (FIG. 53d and Table 18). Similarly, the hydrogels experienced an average recovery (in 80 seconds) to 88%, 90%, 92%, 93%, 83%, 99%, and 87% for Q-Q7, respectively. All hydrogels showed high recovery, especially Q6 with virtually complete recovery of its fluorescence intensity. The hydrogels also displayed varying relative fluorescence pre- and post-bleaching, Y0, after baselining with background and applying the one-phase association equation fit. As expected, start intensities (FIG. 65) and Y0 values were strongly correlated with R²=0.97. The Y0 values of the hydrogels possessed a positive correlation with their Rosetta energy scores (FIG. 53e). The Rosetta energy scores measured here were of the pentameric coiled-coil symmetry and described the lowest pose energy found of a single coiled-coil. Fibril autofluorescence of the Q hydrogels was dependent on the self-assembly of the coiled-coil whereas we have found that other gelation properties of the Q hydrogels such as storage modulus and critical gelation time, were independent of Rosetta score.

TABLE 18
Equation fit averages for hydrogel variant FRAP experiment
using one-phase association equation in GraphPad Prism.

| | Y0 | Plateau | K | Tau | Halftime | Span |
|---|---|---|---|---|---|---|
| Protein | (pixel⁻¹) | (pixel⁻¹) | (s⁻¹) | (s) | (s) | (pixel⁻¹) |
| Q | 14.5 ± | 19.6 ± | 0.1 ± | 14.3 ± | 9.9 ± | 5.1 ± |
| | 0.2 | 0.3 | 0.0 | 0.2 | 0.1 | 0.2 |
| Q2 | 29.4 ± | 41.8 ± | 0.1 ± | 16.0 ± | 11.1 ± | 12.5 ± |
| | 1.6 | 2.8 | 0.0 | 0.6 | 0.4 | 1.3 |
| Q3 | 8.5 ± | 14.4 ± | 0.0 ± | 50.1 ± | 34.8 ± | 5.9 ± |
| | 3.9 | 7.5 | 0.1 | 45.2 | 31.3 | 3.6 |
| Q4 | 45.1 ± | 56.2 ± | 0.1 ± | 8.2 ± | 5.7 ± | 11.1 ± |
| | 0.2 | 0.2 | 0.0 | 0.5 | 0.3 | 0.2 |
| Q5 | 5.3 ± | 6.4 ± | 0.1 ± | 17.7± | 12.3 ± | 1.2 ± |
| | 0.0 | 0.0 | 0.0 | 1.3 | 0.9 | 0.0 |
| Q6 | 34.1 ± | 39.1 ± | 0.1 ± | 7.3 ± | 5.1 ± | 5.1 ± |
| | 4.8 | 5.6 | 0.0 | 2.5 | 1.7 | 0.8 |
| Q7 | 7.05 ± | 10.0 ± | 0.1 ± | 13.9 ± | 9.7 ± | 3.0 ± |
| | 0.0 | 0.0 | 0.0 | 0.3 | 0.2 | 0.0 |

The time to recovery, measured by the halftime parameter in the one-phase association equation in GraphPad Prism, possesses a strong correlation to the temperature dependence coefficient (FIG. 53f) measured in the hydrogel phase diagrams (FIG. 52a and FIG. 64a-d). The strong correlation (R²=0.79) indicates that bleaching of the hydrogels is similar to its temperature responsiveness and an entropic response to a high intensity laser. This also suggests the use of photoexcitation as a stimulus in future explorations of Qhydrogels for triggered drug delivery.

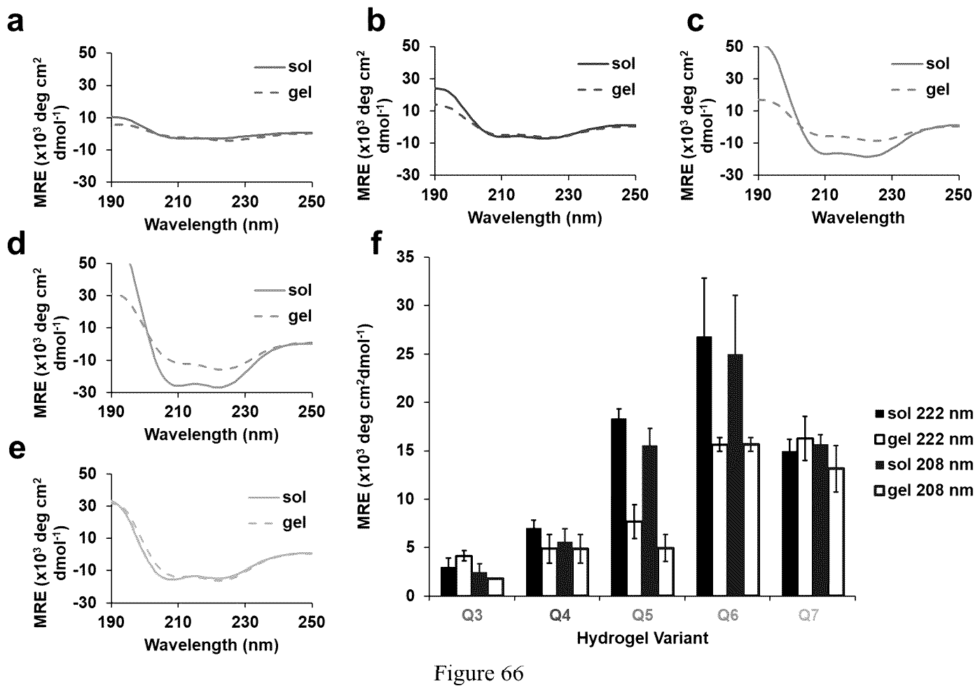

Coiled-coil structure and impact of gelation. To assess the secondary structure of hydrogels, circular dichroism (CD) spectroscopy was employed. New protein hydrogels Q3-7 were subjected to wavelength scans diluted in water to 15 PM. CD spectra of all hydrogels exhibited double minima at 222 nm and 208 nm, indicative of helical secondary structure content (Table 19). Q3 and Q4 possessed reduced minima of −2,500±900 deg cm² dmol⁻¹ at 208 nm and −3,000±900 deg cm² dmol⁻¹ at 222 nm, and −5,600±1,400 deg cm² dmol⁻¹ at 208 nm and 7,100±800 deg cm² dmol⁻¹ at 222 nm, respectively (FIG. 66a-b), similar to the previously characterized hydrogels Q and Q2. However, Q5, Q6, and Q7 possessed significantly enhanced double minima (<10×10¹ deg cm² dmol⁻¹), indicative of a more helical protein system (FIG. 66c-e). Specifically, Q7 exhibited −15,700±1,000 deg cm² dmol⁻¹ at 208 nm and −15,000±1,200 deg cm² dmol⁻¹ at 222 nm, Q5 revealed −15,600±1,800 deg cm² dmol⁻¹ at 208 nm and −18,300±1,000 at 222 nm, and Q6 possessed −25,000±6,100 deg cm² dmol⁻¹ at 208 nm and −26,800±6,000 deg cm² dmol⁻¹ at 222 nm. Regardless, all protein hydrogels experienced a strong reduction in helical signal (FIG. 66f), consistent with our previous CD measurements of Q and Q2 after gelation. Conversely, Q hydrogel variants (with the exception of Q4) experience a significant shift towards a higher 222/208 ratio, indicative of increased α-helicity and overall coiled-coil structure.

TABLE 19
Mean residue ellipticity (MRE) of minima at 222
nm and 208 nm from CD spectra. Results represent
the average of three independent trials.

| | Solution/ | θ₂₂₂ (mdeg · | θ₂₀₈ (mdeg · | |
|---|---|---|---|---|
| Protein | Gel State | cm² · dmol⁻¹) | cm² · dmol⁻¹) | θ₂₂₂/θ₂₀₈ |
| Q3 | Solution | −3,000 ± 1,000 | −2,000 ± 1,000 | 1.2 ± 0.1 |
| | Gel | −4,000 ± 1,000 | −2,000 ± 0 | 2.3 ± 0.3 |
| Q4 | Solution | −7,000 ± 1,000 | −6,000 ± 1,000 | 1.3 ± 0.3 |
| | Gel | −5,000 ± 1,000 | −5,000 ± 1,000 | 1.4 ± 0.2 |
| Q5 | Solution | −18,000 ± 1,000 | −16,000 ± 2,000 | 1.2 ± 0.1 |
| | Gel | −8,000 ± 2,000 | −5,000 ± 1,000 | 1.6 ± 0.1 |
| Q6 | Solution | −27,000 ± 6,000 | −25,000 ± 6,000 | 1.1 ± 0.0 |
| | Gel | −16,000 ± 1,000 | −16,000 ± 1,000 | 1.5 ± 0.1 |
| Q7 | Solution | −15,000 ± 1,000 | −16,000 ± 1,000 | 1.0 ± 0.0 |
| | Gel | −15,000 ± 1,000 | −16,000 ± 2,000 | 1.2 ± 0.1 |

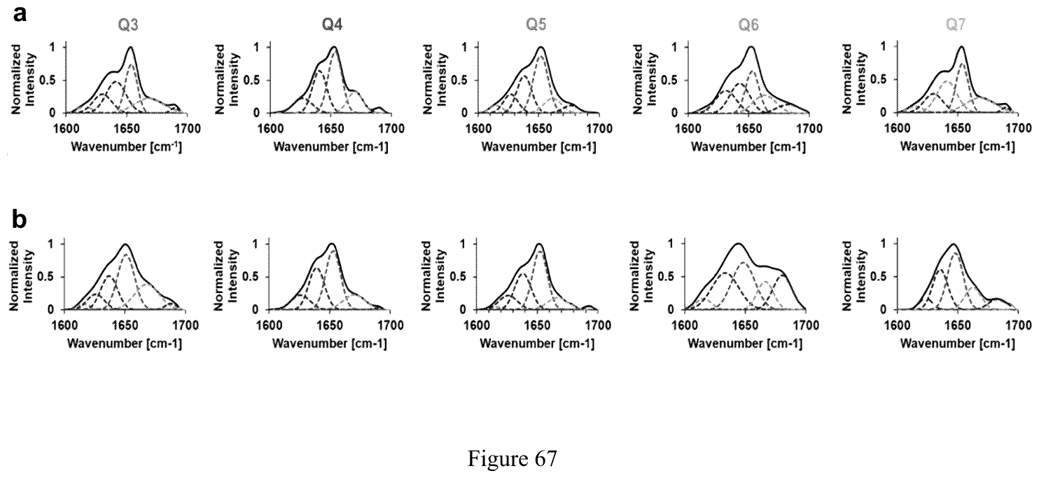

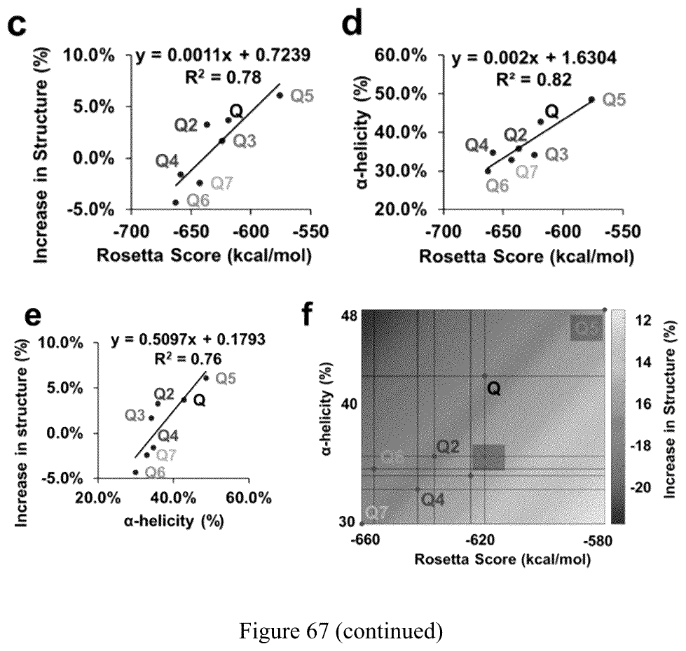

As gelation of the coiled-coil hydrogels is concentration- and buffer-dependent, ATR-FTIR spectroscopy was employed to deconvolute contributions by α-helical, β-sheet, and random coil content (Table 20). Previously, Q and Q2 protein hydrogels exhibited an increase in structured content (α-helical and β-sheet) after incubation at 4° C. and transformation from solution to gel by ATR-FTIR peak deconvolution. Interestingly, we observed a variety of overall changes in structured content in Q3-7 hydrogels in transition from solution (FIG. 67a) to gel (FIG. 67b). The increase in structured content is well-correlated with Rosetta energy scores (R²=0.78) with lower energy scores experiencing an improved change in structured content and higher energy scores experiencing a loss in structured content (FIG. 67c). The Rosetta energy scores also possess a correlation with the α-helicity of the protein as a gel after incubation at 4° C. where lower Rosetta energy scores positively correlate with lower α-helicity (FIG. 67d). Naturally, this indicates a strong positive correlation with gel α-helicity and overall structural transition of the proteins from solution to gel (FIG. 67e). Conversely, α-helical content of the proteins after gelation holds a weak relationship to 1-sheet content (R²=0.03) and to random coil content (R²=0.20), indicating the dependence of this transition on α-helicity.

TABLE 20
ATR-FTIR compositional analysis from Q3-7 protein in solution
and gel states. Summary of secondary structure content uses
the average and standard deviation of the integrated area
of deconvoluted peaks from three independent trials.

| Q Hydrogel | Solution/ | | | Turns/ |
|---|---|---|---|---|
| Variant | Gel State | α-helix (%) | β-sheet(%) | Coils (%) |
| Q3 | Solution | 32.1 ± 6.1 | 46.9 ± 4.2 | 21.0 ± 3.5 |
| | Gel | 34.1 ± 6.1 | 46.5 ± 3.8 | 19.3 ± 3.2 |
| Q4 | Solution | 42.2 ± 8.8 | 45.2 ± 7.2 | 15.2 ± 9.4 |
| | Gel | 34.7 ± 6.0 | 48.6 ± 4.2 | 16.8 ± 3.5 |
| Q5 | Solution | 39.8 ± 0.7 | 44.2 ± 0.2 | 16.1 ± 0.6 |
| | Gel | 41.2 ± 3.8 | 48.6 ± 6.3 | 10.0 ± 8.7 |
| Q6 | Solution | 27.4 ± 6.8 | 57.6 ± 2.1 | 26.7 ± 7.6 |
| | Gel | 33.8 ± 1.6 | 46.9 ± 2.6 | 19.3 ± 2.5 |
| Q7 | Solution | 27.8 ± 7.8 | 40.6 ± 16.0 | 27.0 ± 8.5 |
| | Gel | 32.9 ± 6.3 | 37.6 ± 4.4 | 29.4 ± 6.8 |

The intercorrelation (R²=0.81) of structural transition, α-helicity, and Rosetta energy scores (FIG. 67f) indicate that Rosetta score is capable of a relative structural prediction of coiled-coil protein hydrogels where lower Rosetta energy scores indicate a lower helical content of the protein hydrogel and a loss of structured content and vice versa. Furthermore, Rosetta scores and secondary structure measurements are not strongly correlated to hydrogel strength, crosslinking, or critical gelation time. These relationships indicate the ability to use Rosetta scores to potentially predict structural transitions of coiled-coil protein hydrogels and their relative α-helical content as a hydrogel.

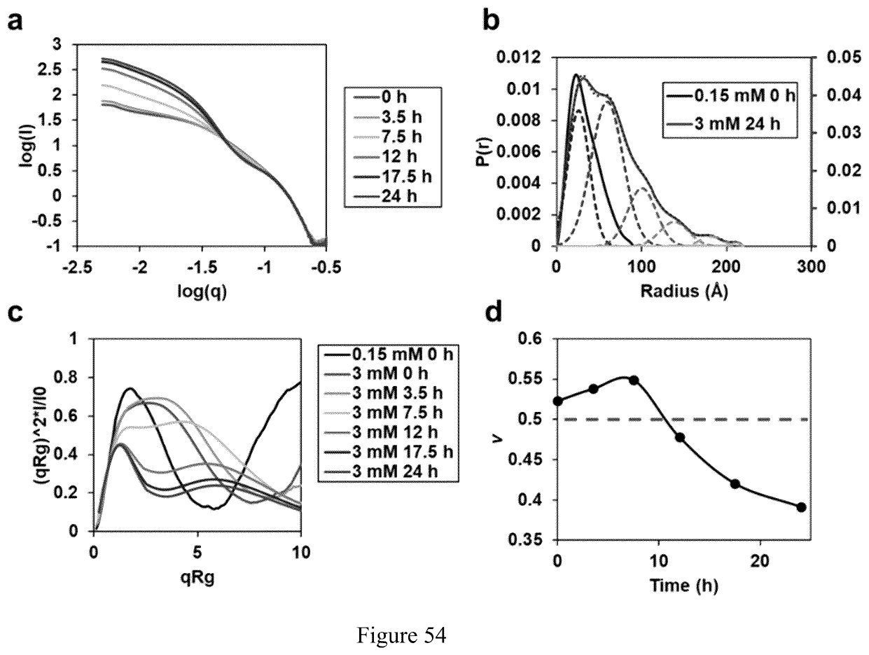

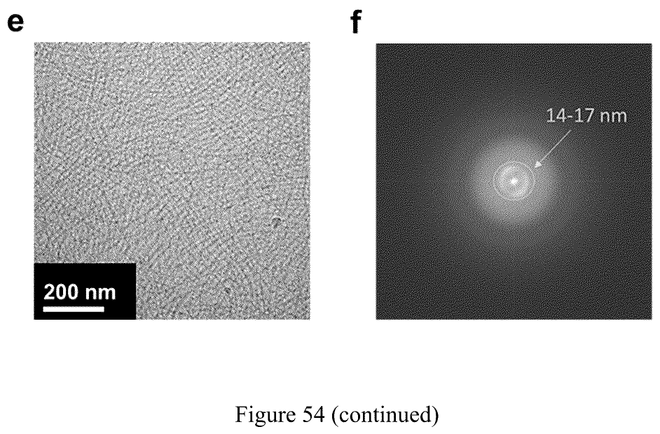

Structural dependence of gelation using Q5. Small angle X-ray light scattering (SAXS) is employed to determine nanoscale structural differences and the supramolecular assembly into nanofibers and physically crosslinked hydrogels. We exploit the fast gelation time of Q5 to explore the transition within a 24 hour window using Life Science X-ray Scattering (LiX) beamline. At all measured timescales, Q5 exhibits an upward tail at the low q range characteristic of attractive interparticle interactions, expected for our self-assembling system (FIG. 54a). Over time, a clear transition in scattering intensity also appears in the length scale between 10 Å and 32 Å, consistent with the interchain distance of a coiled-coil. This interparticle interaction is elucidated by Indirect Fourier Transform (IFT) into the pair distance distribution function, P(r), using primus (ATSAS software) (FIG. 54b).

To understand the distribution of coiled-coils independent of supramolecular assembly and higher order interparticle interaction, a spectra near-representative of the form factor, F(q) was obtained. The P(r) of Q5 at 150 μM concentration and 0 h of incubation at 4° C. (FIG. 54b) indicates a monodisperse distribution centered at 22.5 Å, corresponding to the pore size of a coiled-coil. Upon gelation, new length scales of interparticle interactions are introduced as suggested by the P(r) of Q5 at 3 mM and 24 h of incubation at 4° C. Peaks are deconvoluted using PeakFit software to R²>0.99, with deconvoluted peaks having full-width half maximums (FWHM) at 25.5 Å, 59.2 Å, 100.2 Å, 138.3 Å, 181.8 Å, and 210.5 Å. Interestingly, these correspond to an average step-size increase of 39.1±4.2 Å. Assuming a cylindrical shape from a coiled-coil, this size translates to a cylinder of length 6.8±0.4 nm, which is consistent with the reported length of the parent coiled-coil sequence COMPcc of ˜7 nm, confirming a primarily longitudinal and length-wise assembly.

Using dimensionless Kratky analysis of SAXS measurements (FIG. 54c), Q5 at relatively low molecular weights without incubation at 4° C. generates a well-folded domain indicated by the maximum at low qRg. However, the extended and divergent tails indicate that the protein system possesses flexibility. At high concentrations prior to gelation, Q5 is disordered and partially unfolded, illustrated by the broad curve at 0 h. Over time, the extended plateau becomes diminished and the plot becomes compact, indicating a folded macromolecular structure. Furthermore, the divergence of the Kratky plot at high q and early times (0 h and 3.5 h) indicates flexibility of the protein—a feature that becomes absent at high q and later times (≥7.5 h) demonstrated by the convergence to 0. The presence of two local maximums in the Kratky plot becomes apparent between 12 h and 24 h, consistent with previous SAXS analysis of crosslinked thin fiber gel composed of low molecular weight organic gelators (LMOGs).

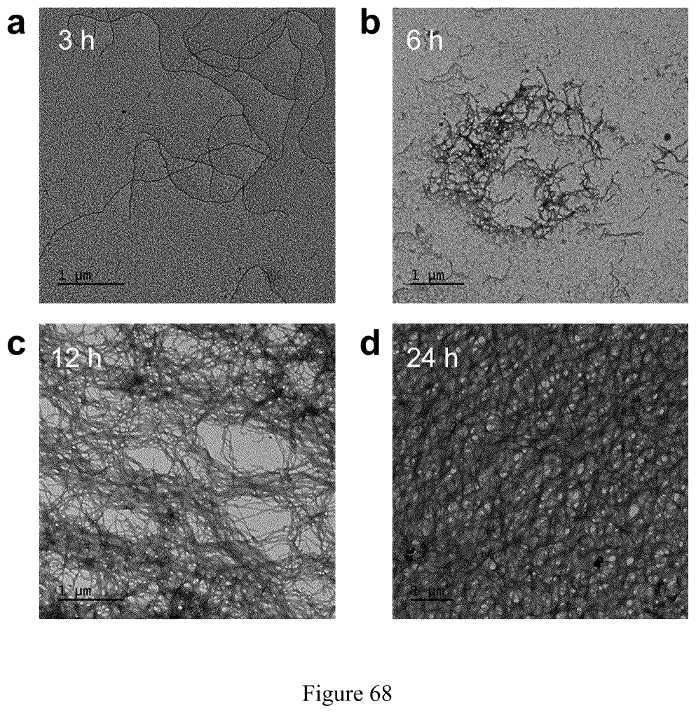

Finally, the relative solvent-chain interaction of the Q5 protein was quantified using the polymer molecular form factor (MFF) fit to solve for the Flory exponent, ν (FIG. 54d). At low times, it was observed Q5 can be considered to possess relatively less favorable interchain interactions where it exhibits values increasingly greater than 0.5 through 7.5 h, whereas at high times, Q5 can be considered to possess relatively more favorable interchain interactions with values increasingly less than 0.5 from 12 h-24 h. The formation of a low-population of protofibrils facilitates high solvent-chain interaction indicated by ν>0.5. Upon formation of a sufficient population of thin fibers, significant hydrophobicity is introduced into the system to cause nanofiber chain collapse and hydrophobicity as indicated by ν<0.5. This is further confirmed by interval TEM measurements of Q5 over time at high concentration (3 mM). At 3 h, a dark intensity in the background is seen as a result of unstructured protein aggregates with ordered nanofiber assembly beginning (FIG. 68a). At 6 h, nanofibers are formed together with a lower background intensity representing the transition away from disordered aggregates (FIG. 68b). At 12 h, a substantial number of long protein fibers form physical crosslinks consistent with ν<0.5 and high chain-chain interaction (FIG. 68c). At 24 h and completion of gel formation, nanofibers appear uniformly physically crosslinked with a high contrast between the light background representative of low or no unordered protein aggregates and dark stained protein fibers (FIG. 68d).

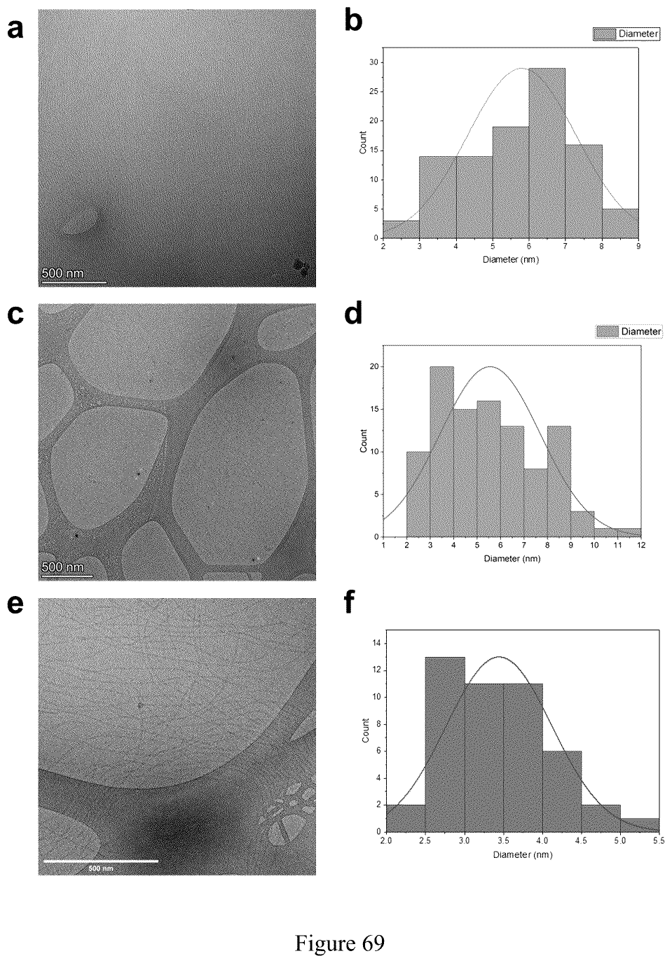

To support SAXS structural measurements, cryo-EM is employed to compare the preserved Q5 gel structure (FIG. 54e). Cryo-EM images of Q5 at 2 mM reveal physically crosslinked diameters averaging 5.8±1.5 nm (n=100) (FIG. 69a-b), significantly lower than as measured by TEM at 22.2±8.4 nm. Moreover, at lower concentrations, Q5 nanofibers exhibit a distribution shift to smaller diameters where fiber diameters are 5.6±2.1 nm (n=100) at 1 mM (FIG. 69c-d) and 3.4±0.7 nm (n=100) at 0.5 mM (FIG. 69e-f) suggesting a concentration dependence for stable lateral fiber assembly. The higher fiber diameters measured in TEM may be explained by collapse and drying effects on the fibers where the cryo-EM sizes are a better representation of the solution state diameters. Fast Fourier Transfer (FFT) (FIG. 54f) shows a concentric atomic spacing 14-17 nm from the center indicating that the fibers are spaced away in this range on average.

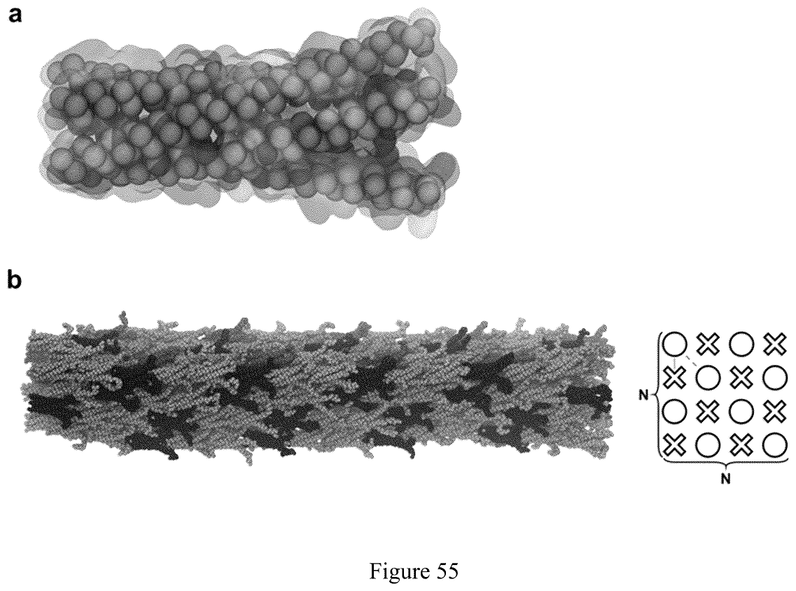

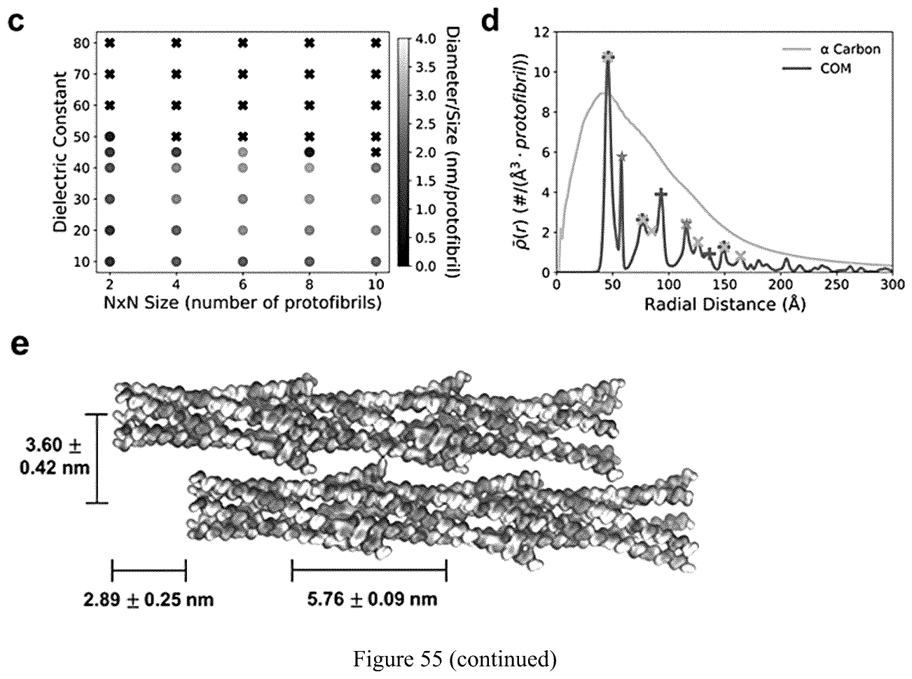

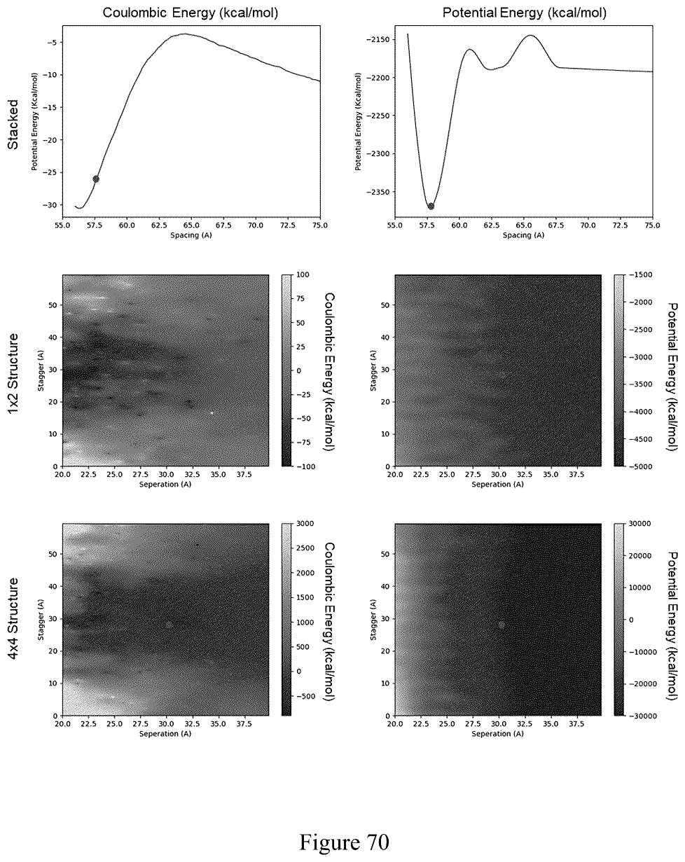

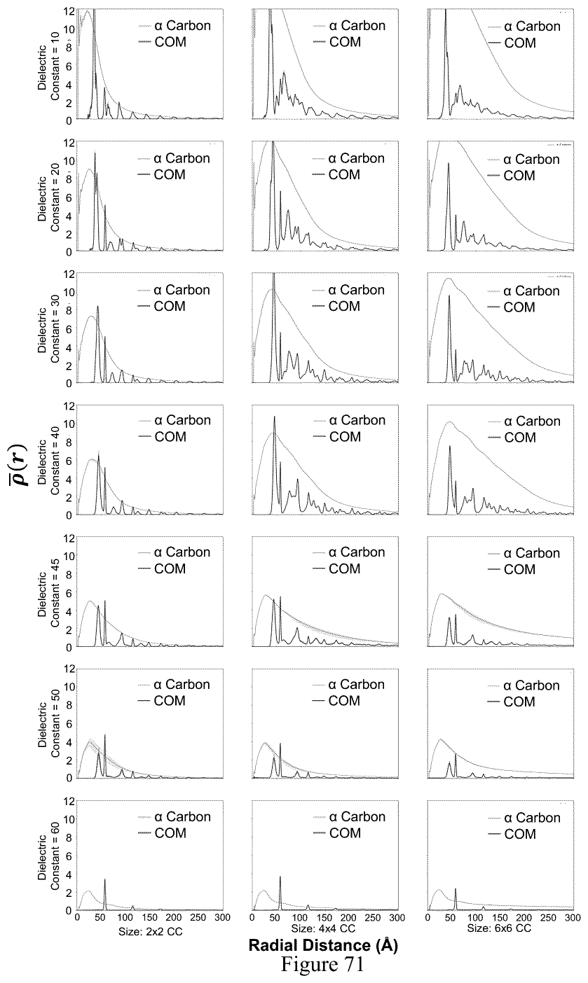

Predicting Q5 fibril stability and morphology from molecular dynamics. Coarse-grained (CG) molecular dynamics (MD) simulations were performed to investigate the importance of electrostatic interactions for physical crosslinking of Q5 protofibrils. The CG model of Q5 was mapped to and trained from atomistic MD statistics using a bottom-up coarse-graining approach (FIG. 55a). CG Q5 coiled-coils were initially stacked into protofibrils, which were arranged into square lattices of varying N×N sizes with adjacent fibrils staggered along the fibril axis direction; an example 4×4 fibril after MD relaxation is shown in FIG. 55b. Each fibril was simulated over a range of dielectric constants (E) to assess the impact of changing electrostatic interaction strengths on fibril stability (FIG. 70). As a metric for stability, we computed the equilibrated diameter of each fibril normalized by the number of protofibrils (FIG. 55c, supporting data in FIG. 71). Fibrils that were stable adopted a mean diameter of 0.53±0.09 nm/protofibril between 40<ϵ<50 while diameters decreased as E decreased, with a mean diameter of 0.44±0.01 nm/protofibril at ϵ=10. Both phenomena indicate that a net attractive electrostatic interaction is present between protofibrils but is insufficient to promote fibrilization when ϵ>50. In addition, a positive correlation was observed between the minimum electrostatic strength necessary for fibril stability and fibril size; only the 2×2 fibril was stable at ϵ=50 while all fibril sizes were stable at ϵ=40. The 4×4 and 6×6 fibrils, which yield diameters (7.33±2.05 nm and 18.00±5.10 nm, respectively) similar to that of the experiments, were stable at a maximum ϵ=45, which is comparable to the average dielectric constant of water (ϵ=80) and protein (ϵ≈15).

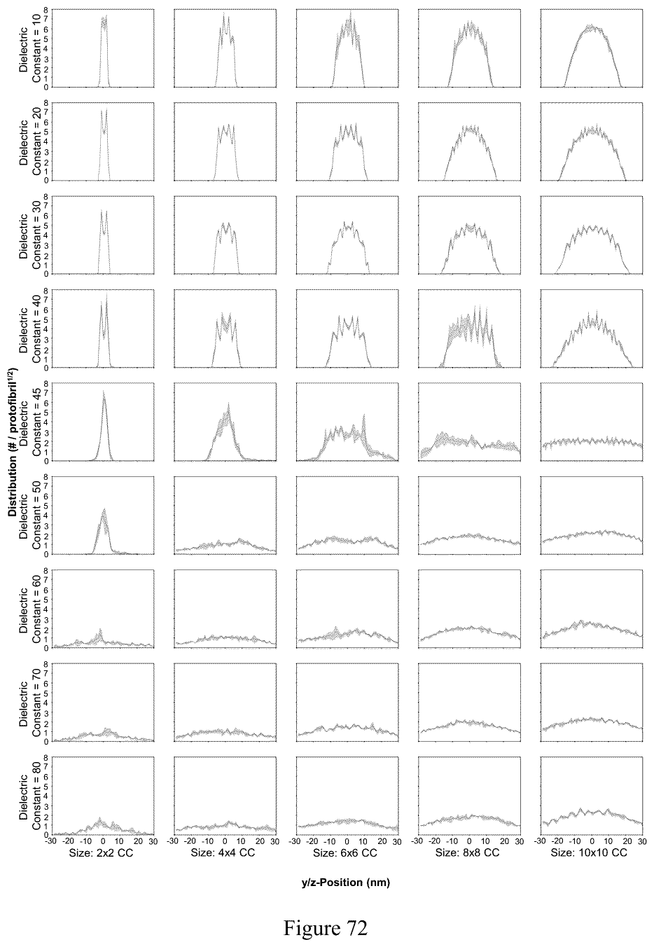

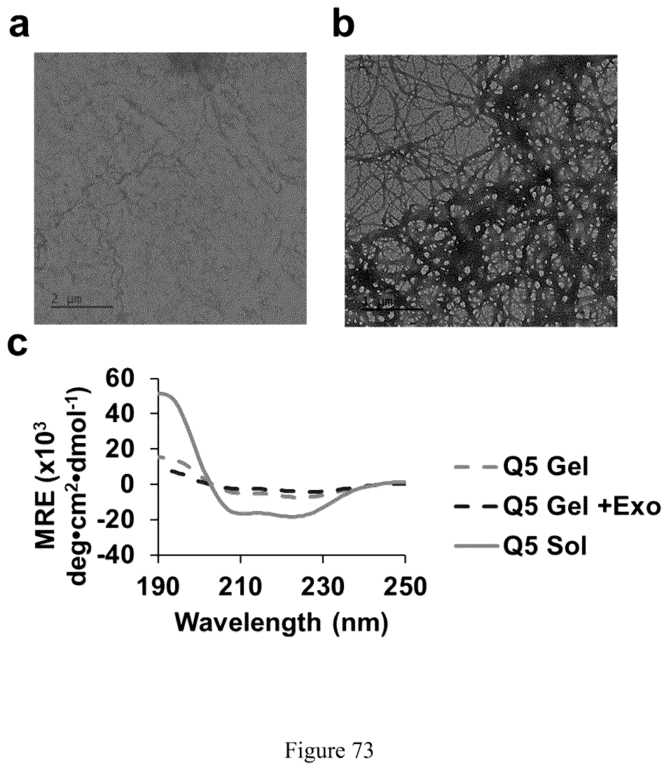

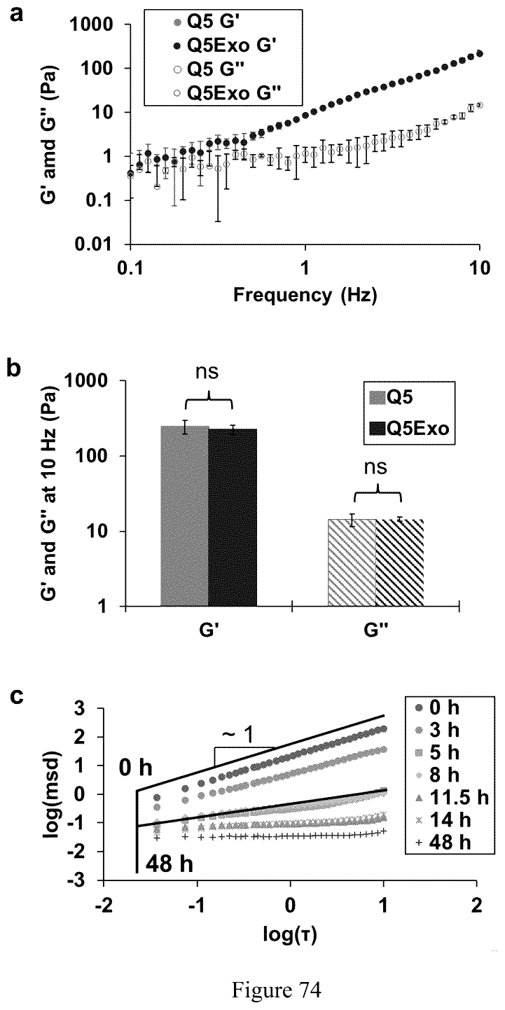

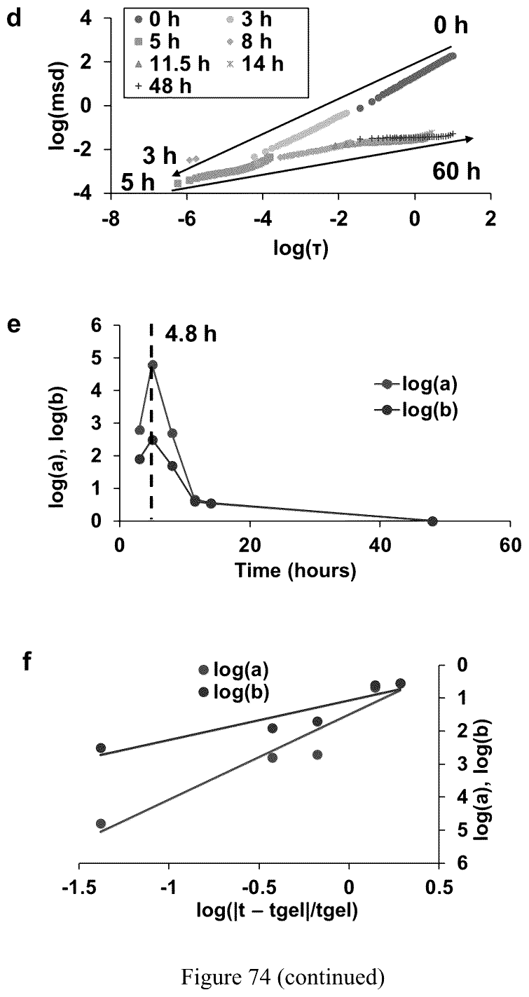

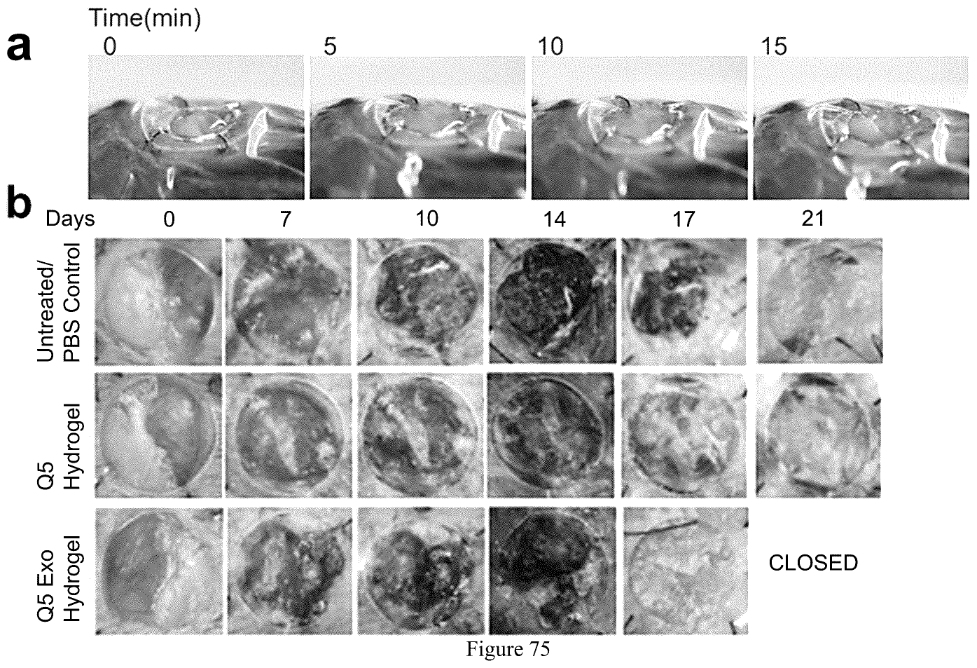

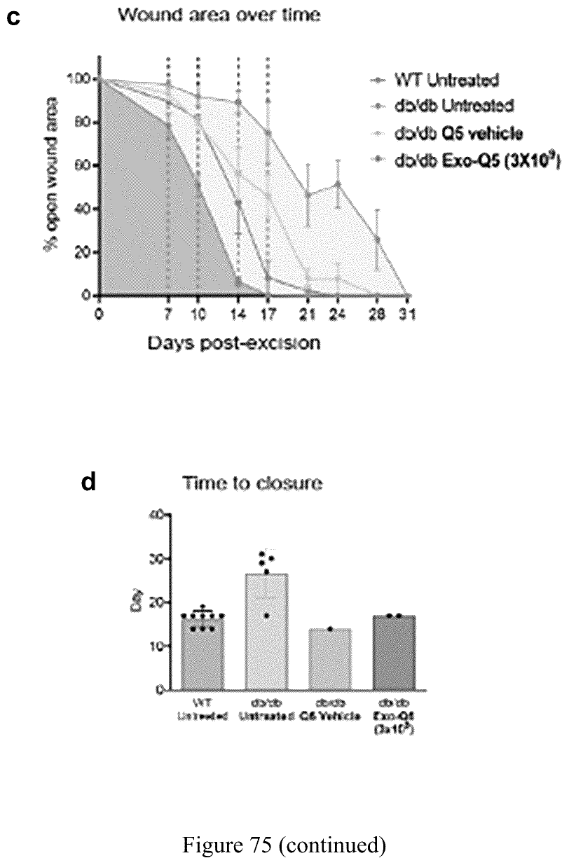

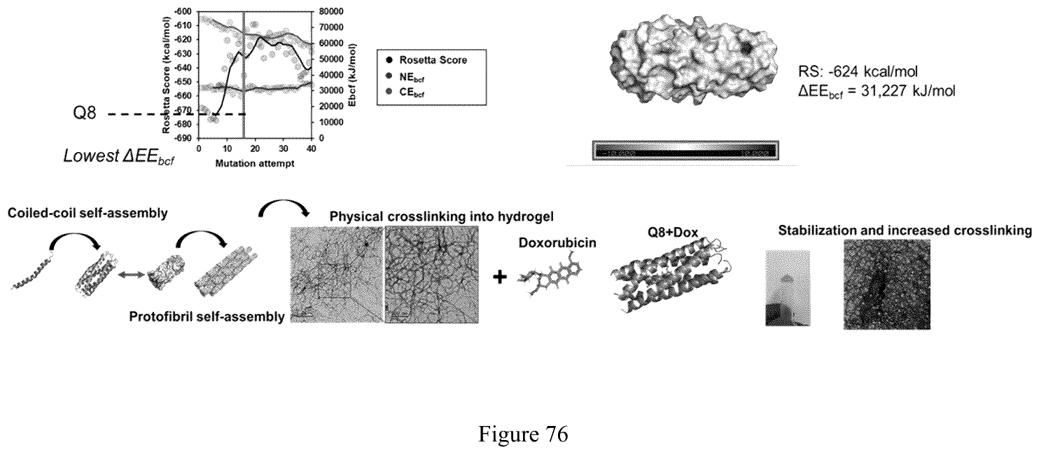

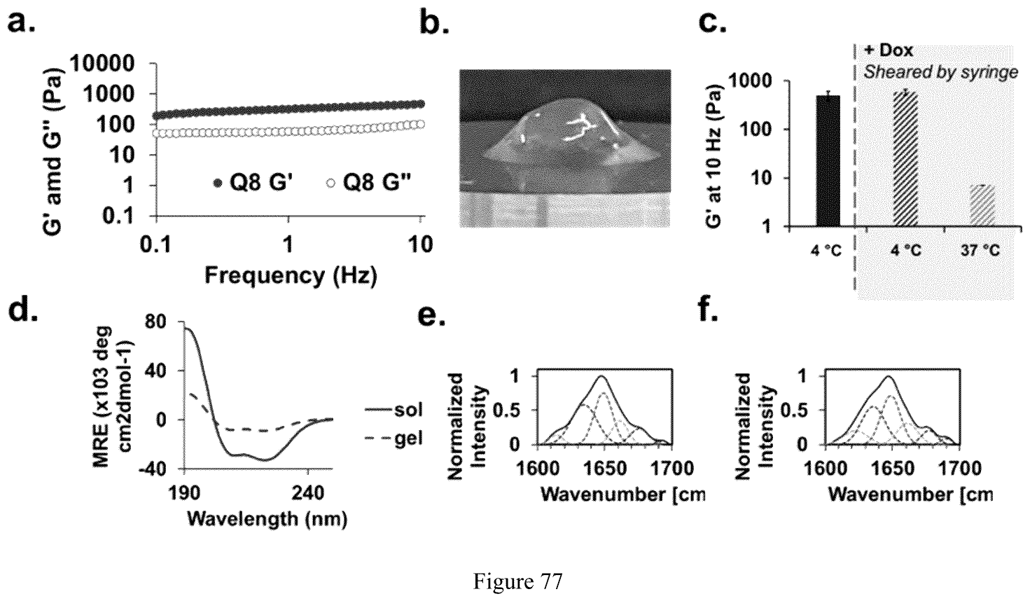

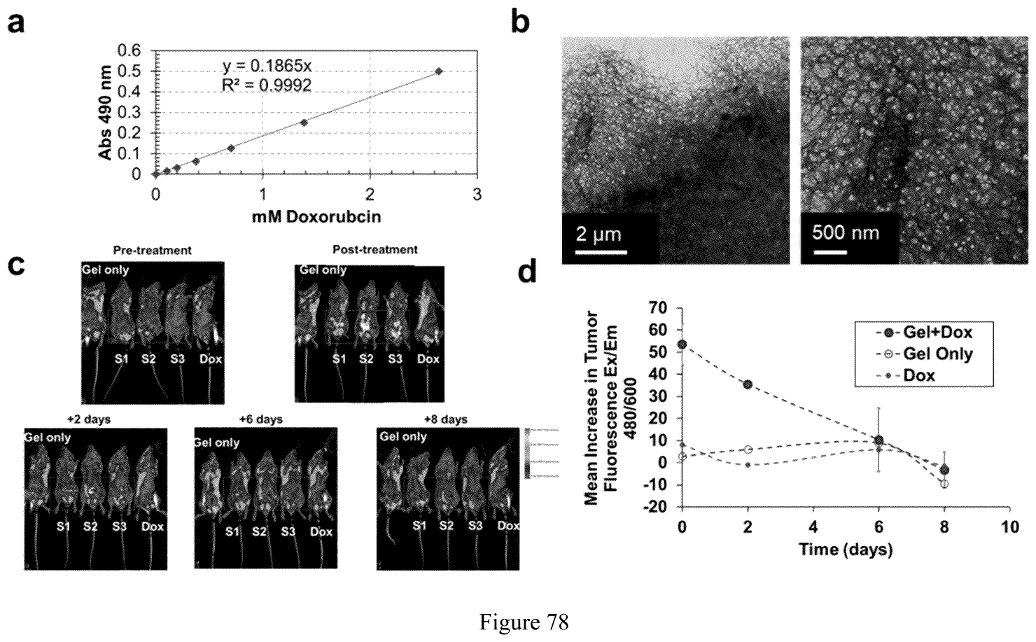

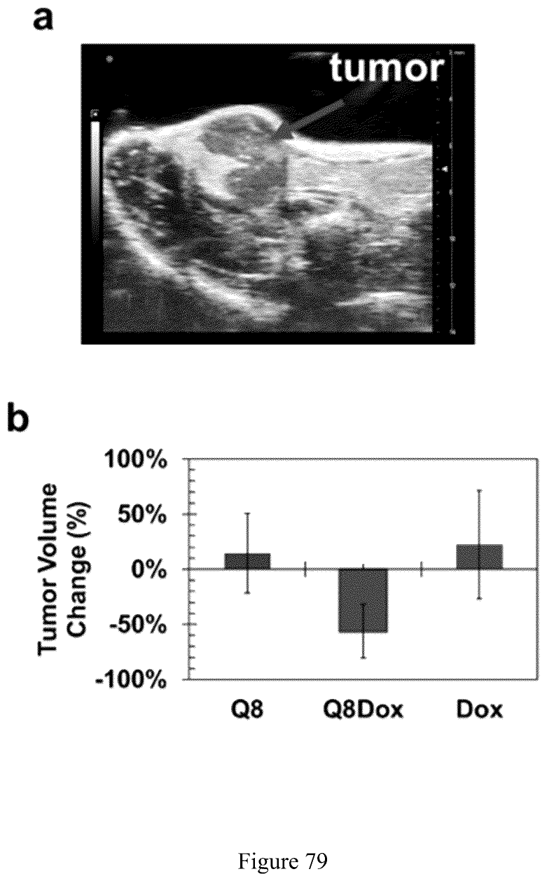

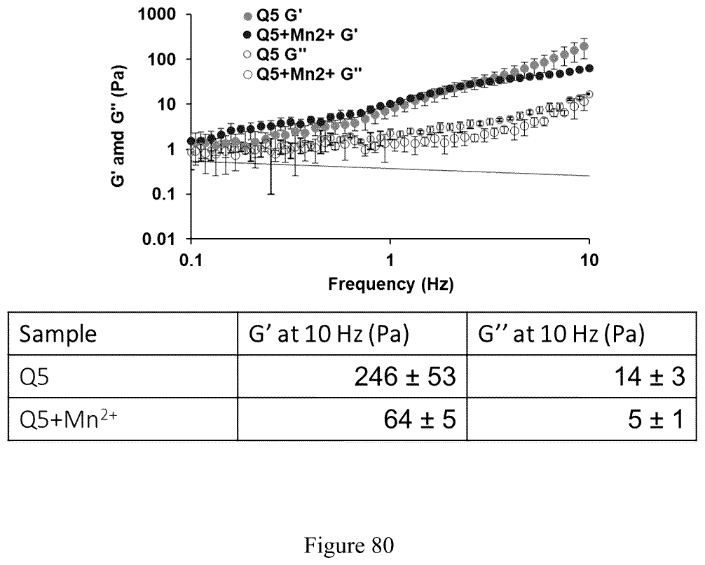

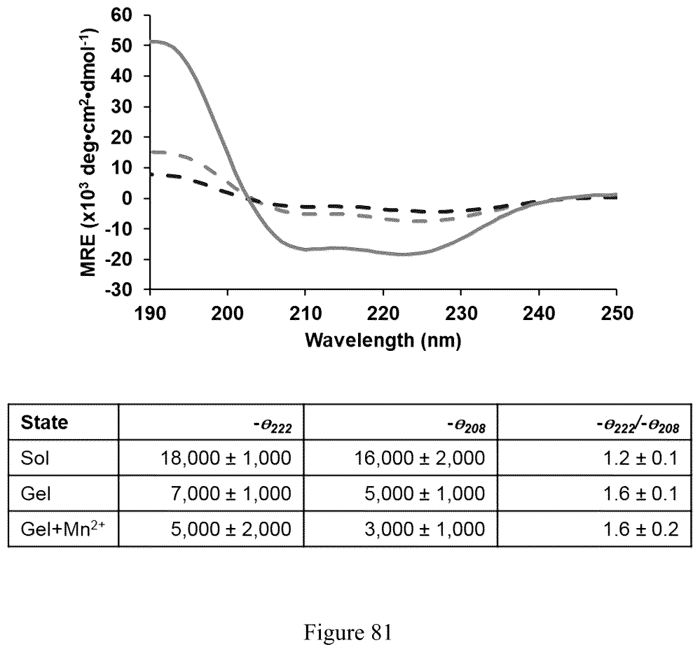

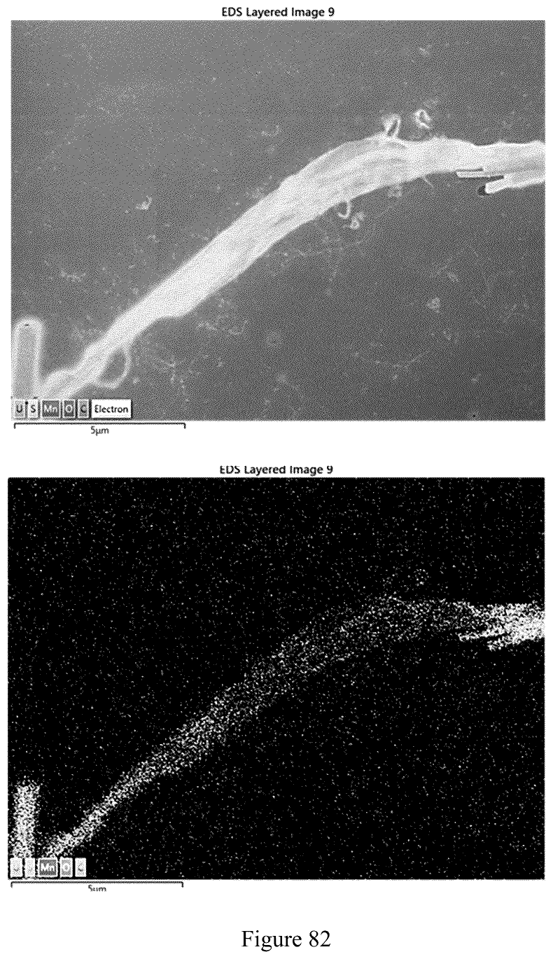

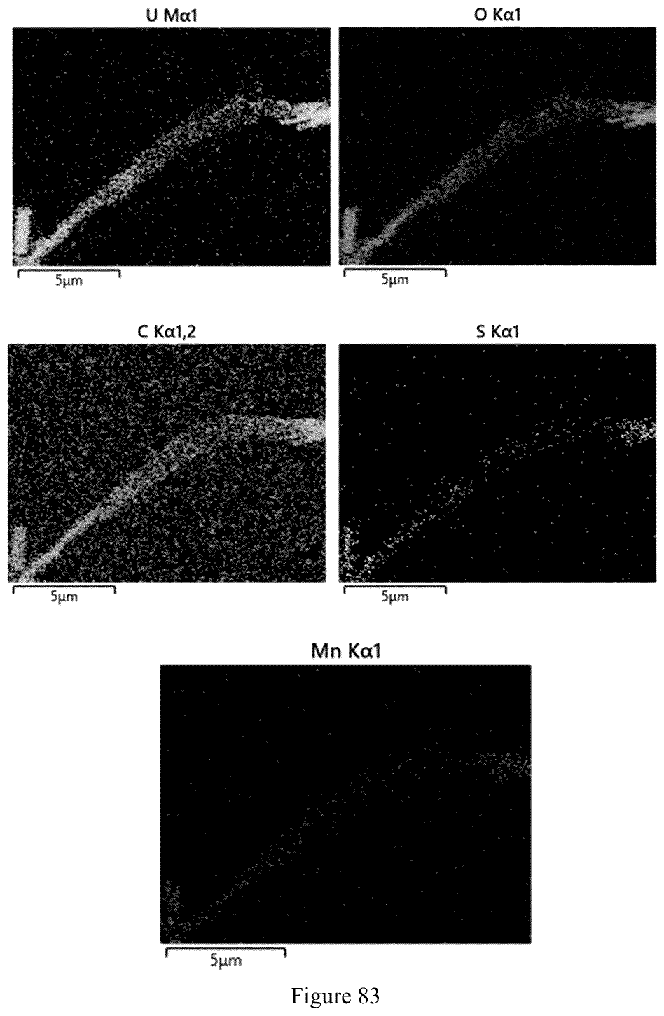

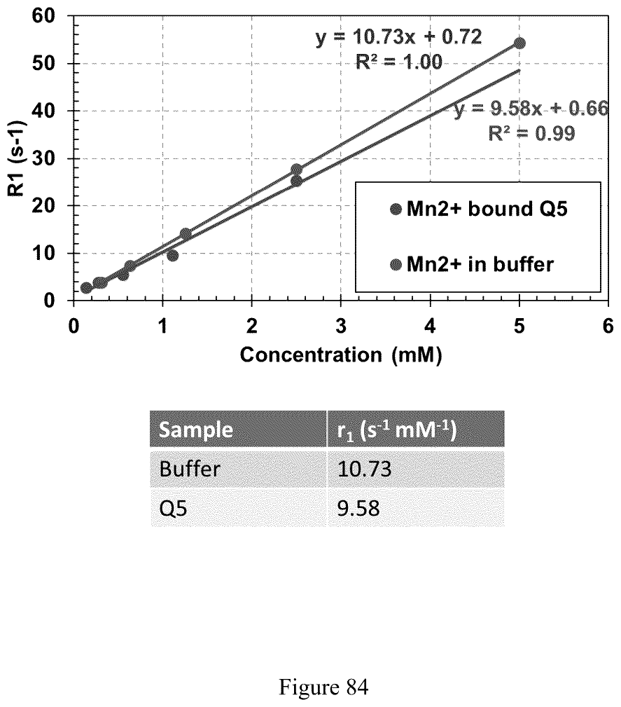

Next, normalized radial density distributions ρ̅(r) were computed for the 4×4 fibrils (FIG. 55d, all other fibril sizes in FIG. 72) to compare with SAXS distributions (FIG. 54b). Both CGMD and SAXS distributions have peaks at distances of ˜40 Å, ˜75 Å, and ˜120 Å, suggesting that the predicted CGMD fibril morphologies are similar to that of experiments. ρ̅(r) was computed using only centers-of-mass of each coiled-coil to reveal the morphological features convolved within the original distribution (FIG. 55d). Here, the blue stars, red plus symbols, and yellow crosses indicate peaks associated with stacked end-to-end coiled-coils (minimum distance of 5.8 nm), coiled-coils in adjacent protofibrils (blue line in FIG. 55b) that are staggered (minimum distance of 4.6 nm), and coiled-coils in diagonally-adjacent protofibrils (red line in FIG. 55b) that are not staggered (minimum distance of 4.6 nm), respectively. The narrowness of the red-marked peaks indicates the stability of staggered protofibrils, likely due to charge complementarity, while the broadening of the yellow-marked peaks suggests that the stabilization afforded by charge complementary is local and does not fully compensate the electrostatic repulsion between non-staggered, diagonally-adjacent protofibrils. While peak positions predicted by CGMD are analogous to that of SAXS experiments, the prominence of these peaks is not as distinct, which is attributed to the limited aspect ratio of our simulated fibrils, i.e., 14:4 (length:width) in the case of the 4×4 fibril.

Overall, these MD simulations suggest that fibrils are stabilized through electrostatic charge complementarity between adjacent protofibrils. This complementarity arose in the staggered square lattice structure present in FIG. 55b, which we depict in FIG. 55e. However, the complementarity-induced net attraction diminishes with size (as suggested by FIG. 55c). This trend likely arises due to the increased ratio of diagonally-adjacent, non-staggered protofibrils to adjacent, staggered protofibrils as fibril width increases. Smaller fibrils, having a higher ratio of adjacent protofibrils to diagonal protofibrils, experience greater net electrostatic attraction compared to larger fibrils, having a smaller adjacent protofibril to diagonal protofibril ratio. As such, a maximum fibril diameter emerges, which the simulations predict to be around 19 nm (i.e., 6×6) in agreement with experimental Q5 diameters. Furthermore, the simulations were performed with the assumption of 0.5 M NaCl solvent and higher salt concentrations (i.e., greater electrostatic screening) are expected to result in smaller fibrils. In addition, while a staggered square lattice structure is assumed, which was both thermodynamically stable and consistent with experimental SAXS data, other possible fibril morphologies cannot be disregarded.

Coiled-coil supramolecular assembly into fibers and hydrogels has not been well understood. Based on recent work showing a correlation between fiber assembly and the difference between electrostatic potential energy on the surface of opposing termini, a model is described herein to predict and design coiled-coil hydrogels with fiber diameters and gelation rates. Also established are the correlations between coiled-coil protein structure, thermostability, and Rosetta energy scores. Furthermore, it was shown that protein structure dictates the ability for coiled-coil protein hydrogels to possess blue autofluorescence, which establishes the coiled-coil proteins designed here to be the first known hydrogels with the capability. Finally, using a combination of SAXS and MD simulations, a molecular understanding of coiled-coils to self-assemble into protofibrils then fibrils and their relative probabilistic geometries was shown, which, demonstrated herein, is due to charge complementarity at both end-to-end and staggered side-to-side coiled-coil interfaces. Overall, this Example provides a top-to-bottom understanding of coiled-coil supramolecular assembly and in turn generates models and tools for their predictive design.

Computational Design of Variant Library. Rosetta suite of macromolecular modeling tools (Version 3.5) was used to model protein mutants and calculate Rosetta scores, with lower energy scores indicating higher stability. The Rosetta Relax protocol was used on protein sequences maintaining the symmetry of COMPcc (PDB: 3V2P) with the all-atom energy score function. Q2 and Q3 were designed previously where Q2 has been characterized as a hydrogel and fiber and Q3 as a fiber. PDB2QR and APBS were used to calculate the electrostatic potential of surface residues, EE_{bcf}, for the N- and C-termini. The difference of the N- and C-terminal EE_{bcf}, dubbed ΔEE_{bcf}, was calculated as described previously. The Q2 sequence was used as an initial input sequence for iterative mutation into sequences Q4 and Q5. Iterative mutations were made through a Monte Carlo search repeated sixty times for 1000 iterations with the goal of minimizing the Rosetta score. The Monte Carlo search used Equation 6 to determine the probability of selecting a mutant with a worse (higher) Rosetta Score (PRS):

$${P}_{RS} = {e}^{\frac{( {RS}_{current} - {RS}_{previous} )}{RT \times C \times {RS}_{previous}}} \qquad Equation 6$$

where RS is the Rosetta score [J/mol], RT [J/mol] is the product of the molar gas constant and temperature, and C is an empirical constant used to constrain the probability criteria during the search (a C value of 3.93×10⁻⁵ [mol/J] was used in our searches). Sequences with the lowest Rosetta scores from each Monte Carlo search were aggregated into a list and residues with the highest likelihood of occurrence at each position were used in the final sequences for Q4 and Q5.

The critical gelation times, t_{c}, of Q, Q2, Q4, and Q5-variants soluble at 2 mM for microrheology measurements—were plotted against ΔEE_{bcf}, with the variants possessing ΔEE_{bcf} values of approximately 31, 41, 45, and 64×10⁴ kJ/mol, respectively. To improve regression model confidence and expand the library of gelation times achieved, ΔEE_{bcf} values between 45 and 64×10⁴ kJ/mol were targeted. Thus, a trimodal Monte Carlo search targeting an improved 1) Rosetta Score, 2) EE_{bcf} at the N-terminus (NEEbcf), and 3) EE_{bcf} at the C-terminus (CEE_{bcf}) were used to acquire sequences for Q6 and Q7 where an improved Rosetta score is a lower value and an improved NEE_{bcf} and CEE_{bcf} are closer in value to the target NEE_{bcf} and CEE_{bcf}. NEEbcf and CEE_{bcf} values of approximately 27×10⁴ kJ/mol and 80×10⁴ kJ/mol were used as target values to acquire a ΔEE_{bcf} in between 45 and 64×10⁴ kJ/mol respectively while possessing an improved Rosetta score. Equation 7 was used to determine the probability of selecting a worse EE_{bcf} (PEE_{bcf}) at the N- or C-terminus:

$${P}_{EEbcf} = {e}^{\frac{- \vert ( {EEbcf}_{current} - {EEbcf}_{previous} ) \vert}{R T \times C \times {EEbcf}_{previous}}} \qquad Equation 7$$

where a C of 1.31×10⁻⁴ [mol/J] and 1.96×10⁻⁴ [mol/J] was used in Equation 7 in our searches for N- and C-terminal EE_{bcf}, respectively. The resulting Q6 gel possessed a ΔEE_{bcf} of 51×10⁴ kJ/mol. Similarly, after characterizing the t_{c} of Q6, a sequence Q7 was selected from the previous search results based on its low Rosetta score and ΔEE_{bcf} existing between 51×10⁴ kJ/mol and 64×10⁴ kJ/mol. Final protein structures were visualized using PyMOL with the APBS plugin.

Protein Expression. All Q variants were expressed as described previously using supplemented M9 media grown from 16 mL starter cultures.

Protein Purification. All Q variants were purified using previously described purification protocols by lysing cells with a Q500 probe sonicator (QSonica), removing cell debris by centrifugation and purifying via a cobalt-charged HiTRAP IMAC FF 5 mL column using an increasing gradient of imidazole.

Rheological Assessment. Rheology with a parallel plate geometry was used to assess mechanical strength and microrheology was used to assess gelation kinetics of Q hydrogel variants, as has been done for Q2 protein previously.

Tube Inversion. Binary sol-gel behavior was assessed using tube inversion. Immediately after concentration to 2 mM, 150 μL of Q hydrogel variants were incubated in 2 mL microtubes at 4° C. for two weeks. Gelation was visually observed by inverting the microtube and inspecting the sample for flow from the top of tube.

Secondary Structure Assessment. Secondary structure and deconvolution of pre- and post-gelation Q variants were assessed by circular dichroism (CD) spectra on a Jasco J-815 CD spectrometer and attenuated total reflectance-Fourier transform infrared (ATR-FTIR) spectra on a Nicolet 6700 Fourier Transform Infrared Spectrometer equipped with a mercury cadmium telluride (MCT)-A detector.

Transmission Electron Microscopy. Transmission electron microscopy (TEM) images were taken with a FEI Talos L120C transmission electron microscope.

Confocal Imaging and Fluorescence Recovery After Photobleaching (FRAP). FRAP experiments were performed with a Stellaris 8 Falcon laser scanning confocal microscope on an inverted DMi8 CS stand equipped with 63X NA 1.4 lens. Images were taken with a laser at 405 nm excitation. A pre-bleach image was taken followed by two bleach images using 100% laser intensity for 1.5 s. Following, 41 images were taken post-bleach at 2 s intervals. To measure recovery, FIJI (version 2.3.0) was used to automate background and recovery imaging following photobleaching. Independent background intensities were fit to a one-phase decay equation in GraphPad Prism. Background loss, as a result of photobleaching, from fit values were added to spot intensity values before being fit to a one-phase association equation in GraphPad Prism.

Small Angle X-ray Scattering (SAXS). SAXS was employed to measure interparticle interactions, structure, and shape of Q5 since it was capable of gelation in the instrument holder within 24 h. SAXS experiments were performed at the National Synchrotron Light Source-II (NSLS-II) Beamline 16-ID (LiX) at Brookhaven National Laboratory.

Cryo-Electron Microscopy. Lacey Carbon 300 mesh copper grids (Ted Pella) were used for cryo-EM imaging on FEI TALOS F200C with Ceta 16M camera at 200 kV accelerating voltage. PELCO easiGlow™ Glow Discharge Cleaning System was also used for the plasma treatment of the grids.

Statistical Analysis. GraphPad Prism (GraphPad Software) was employed for statistical analysis using student's t-test.

All-Atom Molecular Dynamics. The atomic structure of Q5 pentamers (referred to as CC hereafter) was generated using Alphafold Multimer (version 2.1.2) using the full database and maximum template date of Jan. 1, 2018. Two CCs (i.e., ten Q5 monomers) were stacked end-to-end separated by 6 nm. The two CCs were set up in a 20×10×10 nm³ box with periodic boundary conditions and solvated by 0.5 M NaCl in water. The simulation was run using the CHARMM36m force field (February 2021) and GROMACS 2021.1. Initially, energy minimization was done using steepest descent with a tolerance of 500 kJ/mol/nm. Simulations were equilibrated first in the canonical (NVT) ensemble to 277 K (4° C.) for 0.5 ns using the stochastic velocity rescaling thermostat and a damping time of 0.1 ps. Next, the system was equilibrated to 1 bar under the NPT ensemble for 1 ns using the Parrinello-Rahman barostat and a damping time of 20 ps. Finally, simulations were run in the NVT ensemble for 1.2 s at 277 K (4° C.) using stochastic velocity rescaling and a damping time of 2 ps; only the final 1.1 s of data was used for analysis with coordinates saved every 100 ps. For all simulations, a 2 fs timestep was used and the α carbons of residues 14 to 39 of the lower CC were harmonically restrained with a force constant of 1000 kJ/mol/nm². All simulations were repeated across four independent replicas.

Coarse-Grained Model. Coarse-grained (CG) sites were mapped to the α carbon positions of each residue. Two sets of virtual sites were also mapped: intra-CC virtual sites and inter-CC virtual sites. Ten intra-CC virtual sites were defined between CG sites for residues 16, 18, 21, 22, 25, 28, 31, 33, 36, and 38 in adjacent monomers to account for the anisotropic knob-in-hole interactions that maintain the CC structure. One inter-CC virtual sites was defined between residues 12 and 50 at the end-to-end interface to account for end-to-end CC interactions. Four types of potentials defined our CG Hamiltonian: bonded, electrostatic, excluded volume, and virtual site attractions. All intramolecular nonbonded interactions were ignored. Bonded potentials were defined using a heteroelastic network model (HENM) with a cutoff of 19.0 Å using the CG-mapped atomistic trajectories with virtual sites added.

Coarse-Grained Simulations. Fibril CG simulations were run in LAMMPS (2 Jun. 2022). Fibril simulations were prepared in three stages: protofibril geometry optimization, multi-protofibril configurational optimization, and fibril dynamics. First, the energy of a CC pentamer was minimized using the conjugate gradient algorithm and a tolerance of 10⁻⁶ kcal/mol/A. Subsequent protofibrils were built from the geometry optimized structure. Then, an end-to-end stack of five CCs, i.e., a protofibril of length five, was constructed. The center-of-mass distance between CCs was uniformly adjusted between 56 Å and 75 Å to find the inter-CC spacing with minimum energy (FIG. 70). This distance was used to construct all subsequent protofibrils. To construct fibrils, protofibrils were aligned in a square lattice with separation distances between 20 Å and 40 Å while adjacent protofibrils were staggered along the protofibril axis direction for distances between 0 Å and 60 Å. The energy of each configuration was computed to find the radial and staggered distances that yielded the lowest energy (FIG. 70); these two distances were used to prepare the initial configurations of fibrils in the next stage. Finally, fibril simulations were prepared using a square lattice structure of N×N staggered protofibrils of length 14 CCs where N was varied from 2 to 10 protofibrils in increments of 2. For each fibril size, a dielectric constant of 10, 20, 30, 40, 45, 50, 60, 70, or 80 was used. Each system was equilibrated for 10 ns with a time step of 10 fs using the Nosé-Hoover chain thermostat at 277 K and 1 ps damping time. Trajectories were extended for 2 ns and data was collected every 2 ps. All simulations were repeated across three independent replicas.

Radial Density Distributions. The normalized radial density distribution ρ̅(r) for each fibril (FIG. 71) was computed using Equation 8:

$$\overline{ρ} ( r ) = \frac{n ( r )}{4 π {r}^{2} δ r} {N}^{- 2} \qquad Equation 8$$

where r is the pair distance, n(r) is the ensemble-averaged number of particles in the range r to r+δr, δr is the bin width (=0.5 Å), and N is the fibril size (e.g., N=4 for the 4×4 fibril). The distribution function was computed between all CG sites (i.e., α carbon resolution) with all intra-coiled-coil CG pairs subtracted to focus only on inter-coiled-coil CG pairs. Another distribution function using only the centers-of-mass (COM) of each coiled-coil was computed; this distribution was multiplied by a factor of 20,000 such that the magnitude would be comparable to the α carbon distribution for visualization purposes.

Phase Diagram. To estimate fibril diameters, the fibril was translated to the origin and rotated it to align the first two principal axes (computed based on the COM of each CC) to the x- and z-axis, respectively, such that the long direction of the fibril was aligned with the x-axis. This operation was performed on each frame of the trajectory. Next, protofibril-normalized COM histograms in the y and z directions were computed using a bin width of 1 nm (FIG. 72). The difference between the lower (rₗₒ) and upper (rₕᵢ) bounds where the COM histogram exceeded 3.0 #/protofibril^{1/2} was used to define the normalized fibril diameter d̅ (in nm/protofibril) (Equation 9):

$$\overline{d} = \frac{{r}_{h i} - {r}_{l o}}{N} \qquad Equation 9$$

If no lower and upper bounds were detected, the average diameter was treated as 0 nm, i.e., the fibril dissociated. All analysis was done using the Numpy and MDTraj Python packages.

Chemically competent M15MA E. coli cells were gifted from David Tirrell at California Institute of Technology. Bacto-tryptone, sodium chloride (NaCl), yeast extract, tryptic soy agar, ampicillin sodium salt, sodium phosphate dibasic anhydrous (Na₂HPO₄), sodium hydroxide (NaOH), dextrose monohydrate (_{D}-glucose), magnesium sulfate (MgSO₄), calcium chloride (CaCl₂), manganese chloride tetrahydrate (MnCl₂·4H₂O), cobaltous chloride hexahydrate (CoCl₂·6H₂O), isopropyl β-_{D}-1-thiogalactopyranoside (IPTG), Pierce bicinchoninic acid (BCA) assay kit, Pierce snakeskin dialysis tubing 3.5K molecular weight cutoff (MWCO), sodium dodecyl sulfate (SDS), Nunc ninety-six well plates, Molecular Probes FluoSpheres (1.0 μm), phosphotungstic acid, and BD Clay Adams glass microscopy slides were acquired from Thermo Fisher Scientific. The twenty naturally occurring amino acids and thiamine hydrochloride (vitamin B) were purchased from Sigma Aldrich. Hydrochloric acid (HCl), Coomassie® Brilliant Blue G-250 were purchased from VWR. HiTrap FF 5 mL columns for protein purification were purchased from Cytiva Life Sciences. Macrosep and Microsep Advance Centrifugal Devices 3K molecular weight cutoff (MWCO) and 0.2 μm syringe filters were purchased from PALL. Acrylamide/bis solution (30%) 29:1, Mini Trans-Blot filter paper, Trans-Blot Transfer Medium (nitrocellulose membrane), and natural polypeptide sodium dodecyl sulfate-polyacrylamide gel electrophoresis (SDS-PAGE) standard were purchased from Bio-Rad. Imidazole was purchased from Acros Organics. Formvar/carbon-coated copper grids (FCF400-Cu) and 1% uranyl acetate for transmission electron microscopy were purchased from Electron Microscopy Sciences. Borosilicate glass capillaries (0.2 mm×2 mm×75 mm) were purchased from VitroCom. Fast-curing two-component epoxy was acquired from JB Weld.

Protein Expression. Q variants were expressed as described previously. Plasmids encoding the Q variants were designed in pQE60 vectors and purchased from Genscript. Colonies were selected from tryptic soy agar plates possessing chemically transformed M15MA E. coli cells. Protein expression was induced in supplemented M9 media, started in 16 mL of starter culture, using 200 μg/mL IPTG when the optical density at 600 nm (OD₆₀₀) grew to 0.8-1.0. The culture was incubated at 37° C. and 350 rpm with an Innova 42 incubator shaker (New Brunswick), allowing the protein to express for 3 h post-induction. Cells were centrifuged at 5,000×g at 4° C. for 30 minutes in an Avanti J-25 centrifuge (Beckman Coulter) and stored at −20° C. until purification. 12% SDS-PAGE was used to confirm expression.

Protein Purification. Q variants were purified as described previously. Pelleted cells were first resuspended in 40 mL of Buffer A (50 mM Tris-HCl, 500 mM NaCl, pH 8.0) and lysed using a Q500 probe sonicator (QSonica) at 55% amplitude for 2 minutes with 5 s and 5 s off. Lysed cells were pelleted at 11,000×g for 50 minutes to remove cell debris. Lysate was flowed through a cobalt-charged HiTrap IMAC FF 5 mL column and eluted at increasing concentrations of Buffer B (50 mM Tris-HCl, 500 mM NaCl, 500 mM imidazole). Fractions were assessed for purity by 12% SDS-PAGE and pure fractions were dialyzed using 6 consecutive buckets of Buffer A at 5 L volumes in 3.5 kDa MWCO snakeskin tubing. Dialyzed pure protein was then concentrated using 3 kDa MWCO Macrosep and Microsep Advance centrifugal devices (Pall Corporation) to 2 mM within six hours of removal from the dialysis bags. Protein concentration was determined by bicinchoninic acid (BCA) assay with a standard curve made using dilutions of bovine serum albumin (BSA).

Microrheology. Microrheology was used to assess gelation kinetics as described previously. Immediately after concentration to 2 mM, 29.7 uL were aliquoted with 0.3 uL of 1 um diameter FluoSpheres and loaded into glass capillary tubes (VitroCom). Samples were then imaged using an inverted fluorescent microscope (ZEISS Microscopy) at 40× magnification with 2×2 binning. Samples were incubated at 4° C. on a rotisserie at 8 rpm between measurements and imaged incrementally at select time points. Relaxation exponents were tracked until a negligible difference was observed with the relaxation exponent of the previous timepoint. Images were stacked, converted to greyscale, and analyzed with multiple particle tracking (MPT) in MATLAB (Mathworks, R2021a). Prior to other experiments of hydrogels in the gel state, microrheology here was used to confirm its complete transition.

Rheology. Q hydrogel variants were assessed for relative mechanical integrity with a stress-controlled rheometer (Discovery Hybrid Rheometer 2, TA Instruments) equipped with a parallel plate geometry. In parallel with microrheology measurements, 100 μL of each variant was incubated at 4° C. Upon gelation as assessed by microrheology, hydrogels were loaded onto an 8 mm diameter lower and upper plate with a 0.2 mm geometry gap. Storage modulus (G′) and loss modulus (G″) were measured from 0.1-10 Hz with 5% oscillation strain.

Circular Dichroism Spectroscopy. Secondary structure of protein samples pre- and post-gelation was assessed at 15 μM and 4° C. using a Jasco J-815 CD spectrometer with a PTC-423S single position Peltier temperature control system. The secondary structure of protein in solution was measured immediately after concentration to 2 mM, with protein diluted down to 15 μm using water to minimize salt interference. Secondary structure of protein in the gel state was measured under the same conditions after confirming gelation via microrheology. Wavelength scans were performed from 195 to 250 nm at 1 nm step sizes and mean residue ellipticity (MRE) was calculated as described previously.

Attenuated Total Reflectance-Fourier Transform Infrared Spectroscopy. Secondary structure before (solution) and after gelation (gel) was also assessed by peak deconvolution of attenuated total reflectance-Fourier transform infrared (ATR-FTIR) spectra. ATR-FTIR measurements were performed using a Nicolet 6700 Fourier Transform Infrared Spectrometer equipped with a mercury cadmium telluride (MCT)-A detector. Spectra were collected for 5 μL samples of protein at 2 mM from 4000-400 cm⁻¹ with a 4.0 cm⁻¹ resolution, normalized, and buffer-subtracted prior to analysis from 1700-1600 cm⁻¹, corresponding to the amide I region. ATR-FTIR measurements were performed immediately after concentration to 2 mM for solution measurements and after. Peaks were deconvoluted using Gaussian functions in PeakFit software until the goodness of fit reached r²≥0.99.

Transmission Electron Microscopy. Transmission electron microscopy (TEM) images were taken with a FEI Talos L120C transmission electron microscope. Samples were diluted to 50 μM and 3 μL was spotted on Formvar/carbon-coated copper grids followed by a L wash with water, and 3 μL staining with 1% v/v uranyl acetate solution each with incubation times of 1 min. Between steps, filter paper was used to gently wick the grids dry. Following imaging, minimum diameter nanofibers within the physically crosslinked hydrogel were sized in ImageJ software (Version 1.52q).

Small Angle X-ray Scattering (SAXS). All measurements were taken within 12 h of concentration of the protein from the dialysis bag. To study Q5 as an isolated particle, 150 μM (˜1 mg/mL) protein and dialysis buffer (50 mM Tris 500 mM NaCl pH 8.0) was loaded into a flow cell and measured in the q range 0.005⁻¹ to 3.19 Å⁻¹. The Q5 protein transition from solution to hydrogel was measured by incubation at 4° C. of 3 mM of Q5 protein in an eight-slot holder capable of loading 30 μL of each sample. Between measurements, the holder was returned for incubation at 4° C. Independent slots were used to load six samples of the Q5 hydrogel in solution including one for the empty cell and one for the dialysis buffer. Each slot was measured once incrementally over a 24 h incubation window to negate the impact of radiation (except for the measurement made at 24 h which was measured from the previous time point sample due to sample error in the last slot holder, however, a negligible impact of radiation was observed). Similarly, Q5 hydrogel measurements made at 3 mM and 4° C. were measured in the q range 0.005 Å⁻¹ to 3.19 Å⁻¹. Kratky plots were calculated by Guinier analysis of Rg and I(0) values. Flory exponents were calculated using MFF fit by Sosnick group online tool. Pair distance distribution function, P(r), was calculated using primus (ATSAS software).

Cryo-EM. Cryo-EM samples were prepared with the Vitrobot system (Thermo Fisher Scientific) which can help vitrify a thin solution layer at low temperature. Approximately 4 μl of sample solution was applied to the grids in the chamber of Vitrobot at 4° C. and 100% humidity. The sample solution was incubated for 10 s and then the grid was blotted three times. Blotting lasted 1 s for each time. The grid was plunged into the liquid ethane (around −175° C.) quickly to get the extremely fast cooling rate and the thin homogeneous vitreous layer. Then, the grid was transferred into liquid nitrogen. The cryo-EM holder was kept at −175° C. to prevent solvent crystallization during the imaging. Coarse-grained Model Details. Each bond is defined by Equation 10:

U_{bonded}(rᵢⱼ)=Kᵢⱼ(rᵢⱼ−r_{ij,0})² Equation 10
where Kᵢⱼ is the spring constant in kcal/mol/Å², r_{ij,0} is the mean bond distance, and rᵢⱼ is the pair distance between sites i and j. Electrostatic potentials were defined using the Coulombic-Debye potential (Equation 11):

$${U}_{c o u l} ( {r}_{i j} ) = \frac{C {q}_{i} {q}_{j}}{ϵ {r}_{ij}} {e}^{- κ {r}_{ij}} \qquad Equation 11$$

where C is a unit conversion constant, q is the charge in e, ε is the dielectric constant, and κ is the inverse Debye length which was set to 0.234 k⁻¹ based on 0.5 M NaCl at 4° C. Excluded volume potentials were defined based on soft exclusions (Equation 12):

$${U}_{excluded} ( {r}_{ij} ) = D [ 1 + cos ( \frac{π {r}_{ij}}{{r}_{c}} ) ] \qquad Equation 12$$

$${r}_{ij} < {r}_{c}$$

where D was set to 20.0 kcal/mol and re was set to 3.0 based on the minimum intermolecular CG site separation distance observed from the CG-mapped atomistic trajectories. The virtual site potentials were defined for virtual-real site pairs in the form of a Gaussian (Equation 13):

$${U}_{v s} ( {r}_{ij} ) = - A {e}^{- {Br}_{ij}^{2}} \qquad Equation 13$$

where A was set to 10.0 kcal/mol and B was set to 0.4 Å⁻² to ensure enough attractive strength to maintain the CC protofibril structure. Finally, it is noted that this model can be considered an implicit-solvent model as only protein is mapped while using atomistic statistics from solvated protein trajectories.

### Example 6

The following provides examples of peptides and methods of the present disclosure.

Exosomes have become a hot topic for biomedical research including wound healing applications. Exosomes are capable of improving blood circulation and endocrine signaling resulting in enhanced cell regeneration. However, exosomes suffer from retention and bioavailability at a wound site. Hydrogels are a popular tool for sustained drug delivery due to their ability to encapsulate drugs in its network and allow for targeted and or sustained release. Recently, hydrogels have shown to be an effective method improving the ability to provide improved rates of wound healing when combined with exosomes. A series of single-domain protein-based hydrogels capable of physical crosslinking and upper critical solution temperature (UCST) were designed. Using Rosetta-based computational search, we design a hydrogel, Q5, with improved UCST behavior and a significantly enhanced gelation rate was designed. This hydrogel was selected as a candidate for encapsulation and sustained release of exosomes dubbed Q5Exo. Q5Exo exhibits low critical gelation times and significant improvement in wound healing times in a diabetic mouse wound model showing promise as a exosome-based drug delivery tool and for future protein-based exosome design.

To demonstrate the application of designed protein-based coiled-coil hydrogels, Q5 was designed by automating the selection of mutations for improved thermostability using Rosetta-score Monte-Carlo search. Q5 was used as a candidate hydrogel-exosome system for diabetic wound healing. In using the hydrogel-exosome material, Q5Exo, the ability to use protein-based systems for encapsulation of exosomes was demonstrated, as was efficacious improvement of wound healing in diabetic mice, showing promise for future protein-based hydrogels in wound healing applications.

It was found that Q5Exo shows improved efficacy in promoting type 2 mouse diabetic wound closure as compared to exosomes alone and untreated mice, where the time to closure approaches that of wild type or non-diabetic wounds. Intriguingly, Q5 hydrogel alone generates faster time to closure of diabetic wounds, which may indicate efficacy of the gelled Q5 fibers alone in the wound bed, which are then enhanced further with addition of exosomes.

### Example 7

The following provides examples of peptides of the present disclosure.

Described is the generation of a biodegradable, biocompatible completely protein-based hydrogels that can encapsulate and provide sustained delivery of therapeutic molecules to the site of metastatic tumors while also possessing and being applied with fluorescent traceability.

It is hypothesized that a completely protein-based, traceable hydrogels will be able to provide sustained delivery by localized subcutaneous injection near the site of a tumor to counter the low bioavailability of the therapeutic and allowing for accumulation of anticancer agents for improved efficacy and decreased chemotherapeutic cost; the biomaterial will serve as a readout of the drug dose while enabling treatment and reducing toxicity. Even though the present Example describes delivering doxorubicin as a proof of principle, other therapies can be employed.

Q proteins are coiled-coils that possess oppositely charged electrostatic patches at each termini which allow them to further hierarchically assemble into fibers which then physically crosslink into a hydrogel. Due to the hierarchically supramolecular assembly, Qs are capable of dense small hydrophobic chemotherapeutic encapsulation such as doxorubicin. The protofibril assembly lends unto itself the ability to fluoresce, generating an inherent traceable moiety which requires no further synthesis steps. Preliminary data also suggests that the self-assembling nature of computational Q variants possess enhanced gelation properties and thermostability upon doxorubicin encapsulation allowing for sustained drug release via noninvasive subcutaneous injections, serving as a potential theranostic agent. It was also discovered Q-based proteins possess echogenicity in ultrasound. As a result, it is proposed to use ultrasound-guided injection to precisely test the theranostic application of Qs for localized and sustained chemotherapeutic delivery. The advantages of the proposed vehicles relative to current therapies are: 1) injection of the chemotherapeutic-encapsulated hydrogel near the site of the tumor providing localized drug delivery; and 2) independent autofluorescence for traceability of the Q hydrogel assisted by subcutaneous injection.

Use of the materials in this Example may be extended to improve the efficacy of therapeutic agents for metastatic triple negative breast (TNB) cancer patients, such as doxorubicin, using localized drug delivery, enhanced traceability of autofluorescent chemotherapeutic and drug delivery agent that will concentrate and release therapeutic small molecules near the site of tumors. It is hypothesized that the novel, multi-traceable protein-based hydrogel drug delivery agents can: 1) encapsulate doxorubicin and provide a sustained release for increased therapeutic efficacy; and 2) be applied non-invasively by subcutaneous ultrasound-guided injections providing doxorubicin and hydrogel traceability via their autofluorescence. Studies will test the ability of Qs to thermostably reside near the site of a tumor developed by 4T1 TNB cancer cell lines in NOD/SCID/g immune impaired mice. Primary tumors will be propagated in the mammary fat pad where tumor volume over time will be measured by 3D ultrasound.

Although the present disclosure has been described with respect to one or more particular embodiments and/or examples, it will be understood that other embodiments and/or examples of the present disclosure may be made without departing from the scope of the present disclosure.

1. A protein or peptide having or comprising the following sequence:
(SEQ ID NO: 1)
VX₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X
₂₄X₂₅X₂₆X₂₇X₂₈X₂₉X₃₀X₃₁X₃₂X₃₃X₃₄X₃₅X₃₆X₃₇,
wherein
X₁ is A, E, D, R, H, K, Q, N, or S;
X₂ is A, E, N, or Q;
X₃ is A, V, L, I, Q, M, or L_{TF};
X₄ is A, E, R, D, H, I, L, T, K, Q, N, or L_{TF};
X₅ is A, F, Q, R, K, H, D, S, or E;
X₆ is A, L, I, or L_{TF};
X₇ is A, K, or E;
X₈ is A, K, E, D, R, H, Q, or N;
X₉ is A, T, I, L, Q, or L_{TF};
X₁₀ is A, L, I, or L_{TF};
X₁₁ is A, E, D, H, P, I, L, K, Y, N, Q, R, or L_{TF};
X₁₂ is A, Q, H, E, D, K, R, or N;
X₁₃ is A, M, I, L, Q, T, or L_{TF};
X₁₄ is A, L, D, E, K, I, or L_{TF};
X₁₅ is A, E, D, H, Y, I, L, R, K, Q, N, or L_{TF};
X₁₆ is A, E, or Q;
X₁₇ is A, L, M, I, V, or L_{TF};
X₁₈ is A, K, E, D, K, R, H, N, or Q;
X₁₉ is A, N, D, K, R, H, Q, or E;
X₂₀ is A, L, T, I, M, R, or L_{TF};
X₂₁ is A, N, or Q;
X₂₂ is K, A, E, I, L, M, R, H, D, Q, N, S, or L_{TF};
X₂₃ is A, Q, N, I, L, or L_{TF};
X₂₄ is A, L, I, M, T, or L_{TF};
X₂₅ is A, H, Q, R, K, D, N, Y, I, E, L, T, or L_{TF};
X₂₆ is A, D, E, R, K, Q, H, N, or T;
X₂₇ is A, V, I, Q, L, T, or L_{TF};
X₂₈ is A, R, E, D, K, H, N, Q, or T;
X₂₉ is A, H, E, R, D, K, I, L, N, Q, T, Y, or L_{TF};
X₃₀ is L, A, D, K, I, N, Q, or L_{TF};
X₃₁ is A, L, Q, I, or L_{TF};
X₃₂ is E, D, K, H, N, Q, A, L, R, I, Y, or L_{TF};
X₃₃ is A, N, Q, D, E, H, K, R, or S;
X₃₄ is Q, I, L, A, M, or L_{TF};
X₃₅ is S, A, P, or Q;
X₃₆ is A, K, T, D, R, H, N, Q, or E;
X₃₇ is A, L, I, K, D, N, Q, R, or L_{TF},
wherein at least one of X₃, X₄, X₆, X₉, X₁₀, X₁₁, X₁₃, X₁₄, X₁₅, X₁₆, X₁₇, X₂₀, X₂₂, X₂₃, X₂₄, X₂₅, X₂₇, X₂₉, X₃₀, X₃₁, X₃₂, X₃₄, X₃₇ or any L is replaced with a trifluoroleucine residue.

2. The protein or peptide according to claim 1, wherein the sequence is or comprises:
(SEQ ID NO: 3)
VKELLFLKKTAEQMLEELKETNKALHDVRHLLENQSKL;
(SEQ ID NO: 4)
VKEILFLKNTAYQMLLELKETNEALYDIRHLLQQQSKL;
(SEQ ID NO: 5)
VKEITFIKKTIEQIAEEMKEINKAIHDIRHALENISKK;
(SEQ ID NO: 6)
VKEQTFIKNTIEQMAEEIKEINKAIHDIRHQLENMSKQ;
(SEQ ID NO: 7)
VKEITFIKKQIEQIAEEMKEINKAIHDIRHALENISKK;
(SEQ ID NO: 8)
VKEMTFIKKTIEQQAEEMKEINKAIHDIRHALENISKK;
(SEQ ID NO: 9)
VSEITEIKKTIEHIAKEMKEINKAIHTIRHALENIAKN;
or
(SEQ ID NO: 10)
VKEIKFLKNTAPQQLRELQNTNMALQDVRELLQQQSTL,
or a sequence having at least 75-99% identity to any one of the foregoing sequences, wherein one, some, or all the leucine residues of each sequence is replaced with a trifluoroleucine residue.

3. The protein or peptide according to claim 1, wherein the sequence is or comprises:
(SEQ ID NO: 26)
MRGSHHHHHHGSIEGRVX₁X₂X₃X₄X₅X₆X₇X₈X₉X₁₀X₁₁X₁₂X₁₃X₁₄X₁₅X₁₆
X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇

4. The protein or peptide according to claim 3, wherein the protein or peptide has the following sequence or comprises the following sequence:
(SEQ ID NO: 11)
MRGSHHHHHHGSIEGRVKELLFLKKTAEQMLEELKETNKALHDVRHLLE
NQSKL;
(SEQ ID NO: 12)
MRGSHHHHHHGSIEGRVKEILFLKNTAYQMLLELKETNEALYDIRHLLQ
QQSKL;
(SEQ ID NO: 13)
MRGSHHHHHHGSIEGRVKEITFIKKTIEQIAEEMKEINKAIHDIRHALE
NISKK;
(SEQ ID NO: 14)
MRGSHHHHHHGSIEGRVKEQTFIKNTIEQMAEEIKEINKAIHDIRHQLE
NMSKQ;
(SEQ ID NO: 15)
MRGSHHHHHHGSIEGRVKEITFIKKQIEQIAEEMKEINKAIHDIRHALE
NISKK;
(SEQ ID NO: 16)
MRGSHHHHHHGSIEGRVKEMTFIKKTIEQQAEEMKEINKAIHDIRHALE
NISKK;
(SEQ ID NO: 17)
MRGSHHHHHHGSIEGRVSEITEIKKTIEHIAKEMKEINKAIHTIRHALE
NIAKN;
or
(SEQ ID NO: 18)
MRGSHHHHHHGSIEGRVKEIKFLKNTAPQQLRELQNTNMALQDVRELLQ
QQSTL,
or a sequence having at least 75-99% identity to any one of the foregoing sequences, where one, some, or all the leucine residues of each sequence is replaced with a trifluoroleucine residue.

5. A protein or peptide fiber comprising one or more protofibers comprising one or more proteins or peptides according to claim 1.

6. The protein or peptide fiber according to claim 5, wherein one or more compounds are bound to the protein or peptide fiber.

7. The protein or peptide fiber according to claim 6, wherein the one or more compounds are hydrophobic.

8. The protein or peptide fiber according to claim 6, wherein the one or more compounds are dyes, antibiotics, alkaloids, lipids, fatty acids, sugars, amino acids, phenolic compounds, extracellular materials, metals, nucleic acids, and combinations thereof.

9. The protein or peptide fiber according to claim 8, wherein the one or more compounds are extracellular materials and the extracellular materials are exosomes.

10. The protein or peptide fiber according to claim 5, wherein the protein or peptide fiber has a fiber diameter of about 20 nm to about 2 μm.

11. A composition comprising a plurality of protein or peptide fibers according to claim 1.

12. The composition according to claim 11, wherein the plurality of protein or peptide fibers are formulated into a gel.

13. A method for detecting the location of a compound in an individual, wherein the individual has been administered one or more of the compounds bound to one or more protein or peptide fibers according to claim 5, comprising
detecting a signal of the protein or peptide fibers,
wherein the location of the compound is determined by the detection of the signal of the one or more protein or peptide fibers.

14. The method according to claim 13, wherein the signal is detected via MRI or ultrasound.

15. The method according to claim 14, wherein the MRI is ¹⁹F MR spectroscopy.

16. The method according to claim 14, wherein the MRI is ¹H MR spectroscopy.

17. The method according to claim 14, wherein the one or more compounds are therapeutic agents.

18. A method for determining an optimal sequence of a protein to achieve a desired diameter of a fiber formed by the self-assembled protein, comprising:
determining ΔEE_{bcf} of a primary structure of a protein;
determining a stability score of the primary structure; and
using a computational algorithm to optimize a variant structure of the protein by substituting one or more of the solvent-exposed residues of the primary structure to achieve a desired ΔEE_{bcf}, while maintaining or increasing the stability score for the varied structure.

19. The method of claim 18, wherein the computational algorithm is a machine learning algorithm or a Monte Carlo simulation.

20. The method of claim 18, wherein ΔEE_{bcf} is determined by:
$$Δ E {E}_{bcf} = {\sum }_{n = l / 2}^{l} E {E}_{bcf} - {\sum }_{n = 1 + ( tag length )}^{\frac{l}{2} - 1} {EE}_{bcf} ,$$
where n is the sequence position number, l is the length of the sequence, and EE_{bcf} is the electrostatic potential energy of a residue in the b, c, or f helical wheel position.


SEQUENCE LISTING (27 sequences)

SEQ ID NO: 1 - skipped

SEQ ID NO: 2 - protein, 16 aa, synthetic construct
MRGSHHHHHHGSIEGR

SEQ ID NO: 3 - protein, 38 aa, synthetic construct
VKELLFLKKTAEQMLEELKETNKALHDVRHLLENQSKL

SEQ ID NO: 4 - protein, 38 aa, synthetic construct
VKEILFLKNTAYQMLLELKETNEALYDIRHLLQQQSKL

SEQ ID NO: 5 - protein, 38 aa, synthetic construct
VKEITFIKKTIEQIAEEMKEINKAIHDIRHALENISKK

SEQ ID NO: 6 - protein, 38 aa, synthetic construct
VKEQTFIKNTIEQMAEEIKEINKAIHDIRHQLENMSKQ

SEQ ID NO: 7 - protein, 38 aa, synthetic construct
VKEITFIKKQIEQIAEEMKEINKAIHDIRHALENISKK

SEQ ID NO: 8 - protein, 38 aa, synthetic construct
VKEMTFIKKTIEQQAEEMKEINKAIHDIRHALENISKK

SEQ ID NO: 9 - protein, 38 aa, synthetic construct
VSEITEIKKTIEHIAKEMKEINKAIHTIRHALENIAKN

SEQ ID NO: 10 - protein, 38 aa, synthetic construct
VKEIKFLKNTAPQQLRELQNTNMALQDVRELLQQQSTL

SEQ ID NO: 11 - protein, 54 aa, synthetic construct
MRGSHHHHHHGSIEGRVKELLFLKKTAEQMLEELKETNKALHDVRHLLENQSKL

SEQ ID NO: 12 - protein, 54 aa, synthetic construct
MRGSHHHHHHGSIEGRVKEILFLKNTAYQMLLELKETNEALYDIRHLLQQQSKL

SEQ ID NO: 13 - protein, 54 aa, synthetic construct
MRGSHHHHHHGSIEGRVKEITFIKKTIEQIAEEMKEINKAIHDIRHALENISKK

SEQ ID NO: 14 - protein, 54 aa, synthetic construct; identical to SEQ ID NO: 13

SEQ ID NO: 15 - protein, 54 aa, synthetic construct
MRGSHHHHHHGSIEGRVKEITFIKKQIEQIAEEMKEINKAIHDIRHALENISKK

SEQ ID NO: 16 - protein, 54 aa, synthetic construct
MRGSHHHHHHGSIEGRVKEMTFIKKTIEQQAEEMKEINKAIHDIRHALENISKK

SEQ ID NO: 17 - protein, 54 aa, synthetic construct
MRGSHHHHHHGSIEGRVSEITEIKKTIEHIAKEMKEINKAIHTIRHALENIAKN

SEQ ID NO: 18 - protein, 54 aa, synthetic construct
MRGSHHHHHHGSIEGRVKEIKFLKNTAPQQLRELQNTNMALQDVRELLQQQSTL

SEQ ID NO: 19 - protein, 54 aa, synthetic construct
  VARIANT 23: note = X is leucine or trifluoroleucine
  VARIANT 31: note = X is leucine or trifluoroleucine
  VARIANT 34: note = X is leucine or trifluoroleucine
  VARIANT 41: note = X is leucine or trifluoroleucine
  VARIANT 47: note = X is leucine or trifluoroleucine
  VARIANT 48: note = X is leucine or trifluoroleucine
  VARIANT 54: note = X is leucine or trifluoroleucine
MRGSHHHHHHGSIEGRVKEITFXKNTAPQMXREXQETNAAXQDVREXXRQQSKX

SEQ ID NO: 20 - protein, 54 aa, synthetic construct
  VARIANT 20: note = X is leucine or trifluoroleucine
  VARIANT 21: note = X is leucine or trifluoroleucine
  VARIANT 23: note = X is leucine or trifluoroleucine
  VARIANT 31: note = X is leucine or trifluoroleucine
  VARIANT 34: note = X is leucine or trifluoroleucine
  VARIANT 41: note = X is leucine or trifluoroleucine
  VARIANT 47: note = X is leucine or trifluoroleucine
  VARIANT 48: note = X is leucine or trifluoroleucine
  VARIANT 54: note = X is leucine or trifluoroleucine
MRGSHHHHHHGSIEGRVKEXXFXKKTAEQMXEEXKETNKAXHDVRHXXENQSKX

SEQ ID NO: 21 - protein, 61 aa, synthetic construct
MRGSHHHHHHGSIEGRAPQMLRELQETNAALQDVRELLRQQVKEITFLKNTVMESDASGKL

SEQ ID NO: 22 - protein, 60 aa, synthetic construct
MRGSHHHHHHGSIEGRMQLAKHMLKELQKTNALQSVRTLLQQQVEEITQLKETVENSDSS

SEQ ID NO: 23 - protein, 54 aa, synthetic construct
MRGSHHHHHHGSIEGRVKEITFLKNTAPQMLRELQETNAALQDVRELLRQQSKL

SEQ ID NO: 24 - protein, 55 aa, synthetic construct
MRGSHHHHHHGSIEGRVKEITFLKNTAPQMLRELQETNAALQDVRELLRQQSKLN

SEQ ID NO: 25 - protein, 54 aa, synthetic construct
MRGSHHHHHHGSIEGRVKLLFLKKTAEQMLEELKETNKALHDVRHLLENQSKLN

SEQ ID NO: 26 - protein, 54 aa, synthetic construct
  VARIANT 18: note = X is A, E, D, R, H, K, Q, N, or S
  VARIANT 19: note = X is A, E, N, or Q
  VARIANT 20: note = X is A, V, L, I, Q, M, or trifluoroleucine
  VARIANT 21: note = X is A, E, R, D, H, I, L, T, K, Q, N, or trifluoroleucine
  VARIANT 22: note = X is A, F, Q, R, K, H, D, S, or E
  VARIANT 23: note = X is A, L, I, or trifluoroleucine
  VARIANT 24: note = X is A, K, or E
  VARIANT 25: note = X is A, K, E, D, R, H, Q, or N
  VARIANT 26: note = X is A, T, I, L, Q, or trifluoroleucine
  VARIANT 27: note = X is A, L, I, or trifluoroleucine
  VARIANT 28: note = X is A, E, D, H, P, I, L, K, Y, N, Q, R, or trifluoroleucine
  VARIANT 29: note = X is A, Q, H, E, D, K, R, or N
  VARIANT 30: note = X is A, M, I, L, Q, T, or trifluoroleucine
  VARIANT 31: note = X is A, L, D, E, K, I, or trifluoroleucine
  VARIANT 32: note = X is A, E, D, H, Y, I, L, R, K, Q, N, or trifluoroleucine
  VARIANT 33: note = X is A, E, or Q
  VARIANT 34: note = X is A, L, M, I, V, or trifluoroleucine
  VARIANT 35: note = X is A, K, E, D, K, R, H, N, or Q
  VARIANT 36: note = X is A, N, D, K, R, H, Q, or E
  VARIANT 37: note = X is A, L, T, I, M, R, or trifluoroleucine
  VARIANT 38: note = X is A, N, or Q
  VARIANT 39: note = X is K, A, E, I, L, M, R, H, D, Q, N, S, or trifluoroleucine
  VARIANT 40: note = X is A, Q, N, I, L, or trifluoroleucine
  VARIANT 41: note = X is A, L, I, M, T, or trifluoroleucine
  VARIANT 42: note = X is A, H, Q, R, K, D, N, Y, I, E, L, T, or trifluoroleucine
  VARIANT 43: note = X is A, D, E, R, K, Q, H, N, or T
  VARIANT 44: note = X is A, V, I, Q, L, T, or trifluoroleucine
  VARIANT 45: note = X is A, R, E, D, K, H, N, Q, or T
  VARIANT 46: note = X is A, H, E, R, D, K, I, L, N, Q, T, Y, or trifluoroleucine
  VARIANT 47: note = X is L, A, D, K, I, N, Q, or trifluoroleucine
  VARIANT 48: note = X is A, L, Q, I, or trifluoroleucine
  VARIANT 49: note = X is E, D, K, H, N, Q, A, L, R, I, Y, or trifluoroleucine
  VARIANT 50: note = X is A, N, Q, D, E, H, K, R, or S
  VARIANT 51: note = X is Q, I, L, A, M, or trifluoroleucine
  VARIANT 52: note = X is S, A, P, or Q
  VARIANT 53: note = X is A, K, T, D, R, H, N, Q, or E
  VARIANT 54: note = X is A, L, I, K, D, N, Q, R, or trifluoroleucine
MRGSHHHHHHGSIEGRVXXXXXXXXXXXXXXXXXXXXXXXXXXXXXXXXXXXXX

SEQ ID NO: 27 - protein, 54 aa, synthetic construct
  VARIANT 18: note = X is K or S
  VARIANT 20: note = X is L, I, Q, M, or trifluoroleucine
  VARIANT 21: note = X is L, I, T, K, or trifluoroleucine
  VARIANT 22: note = X is F or E
  VARIANT 23: note = X is L, I, or trifluoroleucine
  VARIANT 25: note = X is K or N
  VARIANT 26: note = X is T, Q, or trifluoroleucine
  VARIANT 27: note = X is A, L, I, or trifluoroleucine
  VARIANT 28: note = X is E, P, I, L, Y, or trifluoroleucine
  VARIANT 29: note = X is Q or H
  VARIANT 30: note = X is M, I, L, Q, or trifluoroleucine
  VARIANT 31: note = X is A, L, I, or trifluoroleucine
  VARIANT 32: note = X is E, I, L, R, K, or trifluoroleucine
  VARIANT 34: note = X is L, M, I, or trifluoroleucine
  VARIANT 35: note = X is K or Q
  VARIANT 36: note = X is N or E
  VARIANT 37: note = X is L, T, I, or trifluoroleucine
  VARIANT 39: note = X is K, E, I, L, M, or trifluoroleucine
  VARIANT 41: note = X is L, I, or trifluoroleucine
  VARIANT 42: note = X is H, Q, Y, I, L, or trifluoroleucine
  VARIANT 43: note = X is D or T
  VARIANT 44: note = X is V, I, L, or trifluoroleucine
  VARIANT 46: note = X is H, E, I, L, or trifluoroleucine
  VARIANT 47: note = X is L, A, I, Q, or trifluoroleucine
  VARIANT 49: note = X is E, Q, L, I, or trifluoroleucine
  VARIANT 50: note = X is N or Q
  VARIANT 51: note = X is Q, I, L, M, or trifluoroleucine
  VARIANT 52: note = X is S or A
  VARIANT 53: note = X is K or T
  VARIANT 54: note = X is L, I, K, N, Q, or trifluoroleucine
MRGSHHHHHHGSIEGRVXEXXXXKXXXXXXXXEXXXXNXAXXXXRXXLXXXXXX